(19) United States
(12) Patent Application Publication
(10) Pub. No.: US 2025/0108066 A1
(43) Pub. Date: Apr. 3, 2025

(54) TRINUCLEOTIDE CAP ANALOGS AND METHODS OF USE THEREOF

(71) Applicant: TriLink BioTechnologies, LLC, San Diego, CA (US)

(72) Inventors: Chunping XU, San Diego, CA (US); Michael HOUSTON, San Diego, CA (US); Alexandre LEBEDEV, San Diego, CA (US); Jordana Michelle HENDERSON, La Jolla, CA (US); Andrew UJITA, San Diego, CA (US); Inna KOUKHAREVA, San Diego, CA (US); Chanfeng ZHAO, Rancho Santa Fe, CA (US); Ilya Vladimirovich ILICHEV, San Diego, CA (US)

(73) Assignee: TriLink BioTechnologies, LLC, San Diego, CA (US)

(21) Appl. No.: 18/833,215

(22) PCT Filed: Jan. 25, 2023
(86) PCT No.: PCT/US2023/061255

§ 371 Date: Jul. 25, 2024

Related U.S. Application Data
(60) Provisional application No. 63/267,223, filed on Jan. 27, 2022.
(60) Provisional application No. 63/382,956, filed on Nov. 9, 2022.
(60) Provisional application No. 63/476,787, filed on Dec. 22, 2022.

(51) Int. Cl. A61K 31/7105; C07H 1/04; C07H 21/02; C12N 15/67

(52) U.S. Cl. CPC A61K 31/7105; C07H 1/04; C07H 21/02; C12N 15/67

(57) ABSTRACT

Described herein are novel trinucleotide cap analogs and methods of making and using the same. Also described herein is an RNA molecule comprising a 5′-cap, wherein the 5′-cap includes a trinucleotide cap analog as described herein. Methods of inducing a therapeutic effect in a 5 subject are also described herein, the methods including a step of administering to the subject an RNA molecule including the trinucleotide cap analog.

### CROSS-REFERENCE TO PRIORITY APPLICATIONS

This application claims priority to U.S. Provisional Application No. 63/267,223, filed Jan. 27, 2022; U.S. Provisional Application No. 63/382,956, filed Nov. 9, 2022; and U.S. Provisional Application No. 63/476,787, filed Dec. 22, 2022, the contents of which are incorporated herein by reference in their entireties.

### BACKGROUND

In vitro transcribed messenger RNAs (mRNAs) have numerous in vivo applications, such as vaccination, where mRNA encoding specific antigen(s) is administered to elicit protective immunity in a patient; cell therapy, where mRNA is transfected into cells ex vivo to alter cell phenotype or function prior to delivery of these altered cells to a patient; or replacement therapy, where mRNA encoding a therapeutic protein is administered to the patient.

A primary structural element of an mRNA molecule that is utilized in in vivo applications includes a Cap structure on the 5′-end of the mRNA. Naturally occurring Cap structures include a 7-methylguanosine (^{7m}G or ^{m7}G) linked through a 5′- to 5′-triphosphate chain at the 5′-end of the mRNA molecule. The Cap must be present for the mRNA to retain template activity for protein synthesis. The chemical structure of the Cap can modulate translation efficiency in a cell. Therefore, effective Cap structures are necessary.

### SUMMARY

This summary is a high-level overview of various aspects of the invention and introduces some of the concepts that are further described in the Detailed Description section below. This summary is not intended to identify key or essential features of the claimed subject matter, nor is it intended to be used in isolation to determine the scope of the claimed subject matter. The subject matter should be understood by reference to appropriate portions of the entire specification, any or all drawings, and each claim.

Described herein are trinucleotide cap analogs and methods of making and using the same. Provided herein is a compound of the following formula:

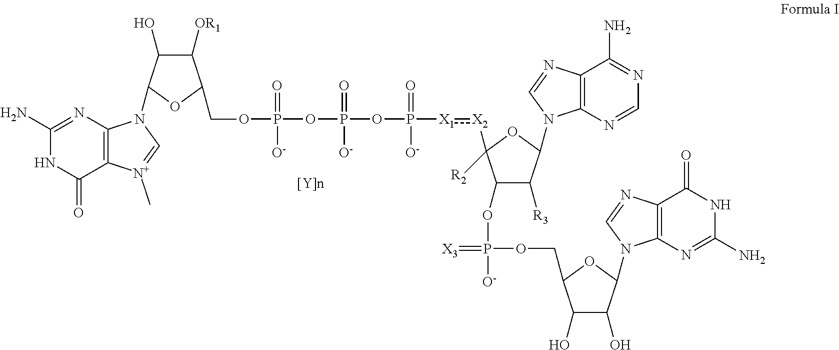

or any stereoisomer thereof, wherein: [symbol] is a single bond or a double bond; R₁ is H or CH₃; R₂ is H and R₃ is OCH₃ or F, or R₂ and R₃ are covalently bonded together and, together with intermediate atoms, form a 2′-O, 4′-C methylene bridge; X₁ is O or CH; X₂ is CH₂ or CH; X₃ is O or S; each independent Y is H⁺ or a cation; and n is 0, 1, 2, 3, or 4, wherein when X₁ is CH, X₂ is CH and [symbol] is a double bond. Optionally, the compound has the following structure:

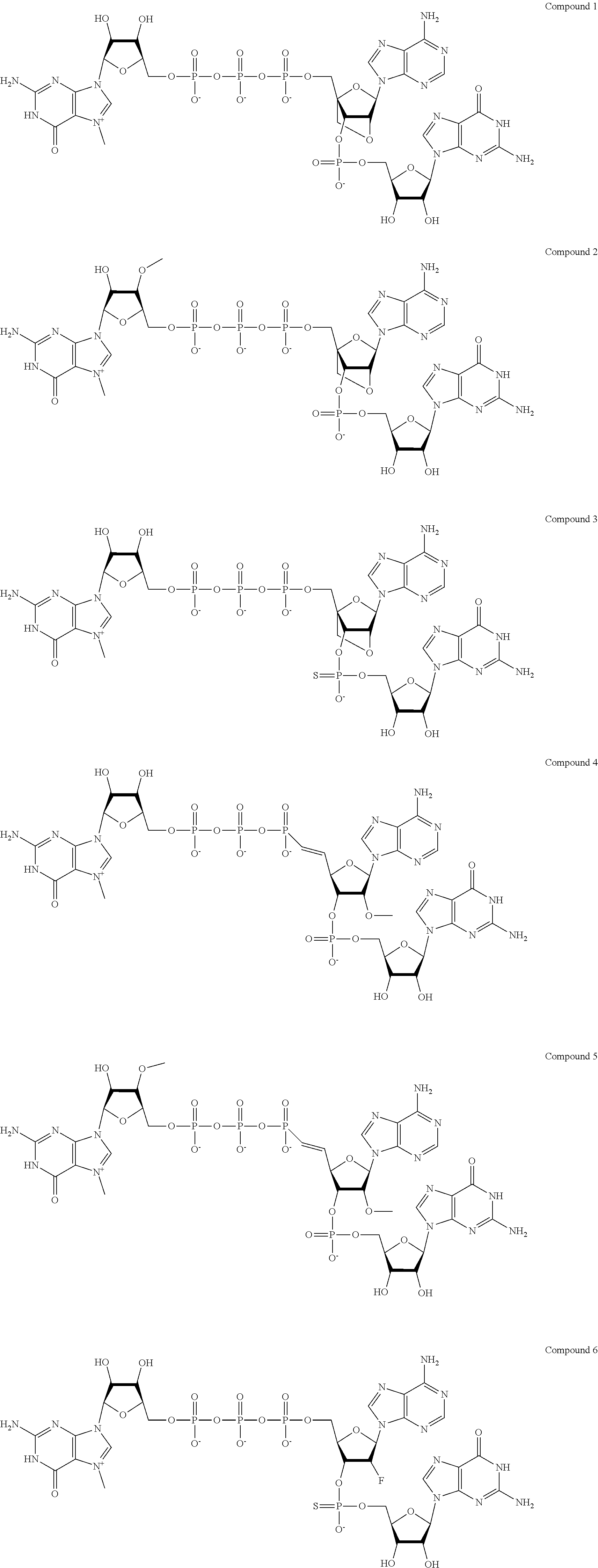

or any stereoisomer thereof.

Also provided herein is a compound of the following formula:

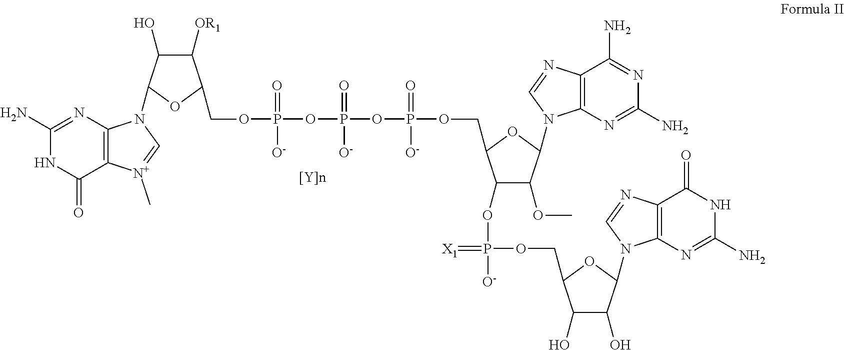

or any stereoisomer thereof, wherein: R₁ is H or CH₃, X₁ is O or S, each independent Y is H⁺ or a cation, and n is 0, 1, 2, 3, or 4, wherein when X₁ is S, R₁ is CH₃. Optionally, the compound has the following structure:

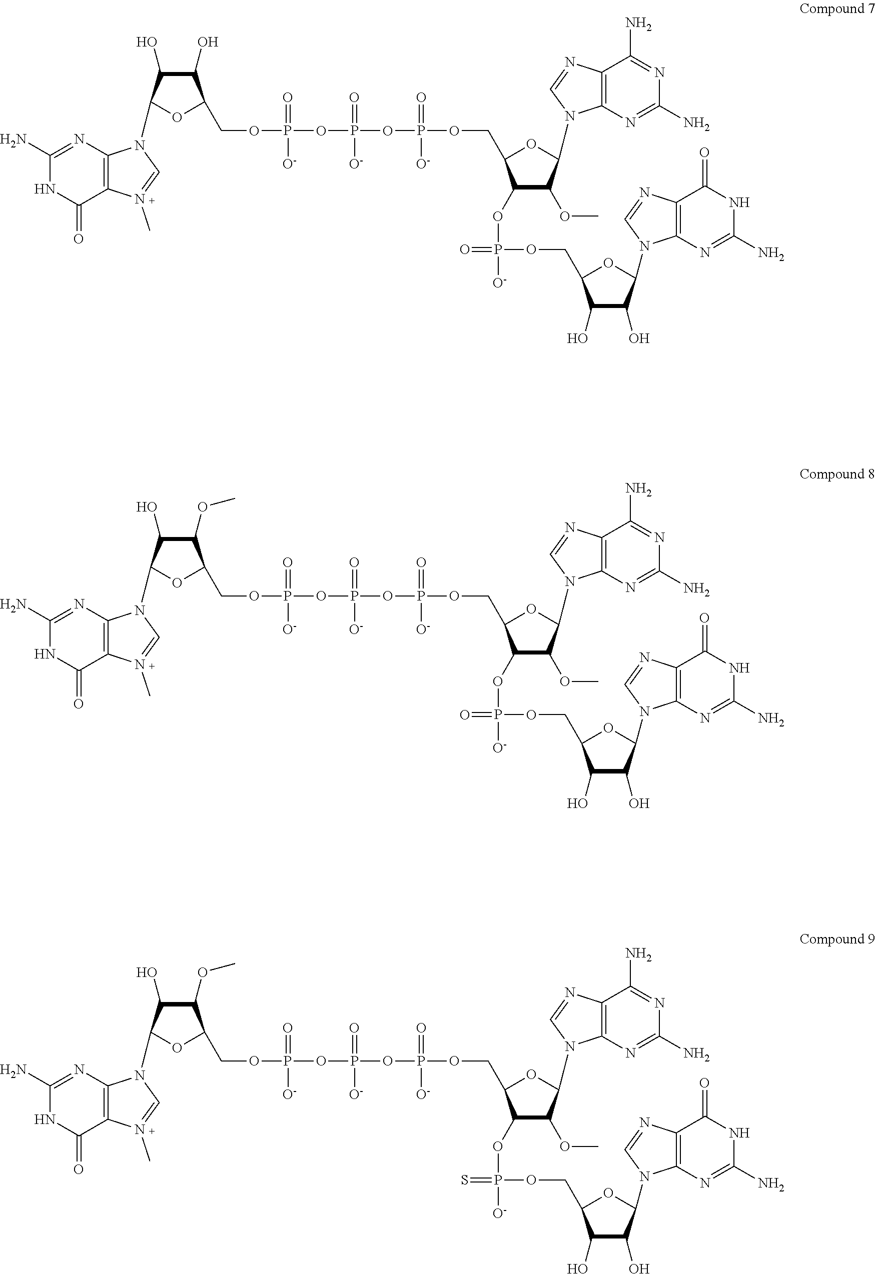

or any stereoisomer thereof.

Also provided herein is a compound of the following formula:

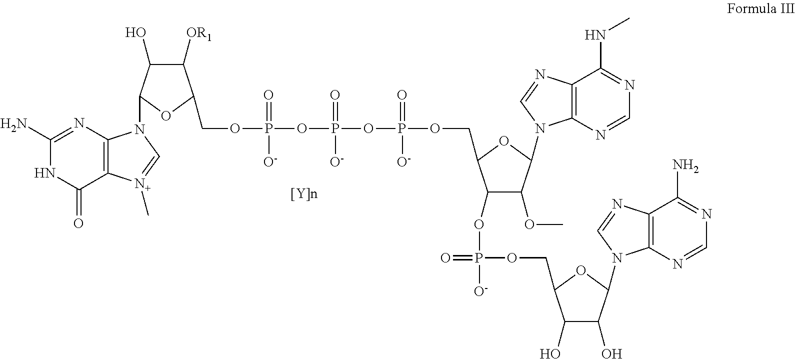

or any stereoisomer thereof, wherein: R₁ is H or CH₃; each independent Y is H⁺ or a cation; and n is 0, 1, 2, 3, or 4. Optionally, the compound has the following structure:

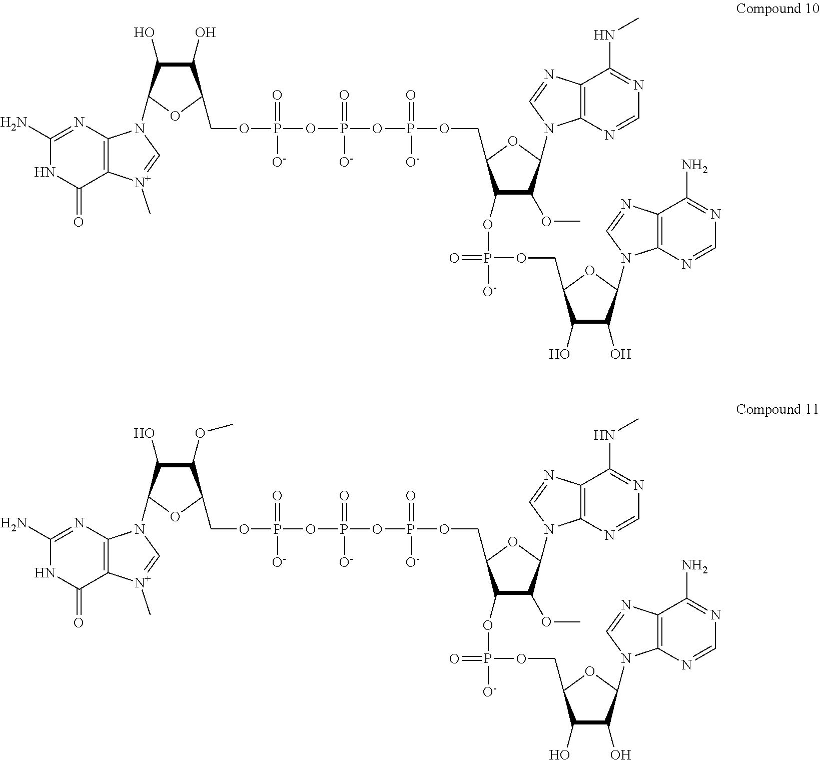

or any stereoisomer thereof.

Additionally provided herein is a compound of the following formula:

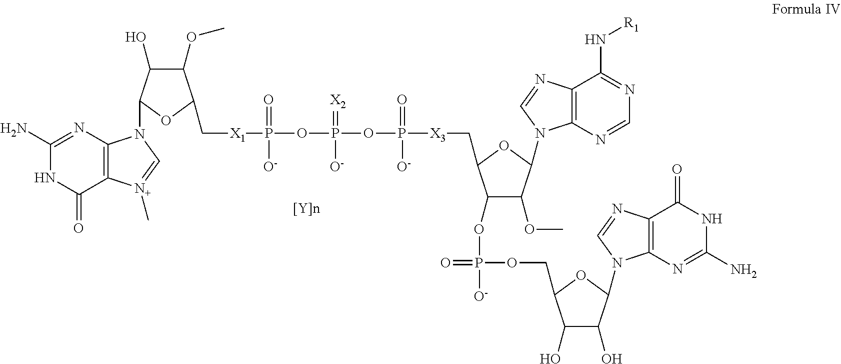

or any stereoisomer thereof, wherein: R₁ is H or CH₃; X₁, X₂, and X₃ are each independently selected from O and S, wherein when one of X₁, X₂, or X₃ is S, the remaining of X₁, X₂, and X₃ are O; each independent Y is H⁺ or a cation; and n is 0, 1, 2, 3, or 4.

Optionally, the compound has the following structure:

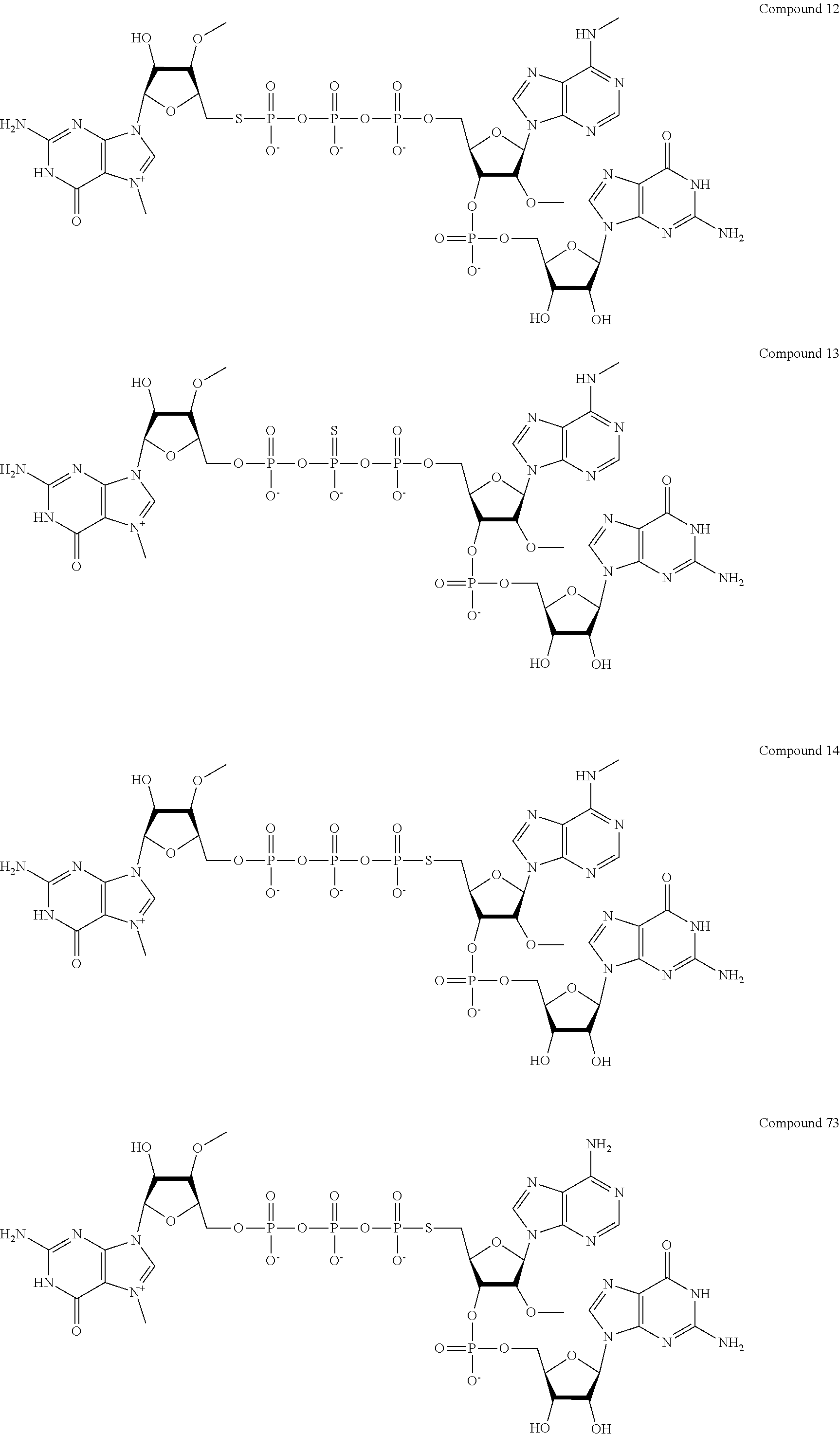

or any stereoisomer thereof.

Further provided herein is a compound of the following formula:

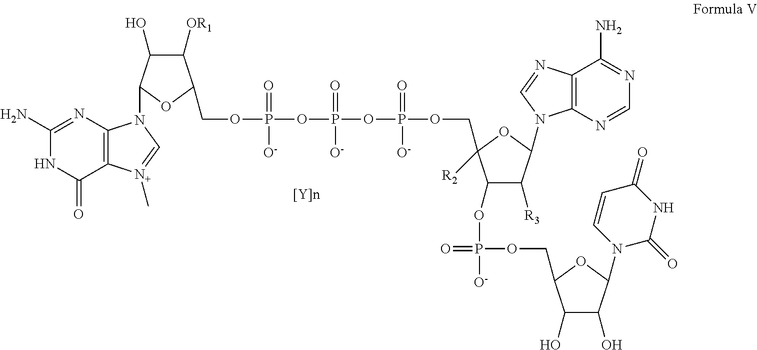

or any stereoisomer thereof, wherein: R₁ is H or CH₃; R₂ is H and R₃ is F, or R₂ and R₃ are covalently bonded together and, together with intermediate atoms, form a 2′-O, 4′-C methylene bridge; each independent Y is H⁺ or a cation; and n is 0, 1, 2, 3, or 4, wherein when R₂ is H and R₃ is F, R₁ is not H. Optionally, the compound has the following structure:

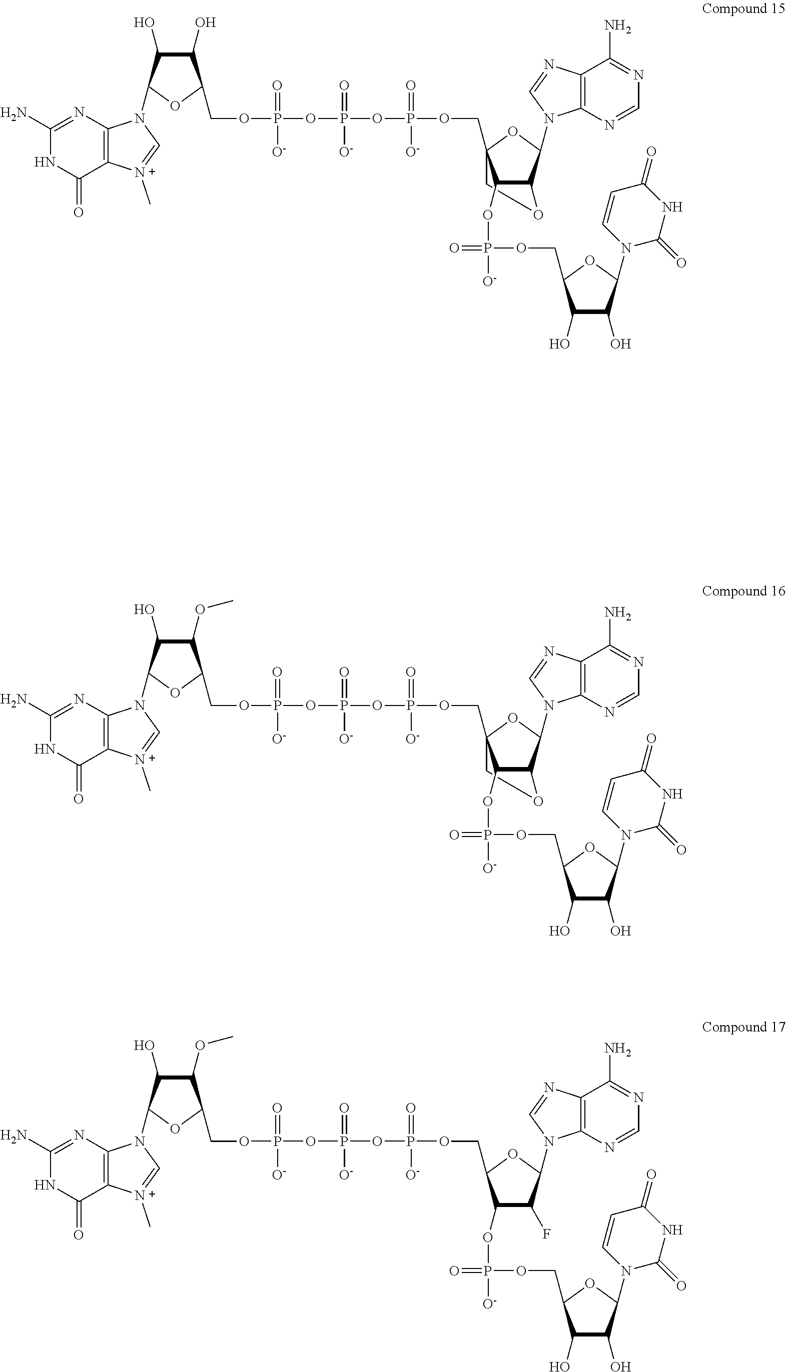

or any stereoisomer thereof.

Also provided herein is a compound of the following formula:

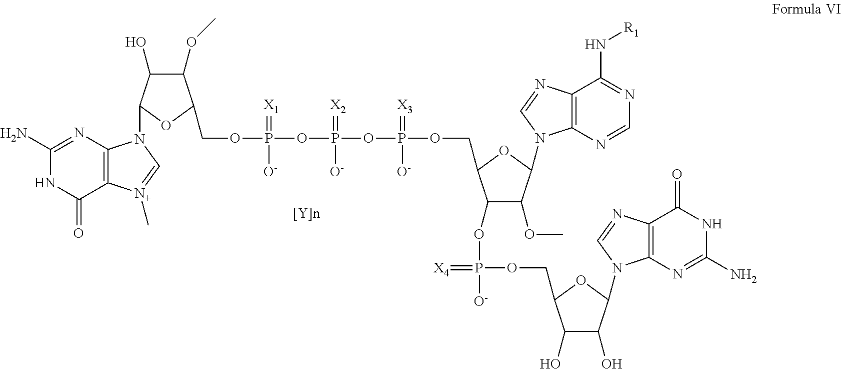

or any stereoisomer thereof, wherein: R₁ is H or CH₃; X₁, X₂, X₃, and X₄ are each independently selected from O and S; each independent Y is H⁺ or a cation; and n is 0, 1, 2, 3, or 4, wherein one of X₁, X₂, X₃, and X₄ is S. Optionally, the compound has the following structure:

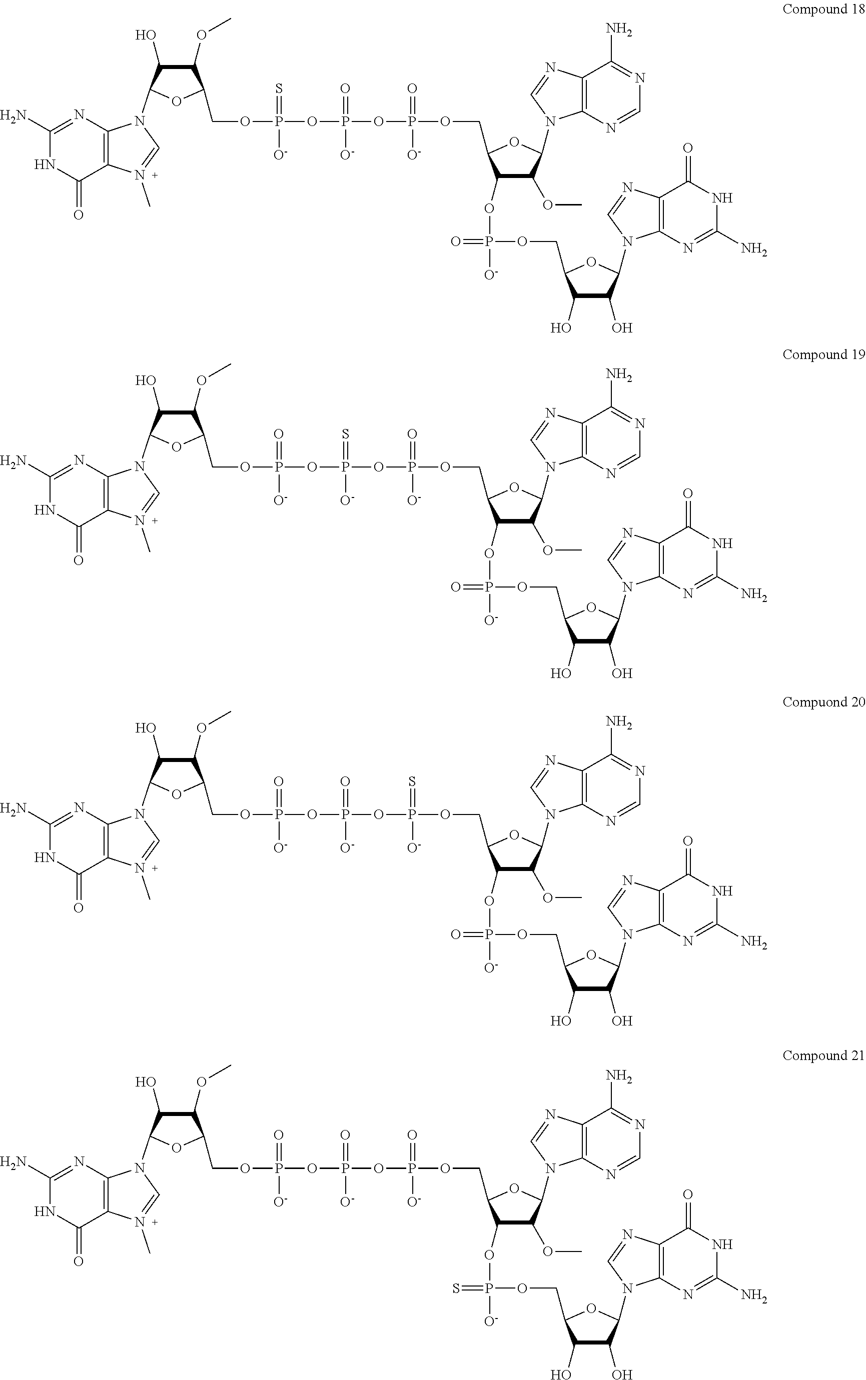

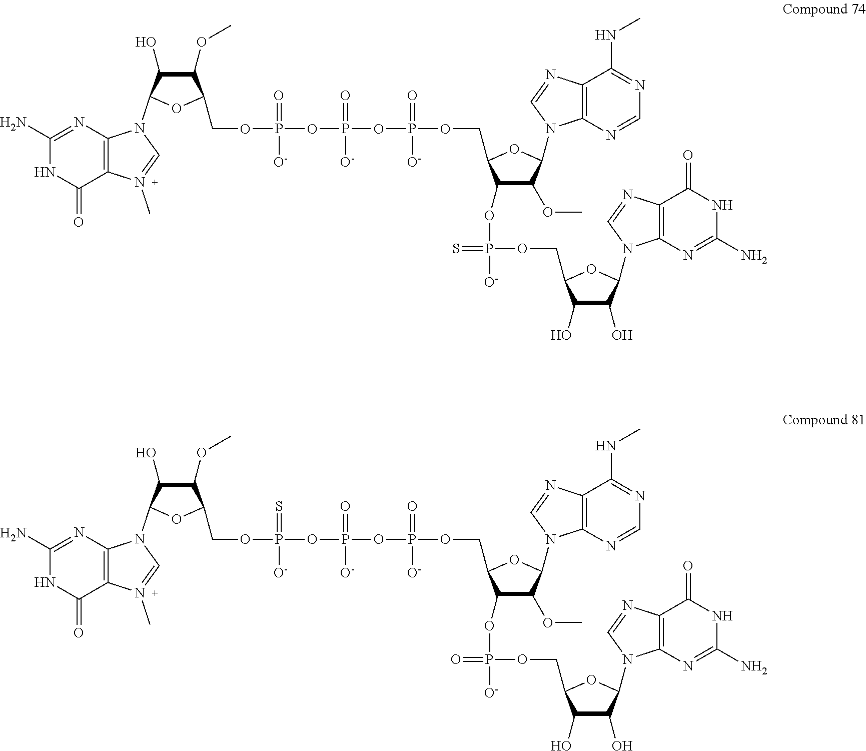

or any stereoisomer thereof.

Further provided herein is a compound of the following formula:

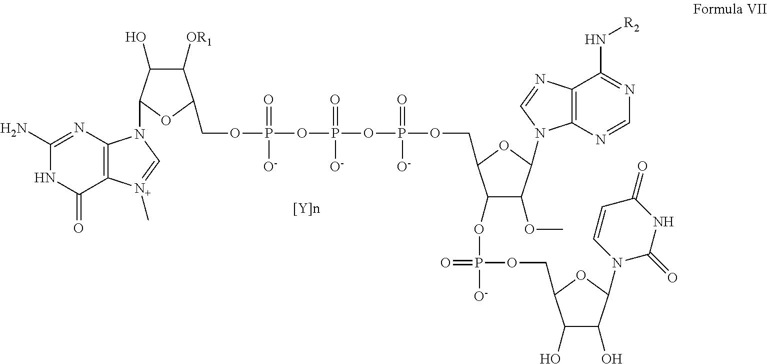

or any stereoisomer thereof, wherein R₁ and R₂ are each independently selected from H and CH₃; each independent Y is H⁺ or a cation; and n is 0, 1, 2, 3, or 4, wherein at least one of R₁ or R₂ is CH₃. Optionally, the compound has the following structure:

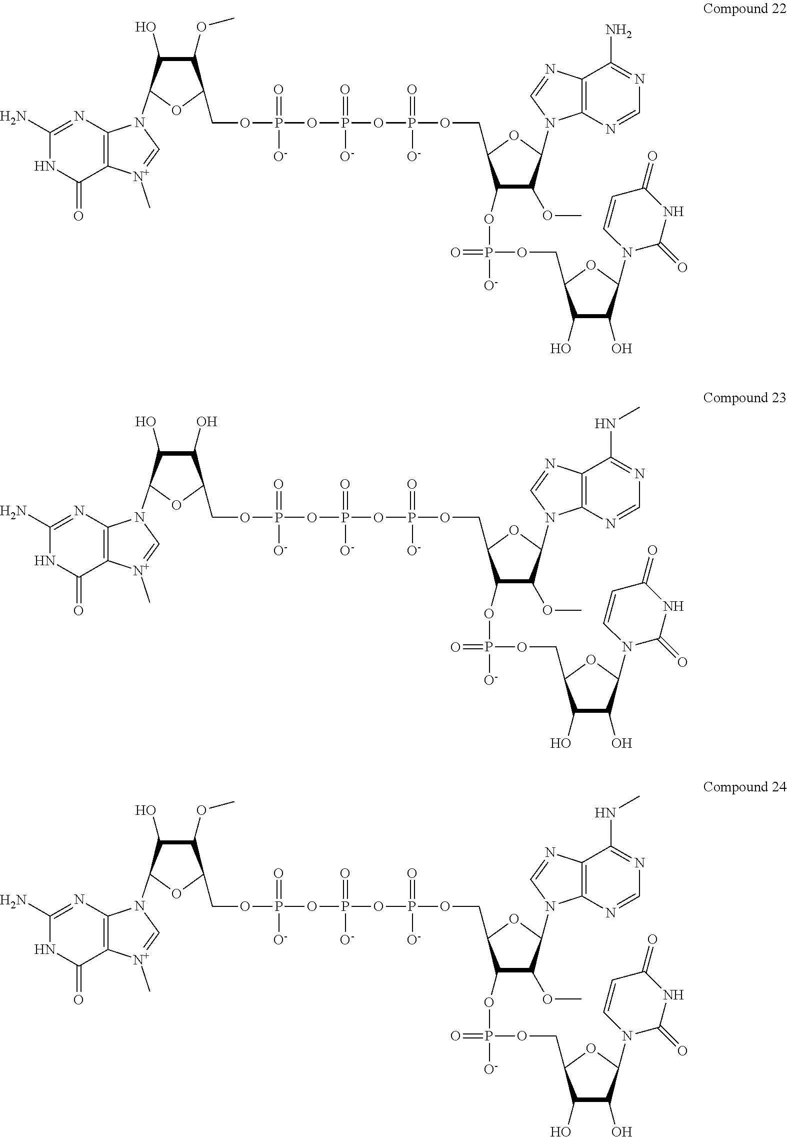

or any stereoisomer thereof.

Also provided herein is a compound has the following formula:

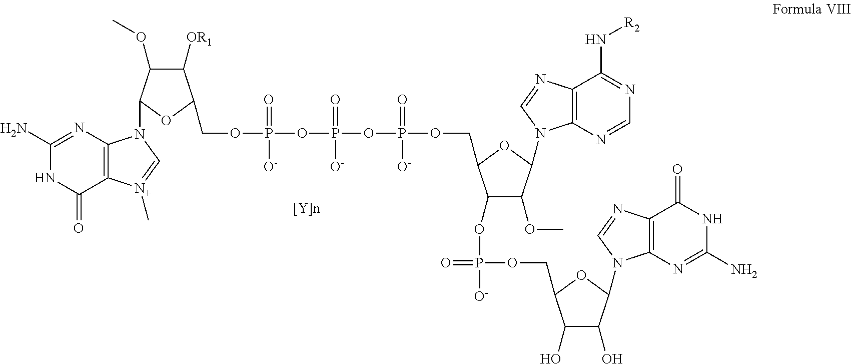

or any stereoisomer thereof, wherein: R₁ and R₂ are each independently selected from H and CH₃; each independent Y is H⁺ or a cation; and n is 0, 1, 2, 3, or 4. Optionally, the compound has the following structure:

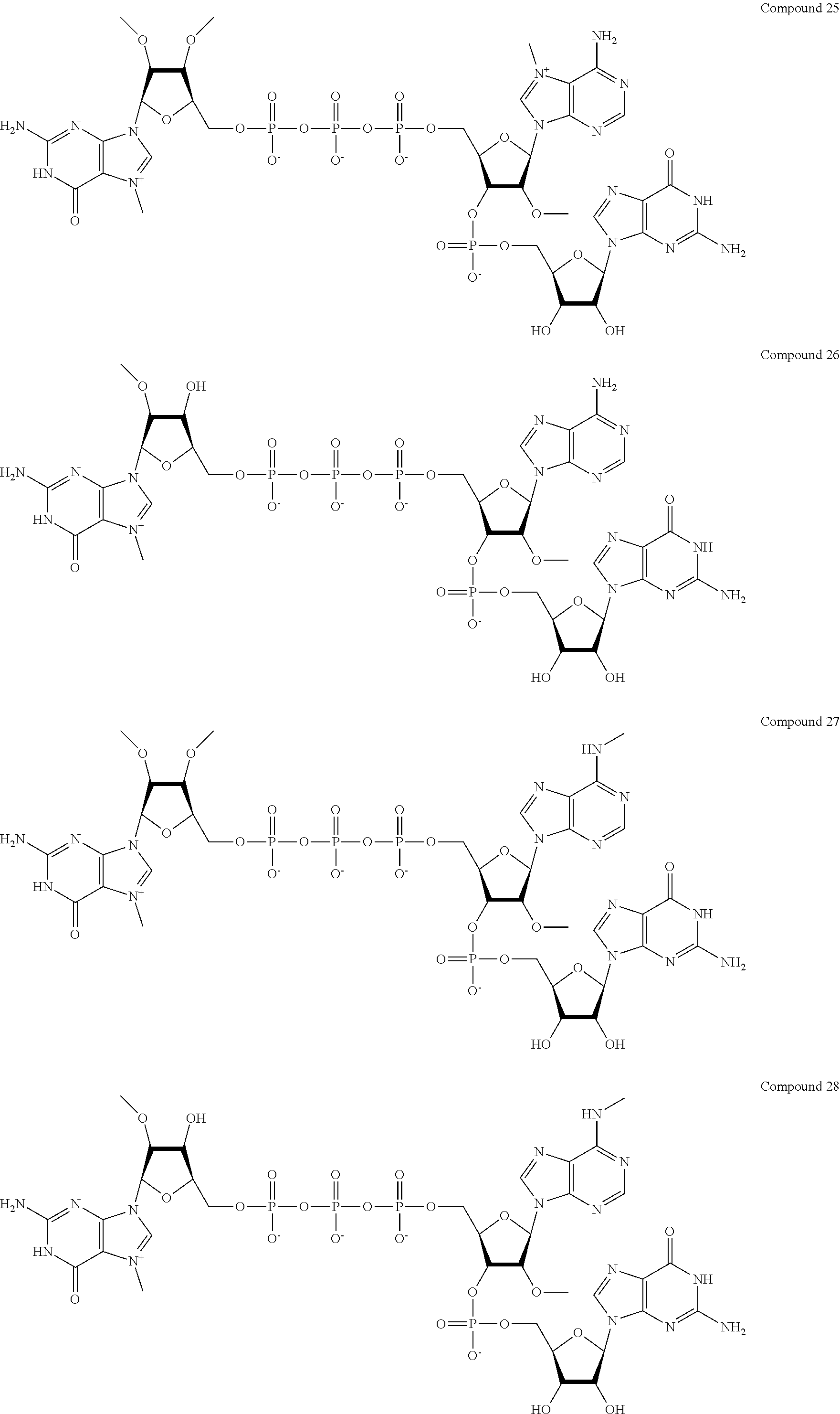

or any stereoisomer thereof.

Additionally provided herein is a compound of the following formula:

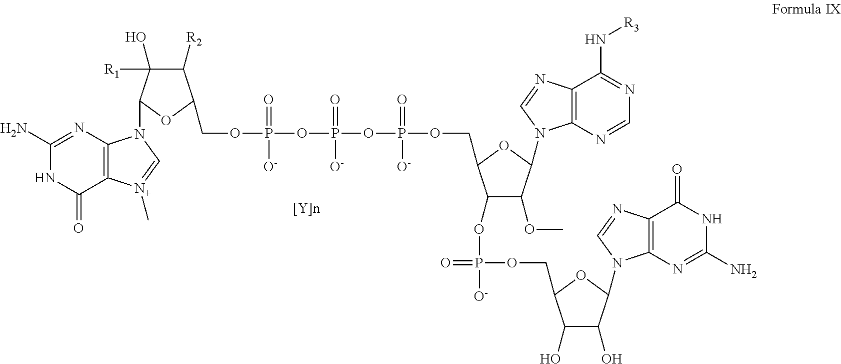

or any stereoisomer thereof, wherein R₁ is H or CH₃; R₂ is OH, F, substituted or unsubstituted alkoxy, or thio; R₃ is H or CH₃; each independent Y is H⁺ or a cation; and n is 0, 1, 2, 3, or 4, wherein when R₂ is OH, R₁ is not H. Optionally, the compound has the following structure:

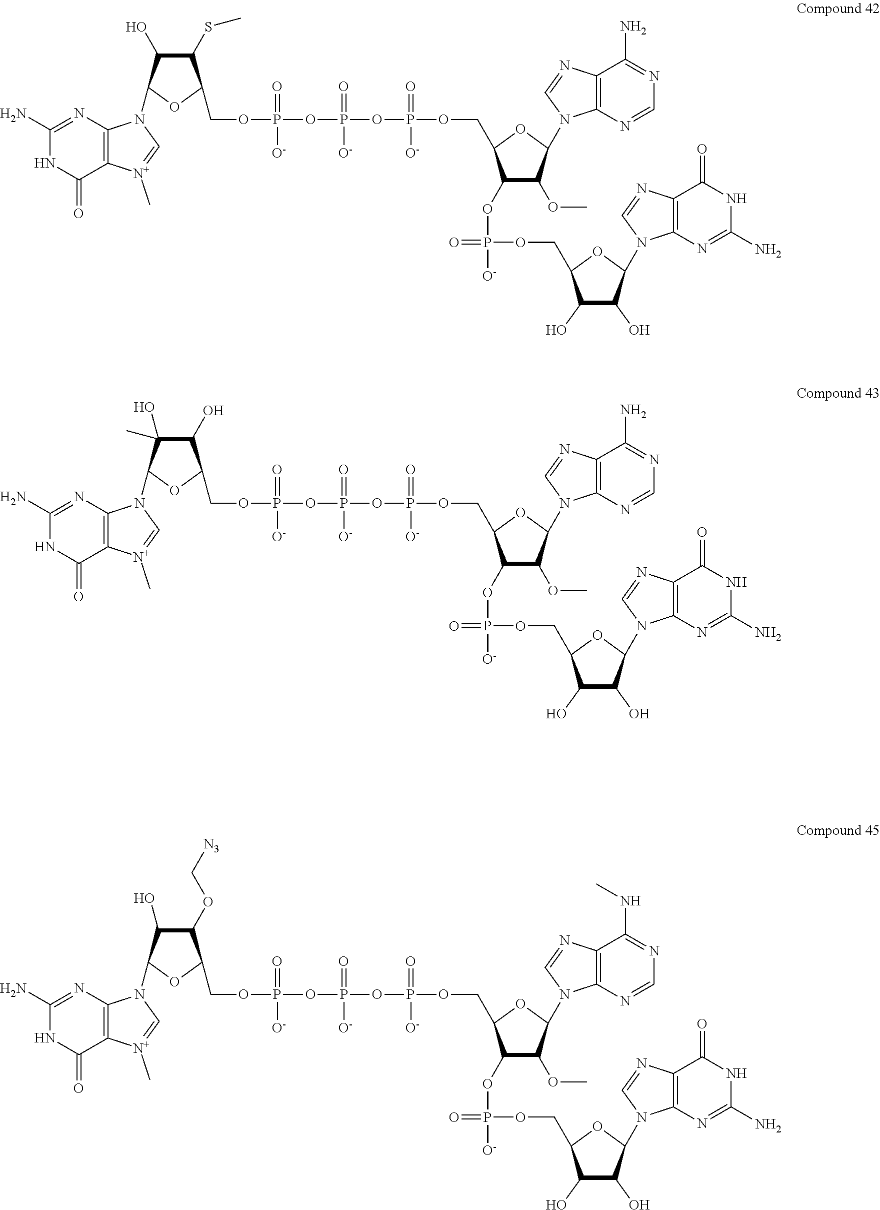

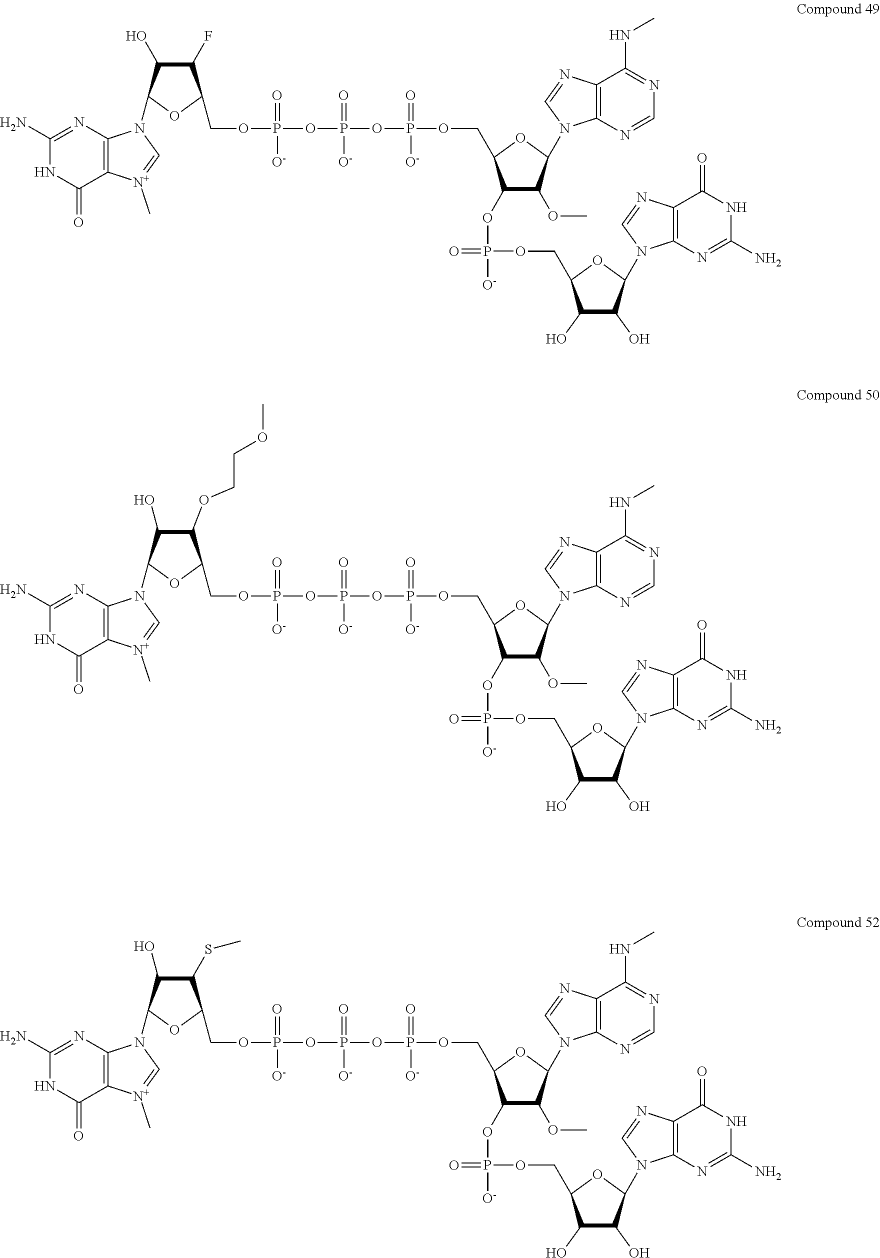

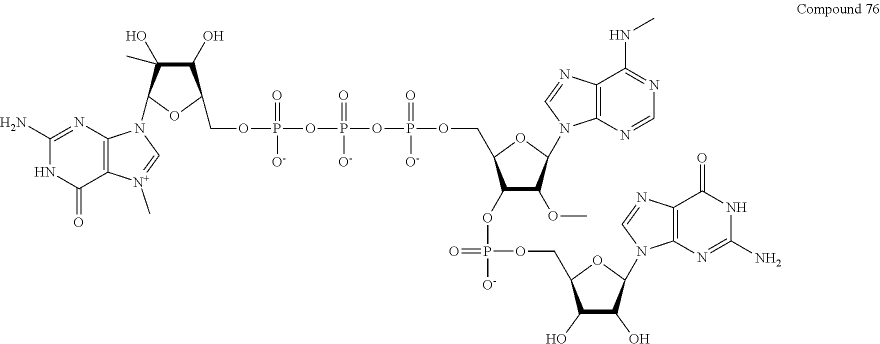

or any stereoisomer thereof.

Also provided herein is a compound of the following formula:

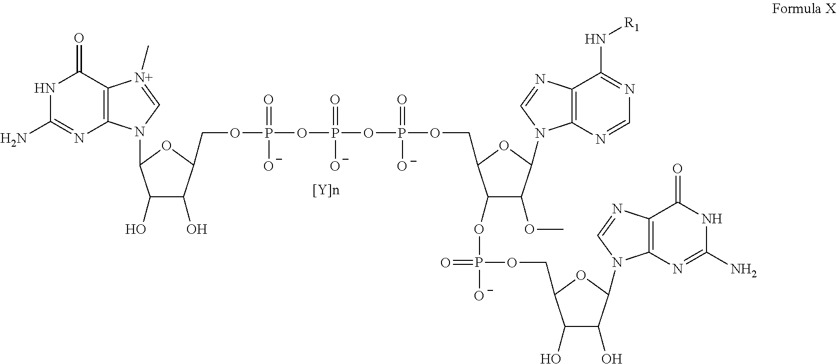

or any stereoisomer thereof, wherein R₁ is H and CH₃; each independent Y is H⁺ or a cation; and n is 0, 1, 2, 3, or 4. Optionally, the compound has the following structure:

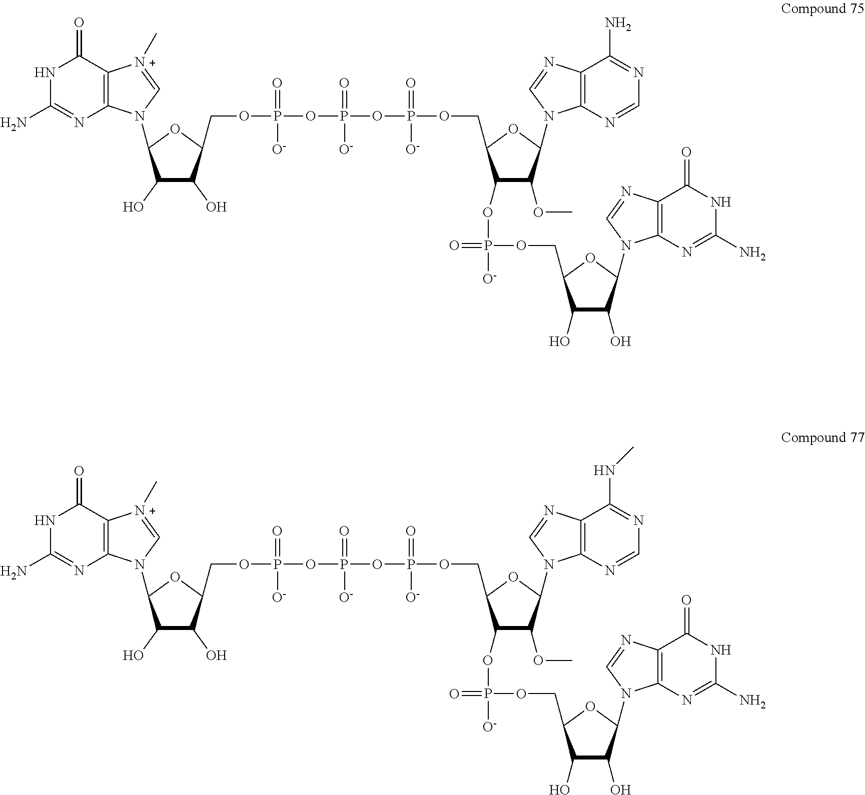

or any stereoisomer thereof.

Additionally provided herein is a compound of the following formula:

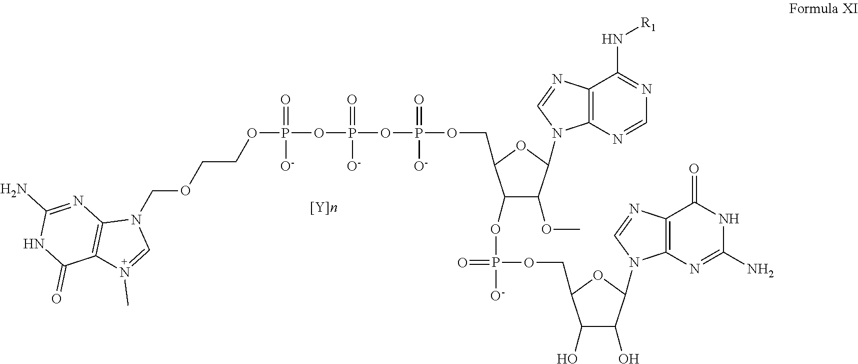

or any stereoisomer thereof, wherein R₁ is H and CH₃; each independent Y is H⁺ or a cation; and n is 0, 1, 2, 3, or 4. Optionally, the compound has the following structure:

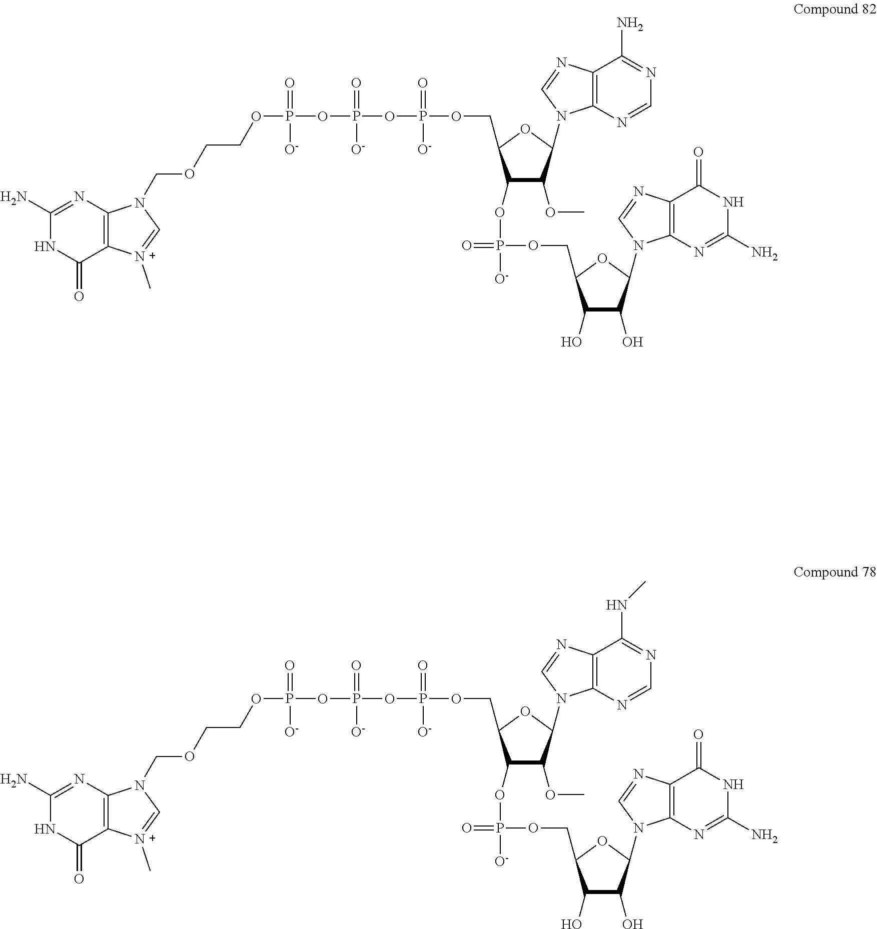

or any stereoisomer thereof.

Also provided herein is a compound of the following formula:

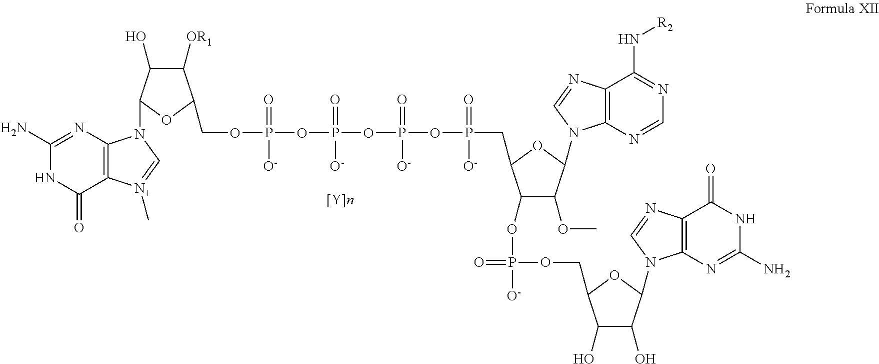

or any stereoisomer thereof, wherein R₁ and R₂ are each independently selected from H and CH₃; each independent Y is H⁺ or a cation; and n is 0, 1, 2, 3, 4, or 5. Optionally, the compound has the following structure:

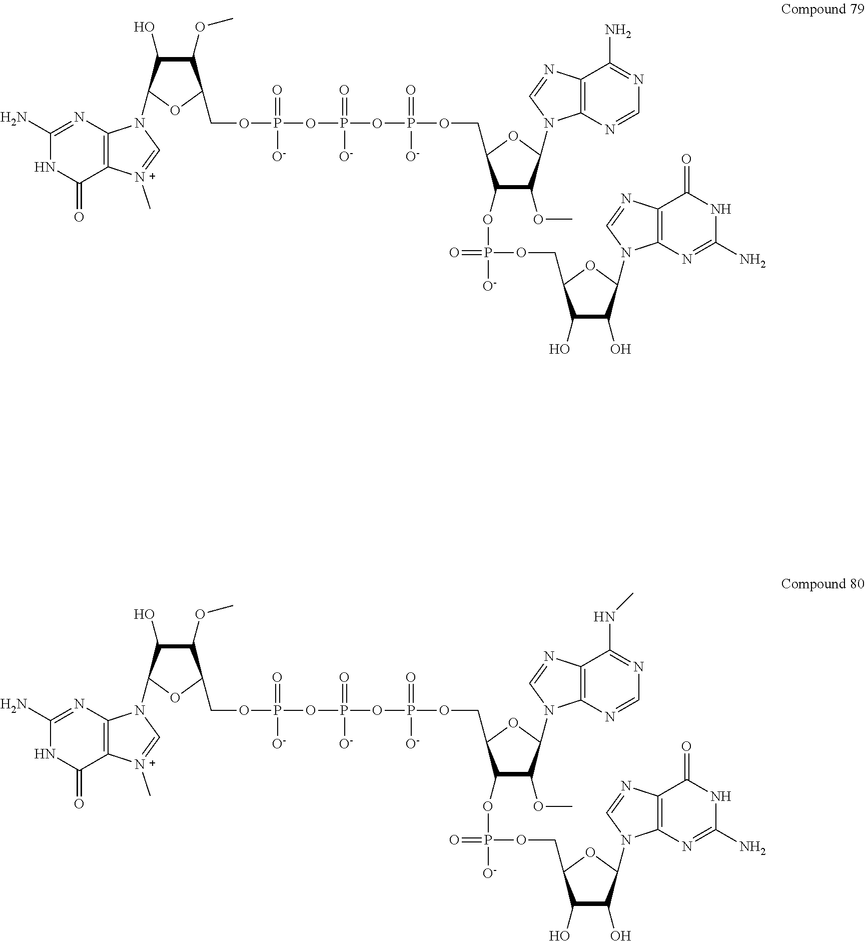

or any stereoisomer thereof.

Also described herein are deuterated forms of the disclosed compounds. For example, provided herein is compound selected from the group consisting of:

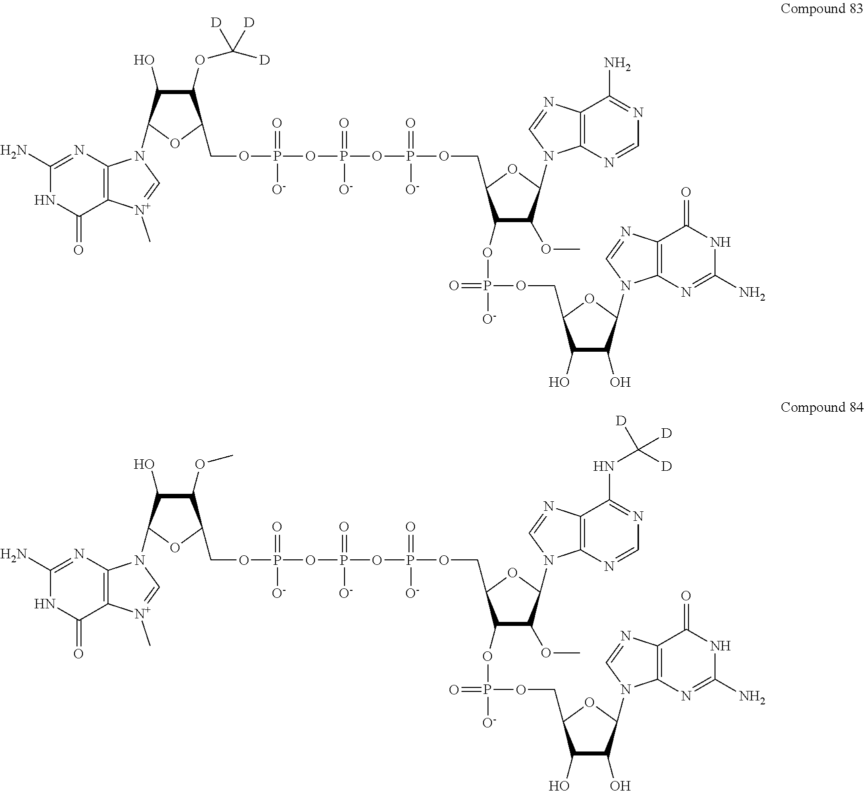

or any stereoisomer thereof.

Also described herein is a pharmaceutical composition, comprising a compound as described herein and a pharmaceutically acceptable carrier. Also provided is an RNA molecule comprising a 5′-cap, wherein the 5′-cap comprises a compound as described herein. Optionally, the RNA molecule is a messenger RNA (mRNA) molecule or a self-amplifying RNA (saRNA) molecule. Methods of inducing a therapeutic effect in a subject are also provided. The methods comprise administering to the subject an RNA molecule (e.g., an mRNA or a saRNA molecule) as described herein. The administered RNA may contain some amount of immunogenic double stranded RNA (dsRNA). The amount of dsRNA is calculated per each ug of administered RNA.

Further provided herein are methods of administering to an animal a therapeutic dose unit of an mRNA molecule comprising a 5′-cap. In some examples, the 5′-cap comprises a compound as described herein. In some examples, the 5′-cap comprises a compound selected from the group consisting of, or any stereoisomer thereof:

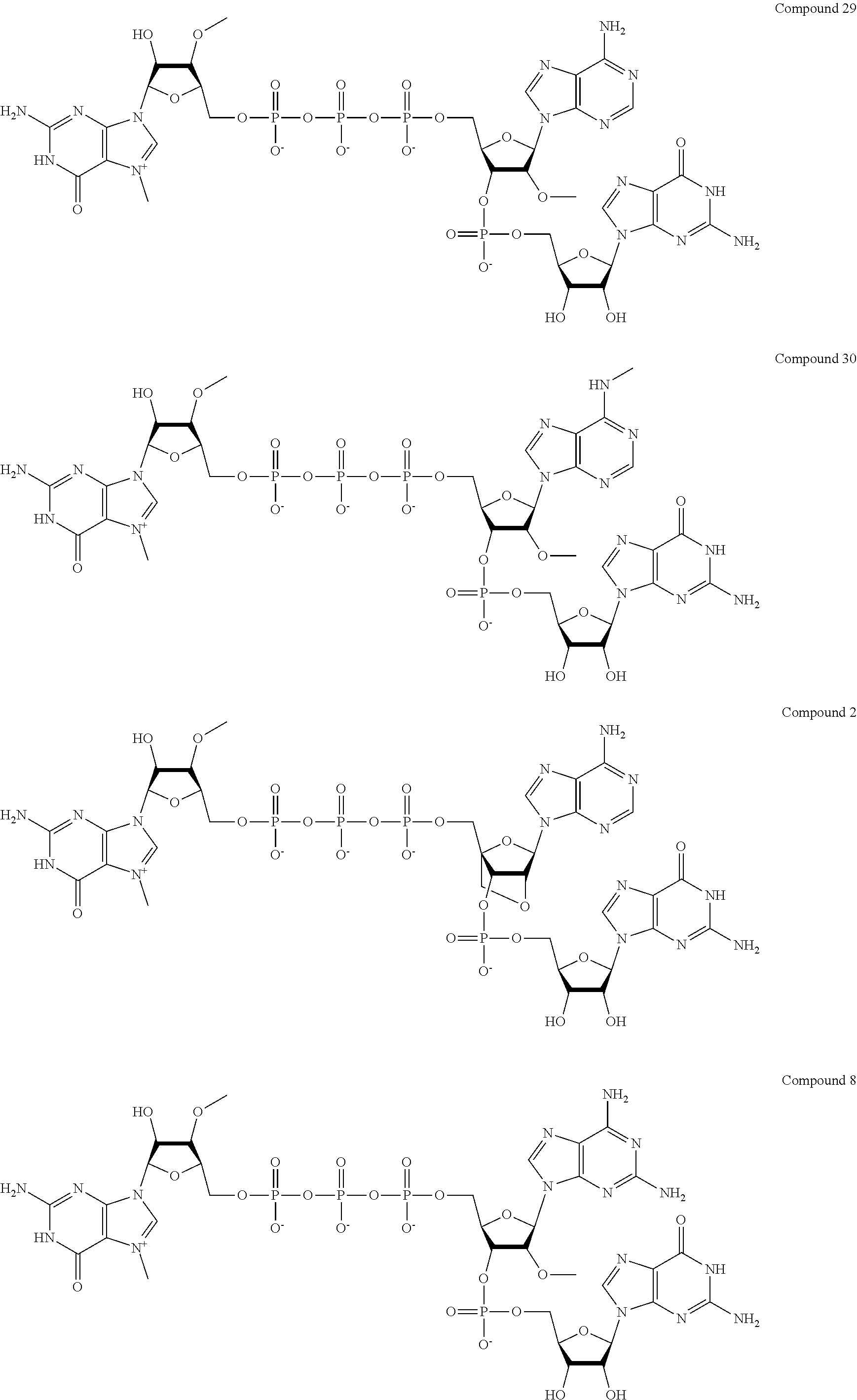

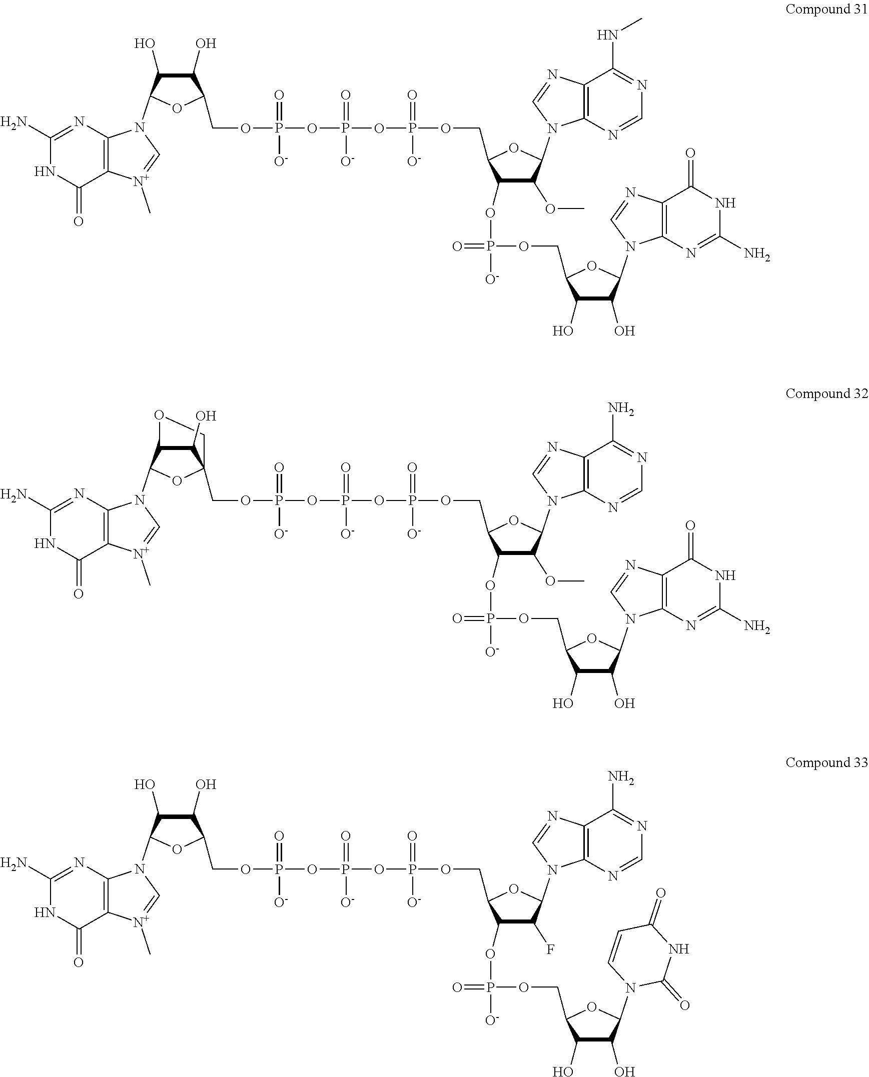

wherein, in some examples, the therapeutic dose unit (e.g., the therapeutic mRNA dose unit) comprises less than 7 ng/μg of double stranded RNA (dsRNA), and wherein, in some examples, the subject exhibits increased tolerability to the administered therapeutic dose unit of the mRNA molecule as compared to an equivalent therapeutic dose unit of the mRNA molecule comprising 7 ng/μg or greater dsRNA. Optionally, the increased tolerability is determined by measuring one or more of body weight, organ weight, aspartate aminotransferase (AST) levels, alanine transaminase (ALT) levels, C-reactive protein (CRP) levels, procalcitonin (PCT) levels, interleukin-6 (IL-6) levels, erythrocyte sedimentation rate (ESR), serum amyloid A levels, and serum ferritin levels prior to the administering and a period of time after the administering. Optionally, the increased tolerability is measured by testing in a standard in vivo assay. In some examples, the increased tolerability is an increase in tolerability of at least 20% to the administered dose unit as measured by testing in a standard in vivo assay, at least 25% to the administered dose unit as measured by testing in a standard in vivo assay, at least 30% to the administered dose unit as measured by testing in a standard in vivo assay, at least 35% to the administered dose unit as measured by testing in a standard in vivo assay, at least 40% to the administered dose unit as measured by testing in a standard in vivo assay, at least 45% to the administered dose unit as measured by testing in a standard in vivo assay, at least 50% to the administered dose unit as measured by testing in a standard in vivo assay, at least 55% to the administered dose unit as measured by testing in a standard in vivo assay, at least 60% to the administered dose unit as measured by testing in a standard in vivo assay, at least 65% to the administered dose unit as measured by testing in a standard in vivo assay, at least 70% to the administered dose unit as measured by testing in a standard in vivo assay, at least 75% to the administered dose unit as measured by testing in a standard in vivo assay, at least 80% to the administered dose unit as measured by testing in a standard in vivo assay, at least 85% to the administered dose unit as measured by testing in a standard in vivo assay, at least 90% to the administered dose unit as measured by testing in a standard in vivo assay, or at least 95% to the administered dose unit as measured by testing in a standard in vivo assay.

Optionally, the therapeutic dose unit of the mRNA molecule comprises less than 6.9 ng/μg of double stranded RNA (dsRNA), less than 6.8 ng/μg of double stranded RNA (dsRNA), less than 6.7 ng/μg of double stranded RNA (dsRNA), less than 6.6 ng/μg of double stranded RNA (dsRNA), less than 6.5 ng/μg of double stranded RNA (dsRNA), less than 6.4 ng/μg of double stranded RNA (dsRNA), less than 6.3 ng/μg of double stranded RNA (dsRNA), less than 6.2 ng/μg of double stranded RNA (dsRNA), less than 6.1 ng/μg of double stranded RNA (dsRNA), less than 6.0 ng/μg of double stranded RNA (dsRNA), less than 5.9 ng/μg of double stranded RNA (dsRNA), less than 5.8 ng/μg of double stranded RNA (dsRNA), less than 5.7 ng/μg of double stranded RNA (dsRNA), less than 5.6 ng/μg of double stranded RNA (dsRNA), less than 5.5 ng/μg of double stranded RNA (dsRNA), less than 5.4 ng/μg of double stranded RNA (dsRNA), less than 5.3 ng/μg of double stranded RNA (dsRNA), less than 5.2 ng/μg of double stranded RNA (dsRNA), less than 5.1 ng/μg of double stranded RNA (dsRNA), less than 5.0 ng/μg of double stranded RNA (dsRNA), less than 4.9 ng/μg of double stranded RNA (dsRNA), less than 4.8 ng/μg of double stranded RNA (dsRNA), less than 4.7 ng/μg of double stranded RNA (dsRNA), less than 4.6 ng/μg of double stranded RNA (dsRNA), less than 4.5 ng/μg of double stranded RNA (dsRNA), less than 4.4 ng/μg of double stranded RNA (dsRNA), less than 4.3 ng/μg of double stranded RNA (dsRNA), less than 4.2 ng/μg of double stranded RNA (dsRNA), less than 4.1 ng/μg of double stranded RNA (dsRNA), less than 4.0 ng/μg of double stranded RNA (dsRNA), less than 3.9 ng/μg of double stranded RNA (dsRNA), less than 3.8 ng/μg of double stranded RNA (dsRNA), less than 3.7 ng/μg of double stranded RNA (dsRNA), less than 3.6 ng/μg of double stranded RNA (dsRNA), less than 3.5 ng/μg of double stranded RNA (dsRNA), less than 3.4 ng/μg of double stranded RNA (dsRNA), less than 3.3 ng/μg of double stranded RNA (dsRNA), less than 3.2 ng/μg of double stranded RNA (dsRNA), less than 3.1 ng/μg of double stranded RNA (dsRNA), less than 3.0 ng/μg of double stranded RNA (dsRNA), less than 2.9 ng/μg of double stranded RNA (dsRNA), less than 2.8 ng/μg of double stranded RNA (dsRNA), less than 2.7 ng/μg of double stranded RNA (dsRNA), less than 2.6 ng/μg of double stranded RNA (dsRNA), less than 2.5 ng/μg of double stranded RNA (dsRNA), less than 2.4 ng/μg of double stranded RNA (dsRNA), less than 2.3 ng/μg of double stranded RNA (dsRNA), less than 2.2 ng/μg of double stranded RNA (dsRNA), less than 2.1 ng/μg of double stranded RNA (dsRNA), less than 2.0 ng/μg of double stranded RNA (dsRNA), less than 1.9 ng/μg of double stranded RNA (dsRNA), less than 1.8 ng/μg of double stranded RNA (dsRNA), less than 1.7 ng/μg of double stranded RNA (dsRNA), less than 1.6 ng/μg of double stranded RNA (dsRNA), less than 1.5 ng/μg of double stranded RNA (dsRNA), less than 1.4 ng/μg of double stranded RNA (dsRNA), less than 1.3 ng/μg of double stranded RNA (dsRNA), less than 1.2 ng/μg of double stranded RNA (dsRNA), less than 1.1 ng/μg of double stranded RNA (dsRNA), less than 1.0 ng/μg of double stranded RNA (dsRNA), less than 0.9 ng/μg of double stranded RNA (dsRNA), less than 0.8 ng/μg of double stranded RNA (dsRNA), less than 0.7 ng/μg of double stranded RNA (dsRNA), less than 0.6 ng/μg of double stranded RNA (dsRNA), less than 0.5 ng/μg of double stranded RNA (dsRNA), less than 0.4 ng/μg of double stranded RNA (dsRNA), less than 0.3 ng/μg of double stranded RNA (dsRNA), less than 0.2 ng/μg of double stranded RNA (dsRNA), or less than 0.1 ng/μg of double stranded RNA (dsRNA).

Further provided herein is a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula, or any stereoisomer thereof:

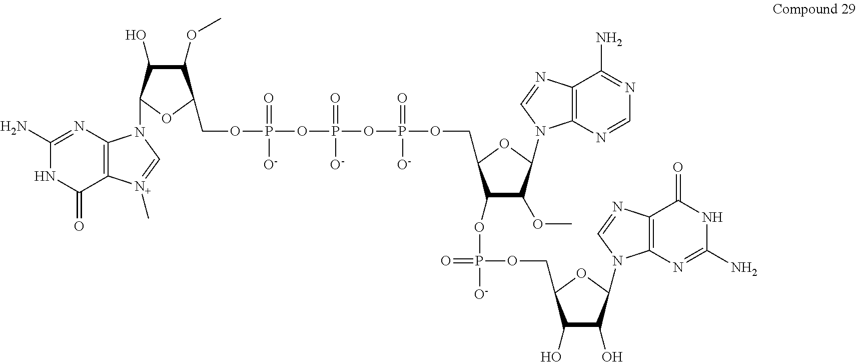

wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula, or any stereoisomer thereof:

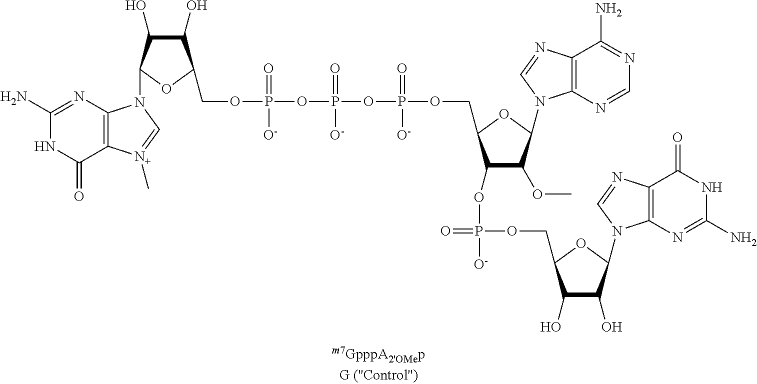

Also provided herein is a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula, or any stereoisomer thereof:

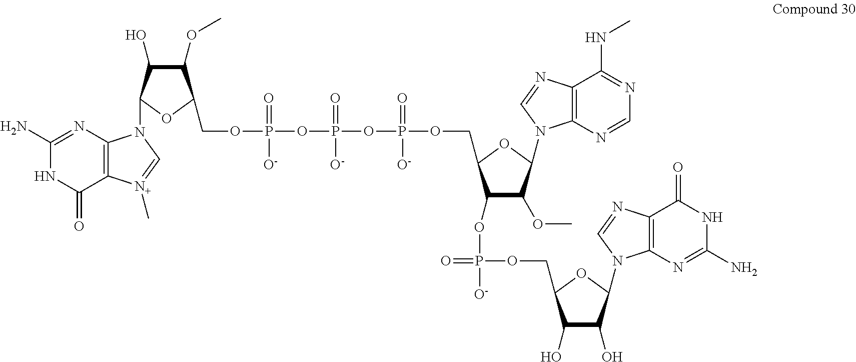

wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula, or any stereoisomer thereof:

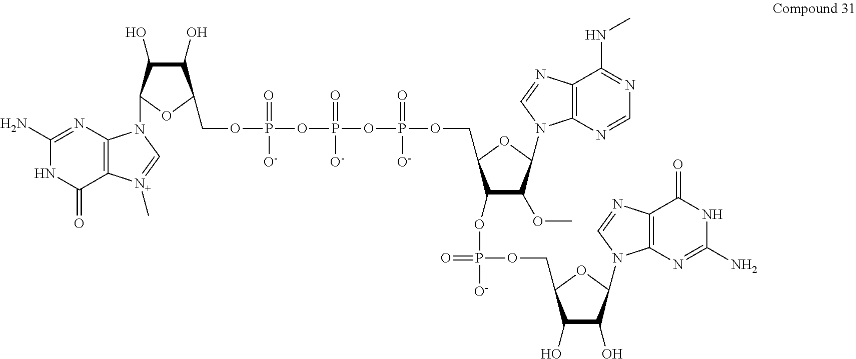

Further provided herein is a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula, or any stereoisomer thereof:

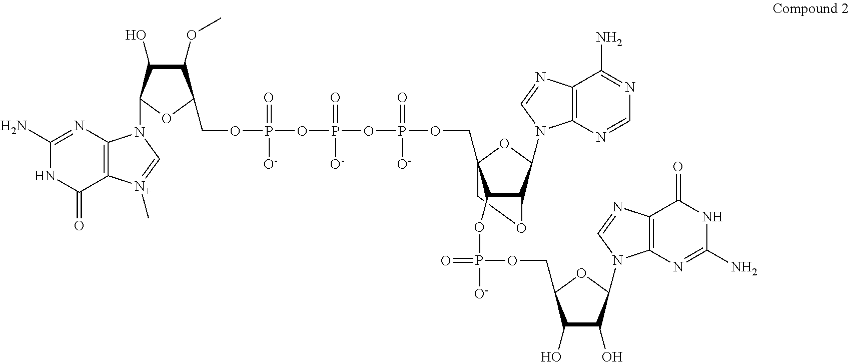

wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula, or any stereoisomer thereof:

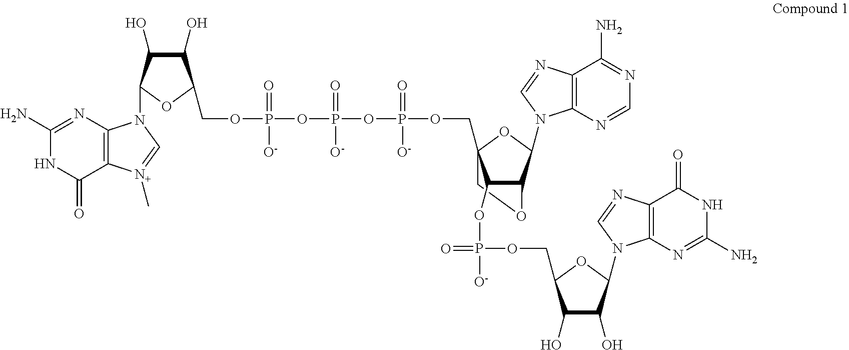

Also provided herein is a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula, or any stereoisomer thereof:

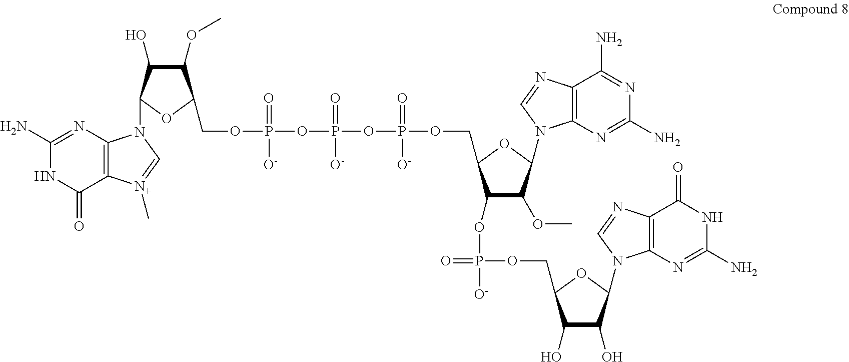

wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula, or any stereoisomer thereof:

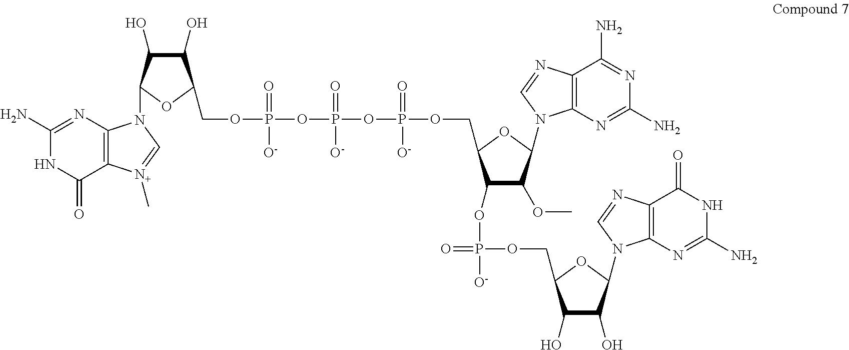

Optionally, the subject exhibits increased tolerability to the administered therapeutic dose unit as compared to the therapeutic dose unit required to elicit the same response in the subject when administered the comparative mRNA, as measured by testing in a standard in vivo assay. In some cases, the increased tolerability is an increase in tolerability of at least 20% to the administered therapeutic dose unit as measured by testing in a standard in vivo assay, at least 25% to the administered therapeutic dose unit as measured by testing in a standard in vivo assay, at least 30% to the administered therapeutic dose unit as measured by testing in a standard in vivo assay, at least 35% to the administered therapeutic dose unit as measured by testing in a standard in vivo assay, at least 40% to the administered therapeutic dose unit as measured by testing in a standard in vivo assay, at least 45% to the administered therapeutic dose unit as measured by testing in a standard in vivo assay, at least 50% to the administered therapeutic dose unit as measured by testing in a standard in vivo assay, at least 55% to the administered therapeutic dose unit as measured by testing in a standard in vivo assay, at least 60% to the administered therapeutic dose unit as measured by testing in a standard in vivo assay, at least 65% to the administered therapeutic dose unit as measured by testing in a standard in vivo assay, at least 70% to the administered therapeutic dose unit as measured by testing in a standard in vivo assay, at least 75% to the administered therapeutic dose unit as measured by testing in a standard in vivo assay, at least 80% to the administered therapeutic dose unit as measured by testing in a standard in vivo assay, at least 85% to the administered therapeutic dose unit as measured by testing in a standard in vivo assay, at least 90% to the administered therapeutic dose unit as measured by testing in a standard in vivo assay, or at least 95% to the administered therapeutic dose unit as measured by testing in a standard in vivo assay.

Optionally, the administered therapeutic dose unit comprises less than 6.9 ng/μg of double stranded RNA (dsRNA), less than 6.8 ng/μg of double stranded RNA (dsRNA), less than 6.7 ng/μg of double stranded RNA (dsRNA), less than 6.6 ng/μg of double stranded RNA (dsRNA), less than 6.5 ng/μg of double stranded RNA (dsRNA), less than 6.4 ng/μg of double stranded RNA (dsRNA), less than 6.3 ng/μg of double stranded RNA (dsRNA), less than 6.2 ng/μg of double stranded RNA (dsRNA), less than 6.1 ng/μg of double stranded RNA (dsRNA), less than 6.0 ng/μg of double stranded RNA (dsRNA), less than 5.9 ng/μg of double stranded RNA (dsRNA), less than 5.8 ng/μg of double stranded RNA (dsRNA), less than 5.7 ng/μg of double stranded RNA (dsRNA), less than 5.6 ng/μg of double stranded RNA (dsRNA), less than 5.5 ng/μg of double stranded RNA (dsRNA), less than 5.4 ng/μg of double stranded RNA (dsRNA), less than 5.3 ng/μg of double stranded RNA (dsRNA), less than 5.2 ng/μg of double stranded RNA (dsRNA), less than 5.1 ng/μg of double stranded RNA (dsRNA), less than 5.0 ng/μg of double stranded RNA (dsRNA), less than 4.9 ng/μg of double stranded RNA (dsRNA), less than 4.8 ng/μg of double stranded RNA (dsRNA), less than 4.7 ng/μg of double stranded RNA (dsRNA), less than 4.6 ng/μg of double stranded RNA (dsRNA), less than 4.5 ng/μg of double stranded RNA (dsRNA), less than 4.4 ng/μg of double stranded RNA (dsRNA), less than 4.3 ng/μg of double stranded RNA (dsRNA), less than 4.2 ng/μg of double stranded RNA (dsRNA), less than 4.1 ng/μg of double stranded RNA (dsRNA), less than 4.0 ng/μg of double stranded RNA (dsRNA), less than 3.9 ng/μg of double stranded RNA (dsRNA), less than 3.8 ng/μg of double stranded RNA (dsRNA), less than 3.7 ng/μg of double stranded RNA (dsRNA), less than 3.6 ng/μg of double stranded RNA (dsRNA), less than 3.5 ng/μg of double stranded RNA (dsRNA), less than 3.4 ng/μg of double stranded RNA (dsRNA), less than 3.3 ng/μg of double stranded RNA (dsRNA), less than 3.2 ng/μg of double stranded RNA (dsRNA), less than 3.1 ng/μg of double stranded RNA (dsRNA), less than 3.0 ng/μg of double stranded RNA (dsRNA), less than 2.9 ng/μg of double stranded RNA (dsRNA), less than 2.8 ng/μg of double stranded RNA (dsRNA), less than 2.7 ng/μg of double stranded RNA (dsRNA), less than 2.6 ng/μg of double stranded RNA (dsRNA), less than 2.5 ng/μg of double stranded RNA (dsRNA), less than 2.4 ng/μg of double stranded RNA (dsRNA), less than 2.3 ng/μg of double stranded RNA (dsRNA), less than 2.2 ng/μg of double stranded RNA (dsRNA), less than 2.1 ng/μg of double stranded RNA (dsRNA), less than 2.0 ng/μg of double stranded RNA (dsRNA), less than 1.9 ng/μg of double stranded RNA (dsRNA), less than 1.8 ng/μg of double stranded RNA (dsRNA), less than 1.7 ng/μg of double stranded RNA (dsRNA), less than 1.6 ng/μg of double stranded RNA (dsRNA), less than 1.5 ng/μg of double stranded RNA (dsRNA), less than 1.4 ng/μg of double stranded RNA (dsRNA), less than 1.3 ng/μg of double stranded RNA (dsRNA), less than 1.2 ng/μg of double stranded RNA (dsRNA), less than 1.1 ng/μg of double stranded RNA (dsRNA), less than 1.0 ng/μg of double stranded RNA (dsRNA), less than 0.9 ng/μg of double stranded RNA (dsRNA), less than 0.8 ng/μg of double stranded RNA (dsRNA), less than 0.7 ng/μg of double stranded RNA (dsRNA), less than 0.6 ng/μg of double stranded RNA (dsRNA), less than 0.5 ng/μg of double stranded RNA (dsRNA), less than 0.4 ng/μg of double stranded RNA (dsRNA), less than 0.3 ng/μg of double stranded RNA (dsRNA), less than 0.2 ng/μg of double stranded RNA (dsRNA), or less than 0.1 ng/μg of double stranded RNA (dsRNA).

Further described herein are methods of synthesizing a trinucleotide compound. In one example, a method of synthesizing a trinucleotide compound comprises mixing an N7-methyl-guanosine-5′-diphosphate with an activating reagent to form a reactive intermediate; reacting the reactive intermediate with an imidazole to form an activated intermediate; and adding a salt reagent (e.g., a chloride salt such as magnesium chloride or zinc chloride) and a dinucleotide to the activated intermediate to form the trinucleotide compound, wherein the method is a one-pot synthesis. In some examples, the method of synthesizing a trinucleotide compound comprises mixing an N7-methyl-guanosine-5′-diphosphate of the following structure:

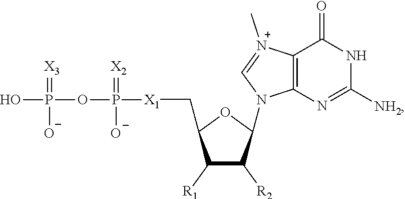

wherein X₁ and X₂ are each independently selected from the group consisting of O and S; X₃ is O; R₁ and R₂ are each independently selected from H, OH, N₃, F, substituted or unsubstituted alkoxy, and thio, wherein R₁ and R₂ optionally combine to form a heterocycle, with an activating reagent and an imidazole to form an activated intermediate; and adding a salt reagent (e.g., a chloride salt such as magnesium chloride or zinc chloride) and a dinucleotide of the following structure:

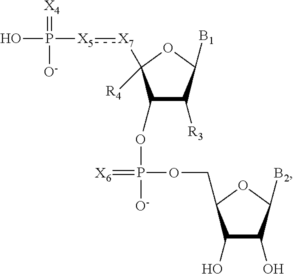

wherein [symbol] is a single bond or a double bond; X₄ and X₅ are each independently selected from the group consisting of O and S; X₅ is O, S, or CH; X₇ is CH or CH₂; R₃ is OH, OMe, F and R₄ is H, or wherein R₃ and R₄ are covalently bonded together and, together with intermediate atoms, form a 2′-O, 4′-C methylene bridge; B₁ and B₂ are each independently selected from the group consisting of a purine ring and a pyrimidine ring, to the activated intermediate to form the trinucleotide compound of the following structure:

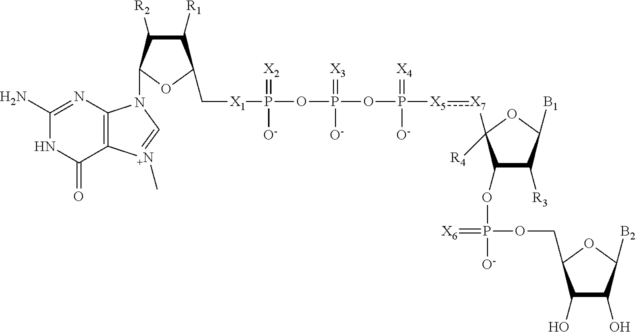

wherein the method is a one-pot synthesis.

In other examples, the method of synthesizing a trinucleotide compound comprises mixing a dinucleotide of the following structure:

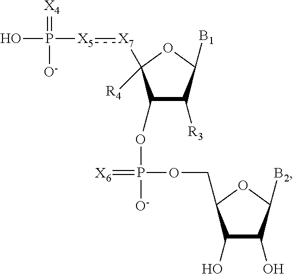

wherein [symbol]
is a single bond or a double bond; X₄ is O; X₆ is O or S; X₅ is O, S, or CH; X₇ is CH or CH₂; R₃ is OH, OMe, F and R₄ is H, or wherein R₃ and R₄ are covalently bonded together and, together with intermediate atoms, form a 2′-O, 4′-C methylene bridge; and B₁ and B₂ are each independently selected from the group consisting of a purine ring and a pyrimidine ring, with an activating reagent and an imidazole to form an activated phosphate imidazolide of the following structure:

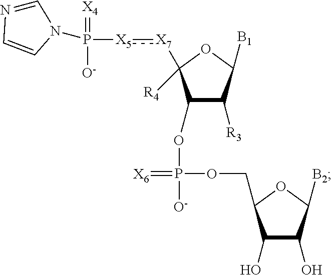

and
adding a salt reagent (e.g., a chloride salt such as magnesium chloride or zinc chloride) and a compound of the following structure:

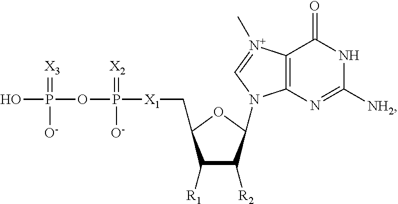

wherein
X₁ and X₂ are each independently selected from the group consisting of O and S; X₃ is S; R₁ and R₂ are each independently selected from H, OH, N₃, F, substituted or unsubstituted alkoxy, and thio, wherein R₁ and R₂ optionally combine to form a heterocycle, to the activated phosphate imidazolide to form the trinucleotide compound of the following structure:

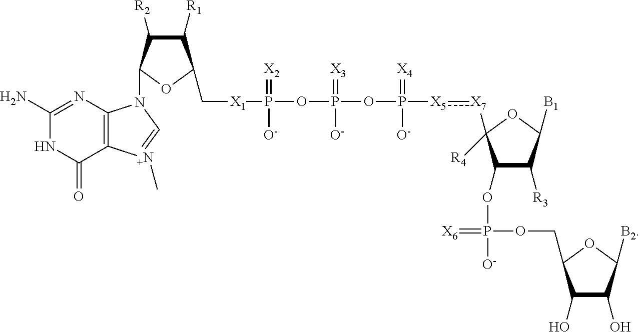

In still other examples, the method of synthesizing a trinucleotide compound comprises mixing a diphosphate dinucleotide of the following structure:

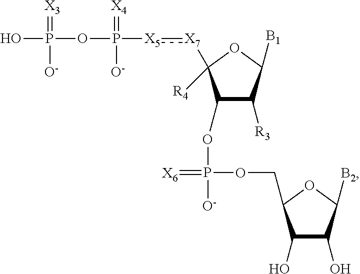

wherein [symbol]
is a single bond or a double bond; X₃ is O; X₄ and X₆ are each independently selected from O and S; X₅ is O, S, or CH; X₇ is CH or CH₂; R₃ is OH, OMe, F and R₄ is H, or wherein R₃ and R₄ are covalently bonded together and, together with intermediate atoms, form a 2′-O, 4′-C methylene bridge; and B₁ and B₂ are each independently selected from the group consisting of a purine ring and a pyrimidine ring, with an activating reagent and an imidazole to form an activated phosphate imidazolide of the following structure:

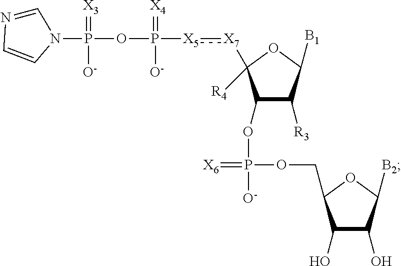

and
adding a salt reagent (e.g., a chloride salt such as magnesium chloride or zinc chloride) and a compound of the following structure:

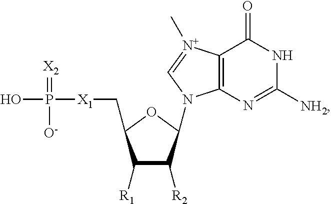

wherein
X₁ and X₂ are each independently selected from O and S; and R₁ and R₂ are each independently selected from H, OH, N₃, F, substituted or unsubstituted alkoxy, and thio, wherein R₁ and R₂ optionally combine to form a heterocycle, to the activated phosphate imidazolide to form the trinucleotide compound of the following structure:

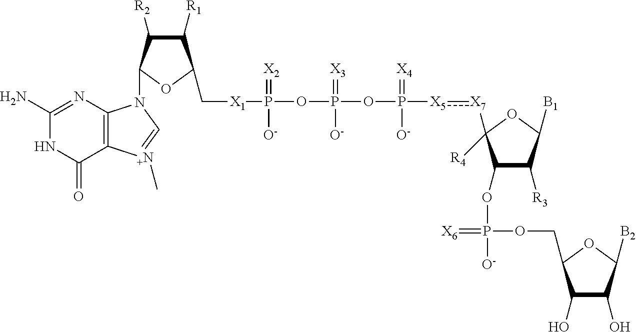

wherein the method is a one-pot synthesis.

The details of one or more embodiments are set forth in the drawings and the description below. Other features, objects, and advantages will be apparent from the description and drawings, and from the claims.

### DESCRIPTION OF THE DRAWINGS

FIG. 1 is a bar graph showing in vitro translation using wheat germ extract, a cell-free translation system, for the cap analogs as described herein. The data demonstrate that protein expression for the cap analogs was comparable to or enhanced as compared to mRNA produced from a comparative cap.

FIG. 2 is a bar graph showing the in vivo luciferase expression in mice injected with LNPs comprising mRNA produced from cap analogs as described herein along with mRNA produced from comparative caps. The data demonstrate that in vivo luciferase expression was comparable to or enhanced as compared to mRNA produced from a comparative cap.

FIG. 3 shows representative animals from a cohort of five animals shown across five imaging time points post LNP:mRNA and luciferin injection. Luminescence intensity scale is on the right.

FIG. 4 contains plots showing that a 3′OMe modification on the ^{m7}G moiety of cap analogs results in increased in vivo translation. ^{m7}GpppA_{2′OMe}pG (Control) was measured relative to ^{m7}G_{3′Ome}pppA_{2′OMe}pG (Compound 29), which is ^{m7}GpppA_{2′OMe}pG (Control) with a 3′OMe group on the ^{m7}G (first column); ^{m7}Gppp^{m6}A_{2′OMe}pG (Compound 31) was measured relative to ^{m7}G_{3′OMe}ppp^{m6}A_{2′OMe}pG (Compound 30), which is ^{m7}Gppp^{m6}A_{2′OMe}pG (Compound 31) with a 3′OMe group on the ^{m7}G (second column); ^{m7}GpppA_{2′,4′-LNA}pG (Compound 1) was measured relative to ^{m7}G_{3′OMe}pppA_{2′,4′-LNA}pG (Compound 2), which is ^{m7}GpppA_{2′,4′-LNA}pG (Compound 1) with a 3′OMe group on the ^{m7}G (third column); and ^{m7}Gppp(diaminopurine)_{2′OMe}pG (Compound 7) was measured relative to ^{m7}G_{3′OMe}ppp(diaminopurine)_{2′OMe}pG (Compound 8), which is ^{m7}Gppp(diaminopurine)_{2′OMe}pG (Compound 7) with a 3′OMe group on the ^{m7}G (fourth column).

### DETAILED DESCRIPTION

Described herein are novel trinucleotide cap analogs and compositions, and methods of using the same. Also described herein is an RNA molecule comprising a 5′-cap, wherein the 5′-cap includes a trinucleotide cap analog as described herein. Methods of inducing a therapeutic effect in a subject are also described herein, the methods including a step of administering to the subject a trinucleotide cap analog or RNA molecule including the trinucleotide cap analog.

The following description recites various examples of the present methods. No particular example is intended to define the scope of the methods. Rather, these are non-limiting, exemplary methods. The description is to be read from the perspective of one of ordinary skill in the art; therefore, information well known to the skilled artisan is not necessarily included.

### I. Definitions

Unless defined otherwise, all terms used herein have the same meaning as are commonly understood by one of skill in the art to which this disclosure belongs. All patents, patent applications and publications referred to throughout the disclosure herein are incorporated by reference in their entirety.

As used in the specification and the appended claims, the singular forms “a,” “an,” and “the” include plural referents unless the context clearly dictates otherwise. For example, reference to “a transcript” or “the transcript” may include a plurality of transcripts.

The use of any and all examples or exemplary language (e.g., “such as”) provided herein, is intended merely to better illustrate the invention and does not pose a limitation on the scope of the invention unless otherwise claimed.

The terms “may,” “may be,” “can,” and “can be,” and related terms are intended to convey that the subject matter involved is optional (that is, the subject matter is present in some examples and is not present in other examples), not a reference to a capability of the subject matter or to a probability, unless the context clearly indicates otherwise.

The terms “optional” and “optionally” mean that the subsequently described event, circumstance, or material may or may not occur or be present, and that the description includes instances where the event, circumstance, or material occurs or is present as well as instances where it does not occur or is not present.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of” and “consisting of” those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

Ranges can be expressed herein as from one particular value, and/or to another particular value. When such a range is expressed, also specifically contemplated and considered disclosed is the range from the one particular value and/or to the other particular value unless the context specifically indicates otherwise. It should be understood that all of the individual values and sub-ranges of values contained within an explicitly disclosed range are also specifically contemplated and should be considered disclosed unless the context specifically indicates otherwise. Further, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e., a single number) can be selected as the quantity, value, or feature to which the range refers.

As used herein in connection with numerical values, the term “about” is intended to describe values either above or below the stated value in a range of approximately +/−10%; in other examples, the values may range in value either above or below the stated value in a range of approximately +/−5%; in other examples, the values may range in value either above or below the stated value in a range of approximately +/−2%; in other examples, the values may range in value either above or below the stated value in a range of approximately +/−1%.

As used herein, the term “cap analog” means a structural derivative of an RNA cap.

As used herein, the term “complement,” “complementary,” or “complementarity” refers to specific base pairing between nucleotides or nucleic acids. Complementary nucleotides are, generally, A and T (or A and U), and G and C. Complementarity, for example, between a capped oligonucleotide primer and a DNA template, may be “complete” or “total” where all of the nucleotide bases of two nucleic acid strands are matched according to recognized base pairing rules, it may be “partial” in which only some of the nucleotide bases of an initiating capped oligonucleotide primer and a DNA template are matched according to recognized base pairing rules, or it may be “absent” where none of the nucleotide bases of two nucleic acid strands are matched according to recognized base pairing rules. Complementarity can also be “substantial complementarity” where the nucleotide bases of two nucleic acids are matched according to recognized base pairing rules, but include one or more mismatches (e.g., 1, 2, 3, 4) from total complementarity.

As used herein, a “deoxyribonuclease (DNase)” is an enzyme that catalyzes the hydrolytic cleavage of phosphodiester linkages in the DNA backbone, thus degrading DNA.

As used herein, the term “hybridize” or “specifically hybridize” refers to a process where initiating trinucleotide primer anneals to a DNA template under appropriately stringent conditions during a transcription reaction. Hybridizations to DNA are conducted with an initiating capped oligonucleotide primer which, in certain embodiments, is 3-10 nucleotides in length including the 5′-5′ inverted cap structure. Nucleic acid hybridization techniques are well known in the art (e.g., Sambrook, et al., Molecular Cloning: A Laboratory Manual, Second Edition, Cold Spring Harbor Press, Plainview, N.Y. (1989); Ausubel, F. M., et al., Current Protocols in Molecular Biology, John Wiley & Sons, Secaucus, N.J. (1994)). As used herein, the term “impurities” refers to substances which differ from the chemical composition of the target material (e.g., mRNA transcripts). Impurities are also referred to as contaminants.

“Inorganic pyrophosphatase” refers to an enzyme that catalyzes the conversion of one ion of pyrophosphate to two phosphate ions, thus inhibiting aggregation and in some instances preventing interaction of pyrophosphate with magnesium ions during T7 transcription reactions.

As used herein, the term “internucleotide linkage” refers to the bond or bonds that connect two nucleosides of an oligonucleotide or nucleic acid and may be a natural phosphodiester linkage or modified linkage.

As used herein, the term “in vitro” refers to a process that takes place outside a living organism (e.g., a multi-cellular organism, such as a human or a non-human animal), for example, in a test tube, culture dish, or elsewhere outside a living organism.

As used herein, the term “in vivo” refers to events that occur within a living organism.

As used herein the term “in vivo assays” refer to methods used to detect and/or measure capacity of one or more of the compounds or molecules including the compounds (e.g., mRNA molecules in, for example, a therapeutic dose) to increase or decrease a property relative to a control (e.g., biomarker levels). Optionally, in vivo assays as described herein can be used to determine a subject's tolerability levels to a given compound or molecule. Exemplary measurements for assessing tolerability include one or more of body weight, organ weight, aspartate aminotransferase (AST) levels, alanine transaminase (ALT) levels, C-reactive protein (CRP) levels, procalcitonin (PCT) levels, interleukin-6 (IL-6) levels, erythrocyte sedimentation rate (ESR), serum amyloid A levels, and serum ferritin levels. Optionally, the in vivo assays can be characterized herein as a “standard in vivo assay” and can optionally be performed using the conditions outlined in Examples 2 and 3 of the present application.

As used herein, the term “label” or “detectable label” refers to any compound or combination of compounds that may be attached or otherwise associated with a molecule so that the molecule can be detected directly or indirectly by detecting the label. A detectable label can be a radioisotope (e.g., carbon, phosphorus, iodine, indium, sulfur, tritium etc.), a mass isotope (e.g., H², C¹³ or N¹⁵), a dye or fluorophore (e.g., cyanine, fluorescein or coumarin), a hapten (e.g., biotin) or any other agent that can be detected directly or indirectly.

As used herein, “locked nucleic acid” (LNA) means a ribonucleotide having a bridge between the 2′O and 4′C methylene bicyclonucleotide monomers. An LNA moiety can have the following structure:

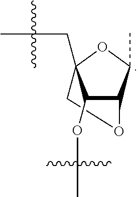

As used herein, “messenger RNA transcript,” or “mRNA transcript,” is a transcript transcribed from a DNA template encoding a desired polypeptide. The mRNA transcript may contain coding and non-coding regions. For example, the DNA template can comprise an RNA polymerase promoter sequence, a 5′ UTR sequence, an open reading frame, and a 3′ UTR sequence. In some examples, the DNA template also comprises a nucleic acid sequence encoding a poly(A) tail.

As used herein, the term “modified NTP” refers to a nucleoside 5′-triphosphate having a chemical moiety group bound at any position or substituted at any position, including the sugar, base, triphosphate chain, or any combination of these three locations.

Optionally, the chemical moiety group may be a group of any nature compatible with the process of transcription. Examples of such NTPs include inosine triphosphate, dihydrouridine triphosphate, 2′-fluoro-2′-deoxycytidine triphosphate, pseudouridine triphosphate, N1-methylpseudouridine triphosphate, and 5-methyluridine triphosphate, and can be found, for example in “Nucleoside Triphosphates and Their Analogs: Chemistry, Biotechnology and Biological Applications,” Vaghefi, M., ed., Taylor and Francis, Boca Raton (2005).

As used herein, the term “modified oligonucleotide” or “modified trinucleotide” includes, for example, an oligonucleotide containing a modified nucleoside, a modified internucleotide linkage, or having any combination of modified nucleosides and internucleotide linkages. Examples of internucleotide linkage modifications include phosphorothioate, phosphotriester and methylphosphonate derivatives (Stec, W. J., et al., Chem. Int. Ed. Engl., 33:709-722 (1994); Lebedev, A. V., et al., E., Perspect. Drug Discov. Des., 4:17-40 (1996); and Zon, et al., U.S. Patent Application No. 20070281308). Other examples of internucleotide linkage modifications may be found in Waldner, et al., Bioorg. Med. Chem. Letters 6:2363-2366 (1996).

As used herein, the term “nucleoside” refers to a nitrogenous base linked to a 5-carbon sugar (e.g., ribose or deoxyribose). The term includes all nucleosides, including all forms of nucleoside bases and furanoses. Base rings include purine and pyrimidine rings. Purine rings include, for example, adenine (also referred to herein as “A”), guanine (also referred to herein as “G”), and N₆-methyladenine (also referred to herein as “^{m6}A”). Pyrimidine rings include, for example, cytosine (also referred to herein as “C”), thymine (also referred to herein as “T”), 5-methylcytosine (also referred to herein as “^{m5}C”), and pseudouracil (also referred to herein as “ψ”). Other nucleosides include, but are not limited to, ribo, 2′-O-methyl or 2′-deoxyribo derivatives of adenosine, guanosine, cytidine, thymidine, uridine, inosine, 7-methylguanosine or pseudouridine.

As used herein, the terms “nucleoside analogs,” “modified nucleosides,” or “nucleoside derivatives” include synthetic nucleosides as described herein. Nucleoside derivatives also include nucleosides having modified base or/and sugar moieties, with or without protecting groups and include, for example, 2′-deoxy-2′-fluorouridine, 5-fluorouridine and the like. The compounds and methods provided herein include such base rings and synthetic analogs thereof, as well as unnatural heterocycle-substituted base sugars, and acyclic substituted base sugars. Other nucleoside derivatives that may be utilized with the present disclosure include, for example, LNA nucleosides, halogen-substituted purines (e.g., 6-fluoropurine), halogen-substituted pyrimidines, N⁶-ethyladenine, N⁴-(alkyl)-cytosines, 5-ethylcytosine, and the like (U.S. Pat. No. 6,762,298).

As used herein, the term “nucleoside triphosphate,” “nucleoside 5′ triphosphate” or “NTP” refers to a nucleoside linked to three phosphate groups. The term encompasses natural NTPs (for example, adenosine triphosphate (ATP), uridine triphosphate (UTP), guanine triphosphate (GTP), and cytosine triphosphate (CTP)) as well as modified NTPs.

As used herein, “oligo dT purification” is an affinity chromatography method for purification of mRNA comprising or including a poly-A tail.

As used herein, “phosphorothioate linkage” refers to a linkage between nucleosides in which the phosphorodiester linkage is modified by replacing one of the oxygen atoms, connected to a phosphorus atom, with a sulfur atom.

A “primary RNA” or “primary RNA transcript” means the RNA molecule that is newly synthesized by an RNA polymerase in vitro and which RNA molecule has a triphosphate on the 5′-carbon of its most 5′ nucleotide.

As used herein, the term “prematurely aborted RNA transcript” refers to incomplete products of an in vitro transcription reaction. Prematurely aborted RNA sequences may be any length that is less than the intended length of the desired transcriptional product.

The term “promoter” as used herein refers to a nucleotide sequence in a DNA template that directs and controls the initiation of transcription of a particular DNA sequence. Promoters are typically immediately adjacent to (or partially overlap with) the DNA sequence to be transcribed. Promoter sequences are typically located directly upstream or at the 5′ end of the transcription initiation site. Nucleotide positions in the promoter are designated relative to the transcriptional start site, where transcription of DNA begins (position+1).

As used herein, the term “purified” or “purify” refers to separating a substance from at least some of the components (e.g., impurities or contaminants) with which it was associated when initially produced. For example, RNA transcripts are purified by removal of contaminating proteins or other undesired nucleic acid species (e.g., double-stranded RNA, DNA, and/or incomplete or aborted RNA transcripts). Purified substances (e.g., capped mRNA transcripts) can be separated from 10%, 20%, 30%, 40%, 50%, 60%, 70%, 80%, 90%, 91%, 92%, 93%, 94%, 95%, 96%, 97%, 98%, 99%, or more than 99% of the other components with which they were initially associated.

As used herein, the term “RNase inhibitor” or “ribonuclease inhibitor” refers to a protein that inhibits RNAse activity for example, during an in vitro transcription reaction.

As used herein, the term “RNA polymerase” refers to an enzyme that synthesizes RNA using a DNA template. For in vitro transcription methods, single subunit phage RNA polymerases derived from T7, T3, SP6, K1-5, K1E, K1F or K11 bacteriophages, or variants thereof, are typically used. This family of polymerases has simple, minimal promoter sequences of about 17 nucleotides which require no accessory proteins and have minimal constraints of the initiating nucleotide sequence.

Salts of one or more compounds as described herein (e.g., primers) can be used in the disclosed methods. The term “salt(s),” as used herein, refers to derivatives of the compounds described herein prepared by the reaction of an acidic or basic moiety of the compound with a mineral or organic acid or base. Optionally, the salts can be pharmaceutically acceptable salts. As used herein, the term “pharmaceutically acceptable salt(s)” refers to those salts of the compounds described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. Salts may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1; and Remington: The Science and Practice of Pharmacy, 23d Edition, Adejare et al. eds., Academic Press (2020); which are incorporated herein by reference in their entireties.)

As used herein, “self-amplifying RNA,” or “saRNA,” is a linear, single-stranded RNA molecule that encodes the gene of interest. saRNA is a type of mRNA, but also includes non-structural proteins that encode a viral replicase. The viral replicase enables the RNA to self-replicate once delivered into the cell.

As used herein, the term “specific” when used in reference to an initiating trinucleotide primer sequence and its ability to hybridize to a DNA template is a sequence that has at least 50% sequence identity with a portion of the DNA template when the initiating trinucleotide primer and DNA strand are aligned. Higher levels of sequence identity include at least 66%, at least 75%, at least 80%, at least 85%, at least 90%, at least 95%, at least 99%, and optionally 100% sequence identity.

As used herein, the term “substantially free” refers to a state in which relatively little or no amount of an undesired substance (e.g., prematurely aborted RNA sequences, DNA, and/or double-stranded RNA) is present in a sample. “Substantially free of impurities” means impurities are present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample. For example, “substantially free of double-stranded RNA” means double-stranded RNA is present at a level less than approximately 5%, 4%, 3%, 2%, 1.0%, 0.9%, 0.8%, 0.7%, 0.6%, 0.5%, 0.4%, 0.3%, 0.2%, 0.1% or less (w/w) in a sample.

As used herein, “tangential flow filtration (TFF)” is a type of filtration wherein the material to be filtered is passed tangentially across a filter rather than through it. In TFF, undesired permeate passes through the filter, while the desired retentate passes along the filter and is collected downstream. In TFF, the desired material is typically contained in the retentate, which is the opposite of what is encountered when performing traditional membrane or dead-end filtration.

As used herein, the term “transcription” refers to enzymatically making or synthesizing RNA that is complementary to a DNA template, thereby producing a number of RNA copies of a DNA sequence. The RNA molecule synthesized in a transcription reaction is an “RNA transcript,” “primary transcript,” or “transcript.” Transcription reactions involving the compositions and methods provided herein employ initiating capped oligonucleotide primers described herein. Transcription of a DNA template may be exponential, nonlinear or linear. A DNA template may be a double-stranded linear DNA, a partially double-stranded linear DNA, circular double-stranded DNA, DNA plasmid, PCR amplified product, or a modified nucleic acid template that is compatible with RNA polymerase.

As used herein, the terms “universal base,” “degenerate base,” “universal base analog” and “degenerate base analog” include, for example, a nucleoside analog with an artificial base which is, in certain embodiments, recognizable by RNA polymerase as a substitute for one of the natural NTPs (e.g., ATP, UTP, CTP and GTP) or other specific NTP. Universal bases or degenerate bases are disclosed in Loakes, D., Nucleic Acids Res., 29:2437-2447 (2001); Crey-Desbiolles, C., et. al., Nucleic Acids Res., 33:1532-1543 (2005); Kincaid, K., et. al., Nucleic Acids Res., 33:2620-2628 (2005); Preparata, F P, Oliver, J S, J. Comput. Biol. 753-765 (2004); and Hill, F., et. al., Proc Natl Acad. Sci. USA, 95:4258-4263 (1998)).

As used herein, the term “unsubstituted” or “unmodified” in the context of the initiating capped oligonucleotide primer and NTPs refers to an initiating capped oligonucleotide primer and NTPs that have not been modified.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

As used herein, the terms “optional” or “optionally” means that the subsequently described event or circumstance can or cannot occur, and that the description includes instances where said event or circumstance occurs and instances where it does not.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the compounds, compositions, or methods disclosed herein.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene9-10 nonyl phenol, sodium deoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

As used herein, terms such as “elevated,” “increased,” “reduced,” and decreased” are generally considered relative to a control or normal state. However, when terms such as “reduce” or “decrease” are used herein relative to treatment, in which they refer to normalizing the level or amount toward the control or normal state and may include a partial or complete normalization.

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents, and are meant to include future updates.

As used herein, the terms “therapeutic agent” include any synthetic or naturally occurring biologically active compound or composition of matter which, when administered to an organism (human or nonhuman animal), induces a desired pharmacologic, immunogenic, and/or physiologic effect by local and/or systemic action. The term therefore encompasses those compounds or chemicals traditionally regarded as drugs, vaccines, and biopharmaceuticals including molecules such as proteins, peptides, hormones, nucleic acids, gene constructs and the like. Examples of therapeutic agents are described in well-known literature references such as the Merck Index (14^{th} edition), the Physicians' Desk Reference (64^{th} edition), and The Pharmacological Basis of Therapeutics (12^{th} edition), and they include, without limitation, medicaments; vitamins; mineral supplements; substances used for the treatment, prevention, diagnosis, cure or mitigation of a disease or illness; substances that affect the structure or function of the body, or pro-drugs, which become biologically active or more active after they have been placed in a physiological environment. For example, the term “therapeutic agent” includes compounds or compositions for use in all of the major therapeutic areas including, but not limited to, adjuvants; anti-infectives such as antibiotics and antiviral agents; analgesics and analgesic combinations, anorexics, anti-inflammatory agents, anti-epileptics, local and general anesthetics, hypnotics, sedatives, antipsychotic agents, neuroleptic agents, antidepressants, anxiolytics, antagonists, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics; antispasmodics, cardiovascular preparations (including calcium channel blockers, beta-blockers, beta-agonists and antiarrythmics), antihypertensives, diuretics, vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; proteins, peptides, and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like), small molecules (e.g., doxorubicin) and other biologically active macromolecules such as, for example, proteins and enzymes. The agent may be a biologically active agent used in medical, including veterinary, applications and in agriculture, such as with plants, as well as other areas. The term “therapeutic agent” also includes without limitation, medicaments; vitamins; mineral supplements; substances used for the treatment, prevention, diagnosis, cure or mitigation of disease or illness; or substances which affect the structure or function of the body; or pro-drugs, which become biologically active or more active after they have been placed in a predetermined physiological environment.

The term “pharmaceutically acceptable” describes a material that is not biologically or otherwise undesirable, i.e., without causing an unacceptable level of undesirable biological effects or interacting in a deleterious manner.

As used herein, the term “derivative” refers to a compound having a structure derived from the structure of a parent compound (e.g., a compound disclosed herein) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include salts, esters, amides, salts of esters or amides, and N-oxides of a parent compound.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “substituted” is contemplated to include all permissible substituents of organic compounds. In a broad aspect, the permissible substituents include acyclic and cyclic, branched and unbranched, carbocyclic and heterocyclic, and aromatic and nonaromatic substituents of organic compounds. Illustrative substituents include, for example, those described below. The permissible substituents can be one or more and the same or different for appropriate organic compounds. For purposes of this disclosure, the heteroatoms, such as nitrogen, can have hydrogen substituents and/or any permissible substituents of organic compounds described herein which satisfy the valences of the heteroatoms. This disclosure is not intended to be limited in any manner by the permissible substituents of organic compounds. Also, the terms “substitution” or “substituted with” include the implicit proviso that such substitution is in accordance with permitted valence of the substituted atom and the substituent, and that the substitution results in a stable compound, e.g., a compound that does not spontaneously undergo transformation such as by rearrangement, cyclization, elimination, etc. It is also contemplated that, in certain aspects, unless expressly indicated to the contrary, individual substituents can be further optionally substituted (i.e., further substituted or unsubstituted).

In defining various terms, “A¹,” “A²,” “A³,” and “A⁴” are used herein as generic symbols to represent various specific substituents. These symbols can be any substituent, not limited to those disclosed herein, and when they are defined to be certain substituents in one instance, they can, in another instance, be defined as some other substituents.

The term “aliphatic” or “aliphatic group,” as used herein, denotes a hydrocarbon moiety that may be straight-chain (i.e., unbranched), branched, or cyclic (including fused, bridging, and spirofused polycyclic) and may be completely saturated or may contain one or more units of unsaturation, but which is not aromatic. Unless otherwise specified, aliphatic groups contain 1-20 carbon atoms. Aliphatic groups include, but are not limited to, linear or branched, alkyl, alkenyl, and alkynyl groups, and hybrids thereof such as (cycloalkyl)alkyl, (cycloalkenyl)alkyl or (cycloalkyl)alkenyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, thio, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

This practice is also used for other groups described herein. That is, while a term such as “cycloalkyl” refers to both unsubstituted and substituted cycloalkyl moieties, the substituted moieties can, in addition, be specifically identified herein; for example, a particular substituted cycloalkyl can be referred to as, e.g., an “alkylcycloalkyl.” Similarly, a substituted alkoxy can be specifically referred to as, e.g., a “halogenated alkoxy,” a particular substituted alkenyl can be, e.g., an “alkenylalcohol,” and the like. Again, the practice of using a general term, such as “cycloalkyl,” and a specific term, such as “alkylcycloalkyl,” is not meant to imply that the general term does not also include the specific term.

The term “cycloalkyl” as used herein is a non-aromatic carbon-based ring composed of at least three carbon atoms. Examples of cycloalkyl groups include, but are not limited to, cyclopropyl, cyclobutyl, cyclopentyl, cyclohexyl, norbornyl, and the like. The term “heterocycloalkyl” is a type of cycloalkyl group as defined above, and is included within the meaning of the term “cycloalkyl,” where at least one of the carbon atoms of the ring is replaced with a heteroatom such as, but not limited to, nitrogen, oxygen, sulfur, or phosphorus. The cycloalkyl group and heterocycloalkyl group can be substituted or unsubstituted. The cycloalkyl group and heterocycloalkyl group can be substituted with one or more groups including, but not limited to, alkyl, cycloalkyl, alkoxy, amino, ether, halide, hydroxy, nitro, silyl, sulfo-oxo, thio, or thiol as described herein.

The terms “alkoxy” and “alkoxyl” as used herein to refer to an alkyl or cycloalkyl group bonded through an ether linkage; that is, an “alkoxy” group can be defined as —OA¹ where A¹ is alkyl or cycloalkyl as defined above. “Alkoxy” also includes polymers of alkoxy groups as just described; that is, an alkoxy can be a polyether such as —OA¹-OA² or —OA¹-(OA²)ₐ-OA³, where “a” is an integer of from 1 to 200 and A¹, A², and A³ are alkyl and/or cycloalkyl groups.

The term “alkenyl” as used herein is a hydrocarbon group of from 2 to 24 carbon atoms with a structural formula containing at least one carbon-carbon double bond. Asymmetric structures such as (A¹A²)C═C(A³A⁴) are intended to include both the E and Z isomers. This can be presumed in structural formulae herein wherein an asymmetric alkene is present, or it can be explicitly indicated by the bond symbol C═C. The alkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, thio, or thiol, as described herein.

The term “cycloalkenyl” as used herein is a non-aromatic carbon-based ring composed of at least three carbon atoms and containing at least one carbon-carbon double bound, i.e., C═C. Examples of cycloalkenyl groups include, but are not limited to, cyclopropenyl, cyclobutenyl, cyclopentenyl, cyclopentadienyl, cyclohexenyl, cyclohexadienyl, norbornenyl, and the like. The term “heterocycloalkenyl” is a type of cycloalkenyl group as defined above, and is included within the meaning of the term “cycloalkenyl,” where at least one of the carbon atoms of the ring is replaced with a heteroatom such as, but not limited to, nitrogen, oxygen, sulfur, or phosphorus. The cycloalkenyl group and heterocycloalkenyl group can be substituted or unsubstituted. The cycloalkenyl group and heterocycloalkenyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, thio, or thiol as described herein.

The term “alkynyl” as used herein is a hydrocarbon group of 2 to 24 carbon atoms with a structural formula containing at least one carbon-carbon triple bond. The alkynyl group can be unsubstituted or substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, thio, or thiol, as described herein.

The term “cycloalkynyl” as used herein is a non-aromatic carbon-based ring composed of at least seven carbon atoms and containing at least one carbon-carbon triple bound. Examples of cycloalkynyl groups include, but are not limited to, cycloheptynyl, cyclooctynyl, cyclononynyl, and the like. The term “heterocycloalkynyl” is a type of cycloalkenyl group as defined above, and is included within the meaning of the term “cycloalkynyl,” where at least one of the carbon atoms of the ring is replaced with a heteroatom such as, but not limited to, nitrogen, oxygen, sulfur, or phosphorus. The cycloalkynyl group and heterocycloalkynyl group can be substituted or unsubstituted. The cycloalkynyl group and heterocycloalkynyl group can be substituted with one or more groups including, but not limited to, alkyl, cycloalkyl, alkoxy, alkenyl, cycloalkenyl, alkynyl, cycloalkynyl, aryl, heteroaryl, aldehyde, amino, carboxylic acid, ester, ether, halide, hydroxy, ketone, azide, nitro, silyl, sulfo-oxo, thio, or thiol as described herein.

The term “aryl” as used herein is a group that contains any carbon-based aromatic group including, but not limited to, benzene, naphthalene, phenyl, biphenyl, anthracene, and the like. The aryl group can be substituted or unsubstituted. The aryl group can be substituted with one or more groups including, but not limited to, alkyl, cycloalkyl, alkoxy, alkenyl, cycloalkenyl, alkynyl, cycloalkynyl, aryl, heteroaryl, aldehyde, —NH₂, carboxylic acid, ester, ether, halide, hydroxy, ketone, azide, nitro, silyl, sulfo-oxo, thio, or thiol as described herein. The term “biaryl” is a specific type of aryl group and is included in the definition of “aryl.” In addition, the aryl group can be a single ring structure or comprise multiple ring structures that are either fused ring structures or attached via one or more bridging groups such as a carbon-carbon bond. For example, biaryl can be two aryl groups that are bound together via a fused ring structure, as in naphthalene, or are attached via one or more carbon-carbon bonds, as in biphenyl.

The term “aldehyde” as used herein is represented by the formula —C(O)H. Throughout this specification “C(O)” is a short hand notation for a carbonyl group, i.e., C═O.

The terms “amine” or “amino” as used herein are represented by the formula —NA¹A², where A¹ and A² can be, independently, hydrogen or alkyl, cycloalkyl, alkenyl, cycloalkenyl, alkynyl, cycloalkynyl, aryl, or heteroaryl group as described herein. A specific example of amino is —NH₂.

The term “alkylamino” as used herein is represented by the formula —NH(-alkyl) where alkyl is a described herein. Representative examples include, but are not limited to, methylamino group, ethylamino group, propylamino group, isopropylamino group, butylamino group, isobutylamino group, (sec-butyl)amino group, (tert-butyl)amino group, pentylamino group, isopentylamino group, (tert-pentyl)amino group, hexylamino group, and the like.

The term “dialkylamino” as used herein is represented by the formula —N(-alkyl)₂ where alkyl is a described herein. Representative examples include, but are not limited to, dimethylamino group, diethylamino group, dipropylamino group, diisopropylamino group, dibutylamino group, diisobutylamino group, di(sec-butyl)amino group, di(tert-butyl)amino group, dipentylamino group, diisopentylamino group, di(tert-pentyl)amino group, dihexylamino group, N-ethyl-N-methylamino group, N-methyl-N-propylamino group, N-ethyl-N-propylamino group and the like.

The term “carboxylic acid” as used herein is represented by the formula —C(O)OH.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group as described herein. The term “polyester” as used herein is represented by the formula -(A¹O(O)C-A²-C(O)O)ₐ— or -(A¹O(O)C-A²-OC(O))ₐ—, where A¹ and A² can be, independently, an alkyl, cycloalkyl, alkenyl, cycloalkenyl, alkynyl, cycloalkynyl, aryl, or heteroaryl group described herein and “a” is an integer from 1 to 500. “Polyester” is as the term used to describe a group that is produced by the reaction between a compound having at least two carboxylic acid groups with a compound having at least two hydroxyl groups.

The term “ether” as used herein is represented by the formula A¹OA², where A¹ and A² can be, independently, an alkyl, cycloalkyl, alkenyl, cycloalkenyl, alkynyl, cycloalkynyl, aryl, or heteroaryl group described herein. The term “polyether” as used herein is represented by the formula -(A¹O-A²O)ₐ—, where A¹ and A² can be, independently, an alkyl, cycloalkyl, alkenyl, cycloalkenyl, alkynyl, cycloalkynyl, aryl, or heteroaryl group described herein and “a” is an integer of from 1 to 500. Examples of polyether groups include polyethylene oxide, polypropylene oxide, and polybutylene oxide.

The terms “halo,” “halogen,” or “halide,” as used herein can be used interchangeably and refer to F, Cl, Br, or I.

The term “heteroalkyl,” as used herein refers to an alkyl group containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. Heteroalkyls can be substituted as defined above for alkyl groups.

The term “heteroaryl,” as used herein refers to an aromatic group that has at least one heteroatom incorporated within the ring of the aromatic group. Examples of heteroatoms include, but are not limited to, nitrogen, oxygen, sulfur, and phosphorus, where N-oxides, sulfur oxides, and dioxides are permissible heteroatom substitutions. The heteroaryl group can be substituted or unsubstituted. The heteroaryl group can be substituted with one or more groups including, but not limited to, alkyl, cycloalkyl, alkoxy, amino, ether, halide, hydroxy, nitro, silyl, sulfo-oxo, thio, or thiol as described herein. Heteroaryl groups can be monocyclic, or alternatively fused ring systems. Heteroaryl groups include, but are not limited to, furyl, imidazolyl, pyrimidinyl, tetrazolyl, thienyl, pyridinyl, pyrrolyl, N-methylpyrrolyl, quinolinyl, isoquinolinyl, pyrazolyl, triazolyl, thiazolyl, oxazolyl, isoxazolyl, oxadiazolyl, thiadiazolyl, isothiazolyl, pyridazinyl, pyrazinyl, benzofuranyl, benzodioxolyl, benzothiophenyl, indolyl, indazolyl, benzimidazolyl, imidazopyridinyl, pyrazolopyridinyl, and pyrazolopyrimidinyl. Further not limiting examples of heteroaryl groups include, but are not limited to, pyridinyl, pyridazinyl, pyrimidinyl, pyrazinyl, thiophenyl, pyrazolyl, imidazolyl, benzo[d]oxazolyl, benzo[d]thiazolyl, quinolinyl, quinazolinyl, indazolyl, imidazo[1,2-b]pyridazinyl, imidazo[1,2-a]pyrazinyl, benzo[c][1,2,5]thiadiazolyl, benzo[c][1,2,5]oxadiazolyl, and pyrido[2,3-b]pyrazinyl.

The terms “heterocycle” or “heterocyclyl,” as used herein can be used interchangeably and refer to single and multi-cyclic aromatic or non-aromatic ring systems in which at least one of the ring members is other than carbon. Thus, the term is inclusive of, but not limited to, “heterocycloalkyl,” “heteroaryl,” “bicyclic heterocycle,” and “polycyclic heterocycle.” Heterocycle includes pyridine, pyrimidine, furan, thiophene, pyrrole, isoxazole, isothiazole, pyrazole, oxazole, thiazole, imidazole, oxazole, including, 1,2,3-oxadiazole, 1,2,5-oxadiazole and 1,3,4-oxadiazole, thiadiazole, including, 1,2,3-thiadiazole, 1,2,5-thiadiazole, and 1,3,4-thiadiazole, triazole, including, 1,2,3-triazole, 1,3,4-triazole, tetrazole, including 1,2,3,4-tetrazole and 1,2,4,5-tetrazole, pyridazine, pyrazine, triazine, including 1,2,4-triazine and 1,3,5-triazine, tetrazine, including 1,2,4,5-tetrazine, pyrrolidine, piperidine, piperazine, morpholine, azetidine, tetrahydropyran, tetrahydrofuran, dioxane, and the like. The term heterocyclyl group can also be a C2 heterocyclyl, C2-C3 heterocyclyl, C2-C4 heterocyclyl, C2-C5 heterocyclyl, C2-C6 heterocyclyl, C2-C7 heterocyclyl, C2-C8 heterocyclyl, C2-C9 heterocyclyl, C2-C10 heterocyclyl, C2-C11 heterocyclyl, and the like up to and including a C2-C18 heterocyclyl. For example, a C2 heterocyclyl comprises a group which has two carbon atoms and at least one heteroatom, including, but not limited to, aziridinyl, diazetidinyl, dihydrodiazetyl, oxiranyl, thiiranyl, and the like. Alternatively, for example, a C5 heterocyclyl comprises a group which has five carbon atoms and at least one heteroatom, including, but not limited to, piperidinyl, tetrahydropyranyl, tetrahydrothiopyranyl, diazepanyl, pyridinyl, and the like. It is understood that a heterocyclyl group may be bound either through a heteroatom in the ring, where chemically possible, or one of carbons comprising the heterocyclyl ring.

The term “bicyclic heterocycle” or “bicyclic heterocyclyl,” as used herein refers to a ring system in which at least one of the ring members is other than carbon. Bicyclic heterocyclyl encompasses ring systems wherein an aromatic ring is fused with another aromatic ring, or wherein an aromatic ring is fused with a non-aromatic ring. Bicyclic heterocyclyl encompasses ring systems wherein a benzene ring is fused to a 5- or a 6-membered ring containing 1, 2 or 3 ring heteroatoms or wherein a pyridine ring is fused to a 5- or a 6-membered ring containing 1, 2 or 3 ring heteroatoms. Bicyclic heterocyclic groups include, but are not limited to, indolyl, indazolyl, pyrazolo[1,5-a]pyridinyl, benzofuranyl, quinolinyl, quinoxalinyl, 1,3-benzodioxolyl, 2,3-dihydro-1,4-benzodioxinyl, 3,4-dihydro-2H-chromenyl, 1H-pyrazolo[4,3-c]pyridin-3-yl; 1H-pyrrolo[3,2-b]pyridin-3-yl; and 1H-pyrazolo[3,2-b]pyridin-3-yl.

The term “heterocycloalkyl” as used herein refers to an aliphatic, partially unsaturated or fully saturated, 3- to 14-membered ring system, including single rings of 3 to 8 atoms and bi- and tricyclic ring systems. The heterocycloalkyl ring-systems include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited to, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “hydroxyl” or “hydroxyl” as used herein is represented by the formula —OH.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group as described herein.

The term “azide” or “azido” as used herein is represented by the formula —N₃.

The term “nitro” as used herein is represented by the formula —NO₂.

The term “nitrile” or “cyano” as used herein is represented by the formula —CN.

The term “silyl” as used herein is represented by the formula —SiA¹A²A³, where A¹, A², and A³ can be, independently, hydrogen or an alkyl, cycloalkyl, alkoxy, alkenyl, cycloalkenyl, alkynyl, cycloalkynyl, aryl, or heteroaryl group as described herein.

The term “sulfo-oxo” as used herein is represented by the formulas —S(O)A¹, —S(O)₂A¹, —OS(O)₂A¹, or —OS(O)₂OA¹, where A¹ can be hydrogen or an alkyl, cycloalkyl, alkenyl, cycloalkenyl, alkynyl, cycloalkynyl, aryl, or heteroaryl group as described herein. Throughout this specification “S(O)” is a short hand notation for S═O. The term “sulfonyl” is used herein to refer to the sulfo-oxo group represented by the formula —S(O)₂A¹, where A¹ can be hydrogen or an alkyl, cycloalkyl, alkenyl, cycloalkenyl, alkynyl, cycloalkynyl, aryl, or heteroaryl group as described herein. The term “sulfone” as used herein is represented by the formula A¹S(O)₂A², where A¹ and A² can be, independently, an alkyl, cycloalkyl, alkenyl, cycloalkenyl, alkynyl, cycloalkynyl, aryl, or heteroaryl group as described herein. The term “sulfoxide” as used herein is represented by the formula A¹S(O)A², where A¹ and A² can be, independently, an alkyl, cycloalkyl, alkenyl, cycloalkenyl, alkynyl, cycloalkynyl, aryl, or heteroaryl group as described herein.

The term “thio” as used herein is represented by the formula —SA¹, where A¹ can be an alkyl, cycloalkyl, alkenyl, cycloalkenyl, alkynyl, cycloalkynyl, aryl, or heteroaryl group as described herein.

The term “thiol” as used herein is represented by the formula —SH.

“R¹,” “R²,” “R³,” “Rⁿ,” where n is an integer, as used herein can, independently, possess one or more of the groups listed above. For example, if R¹ is a straight chain alkyl group, one of the hydrogen atoms of the alkyl group can optionally be substituted with a hydroxyl group, an alkoxy group, an alkyl group, a halide, and the like. Depending upon the groups that are selected, a first group can be incorporated within second group or, alternatively, the first group can be pendant (i.e., attached) to the second group. For example, with the phrase “an alkyl group comprising an amino group,” the amino group can be incorporated within the backbone of the alkyl group. Alternatively, the amino group can be attached to the backbone of the alkyl group. The nature of the group(s) that is (are) selected will determine if the first group is embedded or attached to the second group.

As described herein, compounds of the disclosure may contain “optionally substituted” moieties. In general, the term “substituted,” whether preceded by the term “optionally” or not, means that one or more hydrogen of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. In is also contemplated that, in certain aspects, unless expressly indicated to the contrary, individual substituents can be further optionally substituted (i.e., further substituted or unsubstituted).

The term “stable,” as used herein, refers to compounds that are not substantially altered when subjected to conditions to allow for their production, detection, and, in certain aspects, their recovery, purification, and use for one or more of the purposes disclosed herein.

The term “leaving group” refers to an atom (or a group of atoms) with electron withdrawing ability that can be displaced as a stable species, taking with it the bonding electrons. Examples of suitable leaving groups include halides and sulfonate esters, including, but not limited to, triflate, mesylate, tosylate, and brosylate.

The terms “hydrolysable group” and “hydrolysable moiety” refer to a functional group capable of undergoing hydrolysis, e.g., under basic or acidic conditions. Examples of hydrolysable residues include, without limitation, acid halides, activated carboxylic acids, and various protecting groups known in the art (see, for example, “Greene's Protective Groups in Organic Synthesis,” P. G. M. Wuts, Wiley, 2014).

Compounds described herein can contain one or more double bonds and, thus, potentially give rise to cis/trans (E/Z) isomers, as well as other conformational isomers. Unless stated to the contrary, the compounds described herein include all such possible isomers, as well as mixtures of such isomers.

Unless stated to the contrary, a formula with chemical bonds shown only as solid lines and not as wedges or dashed lines contemplates each possible isomer, e.g., each enantiomer and diastereomer, and a mixture of isomers, such as a racemic or scalemic mixture. Compounds described herein can contain one or more asymmetric centers and, thus, potentially give rise to diastereomers and optical isomers. Unless stated to the contrary, the present disclosure includes all such possible diastereomers as well as their racemic mixtures, their substantially pure resolved enantiomers, all possible geometric isomers, and pharmaceutically acceptable salts thereof. Mixtures of stereoisomers, as well as isolated specific stereoisomers, are also included. During the course of the synthetic procedures used to prepare such compounds, or in using racemization or epimerization procedures known to those skilled in the art, the products of such procedures can be a mixture of stereoisomers.

Many organic compounds exist in optically active forms having the ability to rotate the plane of plane-polarized light. In describing an optically active compound, the prefixes D and L or R and S are used to denote the absolute configuration of the molecule about its chiral center(s). The prefixes d and 1 or (+) and (−) are employed to designate the sign of rotation of plane-polarized light by the compound, with (−) or meaning that the compound is levorotatory. A compound prefixed with (+) or d is dextrorotatory. For a given chemical structure, these compounds, called stereoisomers, are identical except that they are non-superimposable mirror images of one another. A specific stereoisomer can also be referred to as an enantiomer, and a mixture of such isomers is often called an enantiomeric mixture. A 50:50 mixture of enantiomers is referred to as a racemic mixture. Many of the compounds described herein can have one or more chiral centers and therefore can exist in different enantiomeric forms. When bonds to the chiral atom are depicted as straight lines in the disclosed formulas, it is understood that both the (R) and (S) configurations of the chiral atom, and hence both enantiomers and mixtures thereof, are embraced within the formula. As is used in the art, when it is desired to specify the absolute configuration about a chiral atom, one of the bonds to the chiral atom can be depicted as a wedge (bonds to atoms above the plane) and the other can be depicted as a series or wedge of short parallel lines is (bonds to atoms below the plane). The Cahn-Ingold-Prelog system can be used to assign the (R) or (S) configuration to a chiral atom.

When the disclosed compounds contain one chiral center, the compounds exist in two enantiomeric forms. Unless specifically stated to the contrary, a disclosed compound includes both enantiomers and mixtures of enantiomers, such as the specific 50:50 mixture referred to as a racemic mixture. The enantiomers can be resolved by methods known to those skilled in the art, such as formation of diastereoisomeric salts which may be separated, for example, by crystallization (see, CRC Handbook of Optical Resolutions via Diastereomeric Salt Formation by David Kozma (CRC Press, 2001)); formation of diastereoisomeric derivatives or complexes which may be separated, for example, by crystallization, gas-liquid or liquid chromatography; selective reaction of one enantiomer with an enantiomer-specific reagent, for example enzymatic esterification; or gas-liquid or liquid chromatography in a chiral environment, for example on a chiral support for example silica with a bound chiral ligand or in the presence of a chiral solvent. It will be appreciated that where the desired enantiomer is converted into another chemical entity by one of the separation procedures described above, a further step can liberate the desired enantiomeric form. Alternatively, specific enantiomers can be synthesized by asymmetric synthesis using optically active reagents, substrates, catalysts or solvents, or by converting one enantiomer into the other by asymmetric transformation.

Designation of a specific absolute configuration at a chiral atom in a disclosed compound is understood to mean that the designated enantiomeric form of the compounds can be provided in enantiomeric excess (e.e.). Enantiomeric excess, as used herein, is the presence of a particular enantiomer at greater than 50%, for example, greater than 60%, greater than 70%, greater than 75%, greater than 80%, greater than 85%, greater than 90%, greater than 95%, greater than 98%, or greater than 99%. In one aspect, the designated enantiomer is substantially free from the other enantiomer. For example, the “R” forms of the compounds can be substantially free from the “S” forms of the compounds and are, thus, in enantiomeric excess of the “S” forms. Conversely, “S” forms of the compounds can be substantially free of “R” forms of the compounds and are, thus, in enantiomeric excess of the “R” forms.

When a disclosed compound has two or more chiral atoms, it can have more than two optical isomers and can exist in diastereoisomeric forms. For example, when there are two chiral atoms, the compound can have up to four optical isomers and two pairs of enantiomers ((S,S)/(R,R) and (R,S)/(S,R)). The pairs of enantiomers (e.g., (S,S)/(R,R)) are mirror image stereoisomers of one another. The stereoisomers that are not mirror-images (e.g., (S,S) and (R,S)) are diastereomers. The diastereoisomeric pairs can be separated by methods known to those skilled in the art, for example chromatography or crystallization and the individual enantiomers within each pair may be separated as described above. Unless otherwise specifically excluded, a disclosed compound includes each diastereoisomer of such compounds and mixtures thereof.

The compounds according to this disclosure may form prodrugs at hydroxyl or amino functionalities using alkoxy, amino acids, etc., groups as the prodrug forming moieties. For instance, the hydroxymethyl position may form mono-, di- or triphosphates and again these phosphates can form prodrugs. Preparations of such prodrug derivatives are discussed in various literature sources (examples are: Alexander et al., J. Med. Chem. 1988, 31, 318; Aligas-Martin et al., PCT WO 2000/041531, p. 30). The nitrogen function converted in preparing these derivatives is one (or more) of the nitrogen atoms of a compound of the disclosure.

“Derivatives” of the compounds disclosed herein are pharmaceutically acceptable salts, prodrugs, deuterated forms, radio-actively labeled forms, isomers, solvates and combinations thereof. The “combinations” mentioned in this context are refer to derivatives falling within at least two of the groups: pharmaceutically acceptable salts, prodrugs, deuterated forms, radio-actively labeled forms, isomers, and solvates. Examples of radio-actively labeled forms include compounds labeled with tritium, phosphorous-32, iodine-129, carbon-11, fluorine-18, and the like.

Compounds described herein comprise atoms in both their natural isotopic abundance and in non-natural abundance. The disclosed compounds can be isotopically-labeled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the disclosure include isotopes of hydrogen, carbon, nitrogen, oxygen, phosphorous, fluorine and chlorine, such as ²H, ³H, ³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this disclosure. Certain isotopically-labeled compounds of the present disclosure, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of the present disclosure and prodrugs thereof can generally be prepared by carrying out the procedures below, by substituting a readily available isotopically labeled reagent for a non-isotopically labeled reagent.

The compounds described herein can be present as a solvate. In some cases, the solvent used to prepare the solvate is an aqueous solution, and the solvate is then often referred to as a hydrate. The compounds can be present as a hydrate, which can be obtained, for example, by crystallization from a solvent or from aqueous solution. In this connection, one, two, three or any arbitrary number of solvent or water molecules can combine with the compounds according to the disclosure to form solvates and hydrates.

It is also appreciated that certain compounds described herein can be present as an equilibrium of tautomers. For example, ketones with an α-hydrogen can exist in an equilibrium of the keto form and the enol form.

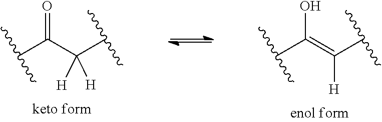 [SMILES: *CC(*)=O ; */C=C(/*)O]

Likewise, amides with an N-hydrogen can exist in an equilibrium of the amide form and the imidic acid form.

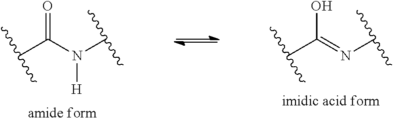 [SMILES: *NC(*)=O ; */N=C(/*)O]

As another example, pyrazoles can exist in two tautomeric forms, N¹-unsubstituted, 3-A³ and N¹-unsubstituted, 5-A³ as shown below.

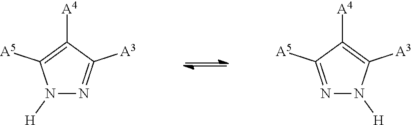

Unless stated to the contrary, the disclosure includes all such possible tautomers.

It is known that chemical substances form solids which are present in different states of order which are termed polymorphic forms or modifications. The different modifications of a polymorphic substance can differ greatly in their physical properties. The compounds according to the disclosure can be present in different polymorphic forms, with it being possible for particular modifications to be metastable. Unless stated to the contrary, the disclosure includes all such possible polymorphic forms.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Strem Chemicals (Newburyport, MA), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and supplemental volumes (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

### II. Compounds

A class of compounds described herein is represented by Formula I:

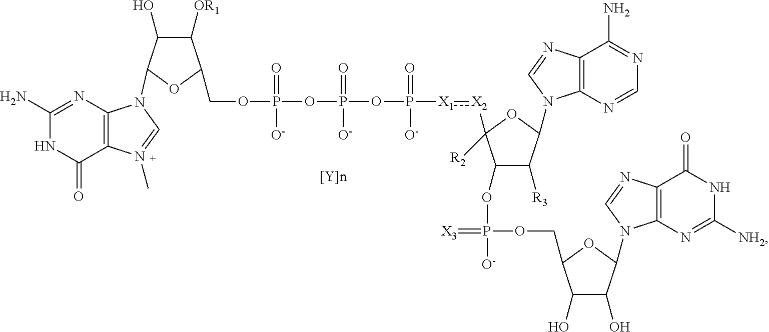

or any stereoisomer thereof.

In Formula I, [symbol] is a single bond or a double bond.

Also in Formula I, R₁ is H or CH₃.

Additionally in Formula I, R₂ is H and R₃ is OCH₃ or F, or R₂ and R₃ are covalently bonded together and, together with intermediate atoms, form a 2′-O, 4′-C methylene bridge.

Further in Formula I, X₁ is O or CH.

Also in Formula I, X₂ is CH₂ or CH.

Additionally in Formula I, X₃ is O or S.

Further in Formula I, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

In Formula I, when X₁ is CH, then X₂ is CH and [symbol] is a double bond.

As understood by those of skill in the art, the structures shown of the compounds described herein are representations of one form of the compound. Although such compounds may be drawn or described in protonated (free acid) form, in ionized (anionic) form, or ionized and in association with one or more cations (salt form), aqueous solutions of such compounds exist in equilibrium among such forms. For example, a phosphate linkage of a compound described herein, in aqueous solution, exists in equilibrium among free acid, anion, and salt forms.

Optionally, Formula I can be depicted in protonated (free acid) form as shown below:

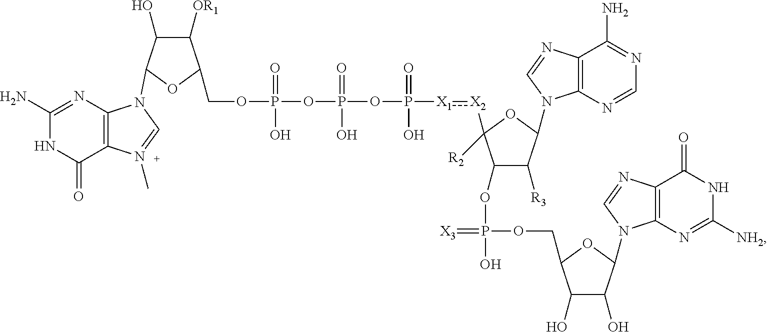

wherein
[symbol] , R₁, R₂, R₃, X₁, X₂, and X₃ are as defined above for Formula I.

Optionally, Formula I can be depicted in ionized (anionic) form as shown below:

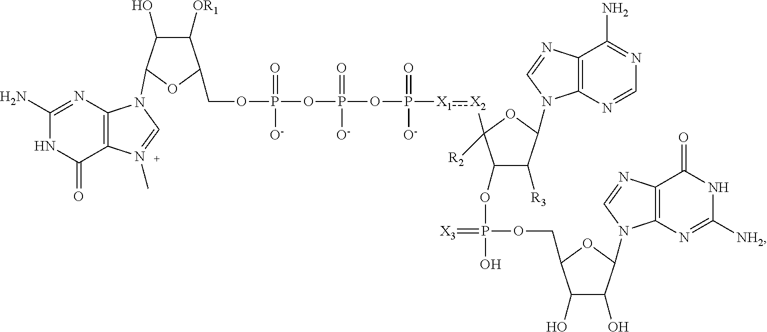

wherein
[symbol] , R₁, R₂, R₃, X₁, X₂, and X₃ are as defined above for Formula I.

Optionally, Formula I can be depicted in ionized and in association with one or more cations (salt) form as shown below, in which compounds of the structure are in association with sodium cations (for representation purposes only and not by way of limitation):

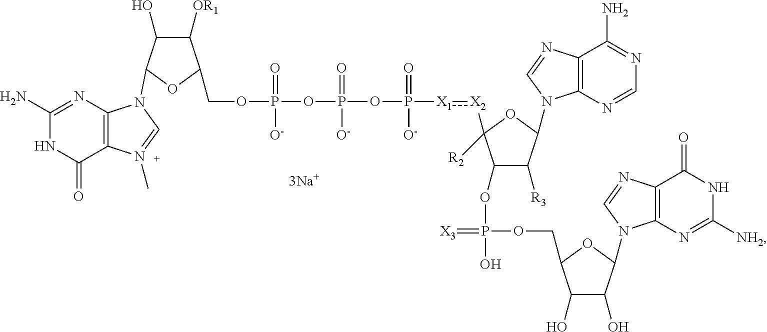

wherein
[symbol] , R₁, R₂, R₃, X₁, X₂, and X₃ are as defined above for Formula I. As understood by those of skill in the art, the cation salt form may exist in which one, two, three, or four cations are present.

Unless otherwise indicated, compounds described herein are intended to include all such forms. Moreover, certain compounds have several such linkages, each of which is in equilibrium. Thus, compounds in solution exist in an ensemble of forms at multiple positions all at equilibrium. Drawn structures necessarily depict a single form. Nevertheless, unless otherwise indicated, such drawings are likewise intended to include corresponding forms. Herein, a structure depicting the free acid of a compound followed by the term “or salts thereof” expressly includes all such forms that may be fully or partially protonated/de-protonated/in association with a cation. In certain instances, one or more specific cation is identified. In certain methods, compounds disclosed herein are in aqueous solution with sodium. In certain methods, compounds are in aqueous solution with potassium. In certain methods, compounds are in aqueous solution with lithium. In certain methods, compounds are in aqueous solution with triethylammonium.

As described above, Formula I can include compounds in which R₂ and R₃ are covalently bonded together and, together with intermediate atoms, form a 2′-O, 4′-C methylene bridge. The 2′-O, 4′-C methylene bridge moiety can also be referred to herein as a linked nucleic acid (LNA) moiety. Optionally, the compounds of Formula I having an LNA moiety are represented by Structure I-A as shown below, or a salt thereof:

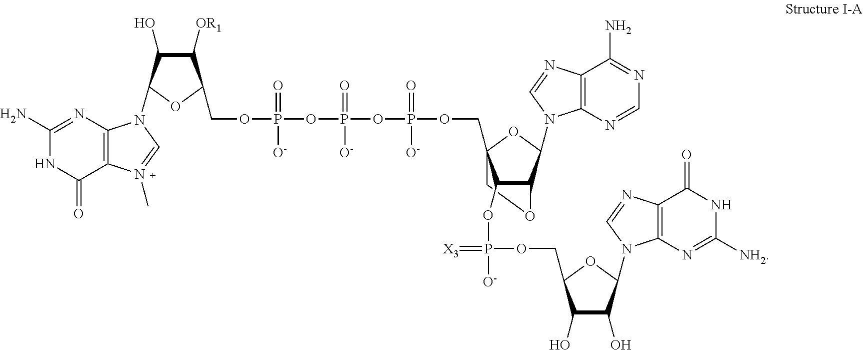

In Structure I-A, R₁ and X₃ are as defined above for Formula I. Examples of Structure I-A include the following compounds and salts thereof:

^{m7}GpppA_{2′,4′-LNA}pG:

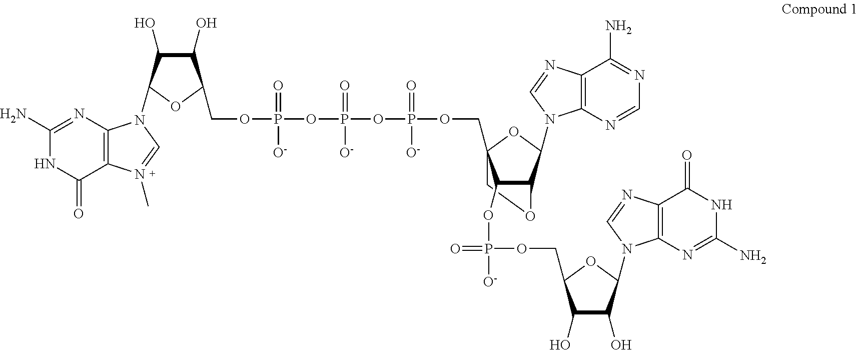

^{m7}G_{3′OMe}pppA_{2′,4′-LNA}pG:

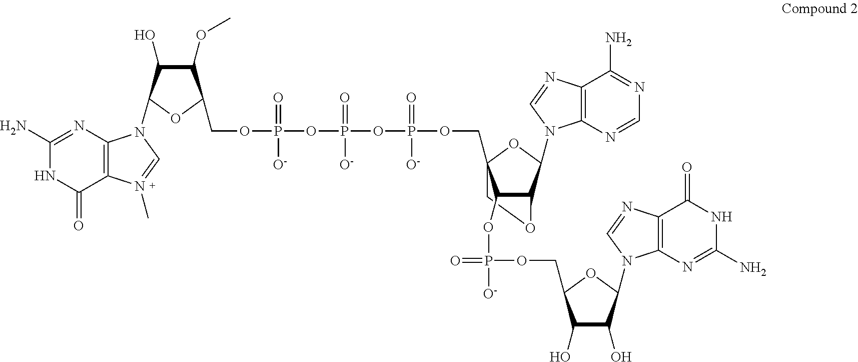

^{m7}GpppA_{2′-4′-LNA}p(s)G:

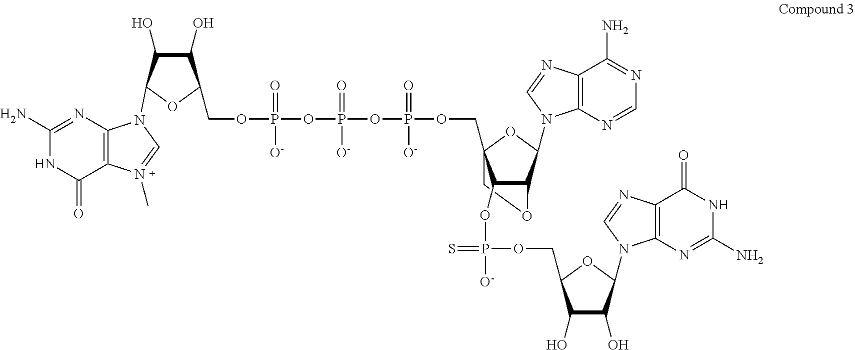

As described above, Formula I can include compounds in which X₁ is CH. In these examples, X₂ is also CH, such that X₁ and X₂ form a vinyl moiety (—CH═CH—). Optionally, the compounds of Formula I having a vinyl moiety are represented by Structure I-B as shown below, or a salt thereof:

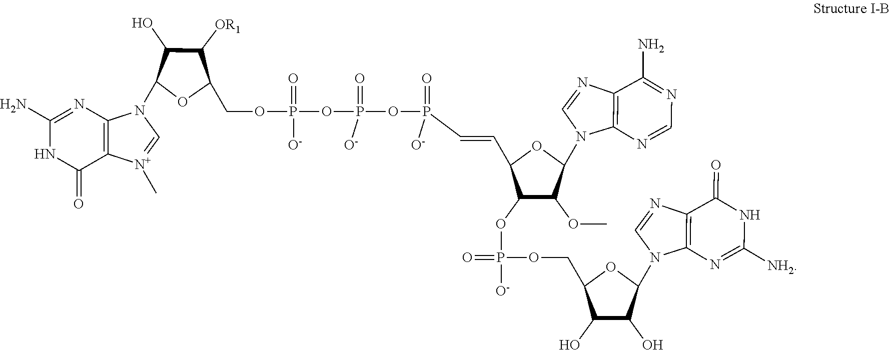

In Structure I-B, R₁ is as defined above for Formula I. Examples of Structure I-B include the following compounds and salts thereof:

^{m7}GpppA_{2′OMe-5′vinyl}pG:

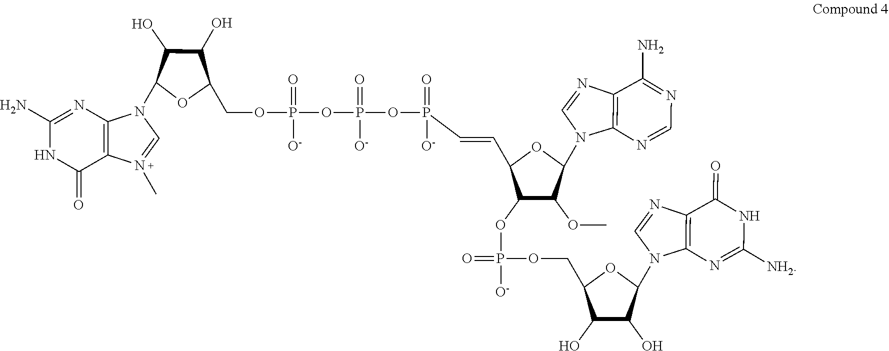

^{m7}G_{3′OMe}pppA_{2′OMe-5′vinyl}pG:

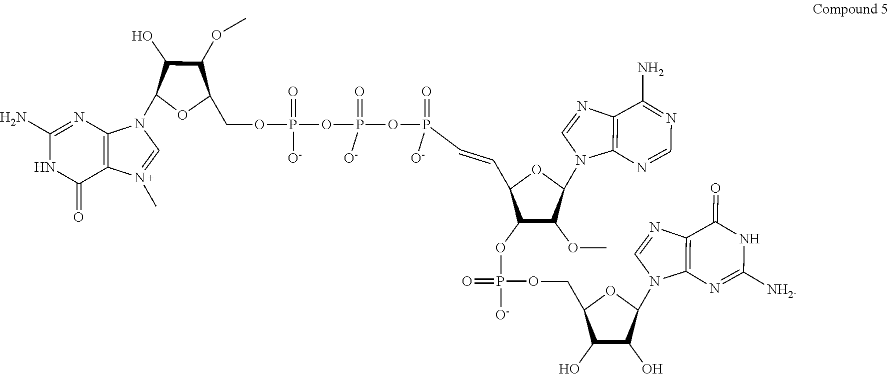

An additional example of Formula I includes Compound 6, as shown below, and salts thereof:

^{m7}GpppA_{2′F}p(s)G*:

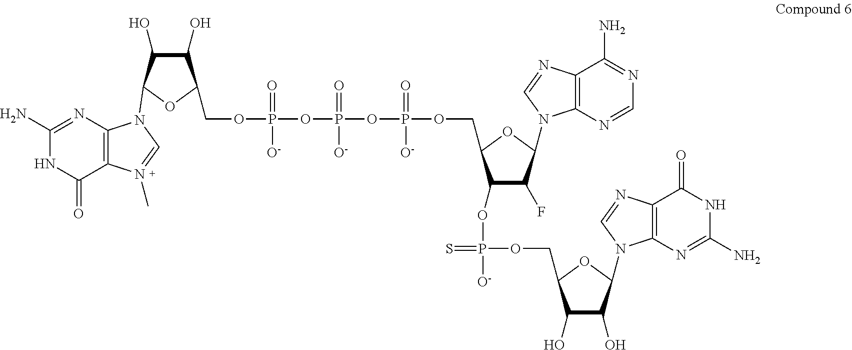

^{m7}GpppA_{2′F}p(s)G contains a chiral phosphorothioate moiety and is separated into two diastereomers: ^{m7}GpppA_{2′F}p(s_{Rp})G and ^{m7}GpppA_{2′F}p(s_{Sp})G

A class of compounds described herein is represented by Formula II:

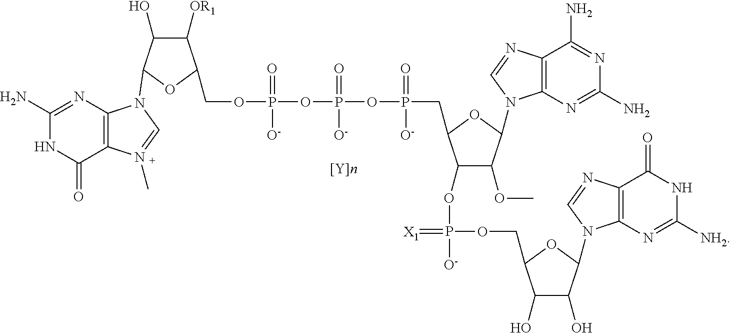

or any stereoisomer thereof.

In Formula II, R₁ is H or CH₃.

Also in Formula II, X₁ is O or S;

Additionally in Formula II, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

Optionally in Formula II, when X₁ is S, R₁ is CH₃.

Optionally, Formula II can be depicted in protonated (free acid) form as shown below:

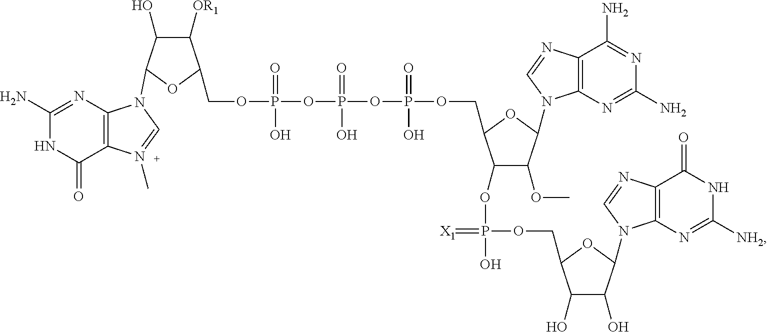

wherein R₁ and X₁ are as defined above for Formula II.

Optionally, Formula II can be depicted in ionized (anionic) form as shown below:

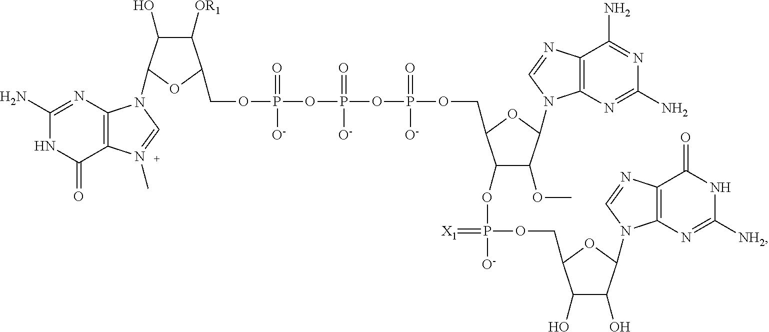

wherein R₁ and X₁ are as defined above for Formula II.

Optionally, Formula II can be depicted in ionized and in association with one or more cations (salt) form as shown below, in which compounds of the structure are in association with sodium cations (for representation purposes only and not by way of limitation):

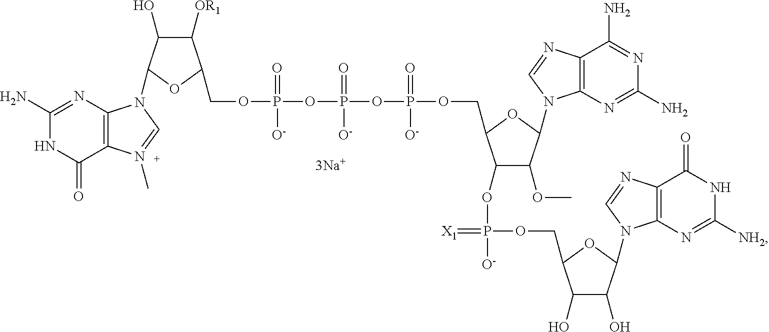

wherein R₁ and X₁ are as defined above for Formula II.

Examples of Formula II include the following compounds and salts thereof:

^{m7}Gppp(diaminopurine)_{2′OMe}pG:

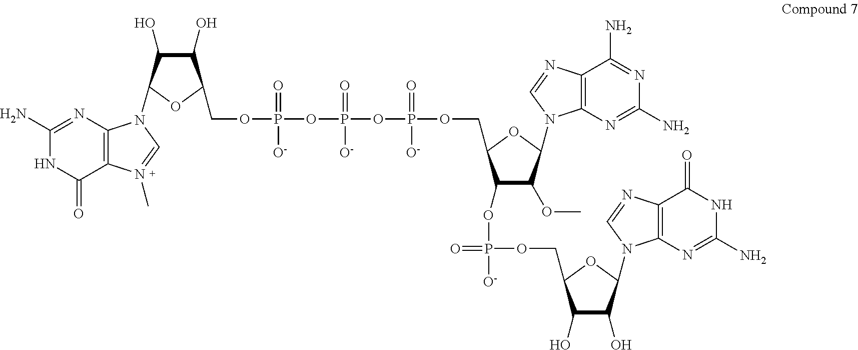

^{m7}G_{3′OMe}ppp(diaminopurine)_{2′OMe}pG:

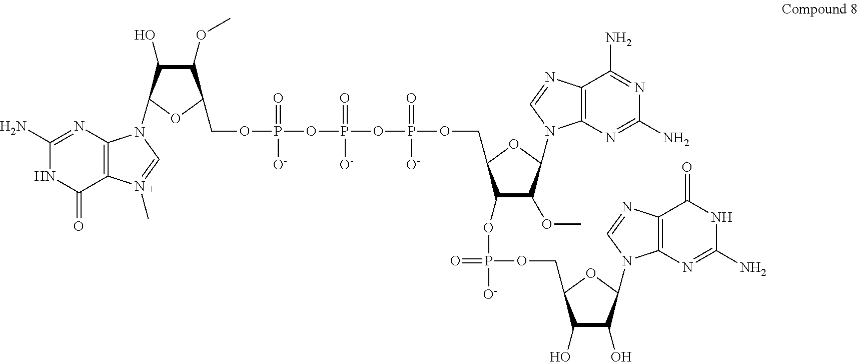

^{m7}G_{3′OMe}ppp(diaminopurine)_{2′OMe}p(s)G*:

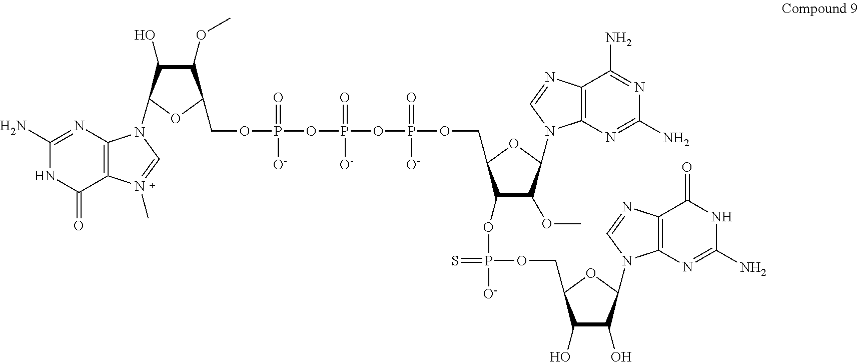

^{m7}G_{3′OMe}ppp(diaminopurine)_{2′OMe}p(s)G contains a chiral phosphorothioate moiety and is separated into two diastereomers: ^{m7}G_{3′OMe}ppp(diaminopurine)_{2′OMe}p(s_{Rp})G and ^{m7}G_{3′OMe}ppp(diaminopurine)_{2′OMe}p(s_{Sp})G

A class of compounds described herein is represented by Formula III:

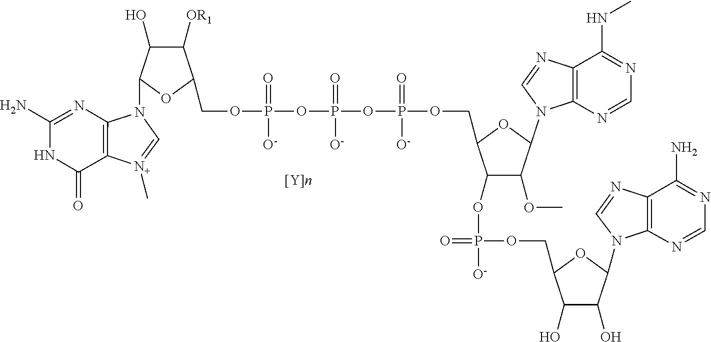

or any stereoisomer thereof.

In Formula III, R₁ is H or CH₃.

Additionally in Formula III, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

Optionally, Formula III can be depicted in protonated (free acid) form as shown below:

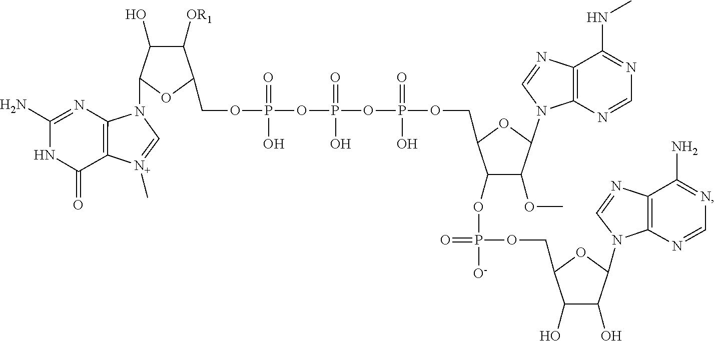

wherein R₁ is as defined above for Formula III.

Optionally, Formula III can be depicted in ionized (anionic) form as shown below:

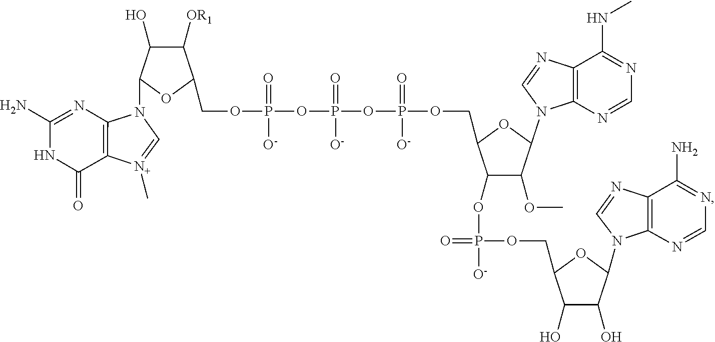

wherein R₁ is as defined above for Formula III.

Optionally, Formula III can be depicted in ionized and in association with one or more cations (salt) form as shown below, in which compounds of the structure are in association with sodium cations (for representation purposes only and not by way of limitation):

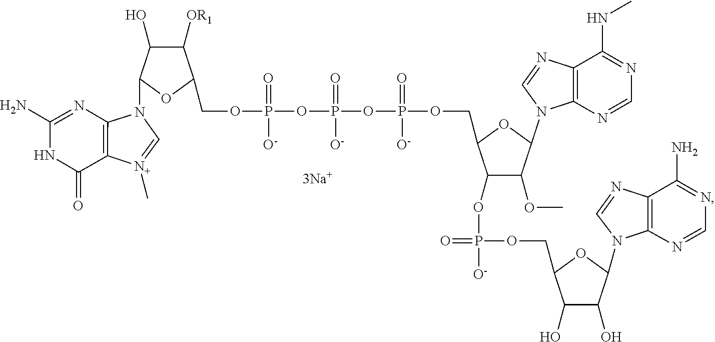

wherein R₁ is as defined above for Formula III.

Examples of Formula III include the following compounds and salts thereof:

^{m7}Gppp^{m6}A_{2′OMe}pA:

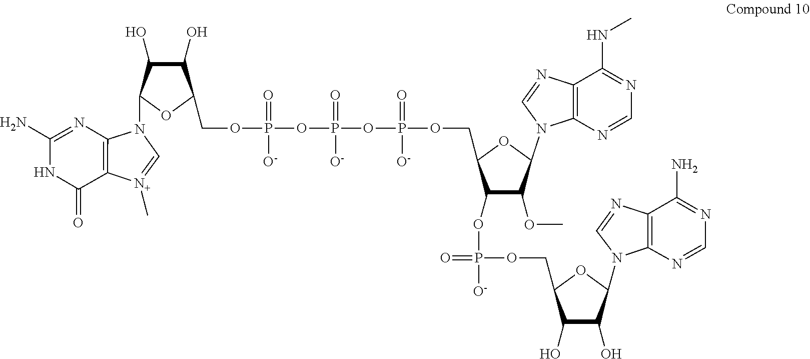

^{m7}G_{3′OMe}ppp^{m6}A_{2′OMe}pA:

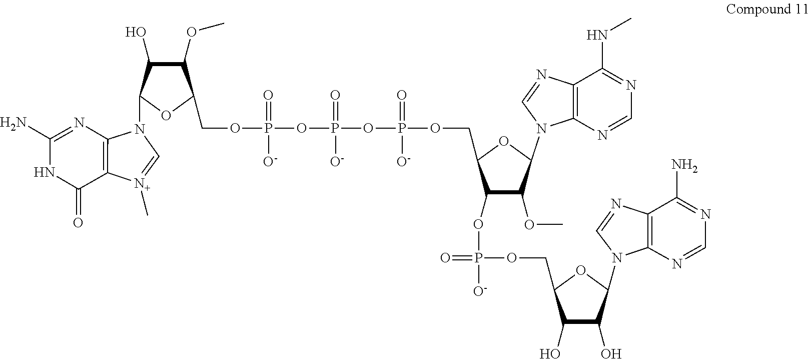

A class of compounds described herein is represented by Formula IV:

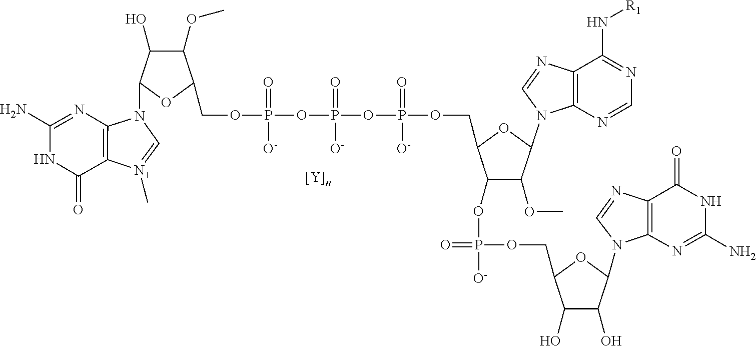

or any stereoisomer thereof.

In Formula IV, R₁ is H or CH₃.

Also in Formula IV, X₁, X₂, and X₃ are each independently selected from O and S, wherein when one of X₁, X₂, or X₃ is S, the remaining of X₁, X₂, and X₃ are O. Optionally, X₁ is S and X₂ and X₃ are both O. Optionally, X₂ is S and X₁ and X₃ are both O. Optionally, X₃ is S and X₁ and X₂ are both O.

Additionally in Formula IV, each independent Y is H⁺ or a cation; and n is 0, 1, 2, 3, or 4.

Optionally, Formula IV can be depicted in protonated (free acid) form as shown below:

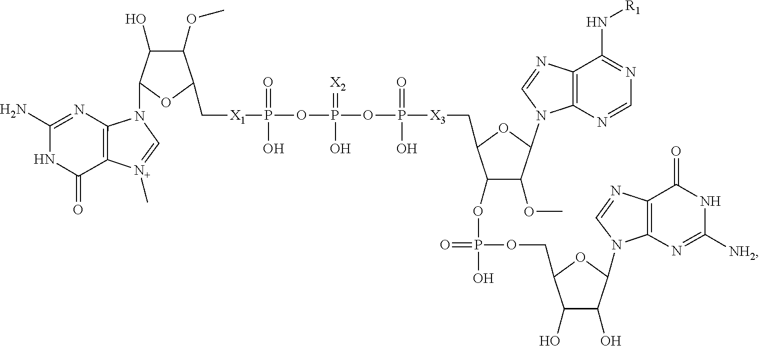

wherein R₁, X₁, X₂, and X₃ are as defined above for Formula IV.

Optionally, Formula IV can be depicted in ionized (anionic) form as shown below:

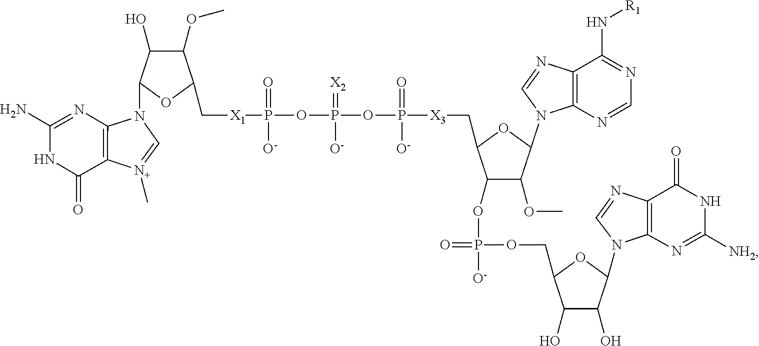

wherein R₁, X₁, X₂, and X₃ are as defined above for Formula IV.

Optionally, Formula IV can be depicted in ionized and in association with one or more cations (salt) form as shown below, in which compounds of the structure are in association with sodium cations (for representation purposes only and not by way of limitation):

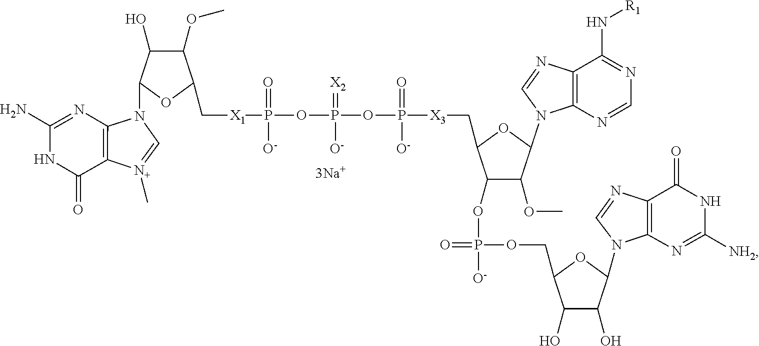

wherein R₁, X₁, X₂, and X₃ are as defined above for Formula IV.

Examples of Formula IV include the following compounds and salts thereof:

^{m7}G_{3′OMe5′-thio}ppp^{m6}A_{2′OMe}pG:

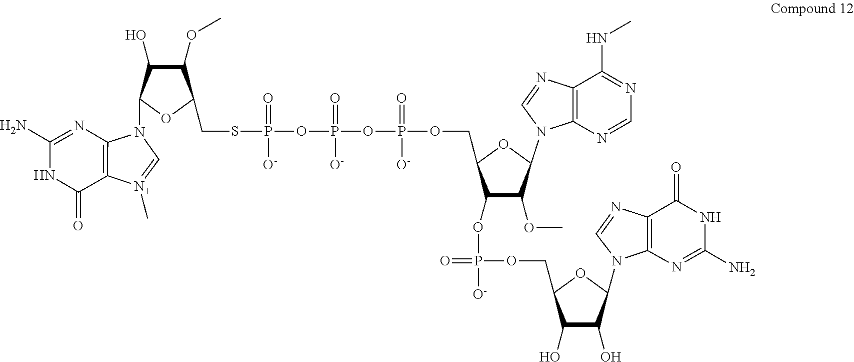

^{m7}G_{3′OMe}pp(s)p^{m6}A_{2′OMe}pG*:

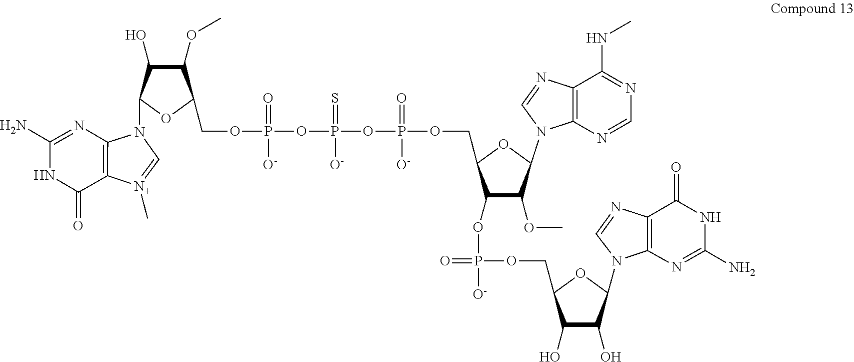

^{m7}G_{3′OMe}pp(s)p^{m6}A_{2′OMe}pG contains a chiral phosphorothioate moiety and is separated into two diastereomers: ^{m7}G_{3′OMe}pp(s_{Rp})p^{m6}A_{2′OMe}pG and ^{m7}G_{3′OMe}pp(s_{Sp})p^{m6}A_{2′OMe}pG
   ^{m7}G_{3′OMe}ppp^{m6}A_{2′OMe5′thio}pG:

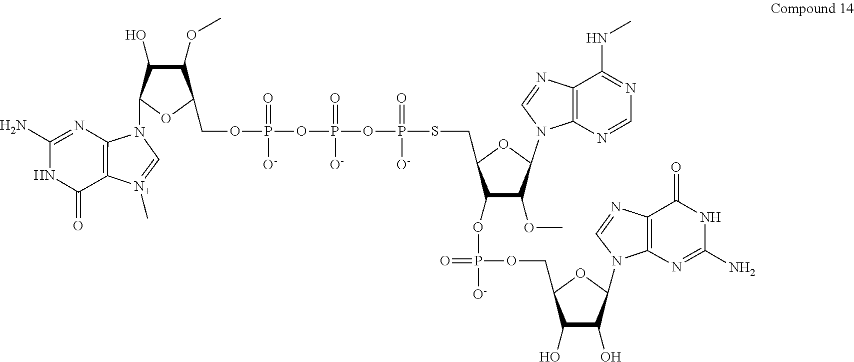

^{m7}G_{3′OMe}pppA_{2′OMe5′thio}pG:

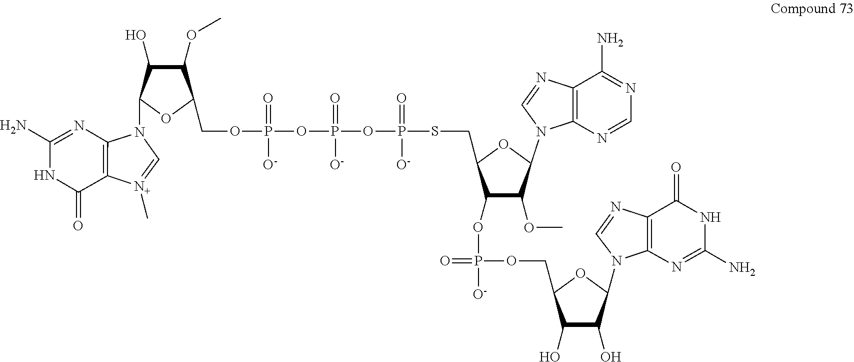

A class of compounds described herein is represented by Formula V:

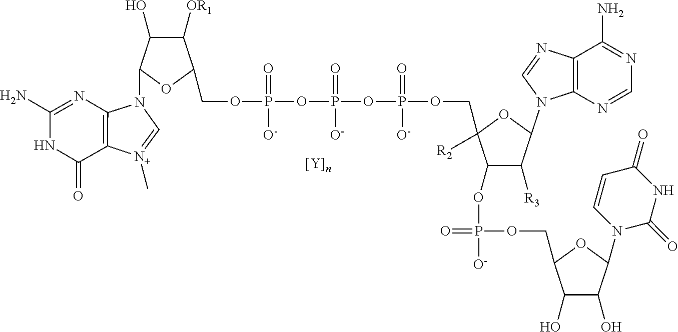

or any stereoisomer thereof.

In Formula V, R₁ is H or CH₃.

Also in Formula V, R₂ is H and R₃ is F, or R₂ and R₃ are covalently bonded together and, together with intermediate atoms, form a 2′-O, 4′-C methylene bridge.

Additionally in Formula V, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

Further in Formula V, when R₂ is H and R₃ is F, R₁ is not H.

Optionally, Formula V can be depicted in protonated (free acid) form as shown below:

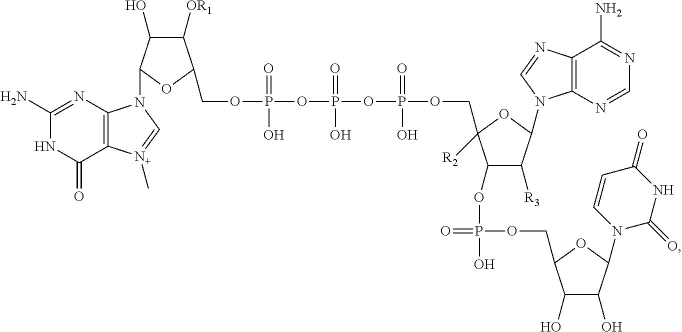

wherein R₁, R₂, and R₃ are as defined above for Formula V.

Optionally, Formula V can be depicted in ionized (anionic) form as shown below:

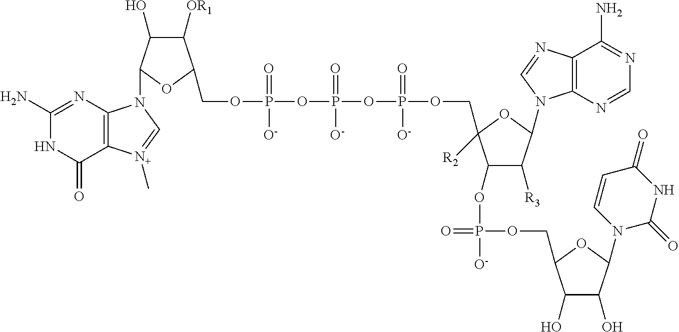

wherein R₁, R₂, and R₃ are as defined above for Formula V.

Optionally, Formula V can be depicted in ionized and in association with one or more cations (salt) form as shown below, in which compounds of the structure are in association with sodium cations (for representation purposes only and not by way of limitation):

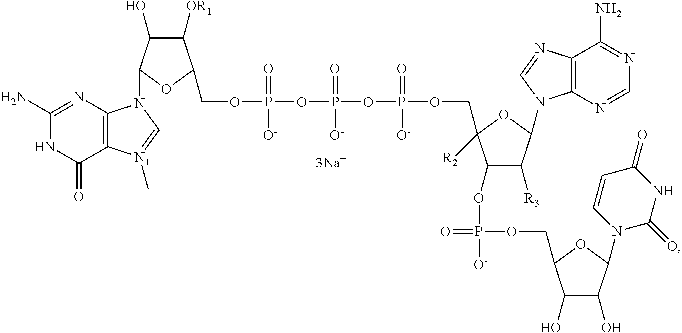

wherein R₁, R₂, and R₃ are as defined above for Formula V.

As described above, Formula V can include compounds in which R₂ and R₃ are covalently bonded together and, together with intermediate atoms, form a 2′-O, 4′-C methylene bridge (also referred to herein as a linked nucleic acid (LNA) moiety). Optionally, the compounds of Formula V having an LNA moiety are represented by Structure V-A as shown below, or a salt thereof:

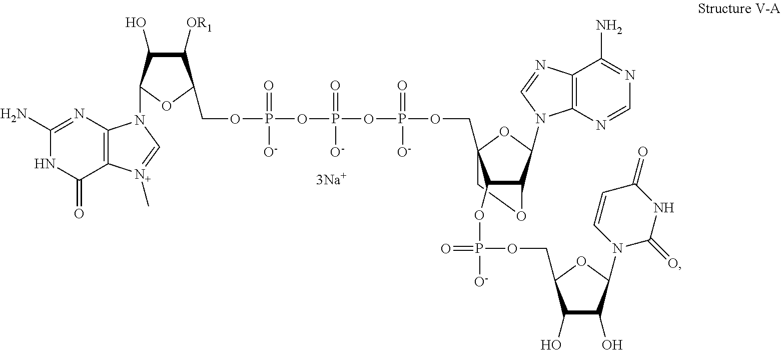

In Structure V-A, R₁ is as defined above for Formula V. Examples of Structure V-A include the following compounds and salts thereof:

^{m7}GpppA_{2′4′LNA}pU:

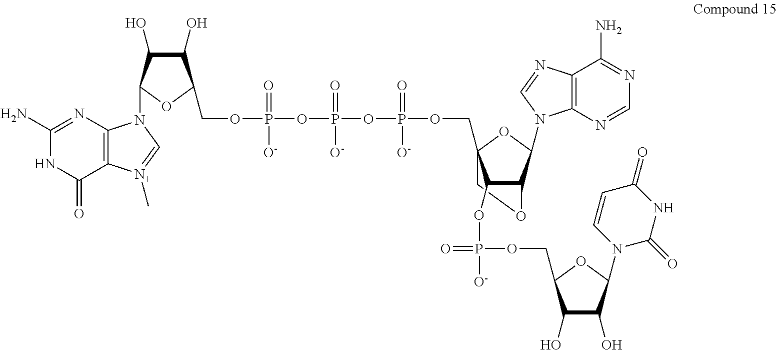

^{m7}G_{3′OMe}pppA_{2′4′LNA}pU:

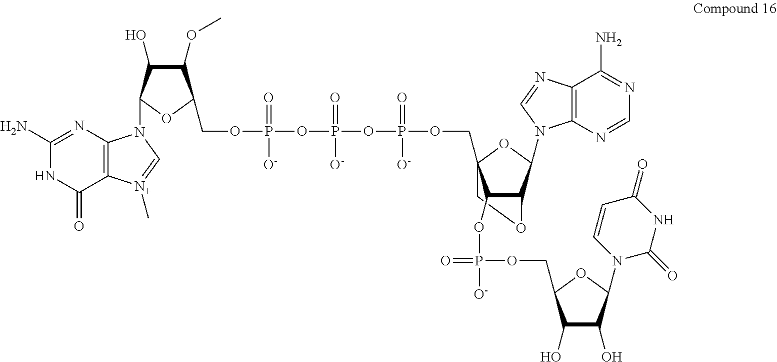

An additional example of Formula V includes Compound 17, as shown below, and salts thereof:

^{m7}G_{3′OMe}pppA_{2′F}pU:

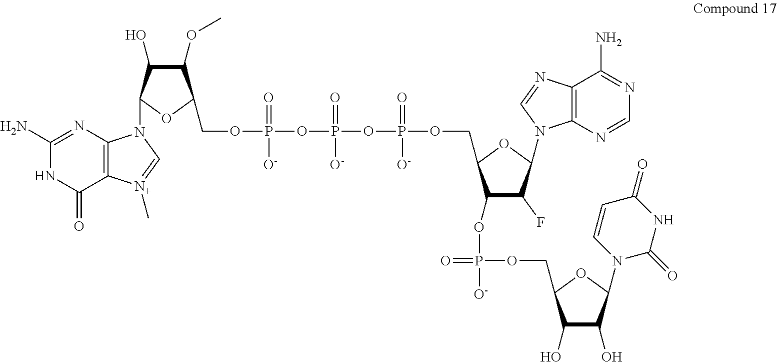

A class of compounds described herein is represented by Formula VI:

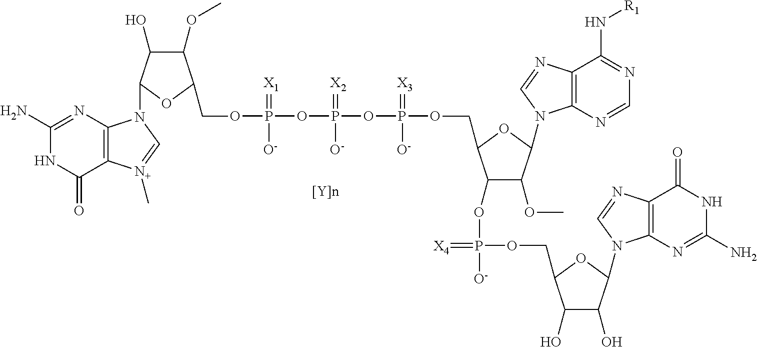

or any stereoisomer thereof.

In Formula VI, R₁ is H or CH₃.

Also in Formula VI, X₁, X₂, X₃, and X₄ are each independently selected from O and S.

Additionally in Formula VI, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

In Formula VI, one of X₁, X₂, X₃, and X₄ is S.

Optionally, Formula VI can be depicted in protonated (free acid) form as shown below:

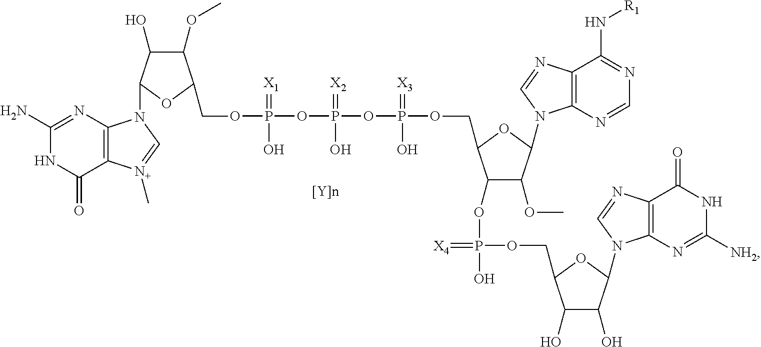

wherein R₁, X₁, X₂, X₃, and X₄ are as defined above for Formula VI.

Optionally, Formula VI can be depicted in ionized (anionic) form as shown below:

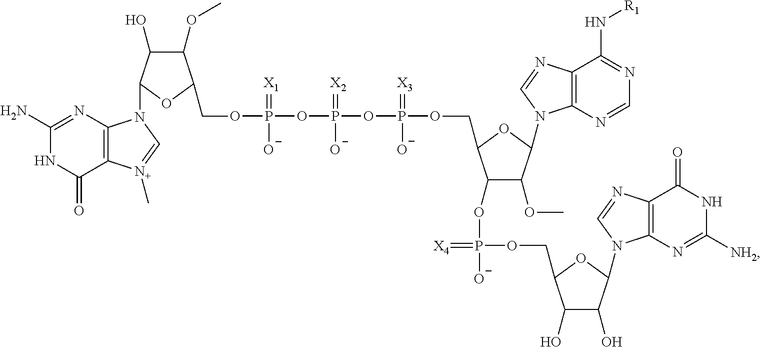

wherein R₁, X₁, X₂, X₃, and X₄ are as defined above for Formula VI.

Optionally, Formula VI can be depicted in ionized and in association with one or more cations (salt) form as shown below, in which compounds of the structure are in association with sodium cations (for representation purposes only and not by way of limitation):

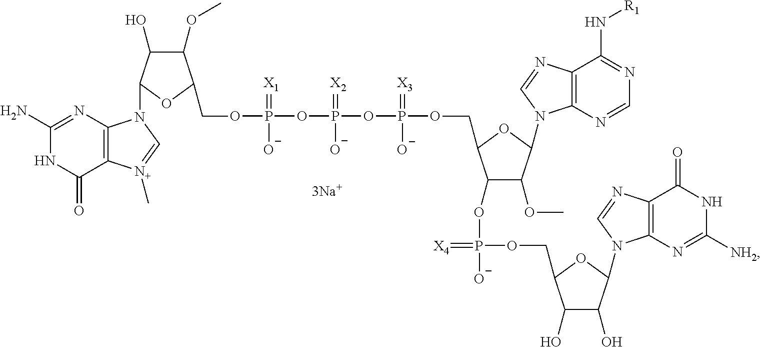

wherein R₁, X₁, X₂, X₃, and X₄ are as defined above for Formula VI.

Examples of Formula VI include the following compounds and salts thereof:

^{m7}G_{3′OMe}p(s)ppA_{2′OMe}pG*:

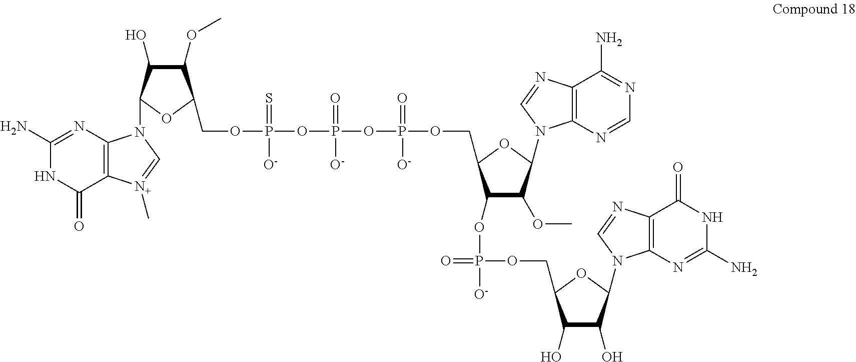

^{m7}G_{3′OMe}p(s)ppA_{2′OMe}pG contains a chiral phosphorothioate moiety and is separated into two diastereomers: ^{m7}G_{3′OMe}p(s_{Rp})ppA_{2′OMe}pG and ^{m7}G_{3′OMe}p(s_{Sp})ppA_{2′OMe}pG
   ^{m7}G_{3′OMe}pp(s)pA_{2′OMe}pG*:

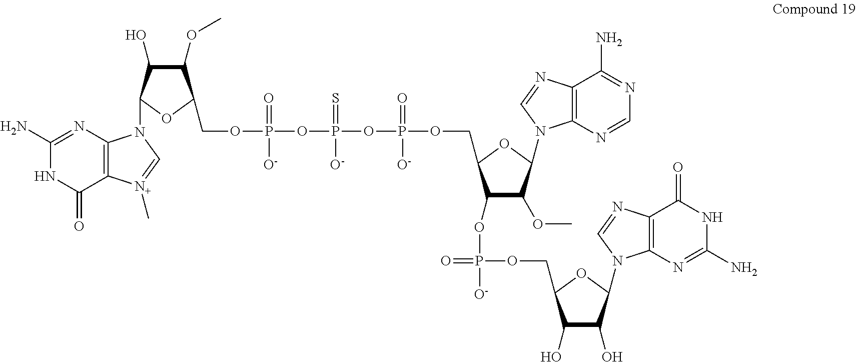

^{m7}G_{3′OMe}pp(s)pA_{2′OMe}pG contains a chiral phosphorothioate moiety and is separated into two diastereomers: ^{m7}G_{3′OMe}pp(s_{Rp})pA_{2′OMe}pG and ^{m7}G_{3′OMe}pp(s_{Sp})pA_{2′OMe}pG
   ^{m7}G_{3′OMe}ppp(s)A_{2′OMe}pG*:

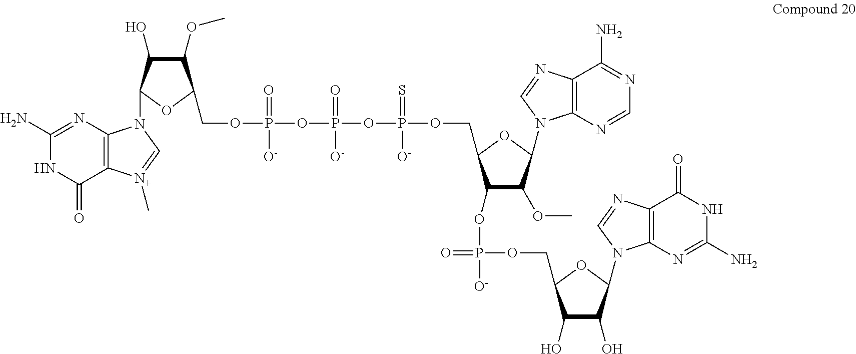

^{m7}G_{3′OMe}ppp(s)A_{2′OMe}pG contains a chiral phosphorothioate moiety and is separated into two diastereomers: ^{m7}G_{3′OMe}ppp(s_{Rp})A_{2′OMe}pG and ^{m7}G_{3′OMe}ppp(s_{Sp})A_{2′OMe}pG
   ^{m7}G_{3′OMe}pppA_{2′OMe}p(s)G*:

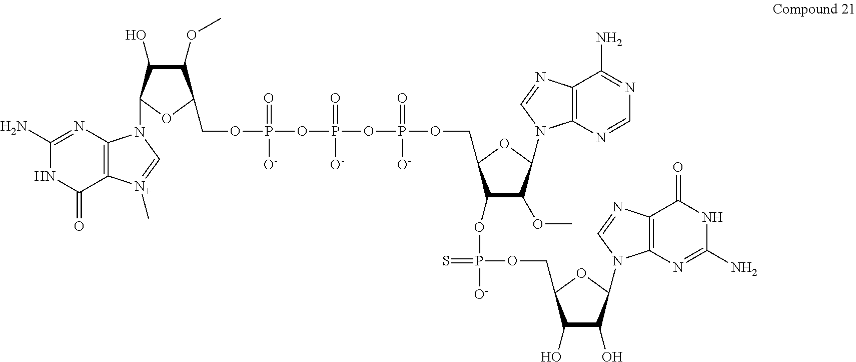

^{m7}G_{3′OMe}pppA_{2′OMe}p(s)G contains a chiral phosphorothioate moiety and is separated into two diastereomers: ^{m7}G_{3′OMe}pppA_{2′OMe}p(s_{Rp})G and ^{m7}G_{3′OMe}pppA_{2′OMe}p(s_{Sp})G
   ^{m7}G_{3′OMe}ppp^{m6}A_{2′OMe}p(s)G*:

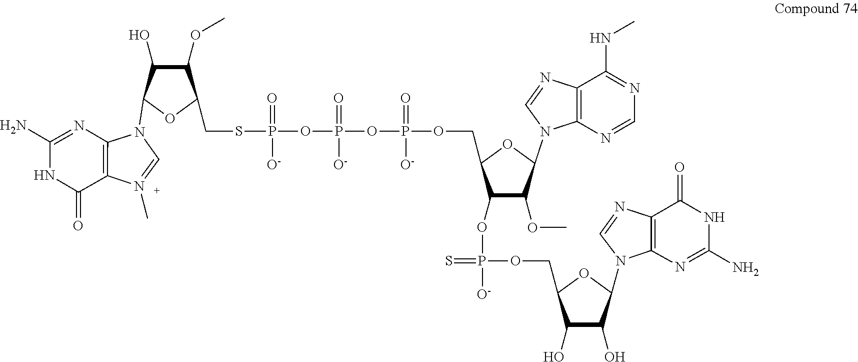

^{m7}G_{3′OMe}ppp^{m6}A_{2′OMe}p(s)G contains a chiral phosphorothioate moiety and is separated into two diastereomers: ^{m7}G_{3′OMe}ppp^{m6}A_{2′OMe}p(s_{Rp})G and ^{m7}G_{3′OMe}ppp^{m6}A_{2′OMe}p(s_{Sp})G
   ^{m7}G_{3′OMe}P(S)Pp^{m6}A_{2′OMe}pG:

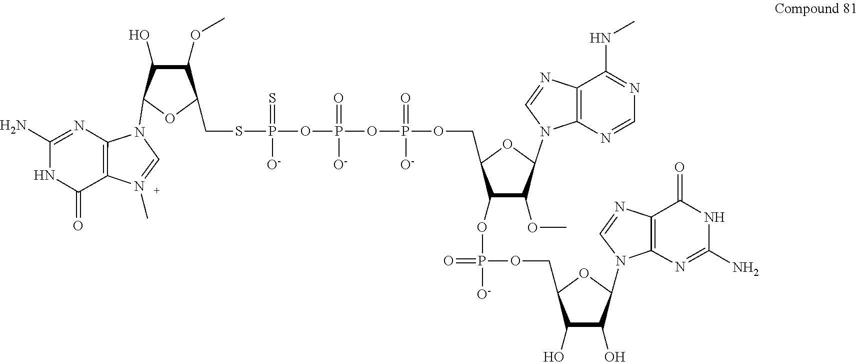

^{m7}G_{3′OMe}p(S)pp^{m6}A_{2′OMe}pG contains a chiral phosphorothioate moiety and is separated into two diastereomers: ^{m7}G_{3′OMe}p(s_{Rp})pp^{m6}A_{2′OMe}pG and ^{m7}G_{3′OMe}p(s_{Sp})pp^{m6}A_{2′OMe}pG

A class of compounds described herein is represented by Formula VII:

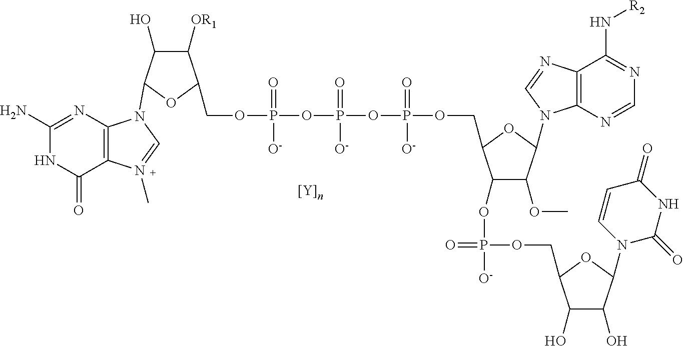

or any stereoisomer thereof.

In Formula VII, R₁ is H or CH₃.

Also in Formula VII, R₂ is H or CH₃.

In Formula VII, at least one of R₁ or R₂ is CH₃. In other words, R₁ and R₂ are not simultaneously H.

Additionally in Formula VII, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

Optionally, Formula VII can be depicted in protonated (free acid) form as shown below:

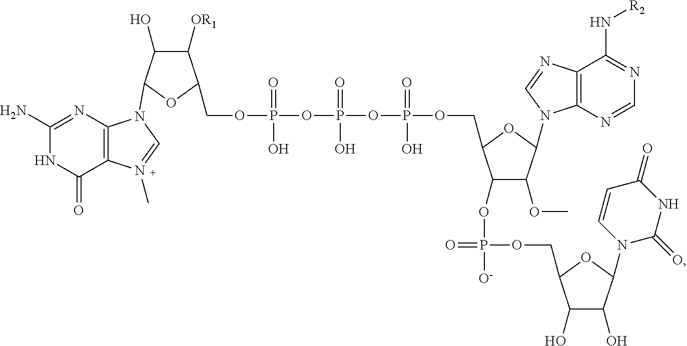

wherein R₁ and R₂ are as defined above for Formula VII.

Optionally, Formula VII can be depicted in ionized (anionic) form as shown below:

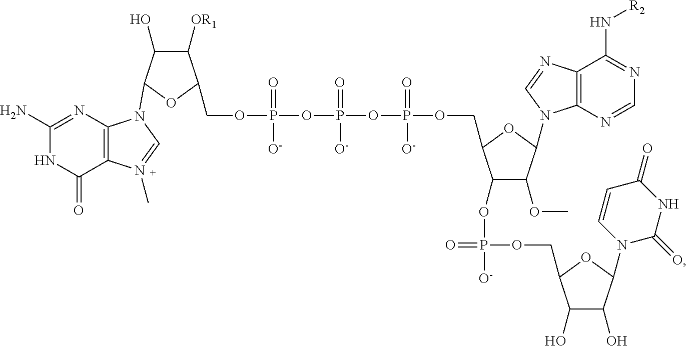

wherein R₁ and R₂ are as defined above for Formula VII.

Optionally, Formula VII can be depicted in ionized and in association with one or more cations (salt) form as shown below, in which compounds of the structure are in association with sodium cations (for representation purposes only and not by way of limitation):

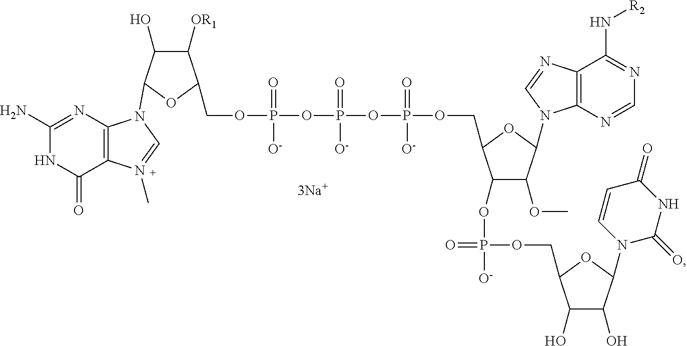

wherein R₁ and R₂ are as defined above for Formula VII.

Examples of Formula VII include the following compounds and salts thereof:

^{m7}G_{3′OMe}pppA_{2′OMe}pU:

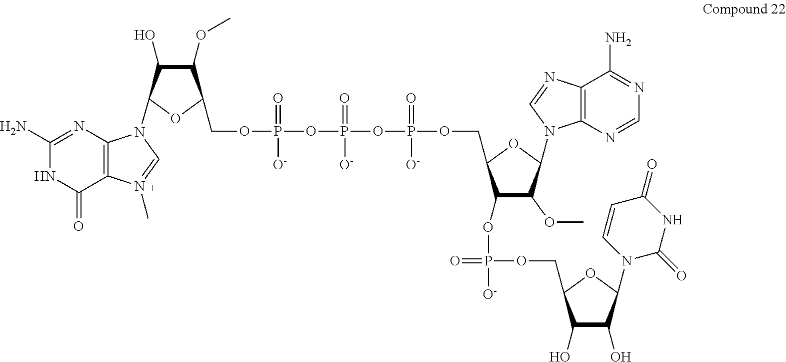

^{m7}Gppp^{m6}A_{2′OMe}pU:

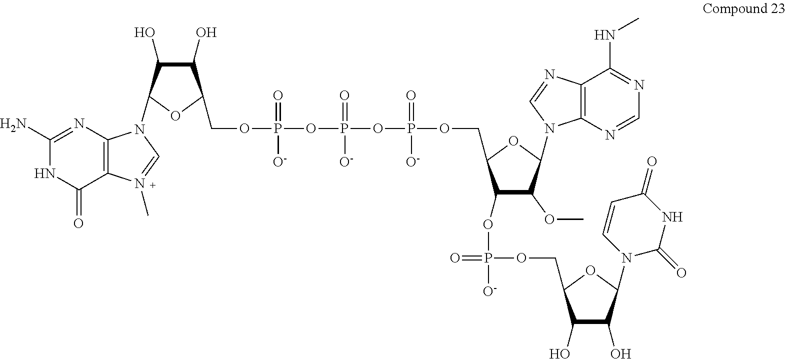

^{m7}G_{3′OMe}ppp^{m6}A_{2′OMe}pU:

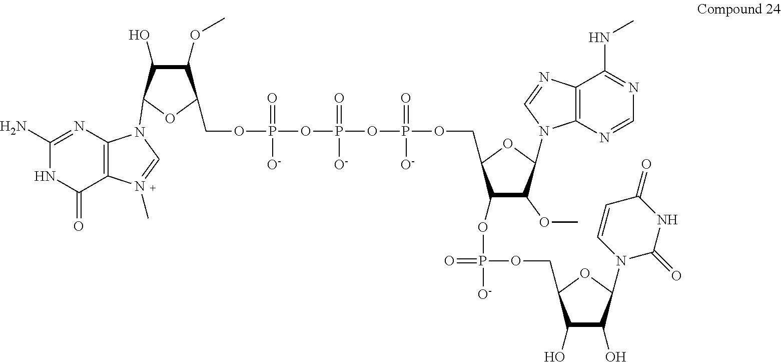

A class of compounds described herein is represented by Formula VIII.

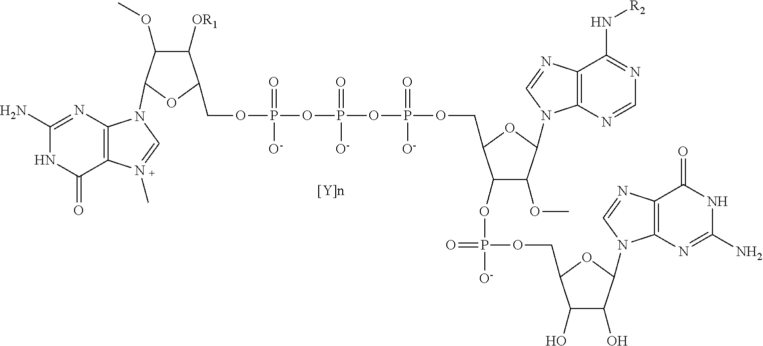

or any stereoisomer thereof.

In Formula VIII, R₁ and R₂ are each independently selected from H and CH₃.

Also in Formula VIII, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

Optionally, Formula VIII can be depicted in protonated (free acid) form as shown below:

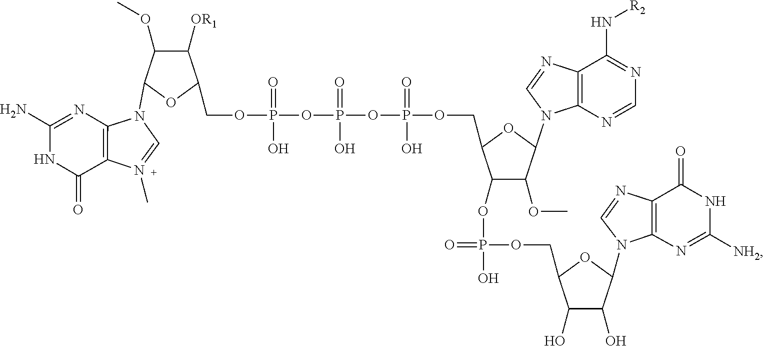

wherein R₁ and R₂ are as defined above for Formula VIII.

Optionally, Formula VIII can be depicted in ionized (anionic) form as shown below:

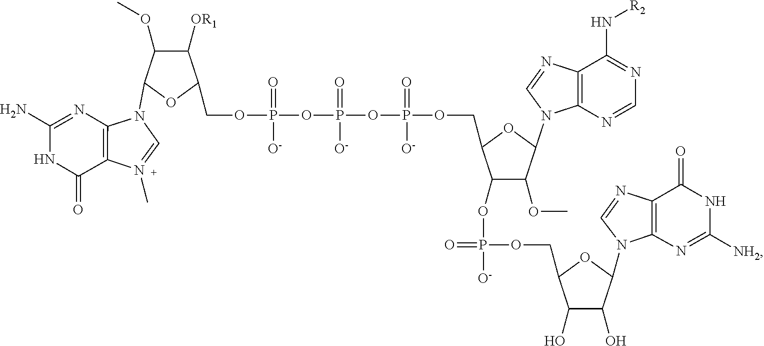

wherein R₁ and R₂ are as defined above for Formula VIII.

Optionally, Formula VIII can be depicted in ionized and in association with one or more cations (salt) form as shown below, in which compounds of the structure are in association with sodium cations (for representation purposes only and not by way of limitation):

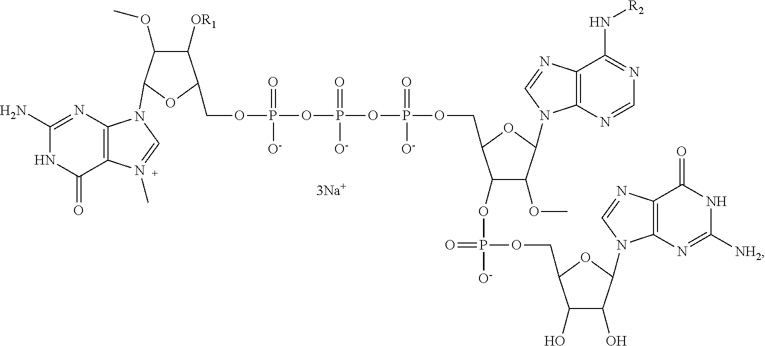

wherein R₁ and R₂ are as defined above for Formula VIII.

Examples of Formula VIII include the following compounds and salts thereof:

^{m7}G_{2′OMe3′OMe}pppA_{2′OMe}pG:

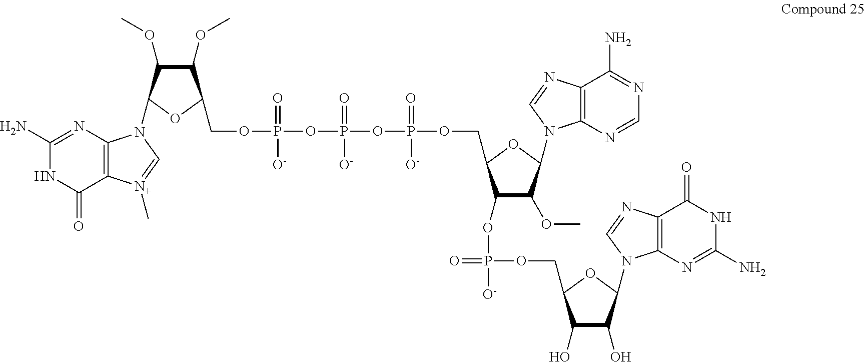

^{m7}G_{2′OMe}pppA_{2′OMe}pG:

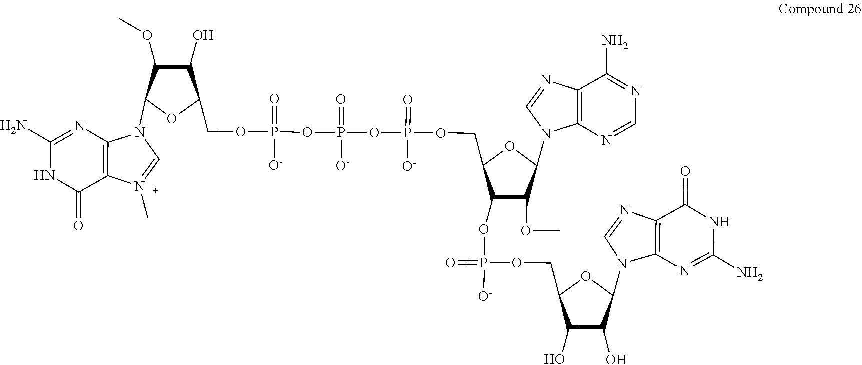

^{m7}G_{2′OMe3′OMe}ppp^{m6}A_{2′OMe}pG:

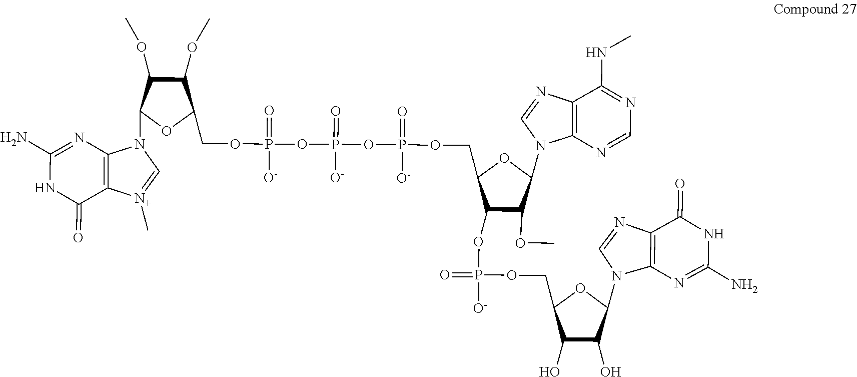

^{m7}G_{2′OMe}ppp^{m6}A_{2′OMe}pG:

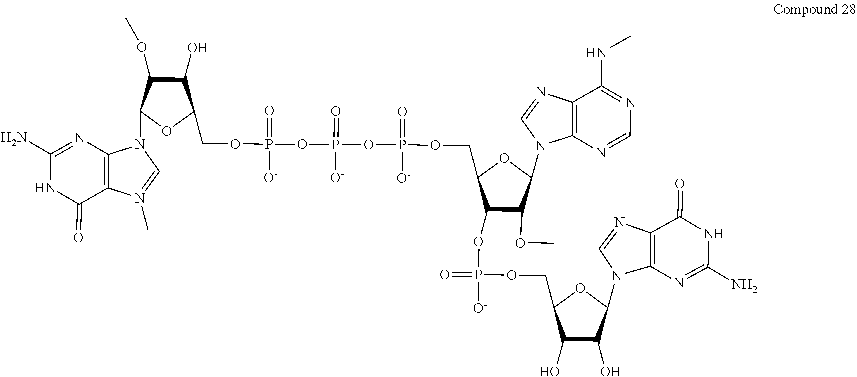

Other compounds described herein for use in certain methods include the following compounds, or any stereoisomer thereof:

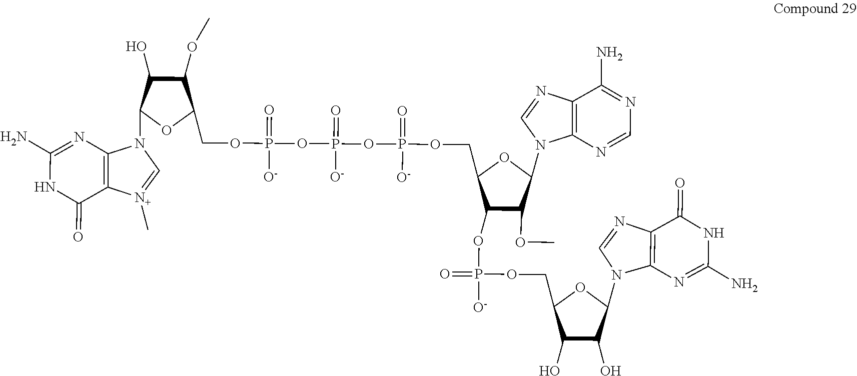

^{m7}G_{3′OMe}pppA_{2′OMe}pG:

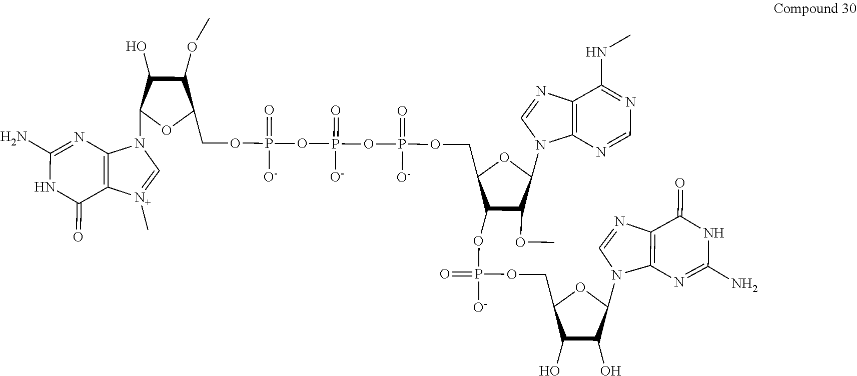

^{m7} Gppp^{m6}A_{2′OMe}pG:

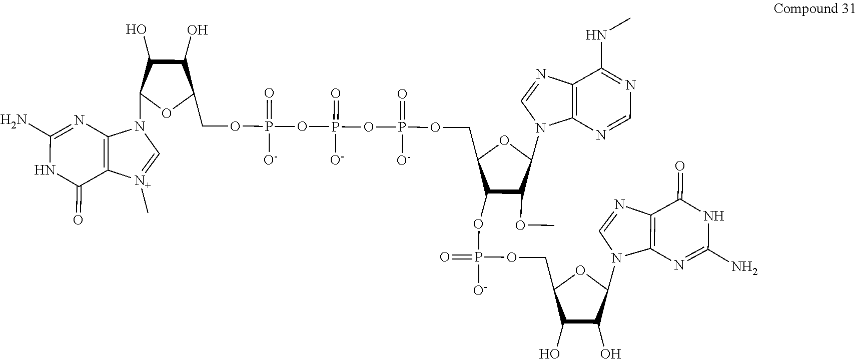

^{m7}G_{2′4′-LNA}pppA_{2′OMe}pG:

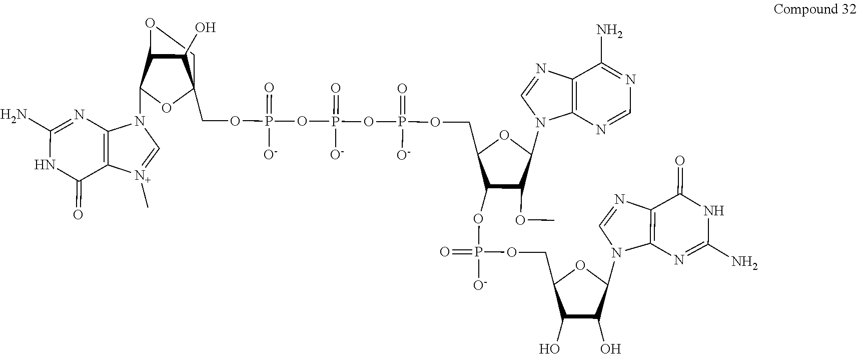

^{m7}GpppA_{2′F}pU:

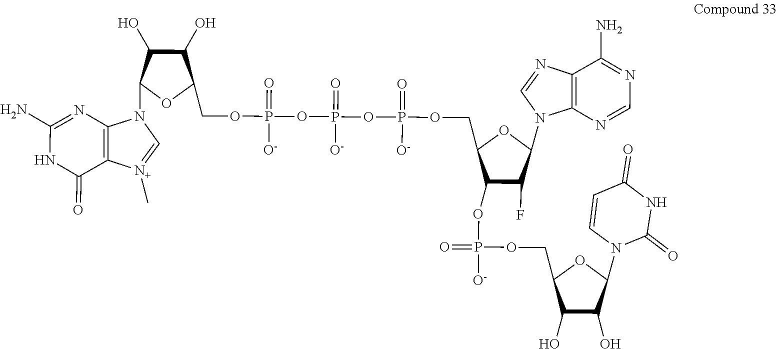

Optionally, the compounds for use in certain methods are not compounds according to Compound 29, Compound 30, Compound 31, Compound 32, and/or Compound 33.

A class of compounds described herein is represented by Formula IX:

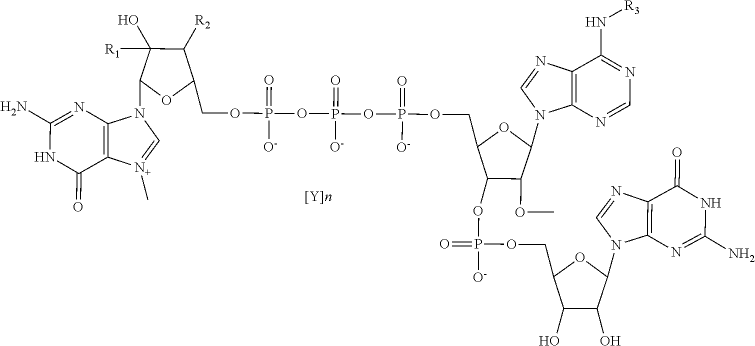

or any stereoisomer thereof.

In Formula IX, R₁ is H or CH₃.

Also in Formula IX, R₂ is OH, F, substituted or unsubstituted alkoxy, or thio.

Optionally, the substituted or unsubstituted alkoxy group can be methoxy, methoxyethoxy, or azidomethoxy.

Also in Formula IX, R₃ is H or CH₃.

In Formula IX, when R₂ is OH, R₁ is not H.

Also in Formula IX, each independent Y is H⁺ or a cation and n is 0, 1, 2, 3, or 4. Optionally, the cation is a pharmaceutically acceptable cation (e.g., Na⁺, K⁺, Li⁺, or TEAH⁺)

Optionally, Formula IX can be depicted in protonated (free acid) form as shown below:

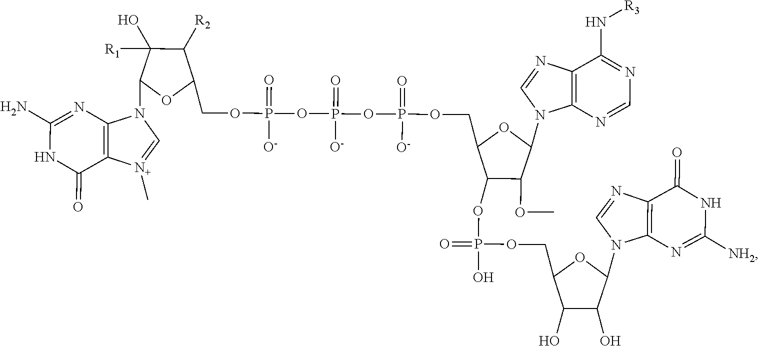

wherein R₁, R₂, and R₃ are as defined above for Formula IX.

Optionally, Formula IX can be depicted in ionized (anionic) form as shown below:

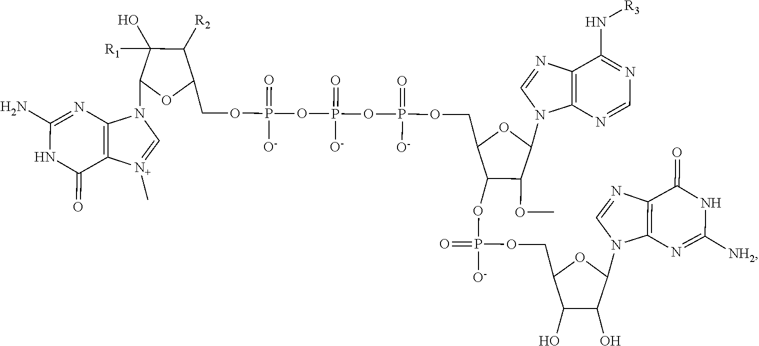

wherein R₁, R₂, and R₃ are as defined above for Formula IX.

Optionally, Formula IX can be depicted in ionized and in association with one or more cations (salt) form as shown below, in which compounds of the structure are in association with sodium cations (for representation purposes only and not by way of limitation):

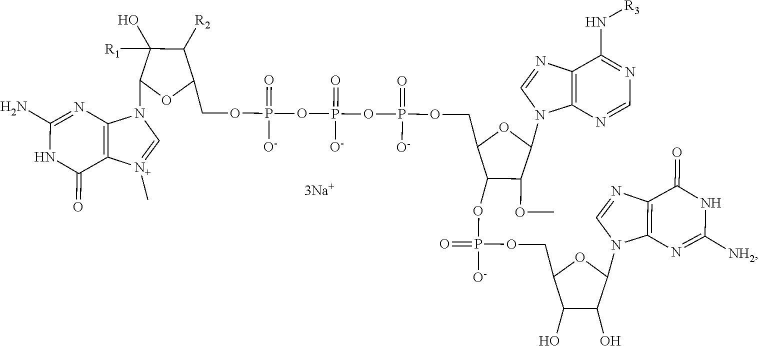

wherein R₁, R₂, and R₃ are as defined above for Formula IX.

Examples of Formula IX include the following compounds and salts thereof:

^{m7}G_{3′SMe}pppA_{2′OMe}pG:

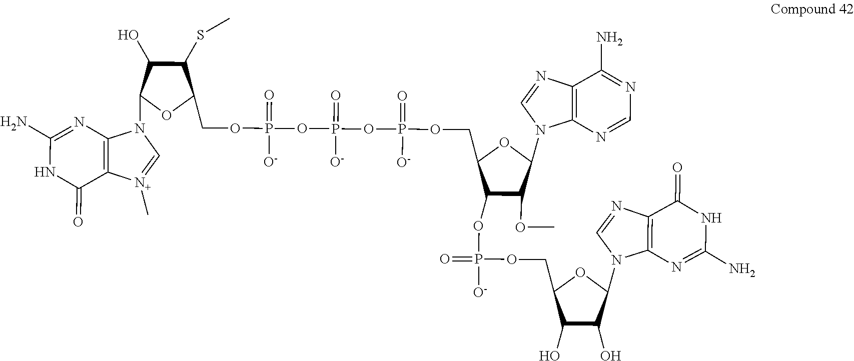

^{m7}G_{2′OH, 2′Me}pppA_{2′OMe}pG:

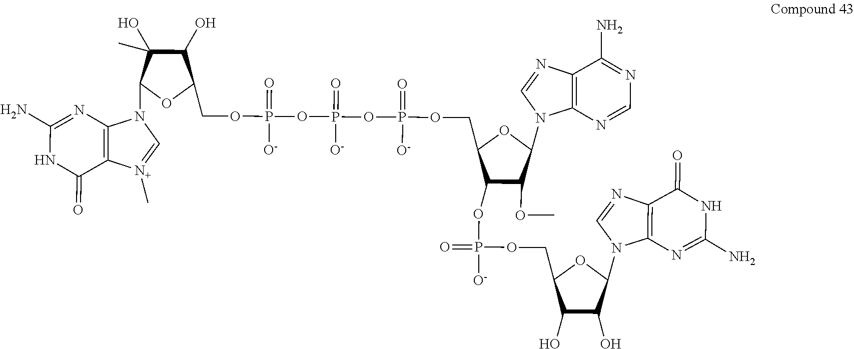

^{m7}G_{3′AZM}ppp^{m6}A_{2′OMe}pG:

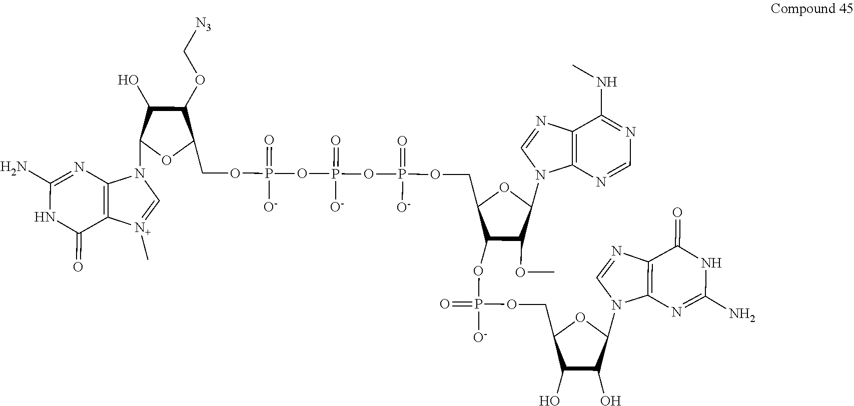

^{m7}G_{3′F}ppp^{m6}A_{2′OMe}pG:

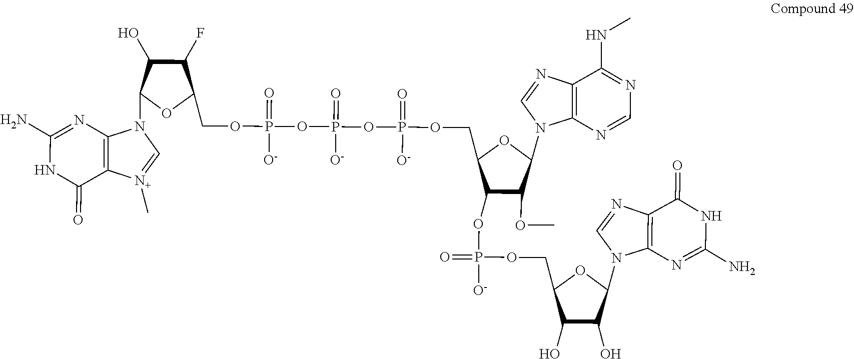

^{m7}G_{3′MOE}ppp^{m6}A_{2′OMe}pG:

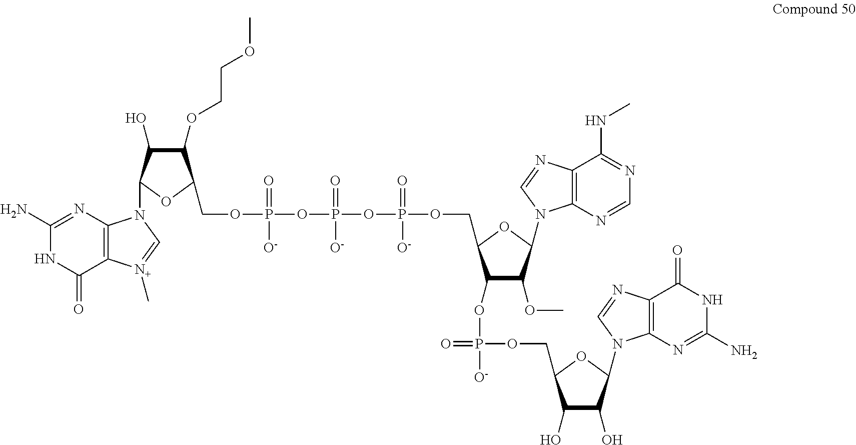

^{m7} G_{3′SMe}ppp^{m6}A_{2′OMe}pG:

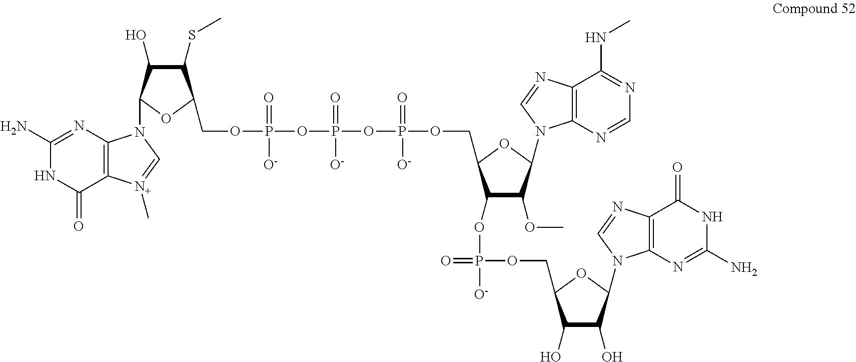

^{m7}G_{2′OH,2′Me}ppp^{m6}A_{2′OMe}pG:

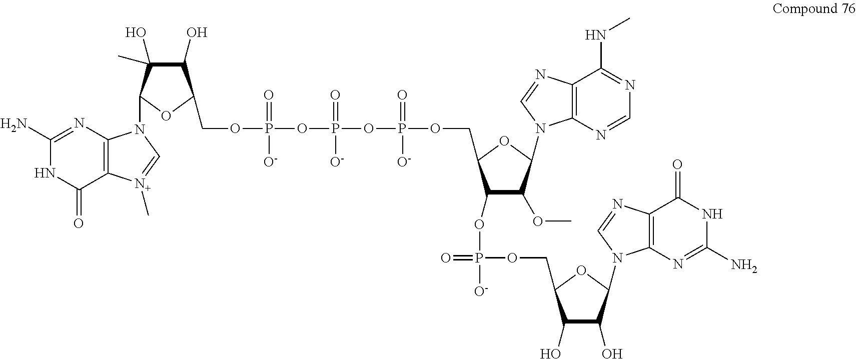

A class of compounds described herein is represented by Formula X:

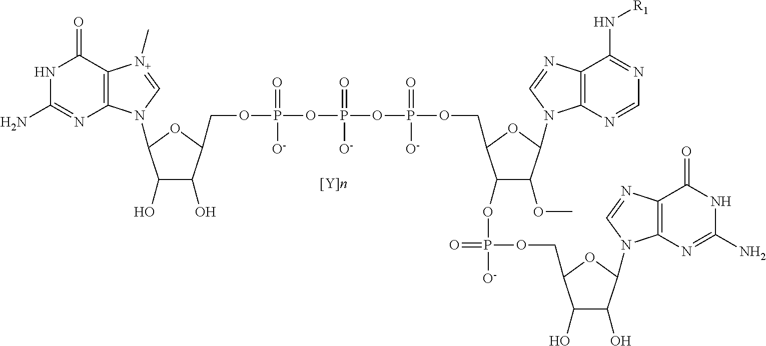

or any stereoisomer thereof.

In Formula X, R₁ is H or CH₃.

Additionally in Formula X, each independent Y is H⁺ or a cation; and n is 0, 1, 2, 3, or 4.

Optionally, Formula X can be depicted in protonated (free acid) form as shown below:

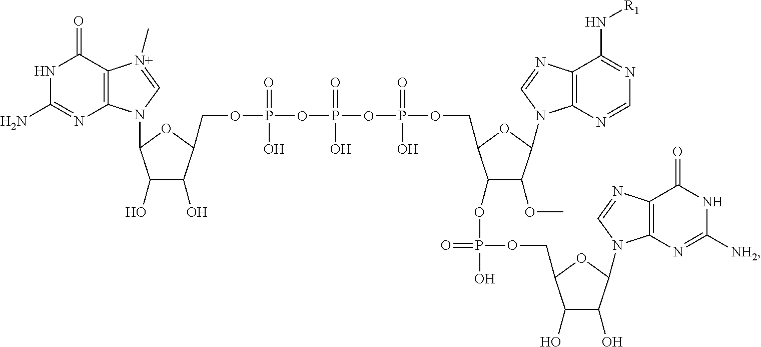

wherein R₁ is as defined above or Formula X.

Optionally, Formula X can be depicted in ionized (anionic) form as shown below:

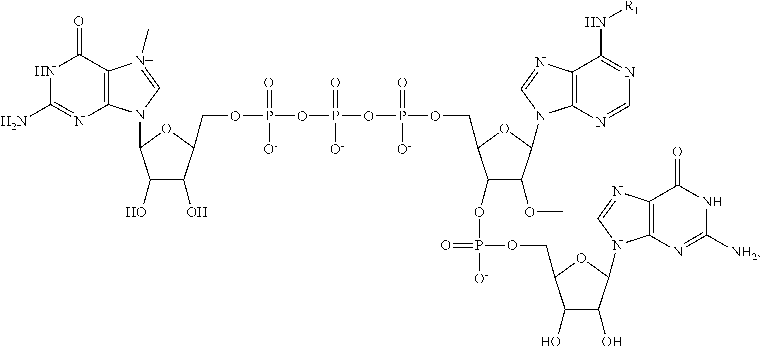

wherein R₁ is as defined above for Formula X.

Optionally, Formula X can be depicted in ionized and in association with one or more cations (salt) form as shown below, in which compounds of the structure are in association with sodium cations (for representation purposes only and not by way of limitation):

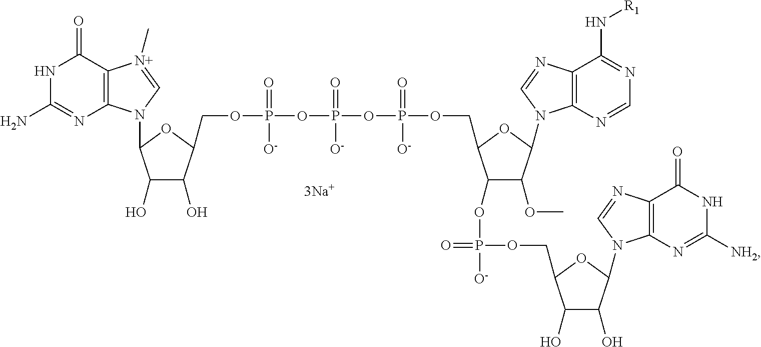

wherein R₁ is as defined above for Formula X.

Examples of Formula X include the following compounds and salts thereof:

^{m7}(L-sugar isomer)GpppA_{2′OMe}pG:

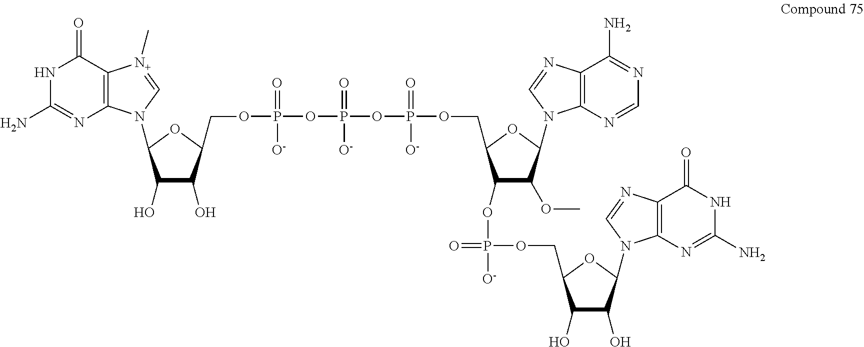

^{m7}(L-sugar isomer)Gppp^{m6}A_{2′OMe}pG:

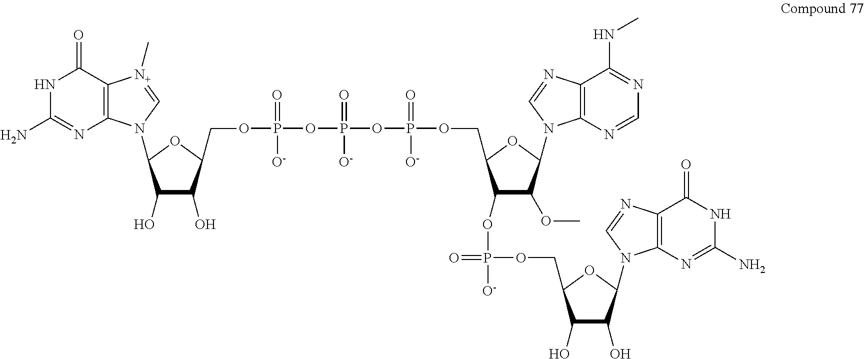

A class of compounds described herein is represented by Formula XI:

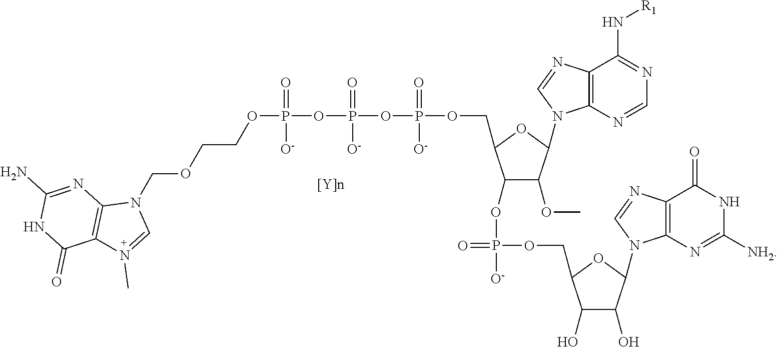

In Formula XI, R₁ is H or CH₃.

Also in Formula XI, each independent Y is H⁺ or a cation and n is 0, 1, 2, 3, or 4. Optionally, the cation is a pharmaceutically acceptable cation (e.g., Na⁺, K⁺, Li⁺, or TEAH⁺).

Optionally, Formula XI can be depicted in protonated (free acid) form as shown below:

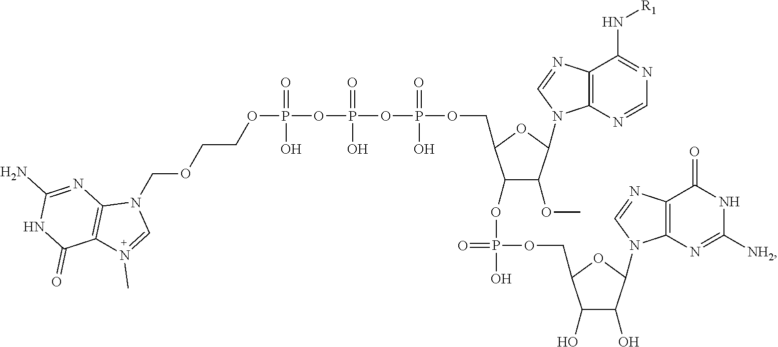

wherein R₁ is as defined above for Formula XI.

Optionally, Formula XI can be depicted in ionized (anionic) form as shown below:

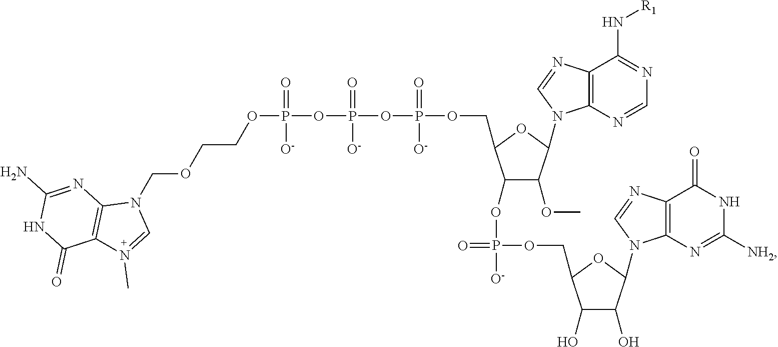

wherein R₁ is as defined above for Formula XI.

Optionally, Formula XI can be depicted in ionized and in association with one or more cations (salt) form as shown below, in which compounds of the structure are in association with sodium cations (for representation purposes only and not by way of limitation):

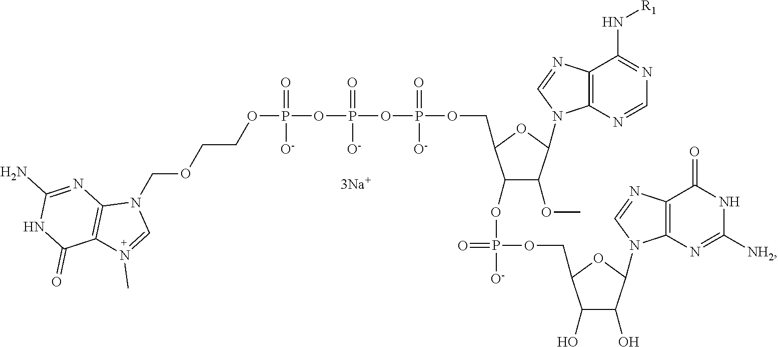

wherein R₁ is as defined above for Formula XI.

Examples of Formula XI include the following compounds and salts thereof:

^{m7}(acyclo)GpppA_{2′OMe}pG:

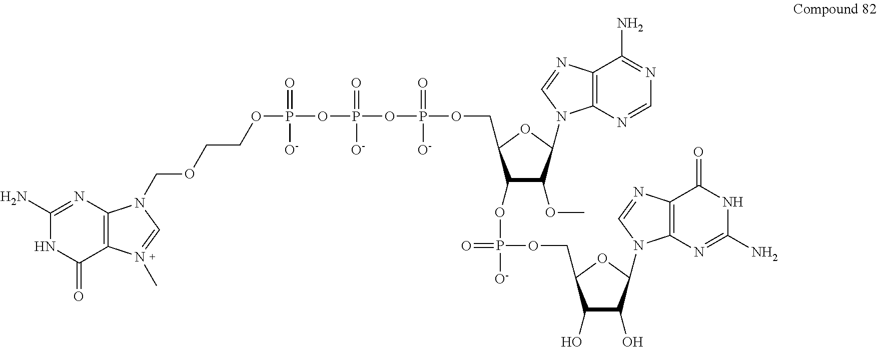

^{m7}(acyclo)Gppp^{m6}A_{2′OMe}pG:

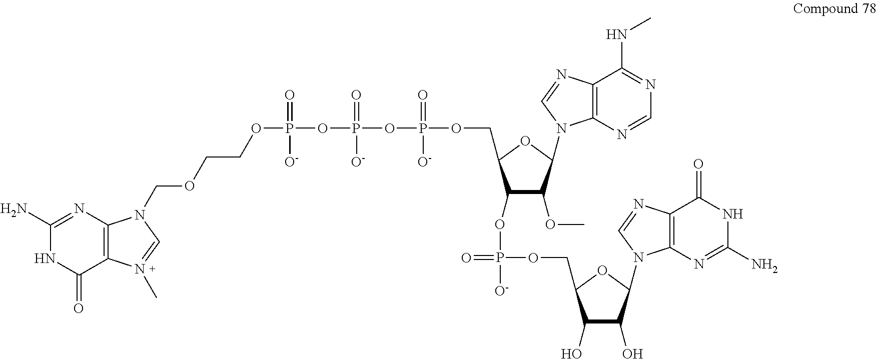

A class of compounds described herein is represented by Formula XII:

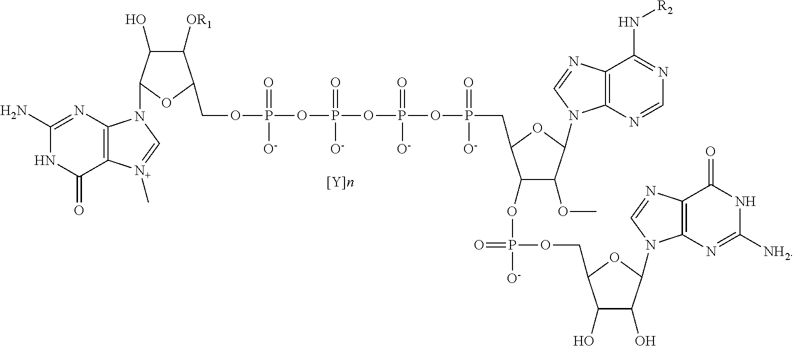

In Formula XII, R₁ and R₂ are each independently selected from H and CH₃.

Also in Formula XII, each independent Y is H⁺ or a cation and n is 0, 1, 2, 3, 4, or 5. Optionally, the cation is a pharmaceutically acceptable cation (e.g., Na⁺, K⁺, Li⁺, or TEAH⁺).

Optionally, Formula XII can be depicted in protonated (free acid) form as shown below:

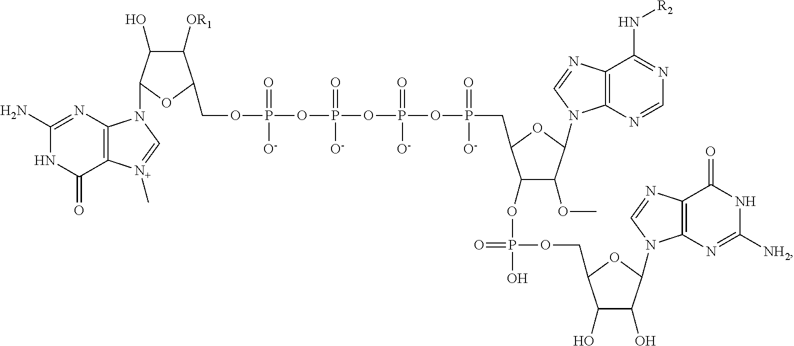

wherein
R₁ and R₂ are as defined above for Formula XII.

Optionally, Formula XII can be depicted in ionized (anionic) form as shown below:

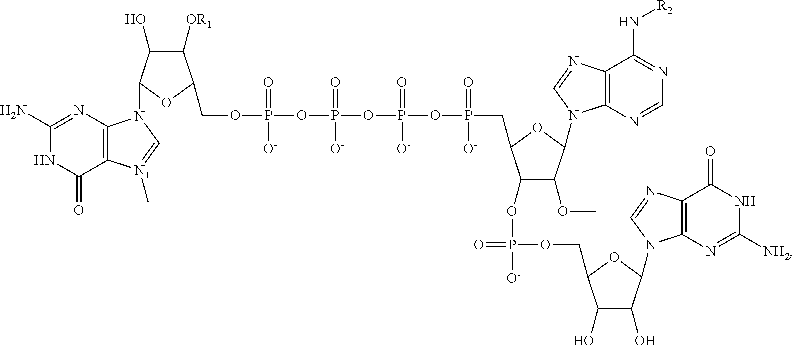

wherein
R₁ and R₂ are as defined above for Formula XII.

Optionally, Formula XII can be depicted in ionized and in association with one or more cations (salt) form as shown below, in which compounds of the structure are in association with sodium cations (for representation purposes only and not by way of limitation):

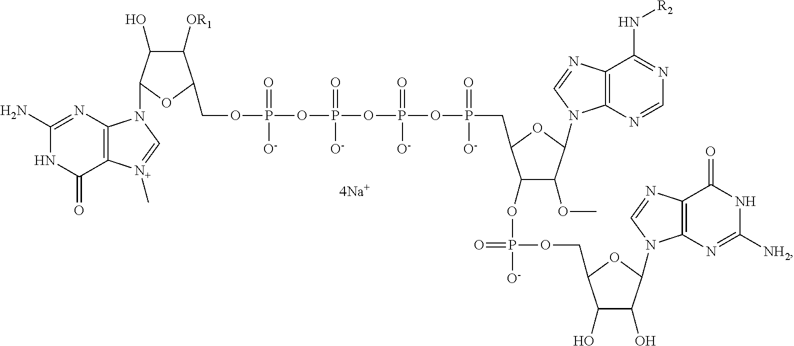

wherein
R₁ and R₂ are as defined above for Formula XII.

Examples of Formula XII include the following compounds and salts thereof:

^{m7}G_{3′OMe}pppA_{2′OMe}pG:

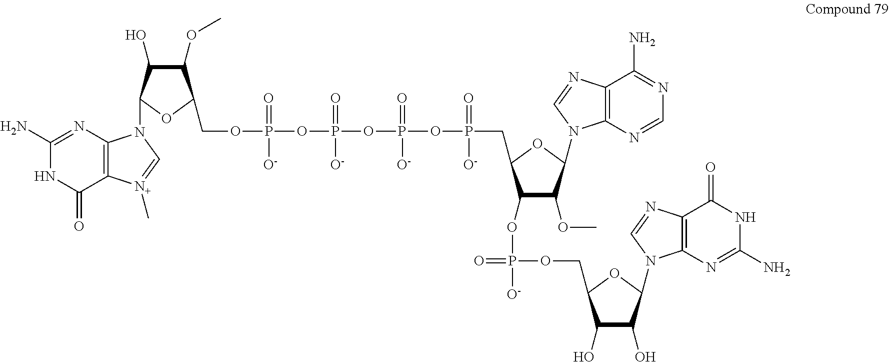

^{m7}G_{3′OMe}pppp^{m6}A_{2′OMe}pG:

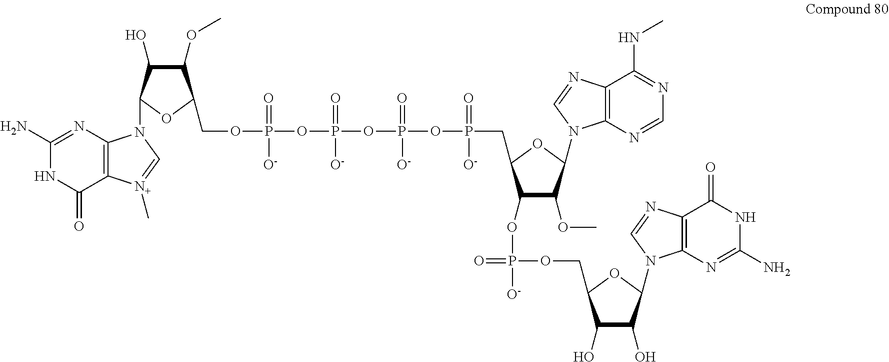

In some examples, the compounds described herein include a deuterated form of the compound, in which at least one (e.g., one or more, two or more, three or more, four or more, or five or more) hydrogen atom is replaced with deuterium. Exemplary deuterated forms include, for example, the following compounds which are shown as representative examples:

^{m7}G_{3′OCD3}pppA_{2′OMe}pG:

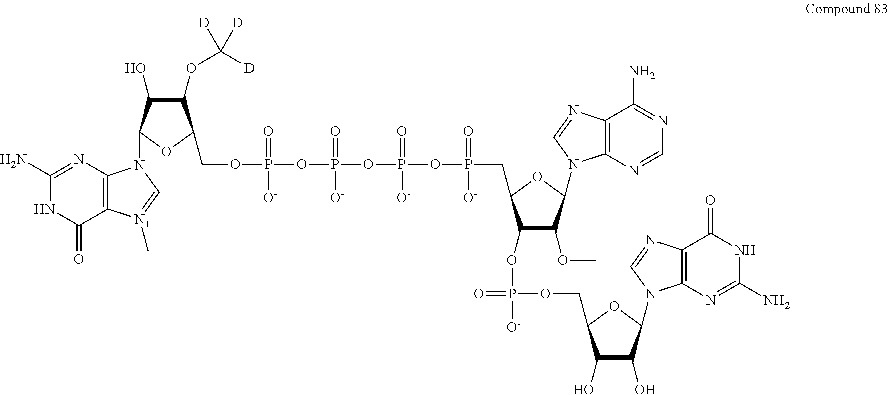

^{m7}G_{3′OMe}ppp^{(CD3)6}A_{2′OMe}pG:

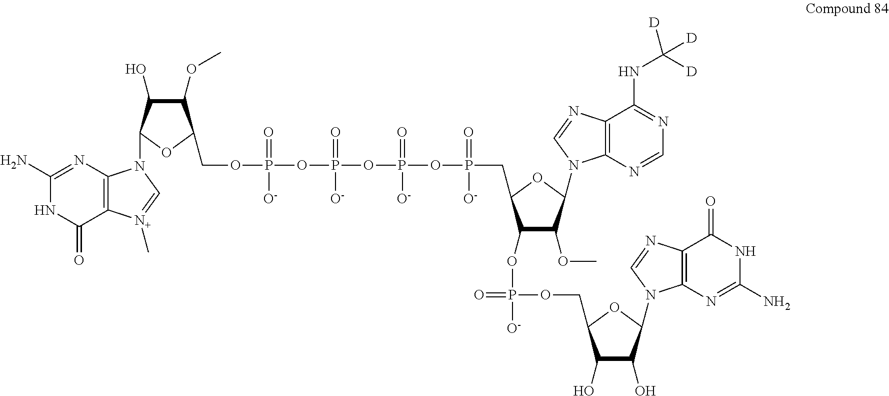

### III. Methods of Making the Compounds

The compounds described herein can be prepared in a variety of ways. The compounds can be synthesized using various synthetic methods. At least some of these methods are known in the art of synthetic organic chemistry. The compounds described herein can be prepared from readily available starting materials. Optimum reaction conditions can vary with the particular reactants or solvent used, but such conditions can be determined by one skilled in the art by routine optimization procedures.

Variations on Formula I, Formula II, Formula III, Formula IV, Formula V, Formula VI, Formula VII, Formula VIII, Formula IX, Formula X, Formula XI, and Formula XII include the addition, subtraction, or movement of the various constituents as described for each compound. Similarly, when one or more chiral centers are present in a molecule, all possible chiral variants are included. Additionally, compound synthesis can involve the protection and deprotection of various chemical groups. The use of protection and deprotection, and the selection of appropriate protecting groups can be determined by one skilled in the art. The chemistry of protecting groups can be found, for example, in Wuts, Greene's Protective Groups in Organic Synthesis, 5th. Ed., Wiley & Sons, 2014, which is incorporated herein by reference in its entirety.

Reactions to produce the compounds described herein can be carried out in solvents, which can be selected by one of skill in the art of organic synthesis. Solvents can be substantially nonreactive with the starting materials (reactants), the intermediates, or products under the conditions at which the reactions are carried out, i.e., temperature and pressure. Reactions can be carried out in one solvent or a mixture of more than one solvent. Product or intermediate formation can be monitored according to any suitable method known in the art. For example, product formation can be monitored by spectroscopic means, such as nuclear magnetic resonance spectroscopy (e.g., ¹H or ¹³C) infrared spectroscopy, spectrophotometry (e.g., UV-visible), or mass spectrometry, or by chromatography such as high-performance liquid chromatography (HPLC) or thin layer chromatography.

The compounds as described herein can be prepared through a two-step process, including an activation stage followed by a coupling stage. In the activation stage, the modified N7-methyl-guanosine-5′-diphosphate (optionally as a triethylammonium salt (TEAH⁺); N7-Me-GDP) compound, wherein R₁ is H or CH₃, is dissolved in a solvent mixture to achieve a desired concentration. An activating reagent, such as 1-(3-dimethylaminopropyl)-3-ethylcarbodiimide hydrochloride salt (EDC·HCl salt), is then added, forming a reactive intermediate in-situ, which is then reacted with imidazole. After a period of time, an activated intermediate is formed and prepared for use in a coupling step. See Scheme A below.

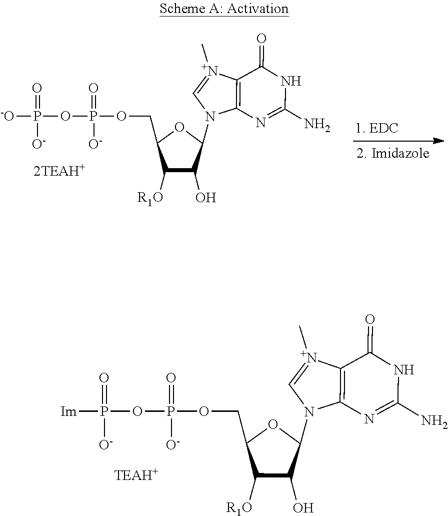

In the coupling step, the activated intermediate is reacted with the desired dinucleotide (wherein R₂ is H and R₃ is OCH₃ or F, or R₂ and R₃ are covalently bonded together and, together with intermediate atoms, form a 2′-O, 4′-C methylene bridge) to couple to the activated intermediate, and the mixture is allowed to react for a period of time. The final product is then obtained, as shown in Scheme B below.

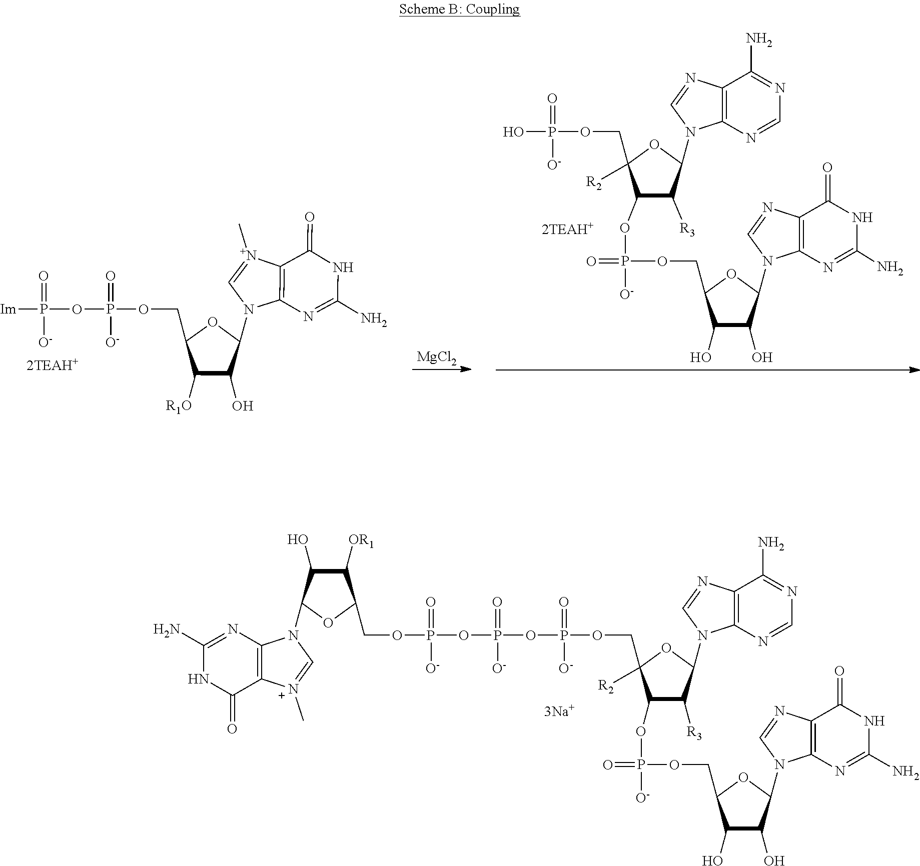

Schemes A and B shown above are provided for representative purposes only, and those of ordinary skill in the art will understand that the schemes can be applied, with modifications within the purview of those of skill in the art along with the disclosures in the Examples section, to any of the Formulae described herein (i.e., Formula I, Formula II, Formula III, Formula IV, Formula V, Formula VI, Formula VII, Formula VIII, Formula IX, Formula X, Formula XI, and Formula XII). By way of example, while Scheme B reflects a dinucleotide including adenosine and guanosine (pApG dinucleotide), the methods described herein can be similarly applied to other dinucleotides (e.g., p^{m6}ApG dinucleotides, pp^{m6}ApG dinucleotides, ppApG dinucleotides, pDAP(diaminopurine)pG dinucleotides, pApA dinucleotides, pApU dinucleotides, and others as described herein). Similarly, the two-step method shown above depicts phosphoro-linkages; however, the two-step method can be similarly applied, using the appropriate reagents, to phosphorothioate linkages according to Formula I, Formula II, Formula IV, and Formula VI.

The synthetic method described herein can be performed as a one-pot synthesis, such that all steps are performed in a single reactor with no isolation of intermediate products during the course of the synthetic method. In some examples, a method of synthesizing a trinucleotide compound comprises mixing an N7-methyl-guanosine-5′-diphosphate of the following structure:

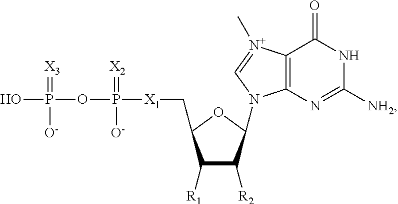

wherein X₁ and X₂ are each independently selected from the group consisting of O and S; X₃ is O; R₁ and R₂ are each independently selected from H, OH, N₃, F, substituted or unsubstituted alkoxy, and thio, wherein R₁ and R₂ optionally combine to form a heterocycle, with an activating reagent and an imidazole to form an activated intermediate; and adding a salt reagent (e.g., a chloride salt, such as MgCl₂ or ZnCl₂) and a dinucleotide of the following structure:

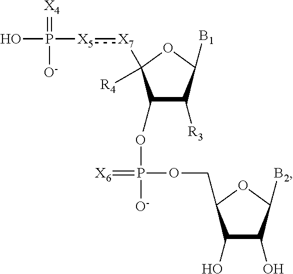

wherein [symbol] is a single bond or a double bond; X₄ and X₆ are each independently selected from the group consisting of O and S; X₅ is O, S, or CH; X₇ is CH or CH₂; R₃ is OH, OMe, F and R₄ is H, or wherein R₃ and R₄ are covalently bonded together and, together with intermediate atoms, form a 2′-O, 4′-C methylene bridge; B₁ and B₂ are each independently selected from the group consisting of a purine ring and a pyrimidine ring, to the activated intermediate to form the trinucleotide compound of the following structure:

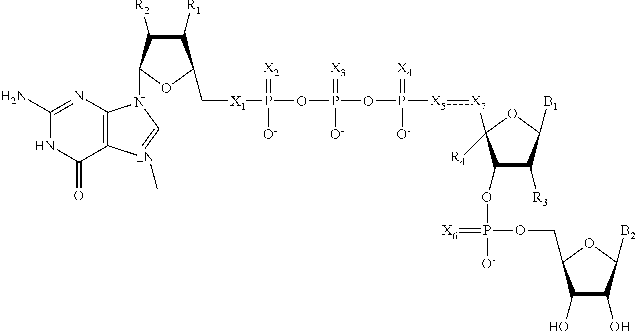

wherein the method is a one-pot synthesis.

In other examples, the method of synthesizing a trinucleotide compound comprises mixing a dinucleotide of the following structure:

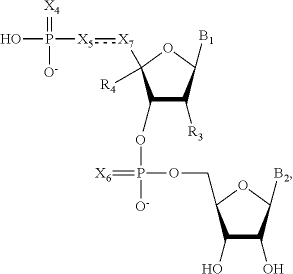

wherein [symbol]
is a single bond or a double bond; X₄ is O; X₆ is O or S; X₅ is O, S, or CH; X₇ is CH or CH₂; R₃ is OH, OMe, F and R₄ is H, or wherein R₃ and R₄ are covalently bonded together and, together with intermediate atoms, form a 2′-O, 4′-C methylene bridge; and B₁ and B₂ are each independently selected from the group consisting of a purine ring and a pyrimidine ring, with an activating reagent and an imidazole to form an activated phosphate imidazolide of the following structure:

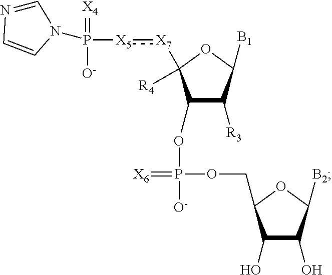

and
adding a salt reagent (e.g., a chloride salt such as magnesium chloride or zinc chloride) and a compound of the following structure:

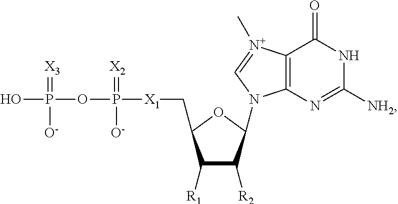

wherein
X₁ and X₂ are each independently selected from the group consisting of O and S; X₃ is S; R₁ and R₂ are each independently selected from H, OH, N₃, F, substituted or unsubstituted alkoxy, and thio, wherein R₁ and R₂ optionally combine to form a heterocycle, to the activated phosphate imidazolide to form the trinucleotide compound of the following structure:

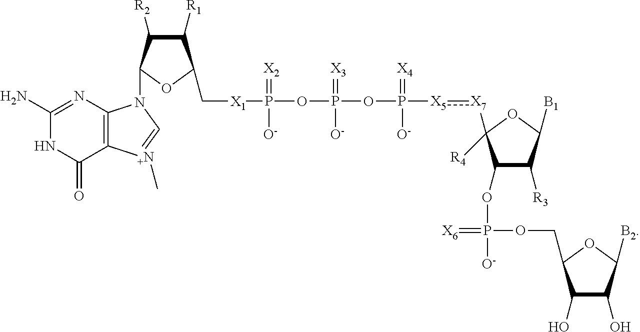

In still other examples, the method of synthesizing a trinucleotide compound comprises mixing a diphosphate dinucleotide of the following structure:

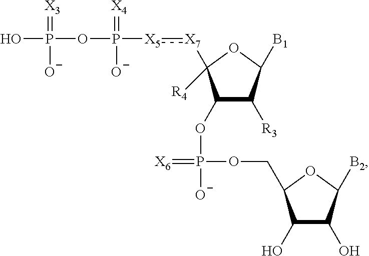

wherein [symbol]
is a single bond or a double bond; X₃ is O; X₄ and X₆ are each independently selected from O and S; X₅ is O, S, or CH; X₇ is CH or CH₂; R₃ is OH, OMe, F and R₄ is H, or wherein R₃ and R₄ are covalently bonded together and, together with intermediate atoms, form a 2′-O, 4′-C methylene bridge; and B₁ and B₂ are each independently selected from the group consisting of a purine ring and a pyrimidine ring, with an activating reagent and an imidazole to form an activated phosphate imidazolide of the following structure:

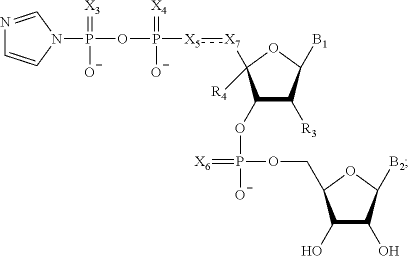

and
adding a salt reagent (e.g., a chloride salt such as magnesium chloride or zinc chloride) and a compound of the following structure:

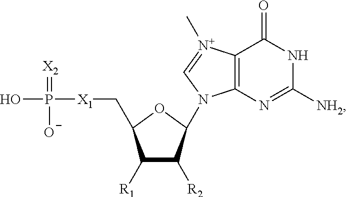

wherein
X₁ and X₂ are each independently selected from O and S; and R₁ and R₂ are each independently selected from H, OH, N₃, F, substituted or unsubstituted alkoxy, and thio, wherein R₁ and R₂ optionally combine to form a heterocycle, to the activated phosphate imidazolide to form the trinucleotide compound of the following structure:

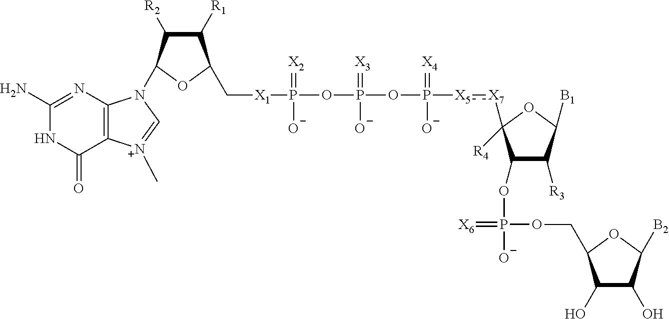

wherein the method is a one-pot synthesis.

Exemplary procedures for synthesizing the compounds as described herein are provided in Example 1 below.

### IV. Pharmaceutical Formulations

The compounds described herein or derivatives thereof can be provided in a pharmaceutical composition. Depending on the intended mode of administration, the pharmaceutical composition can be in the form of solid, semi-solid or liquid dosage forms, such as, for example, tablets, suppositories, pills, capsules, powders, liquids, or suspensions, preferably in unit dosage form suitable for single administration of a precise dosage. The compositions will include a therapeutically effective amount of the compound described herein or derivatives thereof in combination with a pharmaceutically acceptable carrier and, in addition, may include other medicinal agents, pharmaceutical agents, carriers, or diluents. By pharmaceutically acceptable is meant a material that is not biologically or otherwise undesirable, which can be administered to an individual along with the selected compound without causing unacceptable biological effects or interacting in a deleterious manner with the other components of the pharmaceutical composition in which it is contained.

As used herein, the term carrier encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington: The Science and Practice of Pharmacy, 22d Edition, Loyd et al. eds., Pharmaceutical Press and Philadelphia College of Pharmacy at University of the Sciences (2012). Examples of physiologically acceptable carriers include buffers, such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers, such as polyvinylpyrrolidone; amino acids such as glycine, glutamine, asparagine, arginine or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN® (ICI, Inc.; Bridgewater, New Jersey), polyethylene glycol (PEG), and PLURONICS™ (BASF; Florham Park, NJ).

Compositions containing the compound described herein or derivatives thereof suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants, such as preserving, wetting, emulsifying, and dispensing agents. Prevention of the action of microorganisms can be promoted by various antibacterial and antifungal agents, for example, parabens, chlorobutanol, phenol, sorbic acid, and the like. Isotonic agents, for example, sugars, sodium chloride, and the like may also be included. Prolonged absorption of the injectable pharmaceutical form can be brought about by the use of agents delaying absorption, for example, aluminum monostearate and gelatin.

Solid dosage forms for oral administration of the compounds described herein or derivatives thereof include capsules, tablets, pills, powders, and granules. In such solid dosage forms, the compounds described herein or derivatives thereof is admixed with at least one inert customary excipient (or carrier), such as sodium citrate or dicalcium phosphate, or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example, paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid compositions of a similar type may also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others known in the art. They may contain opacifying agents and can also be of such composition that they release the active compound or compounds in a certain part of the intestinal tract in a delayed manner. Examples of embedding compositions that can be used are polymeric substances and waxes. The active compounds can also be in micro-encapsulated form, if appropriate, with one or more of the above-mentioned excipients.

Liquid dosage forms for oral administration of the compounds described herein or derivatives thereof include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like.

Besides such inert diluents, the composition can also include additional agents, such as wetting, emulsifying, suspending, sweetening, flavoring, or perfuming agents.

Suspensions, in addition to the active compounds, may contain additional agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions of the compounds described herein or derivatives thereof for rectal administrations are optionally suppositories, which can be prepared by mixing the compounds with suitable non-irritating excipients or carriers, such as cocoa butter, polyethyleneglycol or a suppository wax, which are solid at ordinary temperatures but liquid at body temperature and, therefore, melt in the rectum or vaginal cavity and release the active component.

Dosage forms for topical administration of the compounds described herein or derivatives thereof include ointments, powders, sprays, inhalants, and skin patches. The compounds described herein or derivatives thereof are admixed under sterile conditions with a physiologically acceptable carrier and any preservatives, buffers, or propellants as may be required. Ophthalmic formulations, ointments, powders, and solutions are also contemplated as being within the scope of the compositions.

Optionally, the compounds described herein can be contained in a drug depot. A drug depot comprises a physical structure to facilitate implantation and retention in a desired site (e.g., a synovial joint, a disc space, a spinal canal, abdominal area, a tissue of the patient, etc.). The drug depot can provide an optimal concentration gradient of the compound at a distance of up to about 0.1 cm to about 5 cm from the implant site. A depot, as used herein, includes but is not limited to capsules, microspheres, microparticles, microcapsules, microfibers particles, nanospheres, nanoparticles, coating, matrices, wafers, pills, pellets, emulsions, liposomes, micelles, gels, antibody-compound conjugates, protein-compound conjugates, or other pharmaceutical delivery compositions. Suitable materials for the depot include pharmaceutically acceptable biodegradable materials that are preferably FDA approved or GRAS materials. These materials can be polymeric or non-polymeric, as well as synthetic or naturally occurring, or a combination thereof. The depot can optionally include a drug pump.

The compositions can include one or more of the compounds described herein and a pharmaceutically acceptable carrier. As used herein, the term pharmaceutically acceptable salt refers to those salts of the compound described herein or derivatives thereof that are, within the scope of sound medical judgment, suitable for use in contact with the tissues of subjects without undue toxicity, irritation, allergic response, and the like, commensurate with a reasonable benefit/risk ratio, and effective for their intended use, as well as the zwitterionic forms, where possible, of the compounds described herein. The term salts refers to the relatively non-toxic, inorganic and organic acid addition salts of the compounds described herein. These salts can be prepared in situ during the isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, methane sulphonate, and laurylsulphonate salts, and the like. These may include cations based on the alkali and alkaline earth metals, such as sodium, lithium, potassium, calcium, magnesium, and the like, as well as non-toxic ammonium, quaternary ammonium, and amine cations including, but not limited to ammonium, tetramethylammonium, tetraethylammonium, methylamine, dimethylamine, trimethylamine, triethylamine, ethylamine, and the like. (See S. M. Barge et al., J. Pharm. Sci. (1977) 66, 1, which is incorporated herein by reference in its entirety, at least, for compositions taught therein.)

Administration of the compounds and compositions described herein or pharmaceutically acceptable salts thereof can be carried out using therapeutically effective amounts of the compounds and compositions described herein or pharmaceutically acceptable salts thereof as described herein for periods of time effective to treat a disorder. The effective amount of the compounds and compositions described herein or pharmaceutically acceptable salts thereof as described herein may be determined by one of ordinary skill in the art.

Those of skill in the art will understand that the specific dose level and frequency of dosage for any particular subject may be varied and will depend upon a variety of factors, including the activity of the specific compound employed, the metabolic stability and length of action of that compound, the species, age, body weight, general health, sex and diet of the subject, the mode and time of administration, rate of excretion, drug combination, and severity of the particular condition.

The precise dose to be employed in the formulation will also depend on the route of administration, and the seriousness of the disease or disorder, and should be decided according to the judgment of the practitioner and each subject's circumstances. Effective doses can be extrapolated from dose-response curves derived from in vitro or animal model test systems. Further, depending on the route of administration, one of skill in the art would know how to determine doses that result in a plasma concentration for a desired level of response in the cells, tissues and/or organs of a subject.

### V. RNA Synthesis

Provided herein are in vitro methods for synthesizing capped RNA transcripts, including capped messenger RNA (mRNA) transcripts. The methods described herein comprise (a) forming a reaction mixture comprising a compound as described herein (also referred to herein as a cap analog), a DNA template, and an RNA polymerase; and (b) incubating the reaction mixture under conditions that allow transcription of the DNA template to produce capped mRNA transcripts. In the methods described herein, the reaction mixture comprises NTPs, including ATP, CTP, GTP and UTP. One or more of the NTPs in the in vitro transcription reaction mixture can be a modified NTP. Exemplary nucleosides with modified bases include, but are not limited to, inosine, 7-deazaguanosine, 7-methylguanosine, dihyrouridine, 2′-O-methylguanosine, 2′-fluoro-2′-deoxycytidine, pseudouridine, N1-methylpseudouridine, 5-methyluridine. In some methods, one or more uridines in the in vitro transcribed RNA are replaced by a modified nucleoside. Optionally, some methods further comprise incubating the reaction mixture comprising the capped mRNA transcripts with a DNase I buffer including Ca²⁺ and DNase I.

Optionally, some methods further comprise subjecting the DNase treated reaction mixture to eliminate proteins from the in vitro transcription reaction. Optionally, some methods further comprise subjecting the DNase treated reaction mixture to phosphatase treatment. Optionally, the method further comprises subjecting the DNase treated reaction mixture to one or more purification steps. The mRNA transcripts produced by the methods described herein can be purified using one or more purification techniques known to those of skill in the art. See, Baronti et al. “A guide to large-scale RNA sample preparation,” Anal. Bioanal. Chem. 410(14): 3239-33252 (2018). For example, the mRNAs can be purified by liquid chromatography (e.g., HPLC, reversed-phase ion pairing HPLC (RP-IP-HPLC), anion-exchange chromatography, cation exchange chromatography, affinity chromatography, size-exclusion chromatography), precipitation, diafiltration, tangential flow filtration, oligo dT chromatography, silica membrane purification, and hydrophobic interaction chromatography, to name a few. The synthesized capped mRNA transcripts can be substantially free of impurities such as DNA, protein, double-stranded RNA and/or incomplete mRNA transcripts.

Also described herein are the resulting RNA molecules comprising a 5′-cap, wherein the 5′-cap comprises a compound as described herein. Optionally, the RNA molecule is a messenger RNA (mRNA) molecule. Optionally, the RNA molecule is a self-amplifying RNA (saRNA) molecule.

Methods of inducing a therapeutic effect in a subject are also provided herein, the methods comprising administering to the subject an RNA molecule as described herein. The administered RNA may contain some amount of immunogenic double stranded RNA (dsRNA). The amount of dsRNA is calculated per each ug of administered RNA.

All 5′ cap analogs described herein are useful for optimized in vivo translation of mRNAs, as further detailed herein.

Further provided herein is a method of administering to an animal a dose of an mRNA molecule comprising a 5′-cap as described herein. Optionally, the 5′-cap comprises a compound selected from the group consisting of:

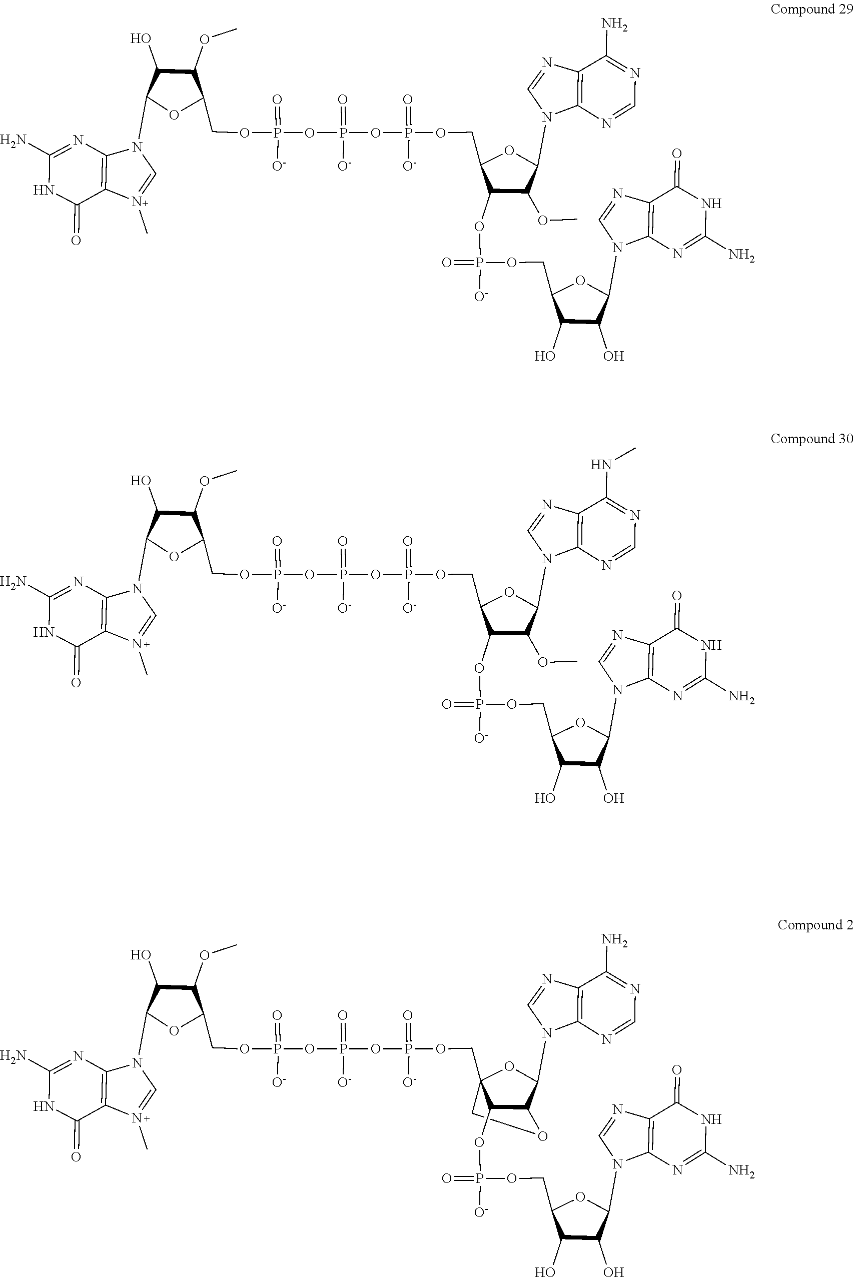

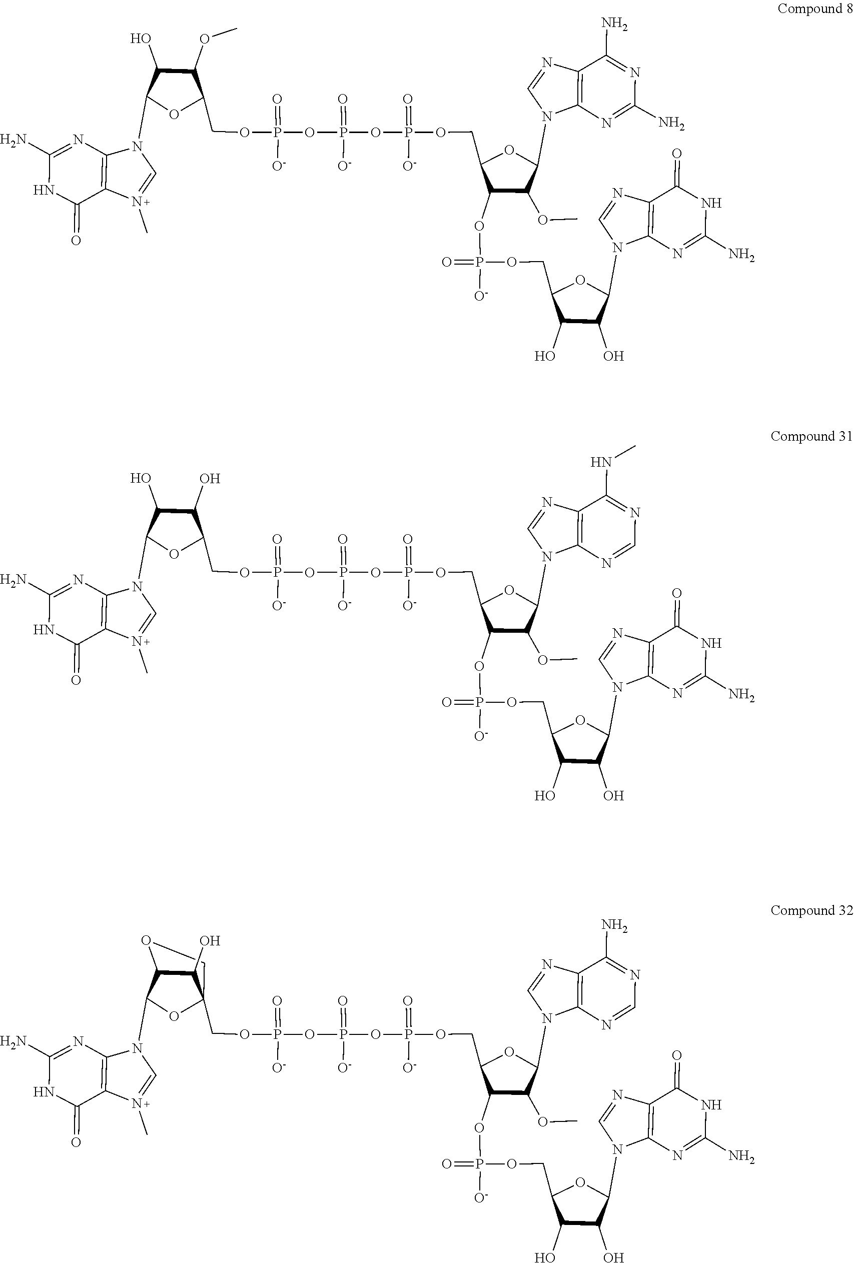

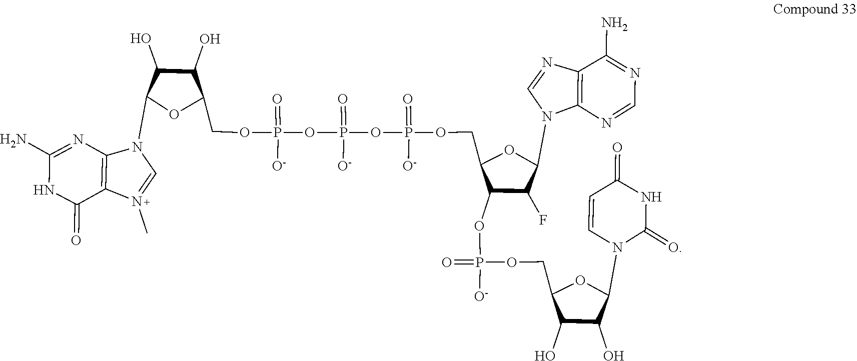

The mRNA dose optionally comprises less than 7 ng of double stranded RNA (dsRNA) per 1 μg of mRNA, wherein the subject exhibits increased tolerability to the administered dose of the mRNA as compared to an equivalent dose of the mRNA comprising 7 ng/μg or greater dsRNA. Optionally, the increased tolerability is determined by measuring one or more of body weight, organ weight, aspartate aminotransferase (AST) levels, alanine transaminase (ALT) levels, C-reactive protein (CRP) levels, procalcitonin (PCT) levels, interleukin-6 (IL-6) levels, erythrocyte sedimentation rate (ESR), serum amyloid A levels, and serum ferritin levels prior to the administering and a period of time after the administering.

In some cases, the increased tolerability is measured by testing in a standard in vivo assay. As described above, the term “in vivo assays” refer to methods used to detect and/or measure capacity of one or more of the compounds or molecules including the compounds (e.g., mRNA molecules in, for example, a therapeutic dose) to increase or decrease a property relative to a control (e.g., biomarker levels). Optionally, in vivo assays as described herein can be used to determine a subject's tolerability levels to a given compound or molecule. Exemplary measurements for assessing tolerability include one or more of body weight, organ weight, aspartate aminotransferase (AST) levels, alanine transaminase (ALT) levels, C-reactive protein (CRP) levels, procalcitonin (PCT) levels, interleukin-6 (IL-6) levels, erythrocyte sedimentation rate (ESR), serum amyloid A levels, and serum ferritin levels. Optionally, the in vivo assays can be characterized herein as a “standard in vivo assay” and can optionally be performed using the conditions outlined in Examples 2 and 3 of the present application.

In some cases, the increased tolerability is an increase in tolerability of at least 20% to the administered dose unit as measured by testing in a standard in vivo assay, at least 25% to the administered dose unit as measured by testing in a standard in vivo assay, at least 30% to the administered dose unit as measured by testing in a standard in vivo assay, at least 35% to the administered dose unit as measured by testing in a standard in vivo assay, at least 40% to the administered dose unit as measured by testing in a standard in vivo assay, at least 45% to the administered dose unit as measured by testing in a standard in vivo assay, at least 50% to the administered dose unit as measured by testing in a standard in vivo assay, at least 55% to the administered dose unit as measured by testing in a standard in vivo assay, at least 60% to the administered dose unit as measured by testing in a standard in vivo assay, at least 65% to the administered dose unit as measured by testing in a standard in vivo assay, at least 70% to the administered dose unit as measured by testing in a standard in vivo assay, at least 75% to the administered dose unit as measured by testing in a standard in vivo assay, at least 80% to the administered dose unit as measured by testing in a standard in vivo assay, at least 85% to the administered dose unit as measured by testing in a standard in vivo assay, at least 90% to the administered dose unit as measured by testing in a standard in vivo assay, or at least 95% to the administered dose unit as measured by testing in a standard in vivo assay.

Optionally, the therapeutic dose unit of the mRNA molecule comprises less than 6.9 ng/μg of double stranded RNA (dsRNA), less than 6.8 ng/μg of double stranded RNA (dsRNA), less than 6.7 ng/μg of double stranded RNA (dsRNA), less than 6.6 ng/μg of double stranded RNA (dsRNA), less than 6.5 ng/μg of double stranded RNA (dsRNA), less than 6.4 ng/μg of double stranded RNA (dsRNA), less than 6.3 ng/μg of double stranded RNA (dsRNA), less than 6.2 ng/μg of double stranded RNA (dsRNA), less than 6.1 ng/μg of double stranded RNA (dsRNA), less than 6.0 ng/μg of double stranded RNA (dsRNA), less than 5.9 ng/μg of double stranded RNA (dsRNA), less than 5.8 ng/μg of double stranded RNA (dsRNA), less than 5.7 ng/μg of double stranded RNA (dsRNA), less than 5.6 ng/μg of double stranded RNA (dsRNA), less than 5.5 ng/μg of double stranded RNA (dsRNA), less than 5.4 ng/μg of double stranded RNA (dsRNA), less than 5.3 ng/μg of double stranded RNA (dsRNA), less than 5.2 ng/μg of double stranded RNA (dsRNA), less than 5.1 ng/μg of double stranded RNA (dsRNA), less than 5.0 ng/μg of double stranded RNA (dsRNA), less than 4.9 ng/μg of double stranded RNA (dsRNA), less than 4.8 ng/μg of double stranded RNA (dsRNA), less than 4.7 ng/μg of double stranded RNA (dsRNA), less than 4.6 ng/μg of double stranded RNA (dsRNA), less than 4.5 ng/μg of double stranded RNA (dsRNA), less than 4.4 ng/μg of double stranded RNA (dsRNA), less than 4.3 ng/μg of double stranded RNA (dsRNA), less than 4.2 ng/μg of double stranded RNA (dsRNA), less than 4.1 ng/μg of double stranded RNA (dsRNA), less than 4.0 ng/μg of double stranded RNA (dsRNA), less than 3.9 ng/μg of double stranded RNA (dsRNA), less than 3.8 ng/μg of double stranded RNA (dsRNA), less than 3.7 ng/μg of double stranded RNA (dsRNA), less than 3.6 ng/μg of double stranded RNA (dsRNA), less than 3.5 ng/μg of double stranded RNA (dsRNA), less than 3.4 ng/μg of double stranded RNA (dsRNA), less than 3.3 ng/μg of double stranded RNA (dsRNA), less than 3.2 ng/μg of double stranded RNA (dsRNA), less than 3.1 ng/μg of double stranded RNA (dsRNA), less than 3.0 ng/μg of double stranded RNA (dsRNA), less than 2.9 ng/μg of double stranded RNA (dsRNA), less than 2.8 ng/μg of double stranded RNA (dsRNA), less than 2.7 ng/μg of double stranded RNA (dsRNA), less than 2.6 ng/μg of double stranded RNA (dsRNA), less than 2.5 ng/μg of double stranded RNA (dsRNA), less than 2.4 ng/μg of double stranded RNA (dsRNA), less than 2.3 ng/μg of double stranded RNA (dsRNA), less than 2.2 ng/μg of double stranded RNA (dsRNA), less than 2.1 ng/μg of double stranded RNA (dsRNA), less than 2.0 ng/μg of double stranded RNA (dsRNA), less than 1.9 ng/μg of double stranded RNA (dsRNA), less than 1.8 ng/μg of double stranded RNA (dsRNA), less than 1.7 ng/μg of double stranded RNA (dsRNA), less than 1.6 ng/μg of double stranded RNA (dsRNA), less than 1.5 ng/μg of double stranded RNA (dsRNA), less than 1.4 ng/μg of double stranded RNA (dsRNA), less than 1.3 ng/μg of double stranded RNA (dsRNA), less than 1.2 ng/μg of double stranded RNA (dsRNA), less than 1.1 ng/μg of double stranded RNA (dsRNA), less than 1.0 ng/μg of double stranded RNA (dsRNA), less than 0.9 ng/μg of double stranded RNA (dsRNA), less than 0.8 ng/μg of double stranded RNA (dsRNA), less than 0.7 ng/μg of double stranded RNA (dsRNA), less than 0.6 ng/μg of double stranded RNA (dsRNA), less than 0.5 ng/μg of double stranded RNA (dsRNA), less than 0.4 ng/μg of double stranded RNA (dsRNA), less than 0.3 ng/μg of double stranded RNA (dsRNA), less than 0.2 ng/μg of double stranded RNA (dsRNA), or less than 0.1 ng/μg of double stranded RNA (dsRNA).

Also described herein are methods for increasing in vivo translation of a polypeptide in a subject. The methods include administering to the subject a therapeutic dose unit comprising an effective amount of an mRNA encoding a polypeptide, wherein the mRNA comprises a 5′-cap having a formula according to any of the compounds as described herein.

In some examples, the methods include administering to the subject a therapeutic dose unit comprising an effective amount of an mRNA encoding a polypeptide, wherein the mRNA comprises a 5′-cap having the following formula ^{m7}G_{3′OMe}pppA¹_{2′OMe}G and wherein A¹ is adenosine, N6-methyladenosine, or diaminopurine. A¹ can optionally include an LNA moiety. The ^{m7}G_{3′OMe}pppA¹_{2′OMe}G compound for use in the methods for increasing translation of a polypeptide in a subject can include one or more of the following compounds:

^{m7}G_{3′OMe}pppA_{2′OMe}pG:

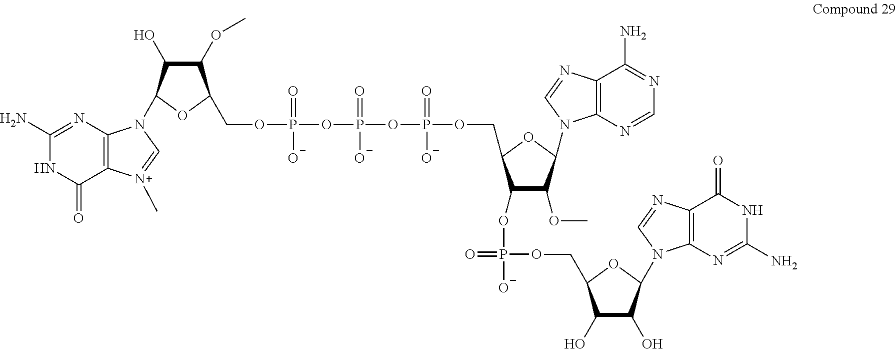

^{m7}G_{3′OMe}ppp^{m6}A_{2′OMe}pG:

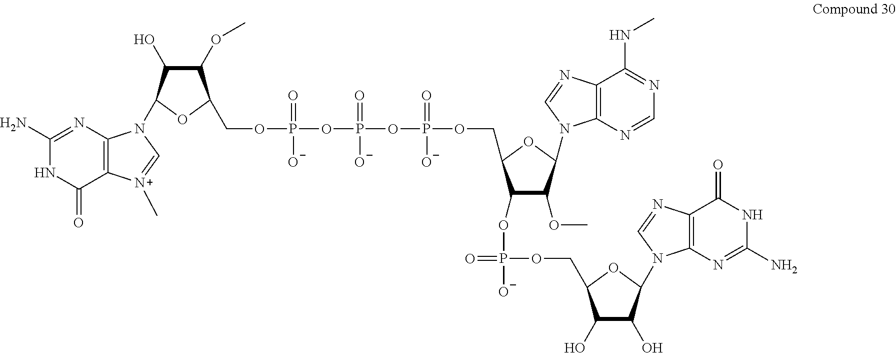

^{m7}G_{3′OMe}pppA_{2′,4′-LNA}pG:

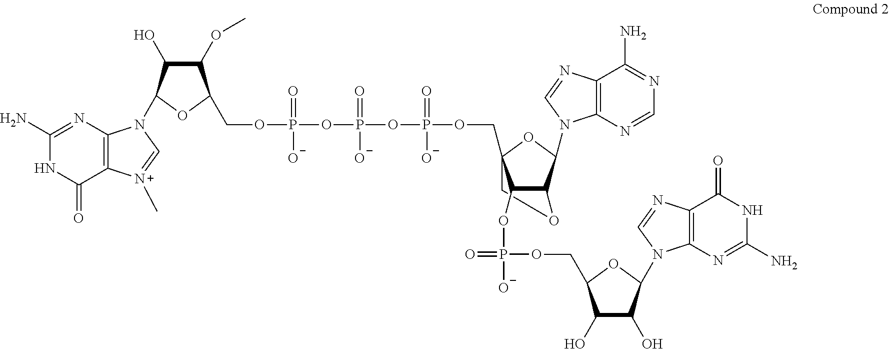

^{m7}G_{3′OMe}ppp(diaminopurine)_{2′OMe}pG:

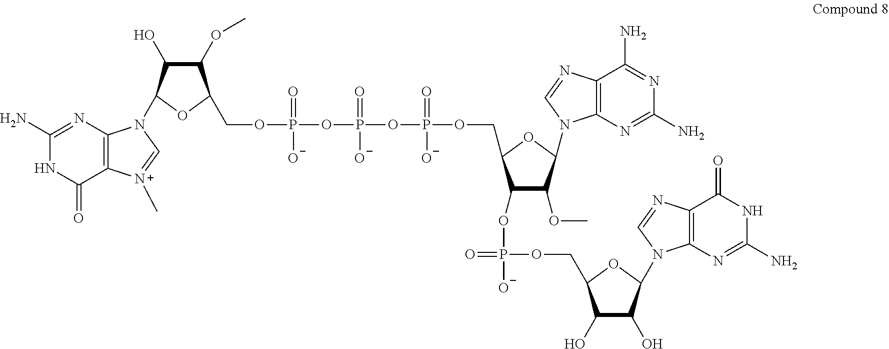

According to the methods described herein, the administered therapeutic dose unit is at least 20% lower than the therapeutic dose unit required to elicit the same response in the subject when administered a comparative mRNA encoding the polypeptide, wherein the comparative mRNA comprises a 5′-cap having the formula ^{m7}GpppA¹_{2′OMe}G, wherein A¹ is adenosine, N6-methyladenosine, or diaminopurine. A¹ can optionally include an LNA moiety. The ^{m7}GpppA¹_{2′OMe}G compound in the comparative mRNA can include one or more of the following compounds:

^{m7}GpppA_{2′OMe}pG:

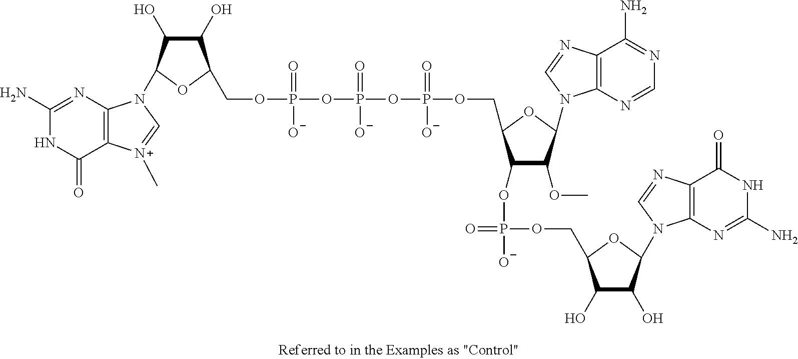

^{m7}Gppp^{m6}A_{2′OMe}pG:

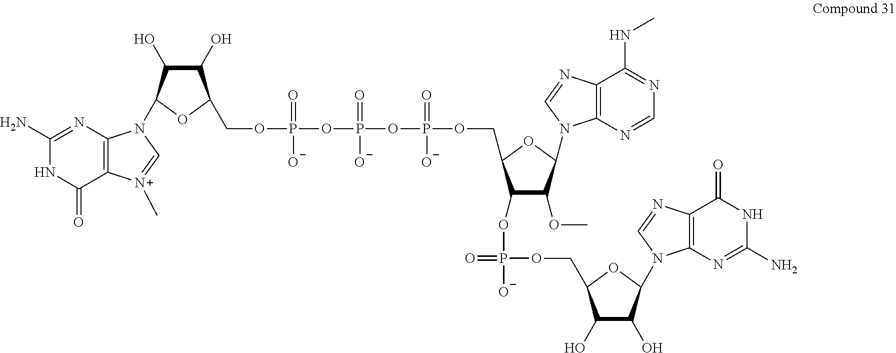

^{m7}GpppA_{2′,4′-LNA}pG:

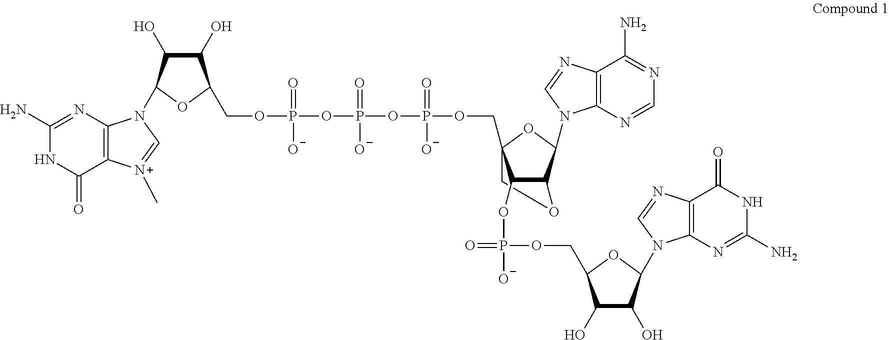

^{m7}Gppp(diaminopurine)_{2′OMe}pG:

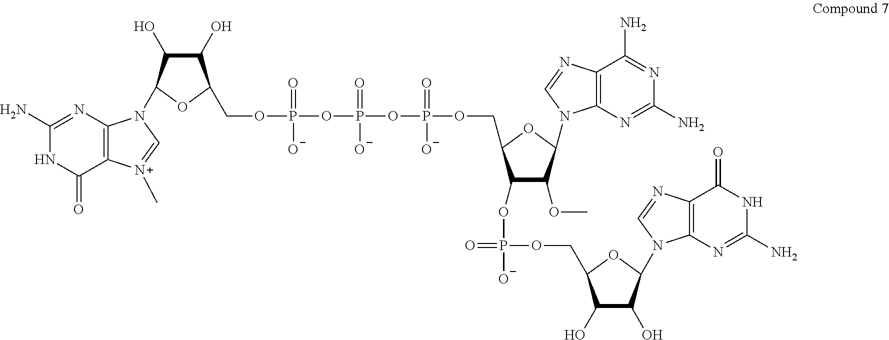

The ^{m7}GpppA¹_{2′OMe}G compound in the comparative mRNA can be selected such that the compound is identical to the administered ^{m7}G_{3′OMe}pppA¹_{2′OMe}G, except the comparative mRNA lacks a 3′ OMe moiety on the ^{m7}G nucleotide.

The administered therapeutic dose unit can be at least 20% lower, at least 30% lower, at least 40% lower, at least 50% lower, at least 60% lower, at least 70% lower, at least 80% lower, at least 90% lower, or at least 95% lower than the therapeutic dose unit required to elicit the same response in the subject when administered a comparative mRNA encoding the polypeptide, at outlined above.

The subject can exhibit increased tolerability to the administered therapeutic dose unit as compared to the therapeutic dose unit required to elicit the same response in the subject when administered the comparative mRNA, as measured by testing in a standard in vivo assay as described above, such as by using the conditions described in the Examples herein.

Optionally, the increased tolerability is an increase in tolerability of at least 20% to the administered therapeutic dose unit as measured by testing in a standard in vivo assay, at least 25% to the administered therapeutic dose unit as measured by testing in a standard in vivo assay, at least 30% to the administered therapeutic dose unit as measured by testing in a standard in vivo assay, at least 35% to the administered therapeutic dose unit as measured by testing in a standard in vivo assay, at least 40% to the administered therapeutic dose unit as measured by testing in a standard in vivo assay, at least 45% to the administered therapeutic dose unit as measured by testing in a standard in vivo assay, at least 50% to the administered therapeutic dose unit as measured by testing in a standard in vivo assay, at least 55% to the administered therapeutic dose unit as measured by testing in a standard in vivo assay, at least 60% to the administered therapeutic dose unit as measured by testing in a standard in vivo assay, at least 65% to the administered therapeutic dose unit as measured by testing in a standard in vivo assay, at least 70% to the administered therapeutic dose unit as measured by testing in a standard in vivo assay, at least 75% to the administered therapeutic dose unit as measured by testing in a standard in vivo assay, at least 80% to the administered therapeutic dose unit as measured by testing in a standard in vivo assay, at least 85% to the administered therapeutic dose unit as measured by testing in a standard in vivo assay, at least 90% to the administered therapeutic dose unit as measured by testing in a standard in vivo assay, or at least 95% to the administered therapeutic dose unit as measured by testing in a standard in vivo assay.

In some cases, the administered therapeutic mRNA dose unit comprises less than 6.9 ng/μg of double stranded RNA (dsRNA), less than 6.8 ng/μg of double stranded RNA (dsRNA), less than 6.7 ng/μg of double stranded RNA (dsRNA), less than 6.6 ng/μg of double stranded RNA (dsRNA), less than 6.5 ng/μg of double stranded RNA (dsRNA), less than 6.4 ng/μg of double stranded RNA (dsRNA), less than 6.3 ng/μg of double stranded RNA (dsRNA), less than 6.2 ng/μg of double stranded RNA (dsRNA), less than 6.1 ng/μg of double stranded RNA (dsRNA), less than 6.0 ng/μg of double stranded RNA (dsRNA), less than 5.9 ng/μg of double stranded RNA (dsRNA), less than 5.8 ng/μg of double stranded RNA (dsRNA), less than 5.7 ng/μg of double stranded RNA (dsRNA), less than 5.6 ng/μg of double stranded RNA (dsRNA), less than 5.5 ng/μg of double stranded RNA (dsRNA), less than 5.4 ng/μg of double stranded RNA (dsRNA), less than 5.3 ng/μg of double stranded RNA (dsRNA), less than 5.2 ng/μg of double stranded RNA (dsRNA), less than 5.1 ng/μg of double stranded RNA (dsRNA), less than 5.0 ng/μg of double stranded RNA (dsRNA), less than 4.9 ng/μg of double stranded RNA (dsRNA), less than 4.8 ng/μg of double stranded RNA (dsRNA), less than 4.7 ng/μg of double stranded RNA (dsRNA), less than 4.6 ng/μg of double stranded RNA (dsRNA), less than 4.5 ng/μg of double stranded RNA (dsRNA), less than 4.4 ng/μg of double stranded RNA (dsRNA), less than 4.3 ng/μg of double stranded RNA (dsRNA), less than 4.2 ng/μg of double stranded RNA (dsRNA), less than 4.1 ng/μg of double stranded RNA (dsRNA), less than 4.0 ng/μg of double stranded RNA (dsRNA), less than 3.9 ng/μg of double stranded RNA (dsRNA), less than 3.8 ng/μg of double stranded RNA (dsRNA), less than 3.7 ng/μg of double stranded RNA (dsRNA), less than 3.6 ng/μg of double stranded RNA (dsRNA), less than 3.5 ng/μg of double stranded RNA (dsRNA), less than 3.4 ng/μg of double stranded RNA (dsRNA), less than 3.3 ng/μg of double stranded RNA (dsRNA), less than 3.2 ng/μg of double stranded RNA (dsRNA), less than 3.1 ng/μg of double stranded RNA (dsRNA), less than 3.0 ng/μg of double stranded RNA (dsRNA), less than 2.9 ng/μg of double stranded RNA (dsRNA), less than 2.8 ng/μg of double stranded RNA (dsRNA), less than 2.7 ng/μg of double stranded RNA (dsRNA), less than 2.6 ng/μg of double stranded RNA (dsRNA), less than 2.5 ng/μg of double stranded RNA (dsRNA), less than 2.4 ng/μg of double stranded RNA (dsRNA), less than 2.3 ng/μg of double stranded RNA (dsRNA), less than 2.2 ng/μg of double stranded RNA (dsRNA), less than 2.1 ng/μg of double stranded RNA (dsRNA), less than 2.0 ng/μg of double stranded RNA (dsRNA), less than 1.9 ng/μg of double stranded RNA (dsRNA), less than 1.8 ng/μg of double stranded RNA (dsRNA), less than 1.7 ng/μg of double stranded RNA (dsRNA), less than 1.6 ng/μg of double stranded RNA (dsRNA), less than 1.5 ng/μg of double stranded RNA (dsRNA), less than 1.4 ng/μg of double stranded RNA (dsRNA), less than 1.3 ng/μg of double stranded RNA (dsRNA), less than 1.2 ng/μg of double stranded RNA (dsRNA), less than 1.1 ng/μg of double stranded RNA (dsRNA), less than 1.0 ng/μg of double stranded RNA (dsRNA), less than 0.9 ng/μg of double stranded RNA (dsRNA), less than 0.8 ng/μg of double stranded RNA (dsRNA), less than 0.7 ng/μg of double stranded RNA (dsRNA), less than 0.6 ng/μg of double stranded RNA (dsRNA), less than 0.5 ng/μg of double stranded RNA (dsRNA), less than 0.4 ng/μg of double stranded RNA (dsRNA), less than 0.3 ng/μg of double stranded RNA (dsRNA), less than 0.2 ng/μg of double stranded RNA (dsRNA), or less than 0.1 ng/μg of double stranded RNA (dsRNA).

### VI. Kits

Also provided herein are kits for performing transcription. A kit can include any of the compounds or compositions described herein. For example, a kit can include one or more compounds of Formula I, Formula II, Formula III, Formula IV, Formula V, Formula VI, Formula VII, Formula VIII, Formula IX, Formula X, Formula XI, and/or Formula XII. A kit can further include one or more additional reagents, such as reagents used for the synthesis of RNA. Optionally, a kit can contain a compound as described herein (also referred to herein as a cap analog), a container, and one or more reagents selected from one or more unmodified NTPs, one or more modified NTPs, an RNA polymerase, a reaction buffer, magnesium, and a DNA template. A kit can additionally include directions for use of the kit (e.g., instructions for performing RNA synthesis), a means for administering transcribed mRNA (e.g., a syringe), and/or a carrier.

### EXAMPLES

### Example 1: Compound Synthesis

The compounds described herein were prepared using the general procedures outlined below. The structures depicted herein may represent one particular isomer (e.g., one diastereomer). However, as noted above, the depicted structure may represent a mixture of isomers. By way of example, thiolated compounds as described herein (e.g., Compound 3, Compound 6, Compound 9, Compound 13, Compound 18, Compound 19, Compound 20, Compound 21, and Compound 74) include two diastereomers. In some instances, the two diastereomers were separated and tested separately (as indicated in the Examples below). However, unless otherwise indicated, the diastereomers were not separated prior to testing. Synthesis of Compounds According to Formulae I, II, IV, VI, VIII, IX, X, and XI

Compounds according to Formulae I, II, IV, and VI, terminating in a guanosine nucleotide, were prepared according to the general method shown in Scheme 1. Compounds according to Formula VIII are prepared similarly (wherein the position corresponding to R₂ in Scheme 1 can be OCH₃), along with compounds according to Formulae IX, X, and XI.

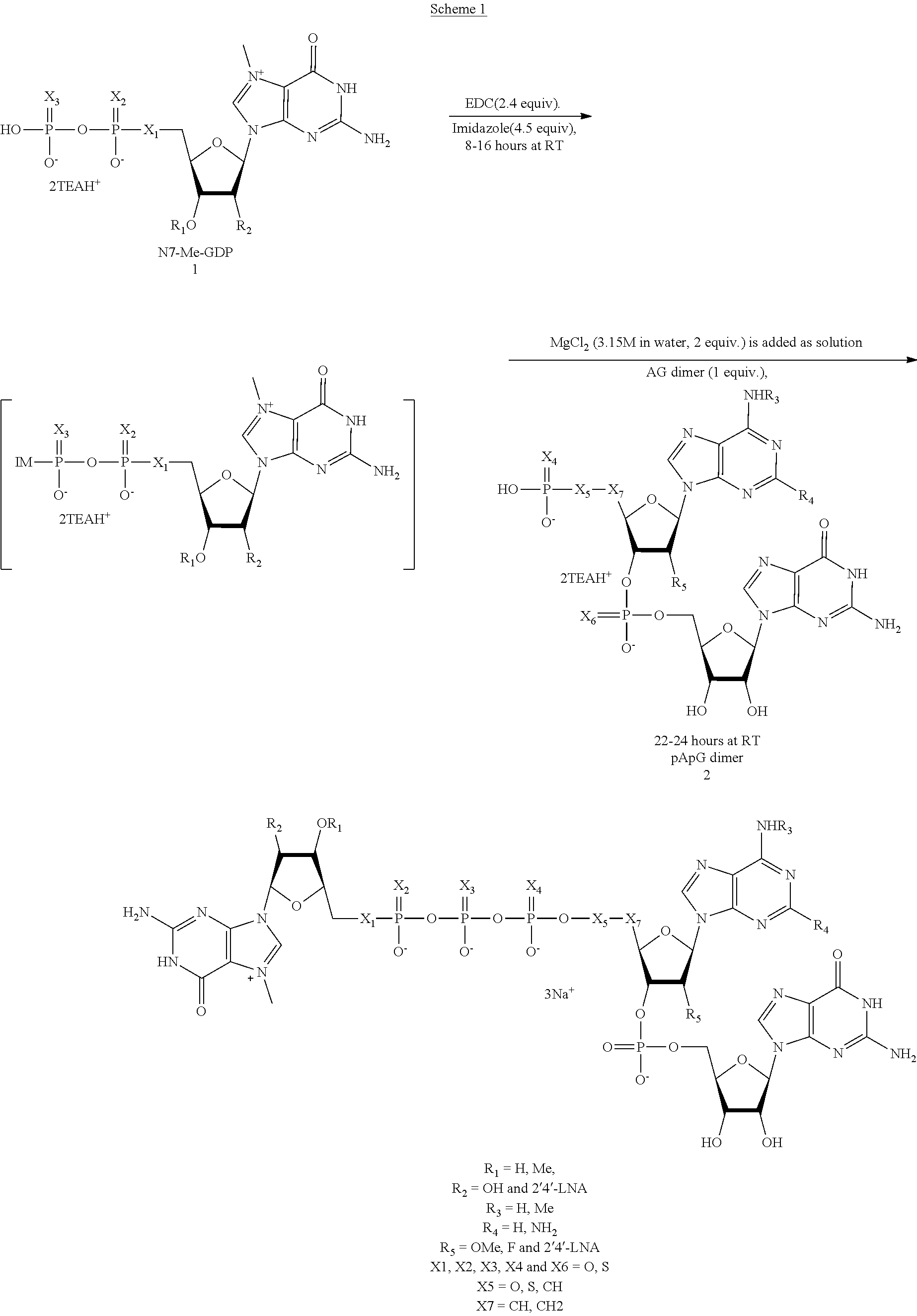

### General Procedure 1:

To a stirred solution of N7-Me-GDP 1 (triethylammonium (TEA) salt, 1.5 mol equivalent based on pApG dinucleotide 2) in a 10% water/DMSO solution (10 mL per each gram of N7-Me-GDP 1) at room temperature was added 1-(3-dimethylaminopropyl)-3-ethylcarbodiimide hydrochloride (EDC·HCl salt; 2.4 mole equivalent based on pApG dinucleotide 2), followed by imidazole (4.5 mole equivalent based on pApG dinucleotide 2). The resulting mixture was allowed to stir at room temperature overnight (approximately 16-24 hours). The second day, magnesium chloride (3.15 M in water, 2.0 mole equivalent based on pApG dinucleotide 2) was added to the above solution, followed by the pApG dinucleotide 2 (TEA salt, 1 mole equivalent). The resulting solution was allowed to stir at room temperature overnight (approximately 16 to 24 hours). The crude reaction mixture was then diluted with 10× water and purified by anion exchange chromatography (Q Sepharose Fast Flow (QFF) Resin, 20% acetonitrile in water as buffer A, 1.5 M trimethylamine acetate (TEAA) in water as buffer B, using a linear gradient from 25% to 45% buffer B for 4 column volumes (CV) and holding at 45% for 1.5 CV). The desired product was pooled and concentrated under vacuum, and the final product was precipitated as a sodium salt with sodium acetate and 95% absolute ethanol in water.

The syntheses of the following compounds followed the procedure described above and depicted in Scheme 1.

^{m7}Gppp^{m6}A_{2′OMe}pG (Compound 31):

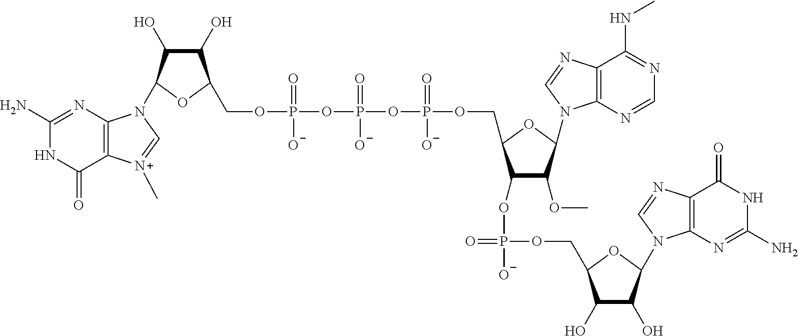

The compound ^{m7}Gppp^{m6}A_{2′OMe}pG (Compound 31) shown above was synthesized as described according to General Procedure 1 from N7-Me-GDP (0.40 g, 0.75 mmol), EDC (0.19 g, 0.975 mmol) and imidazole (0.07 g, 1.0 mmol) in 4.88 ml 10% water in DMSO, 3.15 M magnesium chloride (0.32 mL, 1 mmol) and p(m6)ApG (0.46 g, 0.5 mmol). Solid product (0.421 g) was obtained (yield: 68.6%) NMR (500 MHz, D₂O) δ 8.25 (s, 1H), 7.99 (s, 1H), 7.88 (s, 1H), 5.96 (d, J=5.2 Hz, 1H), 5.80 (d, J=3.5 Hz, 1H), 5.78 (d, J=5.3 Hz, 1H), 4.87-4.91 (m, 1H), 4.66-4.68 (m, 1H), 4.45-4.49 (m, 3H), 4.38-4.41 (m, 3H), 4.28-4.36 (m, 4H), 4.17-4.25 (m, 4H), 3.97 (s, 3H), 3.46 (s, 3H), 2.98 (s, 3H). ³¹P NMR (200 MHz, D₂O) 0.38 (s, 1P), −10.85 (d, J=12.1, 1P), −10.94 (d, J=12.1, 1P), −22.22 (t, J=18.2, 1P). MS m/z=1159.2.

^{m7}G_{3′OMe}ppp^{m6}A_{2′OMe}pG (Compound 30):

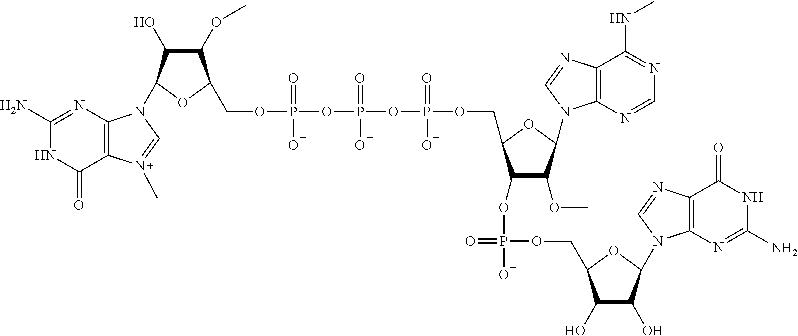

The compound ^{m7}G_{3′OMe}ppp^{m6}A_{2′OMe}pG (Compound 30) shown above was synthesized as described according to General Procedure 1 from N7-Me-3′-Ome-GDP (2.86 g, 4.5 mmol), EDC (1.12 g, 5.85 mmol) and imidazole (0.41 g, 6.0 mmol) in 28.6 ml 30% water in DMSO, 3.15 M magnesium chloride (1.90 mL, 6.0 mmol) and p(m6)ApG (2.76 g, 3.0 mmol). 3.092 g solid product was obtained (yield: 83%). 1H NMR (500 MHz, D₂O) δ 8.30 (s, 1H), 8.09 (s, 1H), 7.94 (s, 1H), 6.00 (d, J=5.5 Hz, 1H), 5.80-5.83 (m, 2H), 4.91-4.94 (m, 1H), 4.65-4.66 (m, 1H), 4.49-4.52 (m, 2H), 4.36-4.42 (m, 5H), 4.18-4.33 (m, 4H), 4.08-4.10 (m, 1H), 4.01 (s, 3H), 3.48-3.47 (m, 6H), 3.08 (s, 3H). ³¹P NMR (200 MHz, D₂O) 0.38 (s, 1P), −10.96 (d, J=4.0, 1P), −11.04 (d, J=8.1, 1P), −22.27 (t, J=18.2, 1P). MS m/z=1173.2.

^{m7}GpppA_{2′,4′-LNA}pG (Compound 1):

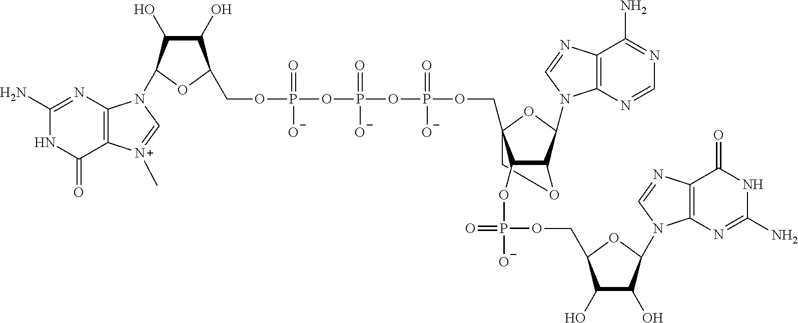

Compound 1 was synthesized as described according to General Procedure 1 from N7-Me-GDP (0.30 g, 0.49 mmol), EDC (0.151 g, 0.79 mmol) and imidazole (0.101 g, 1.48 mmol) in 3.0 ml 10% water in DMSO, magnesium chloride (62.4 mg in 0.21 mL water, 0.66 mmol) and p(LNA)ApG (0.298 g, 0.328 mmol). Solid product (0.283 g) was obtained (yield: 71%). ¹H NMR (500 MHz, D₂O): δ 8.07 (s, 1H), 7.95 (s, 1H), 7.75 (s, 1H), 5.85 (d, J=4.2 Hz, 1H), 5.69 (d, J=5.5 Hz, 2H), 4.69 (m, 1H), 4.53-4.28 (m, 15H), 4.08 (s, 3H). ³¹P NMR (200 MHz, D₂O): δ −1.47 (1P), −10.98 (2P), −22.41 (1P). MS (ESI) calculated for C₃₂H₄₂N₁₅O₂₄P₄ 1144.66, found 1142.9 [M−H]⁻.

^{m7}G_{3′OMe}pppA_{2′,4′-LNA}pG (Compound 2):

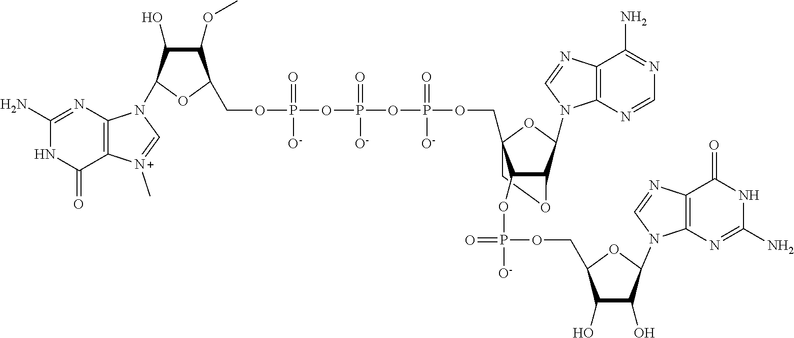

Compound 2 was synthesized as described according to General Procedure 1 from N7-Me-3′-Ome-GDP (0.30 g, 0.459 mmol), EDC (0.141 g, 0.734 mmol) and imidazole (0.0938 g, 1.377 mmol) in 3.0 ml 1000 water in DMSO, magnesium chloride (58.3 mg in 0.19 mL water, 0.612 mmol) and p(LNA)ApG (0.277 g, 0.306 mmol). Solid product (0.2573 g) was obtained (yield: 69%). ¹H NMR (500 MHz, D₂O): δ 8.06 (s, 1H). 7.95 (s, 1H), 7.75 (s, 1H), 5.82 (d, J=4.7 Hz, 1H), 5.68 (m, 2H), 4.90 (m, 1H), 4.69-4.11 (m, 13H), 4.09 (s, 3H), 3.49 (s, 3H). ³¹p NMR (200 MHz, D₂O): δ −1.46 (1P), −10.98 (2P), −22.30 (1P). MS (ESI) calculated for C₃₃H₄₄N₁₅O₂₄P₄ 1158.69, found 1156.4 [M−H]⁻.

^{m7}GpppA_{2′-4′-LNA}p(s)G (Compound 3):

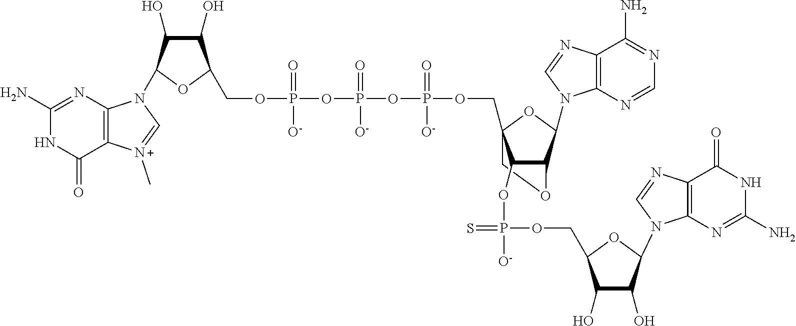

Compound 3 was synthesized as described according to General Procedure 1 from N7-Me-GDP (0.2 g, 0.327 mmol), EDC (0.1 g, 0.523 mmol) and imidazole (0.067 g, 0.981 mmol) in 2.0 ml 10% water in DMSO, magnesium chloride (50.9 mg, 0.536 mmol) and p(LNA)Ap(s)G (0.2 g, 0.218 mmol). Solid product (149.5 mg) was obtained (yield: 56). The two diastereomers were purified by RP-HPLC and the NMR/MS data are shown below.

^{m7}GpppA_{2′-4′-LNA}p(s)G: D1

Final yield—137.3 μmol; NMR (500 MHz, D₂O) δ 8.12 (s, 1H), 7.98 (s, 1H), 7.83 (s, 1H), 5.86 (d, J=4.1, 1H), 5.76 (s, 1H), 5.72 (d, J=4.6, 1H), 4.97 (s, 2H), 4.49-4.57 (m, 3H), 4.40-4.48 (m, 5H), 4.15-4.36 (m, 8H), 4.08 (s, 3H); 31P NMR (200 MHz, D₂O) 55.13 (1P), −11.02 (2P), −22.39 (1P), MS m/z=1159.8 (M−H)

^{m7}GpppA_{2′-4′-LNA}p(s)G: D2

Final yield—130.4 μmol; NMR (500 MHz, D₂O) δ 8.11 (s, 1H), 7.95 (s, 1H), 7.86 (s, 1H), 5.84 (d, J=4.2, 1H), 5.68 (m, 2H), 4.90 (m, 2H), 4.50-4.57 (m, 3H), 4.41-4.46 (m, 3H), 4.25-4.33 (m, 6H), 4.13-4.19 (m, 2H), 4.09 (s, 3H); 31P NMR (200 MHz, D₂O) 56.19 (1P), −11.00 (2P), −22.42 (1P), MS m/z=1157.9 (M−H)

^{m7}GpppA_{2′OMe-5′vinyl}pG (Compound 4):

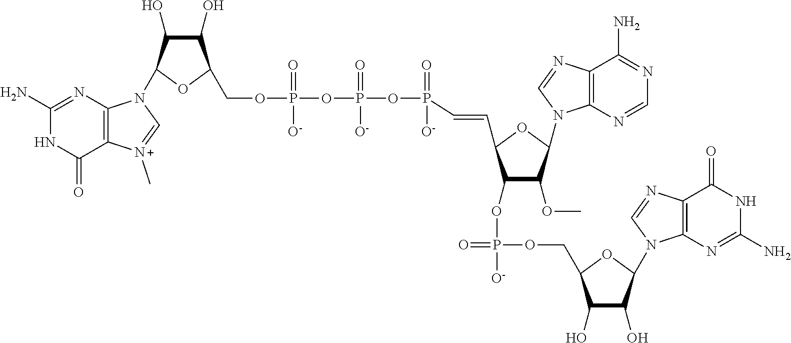

Compound 4 was synthesized as described according to General Procedure 1 from N7-Me-GDP (0.0716 g, 0.117 mmol), EDC (0.0358 g, 0.187 mmol) and imidazole (0.024 g, 0.351 mmol) in 0.7 ml 10% water in DMSO, 3.15 M magnesium chloride (0.05 mL, 0.156 mmol) and 5′-vinyl phosphonate AG (0.07 g, 0.078 mmol). Solid product (67 mg) was obtained (yield: 71%). ¹H NMR (500 MHz, D₂O): δ 8.26 (s, 1H), 8.11 (s, 1H), 7.92 (s, 1H), 6.62 (m, 1H), 6.21 (t, J=17.6 Hz, 1H), 6.00 (d, J=5.9 Hz, 1H), 5.79 (m, 2H), 4.54-4.35 (m, 7H), 4.16 (m, 3H), 4.02 (s, 3H), 3.39 (s, 3H). ³¹P NMR (200 MHz, D₂O): δ 3.74 (1P), −0.40 (1P), −10.94 (2P), −22.36 (1P). MS (ESI) calculated for C₃₃H₄₃N₁₅O₂₃P₄ 1141.68, found 1140.7 [M−H]⁻.

^{m7}G_{3′-OMe}pppA_{2′OMe-5′vinyl}pG (Compound 5):

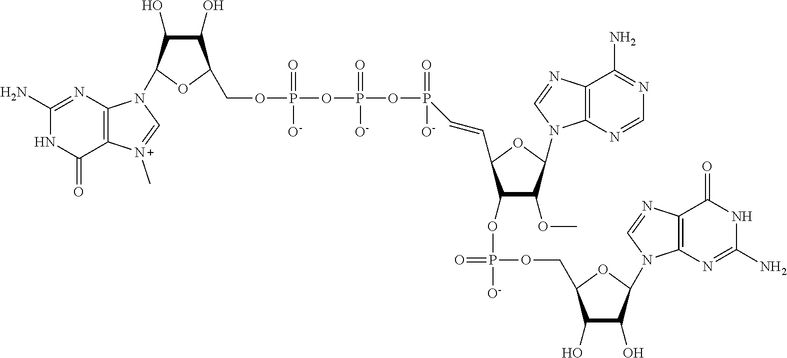

Compound 5 was synthesized as described according to General Procedure 1 from N7-Me-3′-Ome-GDP (0.076 g, 0.117 mmol), EDC (0.0358 g, 0.187 mmol) and imidazole (0.024 g, 0.351 mmol) in 0.7 ml 10% water in DMSO, 3.15 M magnesium chloride (0.05 mL, 0.156 mmol) and 5′-vinyl phosphonate AG (0.07 g, 0.078 mmol). Solid product (70 mg) was obtained (yield: 73%). ¹H NMR (500 MHz, D₂O): δ 8.27 (s, 1H), 8.11 (s, 1H), 7.93 (s, 1H), 6.62 (m, 1H), 6.21 (t, J=17.5 Hz, 1H), 6.00 (d, J=5.9 Hz, 1H), 5.82 (d, J=5.45 Hz, 1H), 5.71 (d, J=4.25 Hz, 1H), 4.64 (m, 1H), 4.48 (m, 1H), 4.43 (m, 1H), 4.35-4.23 (m, 3H), 4.15 (m, 3H), 4.08 (m, 1H), 4.02 (s, 3H), 3.46 (s, 3H), 3.40 (s, 3H). ³¹P NMR (200 MHz, D₂O): δ 3.85 (1P), −0.39 (1P), −10.91 (2P), −22.30 (1P). MS (ESI) calculated for C₃₄H₄₅N₁₅O₂₃P₄ 1155.71, found 1155.5 [M−H]⁻.

^{m7}GpppA_{2′F}p(s)G (Compound 6):

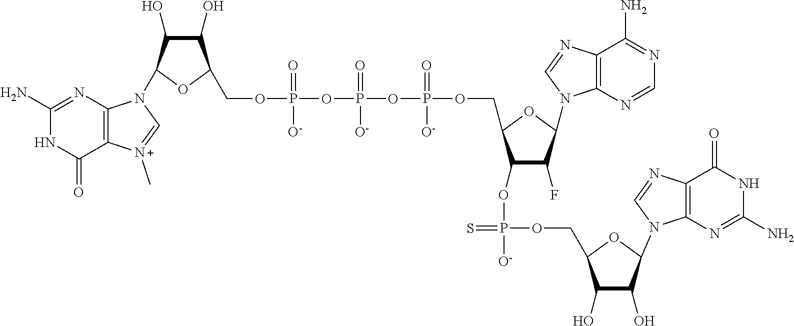

Compound 6 was synthesized as described according to General Procedure 1 from N7-Me-GDP (0.2 g, 0.327 mmol), EDC (0.100 g, 0.523 mmol) and imidazole (0.067 g, 0.981 mmol) in 2.0 ml 10% water in DMSO, magnesium chloride (50.9 mg, 0.536 mmol) and pA_{2′F}p(s)G (0.198 g, 0.218 mmol). Solid product (155 mg) was obtained (yield: 62%). The two diastereomers were purified by RP-HPLC and the NMR/MS data are shown below.

^{m7}GpppA_{2′F}p(s)G: D1

Final yield—24.4 μmol; NMR (500 MHz, D₂O) δ 8.24 (s, 1H), 7.99 (s, 1H), 7.81 (s, 1H), 6.09 (d, J=15.9 Hz, 1H), 5.88 (d, J=3.9 Hz, 1H), 5.70 (d, J=6.5 Hz, 1H), 5.49-5.38 (m, 1H), 5.05-5.01 (m, 1H), 5.56 (t, J=4.5 Hz, 2H), 4.49-4.33 (m, 7H), 4.26-4.20 (m, 4H), 4.04 (s, 3H)

31P NMR (200 MHz, D₂O) 55.72 (1P), −10.93 (1P), −10.05 (1P), −22.40 (1P), MS m/z=1149.0 (M−H).

^{m7}GpppA_{2′F}p(s)G: D2

Final yield—29.8 μmol; NMR (500 MHz, D₂O) δ 8.24 (s, 1H), 7.99 (s, 1H), 7.86 (s, 1H), 6.10 (d, J=16.0 Hz, 1H), 5.88 (d, J=3.9 Hz, 1H), 5.70 (d, J=5.5 Hz, 1H), 5.42-5.31 (m, 1H), 5.07-4.99 (m, 1H), 4.57-4.44 (m, 6H), 4.42-4.21 (m, 7H), 4.19-4.16 (m, 1H), 4.04 (s, 3H) 31P NMR (200 MHz, D₂O) 57.23 (1P), −10.97 (2P), −22.38 (1P), MS m/z=1148.8 (M−H).

^{m7}Gppp(diaminopurine)_{2′OMe}pG (Compound 7):

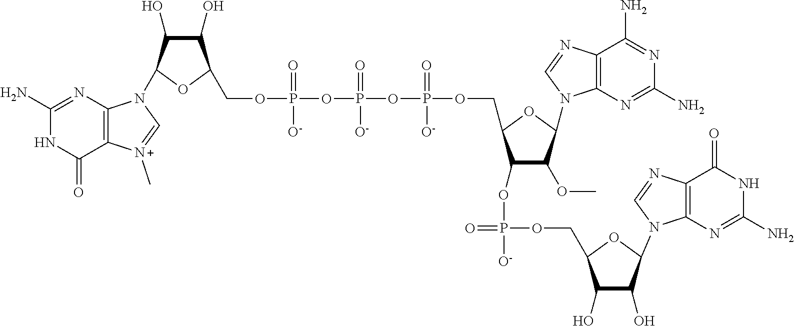

Compound 7 was synthesized as described according to General Procedure 1 from N7-Me-GDP (0.219 g, 0.358 mmol), EDC (0.11 g, 0.574 mmol) and imidazole (0.073 g, 1.076 mmol) in 2.2 ml 10% water in DMSO, 3.15 M magnesium chloride (0.15 mL, 0.478 mmol) and DAP-G (0.22 g, 0.239 mmol). 191 mg solid product was obtained (yield: 65%). ¹H NMR (500 MHz, D₂O): δ 8.02 (s, 1H), 7.98 (s, 1H), 5.82 (m, 3H), 4.90 (m, 1H), 4.55 (m, 1H), 4.52-4.20 (m, 13H), 4.01 (s, 3H), 3.45 (s, 3H). ³¹P NMR (200 MHz, D₂O): δ −0.35 (1P), −10.84 (2P), −22.32 (1P).

^{m7}G_{3′OMe}ppp(diaminopurine)_{2′OMe}pG (Compound 8):

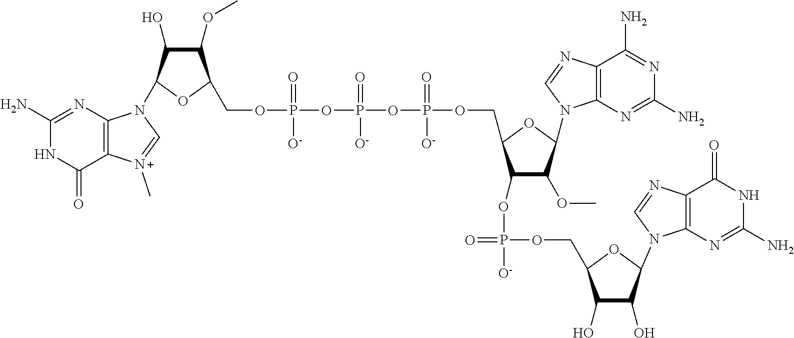

Compound 8 was synthesized as described according to General Procedure 1 from N7-Me-3′-Ome-GDP (0.233 g, 0.358 mmol), EDC (0.11 g, 0.574 mmol) and imidazole (0.073 g, 1.076 mmol) in 2.2 ml 10% water in DMSO, 3.15 M magnesium chloride (0.15 mL, 0.478 mmol) and DAP-G (0.22 g, 0.239 mmol). Solid product (202 mg) was obtained (yield: 68%). ¹H NMR (500 MHz, D₂O): δ 8.02 (s, 1H), 7.98 (s, 1H), 5.82 (m, 3H), 4.93 (m, 1H), 4.69 (m, 1H), 4.49 (m, 1H), 4.42-4.10 (m, 11H), 3.68 (s, 3H), 3.48 (s, 3H), 3.46 (s, 3H). ³¹P NMR (200 MHz, D₂O): δ −0.34 (1P), −10.94 (2P), −22.28 (1P).

^{m7}G_{3′OMe}ppp(diaminopurine)_{2′OMe}p(s)G (Compound 9):

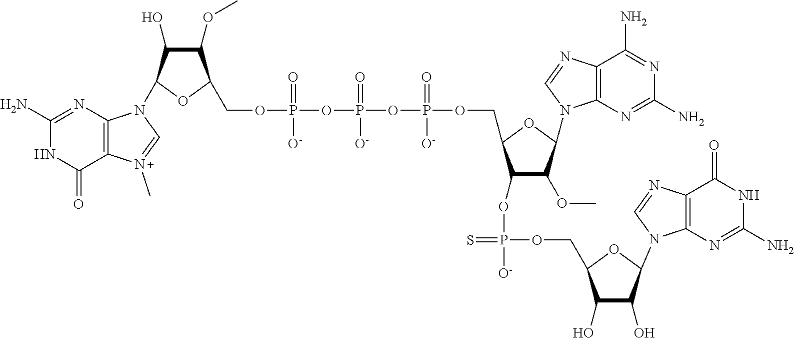

Compound 9 was synthesized as described according to General Procedure 1 from N7-Me-3′-OMe-GDP (0.201 g, 0.327 mmol), EDC (0.100 g, 0.523 mmol) and imidazole (0.067 g, 0.981 mmol) in 2.0 ml 10% water in DMSO, magnesium chloride (50.9 mg, 0.536 mmol) and pDAPm p(s)G (0.204 g, 0.218 mmol). Solid product obtained (65.4 mg) post anion exchange (Q Sepharose Fast Flow) and Anion exchange (Q Sepharose High Performance) purification was obtained (yield: 25.2%). ¹H NMR (500 MHz, D₂O): δ 8.05 (s, 1H), 8.01-8.00 (m, 2H), 5.86-5.79 (m, 3H), 5.11-5.06 (m, 1H), 4.64 (t, J=4.5 Hz, 1H), 4.52-4.15 (m, 11H), 4.08-4.05 (m, 1H), 4.02 (d, J=8.25 Hz, 3H), 3.46-3.44 (m, 4H), 3.40 (s, 2H).). ³¹P NMR (200 MHz, D₂O): δ 56.76 (1P), 56.06 (1P), −10.91-−11.09 (4P), −22.27-−22.49 (2P).). MS m/z=1189.8 (M−H)

^{m7}G_{3′Ome}pppA_{2′OMe}p(s)G (Compound 21):

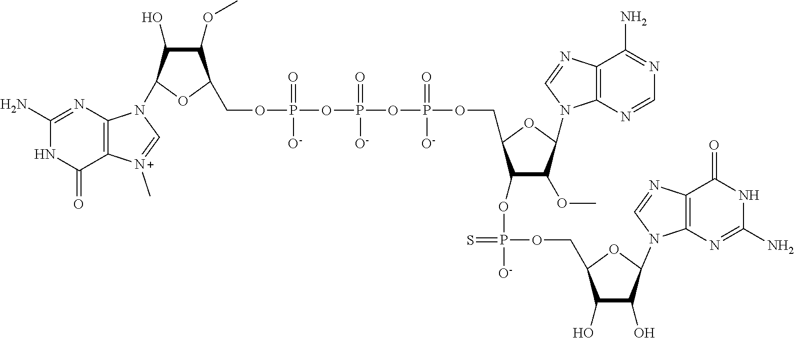

Compound 21 (two diastereomers) was synthesized as described according to General Procedure 1. The two diastereomers were prepared from a pA(ps)G dinucleotide, as shown below, that was made as a mixture of two diastereomers, D1 and D2 (chiral P atom generating the D1 and D2 diastereomers is indicated with an asterisk in the structure below). The diastereomers were resolved by reverse phase chromatography using a Daisogel 150×20 mm SP-100-15-ODS-P column (Torrance, CA), with the following separation conditions: Buffer A: 100 mM triethylammonium bicarbonate (TEAB), Buffer B: acetonitrile (ACN). Gradient: 0% B×1.5 column volumes (CV), 0-12.5% B×10 CV. The D1 diastereomer (referred to as pA(ps)G D1) eluted first from the reverse phase chromatography column and the D2 diastereomer (referred to as pA(ps)G D2) eluted second from the reverse phase chromatography column. The final diastereomer purity for each was >99%.

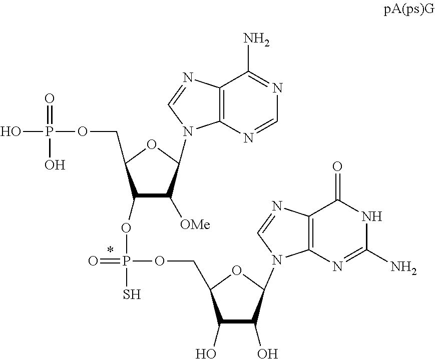

The resulting diastereomers of Compound 21 are indicated as Compound 21 D1 and Compound 21 D2.

Compound 21 D1 was prepared from N7-Me-3′-OMe-GDP (0.119 g, 0.202 mmol), EDC (0.05 g, 0.262 mmol) and imidazole (0.018 g, 0.269 mmol) in 6.6 ml 10% water in DMSO, magnesium chloride (0.085 mL, 0.269 mmol) and pA(ps)G D1 (0.1346 g, 0.134 mmol). The resulting compound was isolated and collected as a solid product (61.7 mg; yield: 37%). 1H NMR (500 MHz, D₂O) δ 8.38 (s, 1H), 8.12 (s, 1H), 8.05 (s, 1H), 6.00 (d, J=6.0 Hz, 1H), 5.86 (d, J=5.9 Hz, 1H), 5.83 (d, J=4.3 Hz, 1H), 5.11-5.08 (m, 1H), 4.82-4.73 (m, 1H), 4.56-4.52 (m, 2H), 4.48 (t, J=5.5 Hz, 1H), 4.38-4.35 (m, 3H), 4.32-4.23 (m, 4H), 4.20-4.17 (m, 1H), 4.10 (t, J=4.6 Hz, 1H), 4.03 (s, 3H), 3.47 (m, 6H). 31P NMR (200 MHz, D₂O) 56.05 (s, 1P), −11.01, −11.10 (overlapping doublet, 2P), −22.33 (t, J=18.2, 1P). MS m/z=1173.8 (M−H).

The method was repeated using pA(ps)G D2 to isolate Compound 21 D2. Specifically, the synthesis was performed using N7-Me-3′-OMe-GDP (0.187 g, 0.317 mmol), EDC (0.079 g, 0.413 mmol) and imidazole (0.029 g, 0.423 mmol) in 1.87 ml 10% water in DMSO, magnesium chloride (0.134 mL, 0.423 mmol) and pA(ps)G D2 (0.2173 g, 0.212 mmol). Solid product (0.124 mg) was obtained (yield: 47%). NMR (500 MHz, D₂O) δ 8.38 (s, 1H), 8.11 (s, 1H), 8.01 (s, 1H), 6.03 (d, J=5.8 Hz, 1H), 5.84 (m, 2H), 5.08-5.06 (m, 1H), 4.66 (m, 1H), 4.58 (m, 2H), 4.52-4.50 (t, J=4.9 Hz, 1H), 4.45-4.43 (T, J=5.4 Hz, 1H), 4.40-4.20 (m, 8H), 4.11-4.09 (m, 1H), 4.04 (s, 3H), 3.47 (s, 3H), 3.44 (m, 3H). 31P NMR (200 MHz, D₂O) 56.68 (s, 1P), −10.98, −11.07 (overlapping doublet, 2P), −22.29 (t, J=20.2, 1P). MS m/z=1174.0 (M−H).

^{m7}G_{2′OMe3′OMe}pppA_{2′OMe}pG (Compound 25):

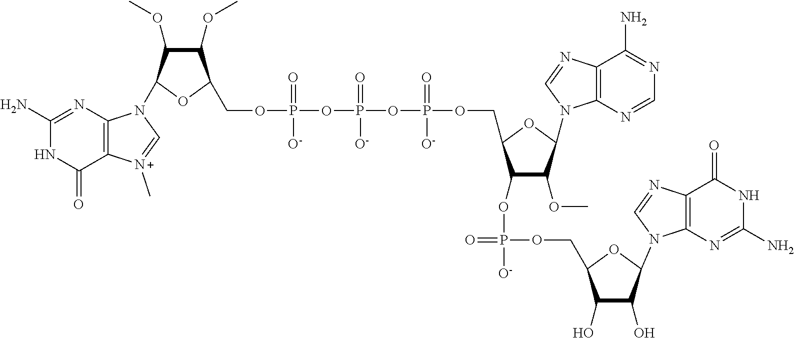

Compound 25 was synthesized as described according to General Procedure 1 from N-7-Me-2′, 3′-di-OMe-GDP (0.5 g, 0.36 mmol), EDC (0.12 g, 0.6 mmol) and imidazole (0.10 g, 1.4 mmol) in 5 mL 30% water in DMSO, magnesium chloride (0.12 mL of 3.15 MgCl₂ solution, 0.48 mmol) and pA_{2′OMe}pG (0.2 g, 0.24 mmol). Solid product (0.16 g) was obtained (yield 57%). ¹H NMR δ 8.32 (s, 1), 8.06 (s, 1), 7.92 (s, 1), 5.94 (d, 1, J=10 Hz), 5.80 (d, 1, J=10 Hz), 5.79 (d, 1, J=10 Hz), 4.89 (m, 1), 4.47 (m, 2), 4.37 (t, 1, J=5 Hz), 4.33 (m, 4), 4.24 (m, 2), 4.14 (m, 4), 4.02 (s, 3), 3.48 (s, 3), 3.42 (s, 3), 3.40 (s, 3). 31P δ −0.42 (1P), −11.12 (2P), −22.43 (1P); MS m/z=1172.0 (M−H).

^{m7}G_{2′OMe}pppA_{2′OMe}pG (Compound 26):

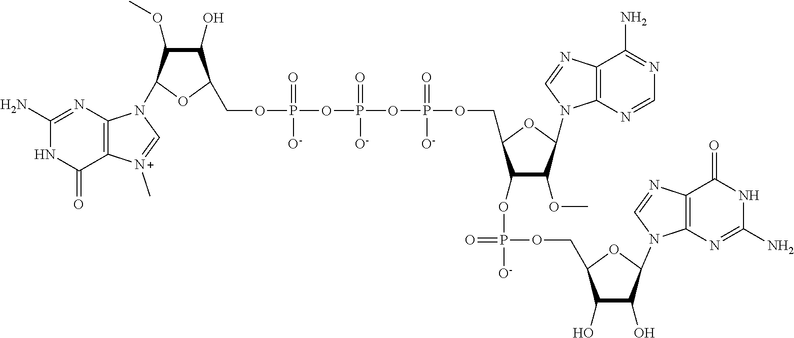

Compound 26 was synthesized as described according to General Procedure 1 from N-7-Me-2′-OMe-GDP (0.20 g, 0.3 mmol), EDC (0.091 g, 0.47 mmol) and imidazole (0.06 g, 0.89 mmol) in 2.2 mL 10% water in DMSO, magnesium chloride (0.13 mL of 3.15 MgCl₂ solution, 0.42 mmol) and pA_{2′OMe}pG (0.19 g, 0.21 mmol). Solid product (0.21 g) was obtained (yield 78%). 1H NMR δ 8.34 (s, 1H), 8.09 (s, 1H), 7.92 (s, 1H), 5.96 (d, 1H, J=9 Hz), 5.86 (d, 1H, J=5 Hz), 5.80 (d, 1H, J=9 Hz), 4.90 (m, 1H), 4.51 (d, 1H, J=5 Hz), 4.48 (m, 2H), 4.38 (m, 2H), 4.33 (m, 1H), 4.27 (m, 2H), 4.23-4.16 (m, 5H), 4.02 (s, 3H), 3.52 (s, 3H), 3.41 (s, 3H). 31P δ −0.43 (1P), −11.06 (2P), −22.39 (1P); MS m/z=1157.9 (M−H).

^{m7}G_{2′OMe3′OMe}ppp^{m6}A_{2′OMe}pG (Compound 27):

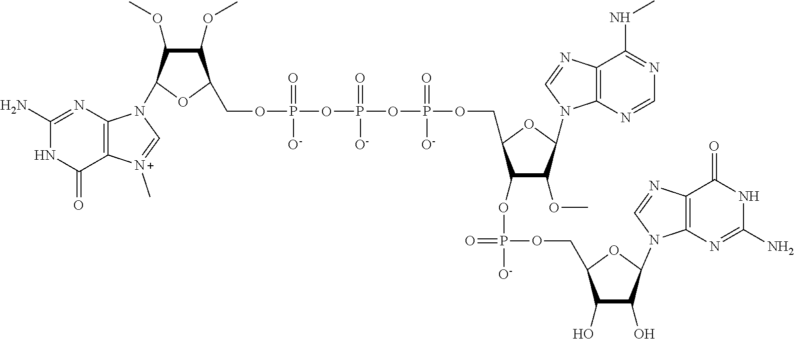

Compound 27 was synthesized as described according to General Procedure 1 from N-7-Me-2′, 3′-di-OMe-GDP (0.5 g, 0.36 mmol), EDC (0.12 g, 0.6 mmol) and imidazole (0.10 g, 1.4 mmol) in 5 mL 30% water in DMSO, magnesium chloride (0.12 mL of 3.15 MgCl₂ solution, 0.48 mmol) and p^{m6}A_{2′OMe}pG (0.22 g, 0.24 mmol). Solid product (0.18 g) was obtained (yield 64%). ¹H NMR δ 8.26 (s, 1), 8.07 (s, 1), 7.70 (s, 1) 5.94 (d, 1, J=10 Hz), 5.80 (d, 1, J=10 Hz), 5.76 (d, 1 j=5 Hz), 4.89 (m, 1), 4.73, (m, 1), 4.21 (m, 1), 4.18 (m, 2), 4.14 (t, 2, J=5 Hz), 3.99 (s, 3) 3.49 (s, 3), 3.42 (s, 3), 3.08 (br s, 3). 31P δ −0.42 (1P), −11.12 (2P), −22.43 (1P); MS m/z=1172.0 (M−H).

^{m7}G_{2′OMe}ppp^{m6}A_{2′OMe}pG (Compound 28):

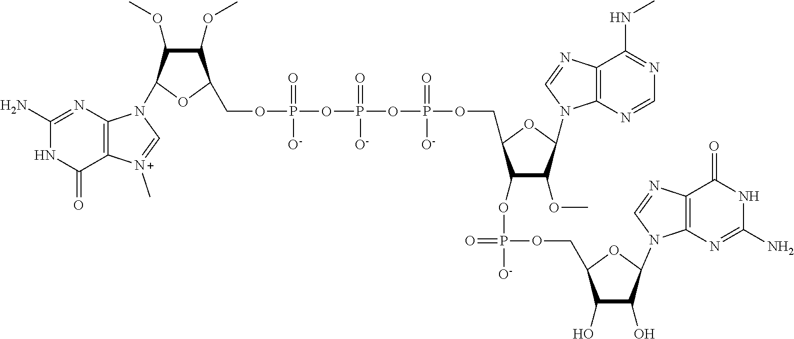

Compound 28 was synthesized as described according to General Procedure 1 from N-7-Me-2′-OMe-GDP (0.20 g, 9.3 mmol), EDC (9.091 g, 0.47 mmol) and imidazole (0.06 g, 0.89 mmol) in 2.2 mL 10% water in DMSO, magnesium chloride (0.13 mL of 3.15 MgCl₂ solution, 0.42 mmol) and p^{m6}A_{2′OMe}pG (0.19 g, 0.21 mmol). Solid product (0.17 g) was obtained (yield 68%). ¹H NMR δ 8.28 (s, 1H), 8.09 (br s, 1H), 7.92 (s, 1H), 5.96 (d, 1H, J=5 Hz), 5.84 (d, 1H, J=5 Hz), 5.82 (d, 1, J=9 Hz), 4.91 (m, 1H), 4.49 (m, 3H), 4.39 (m, 2H), 4.25 (m, 2H), 4.20-4.15 (m, 6H), 4.00 (s, 3H), 3.54 (s, 3H), 3.46 (s, 3H), 3.09 (br s, 3H). 31P δ −0.40 (1P), −11.06 (2P), −22.37 (1P); MS m/z=1174 (M+H).

^{m7}G_{3′SMe}pppA_{2′OMe}pG (Compound 42):

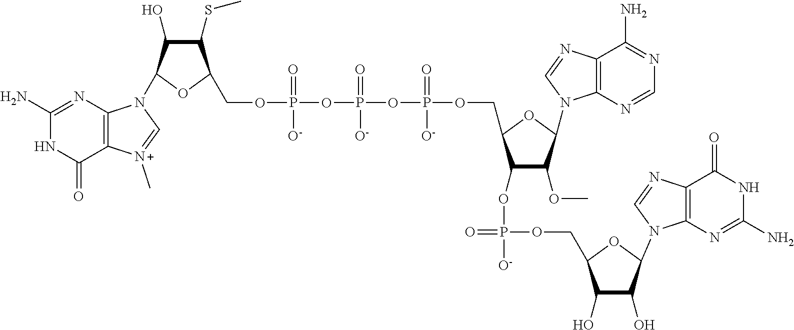

Compound 42 was synthesized as described according to General Procedure 1 from N7-Me-3′-SMe-GDP (0.18 g, 0.29 mmol), EDC (0.09 g, 0.47 mmol) and imidazole (0.06 g, 0.90 mmol) in 1.8 mL 10% water in DMSO, magnesium chloride (0.123 mL of 3.15 M MgCl₂ solution, 0.39 mmol) and pAₘpG (0.18 g, 0.19 mmol). Solid product (0.161 g, 0.130 mmol) was obtained (yield: 67%). ¹H NMR (500 MHz, D₂O) δ 8.37 (s, 1H), 8.10 (s, 1H), 7.92 (s, 1H), 5.98 (d, J=5.8, 1H), 5.81 (m, 2H), 4.92 (m, 2H), 4.57 (d, J=4.7, 2H), 4.47-4.51 (m, 4H), 4.41 (t, J=5.2, 1H), 4.29-4.40 (m, 3H), 4.15-4.24 (m, 5H), 4.01 (s, 3H), 3.42 (s, 3H), 2.12 (s, 3H). ³¹P NMR (200 MHz, D₂O) −0.41 (1P), −11.04 (1P), −11.35 (1P), −22.32 (1P). MS m/z=1173.9 (M−H).

^{m7}G_{2′OH,2′Me}pppA_{2′OMe}pG (Compound 43):

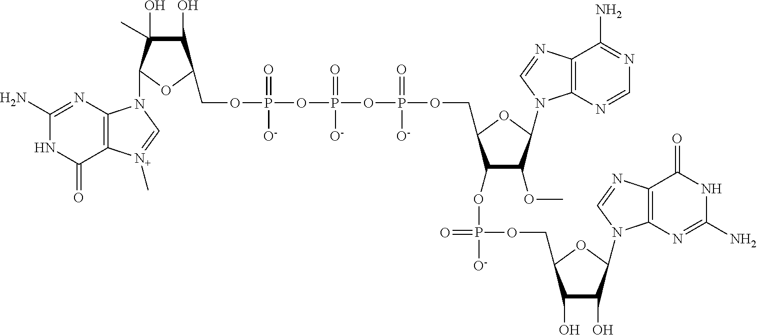

Compound 43 was synthesized according to general procedure 1 from N7-Me-2′Me 2′OH-GDP (0.502 g, 0.85 mmol), EDC (0.230 g, 1.2 mmol) and imidazole (0.153 g, 2.25 mmol) in 5 mL 10% water in DMSO, magnesium chloride (0.317 mL of 3.15 M of MgCl₂ Solution, 1 mmol) and pAmpG (0.454 g, 0.5 mmol). Solid product (0.437 g) was obtained (75% yield).

¹H NMR (500 MHz, D₂O): δ 8.36 (S, 1H), 8.02 (s, 1H), 7.93 (s, 1H), 5.96 (d, J=5.7 Hz, 1H), 5.82 (s, 1H), 5.81 (s, 1H), 4.91 (m, 1H), 4.49 (m, 3H), 4.39 (t, J=5.1 Hz, 1H), 4.34 (m, 1H), 4.29 (m, 3H), 4.20 (m, 3H), 4.14 (m, 1H), 4.03 (s, 3H), 3.41 (s, 3H), 1.02 (s, 3H). ³¹P NMR (200 MHz, D₂O): δ −0.39 (1P), −11.0 (2P), −22.3 (1P). MS m/z=1158.9 (M−H).

^{m7}G_{3′AZM}ppp^{m6}A_{2′OMe}pG (Compound 45):

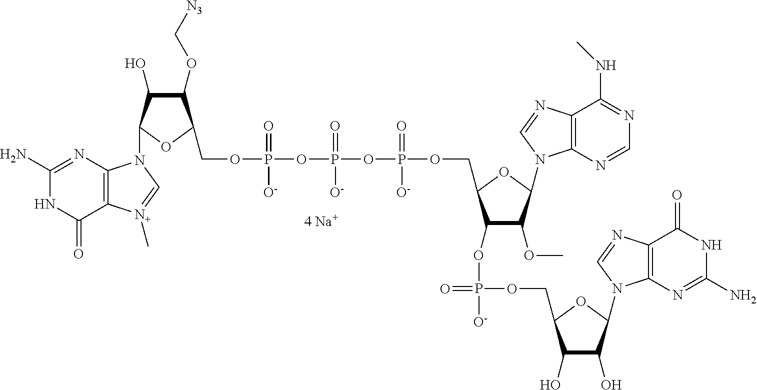

m⁷G_{3′AZM}ppp^{m6}A_{2′OMe}pG was synthesized as described according to General Procedure 1 from N-7-Me-3′-AZM-GDP (0.20 g, 0.3 mmol), EDC (0.098 g, 0.51 mmol), and imidazole (0.082 g, 1.2 mmol) in 3.0 mL 30% water in DMSO, magnesium chloride (0.16 mL of 3.15 MgCl₂ solution, 0.5 mmol) and pA_{2′OMe}pG (0.23 g, 0.25 mmol). Solid product (0.086 g) was obtained (yield 24%). ¹H NMR: δ 8.28 (s, 1H), 8.08 (s, 1H), 7.91 (s, 1H), 5.97 (d, 1H, J=10 Hz), 5.82-5.80 (m, 2H), 4.91-4.88 (m, 2H), under D₂O (3H), 4.63 (t, 1H, J=5 Hz), 4.50-4.46 (m, 2H), 4.43 (s, 1H), 4.39-4.30 (m, 5H), 4.24-4.17 (m, 4H), 3.99 (s, 3H), 3.45 (s, 3H), 3.08 (s, 3H). ³¹P NMR δ −0.40 (1P), −11.08 (2P), −22.42 (1P). [M−3H⁺4Na]⁺ Expected Mass: 1303.12; [M−3H⁺4Na]⁺ Detected: m/z=1302.5 (M−3H⁺4Na).

^{m7}G_{3′F}ppp^{m6}A_{2′OMe}pG (Compound 49):

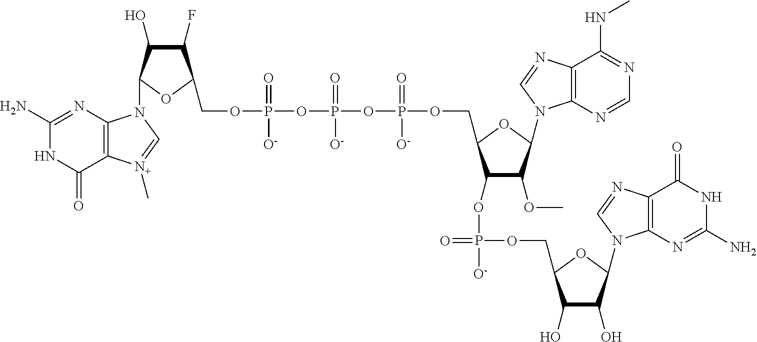

Compound 49 was synthesized as described according to General Procedure 1 from N-7-Me-3′-F-GDP (0.20 g, 0.27 mmol), EDC (0.083 g, 0.43 mmol) and imidazole (0.055 g, 0.81 mmol) in 2.2 mL 10% water in DMSO, magnesium chloride (0.12 mL of 3.15 MgCl₂ solution, 0.38 mmol) and p^{m6}A_{2′OMe}pG (0.176 g, 0.19 mmol). Solid product (0.12 g) was obtained (yield 55%). ¹H NMR δ 8.27 (s, 1H), 8.09 (br s, 1H), 7.92 (s, 1H), 5.96 (d, 1H, J=9 Hz), 5.82 (d, 1, J=5 Hz), 5.80 (d, 1H, J=9 Hz), 5.32 (d, 1H, J=5 Hz), 5.22 (d, 1, J=5 Hz), 4.89 (m, 1H), 4.72 (m, 1H), 4.68 (m, 1H), 4.48 (m, 2H), 4.38 (t, 1H, J=5 Hz), 4.30-4.20 (M, 7H), 4.02 (s, 3H), 3.45 (s, 3H), 3.10 (br s, 3H). MS m/z=1159.7 (M+H).

^{m7}G_{3′MOE}ppp^{m6}A_{2′OMe}pG (Compound 50):

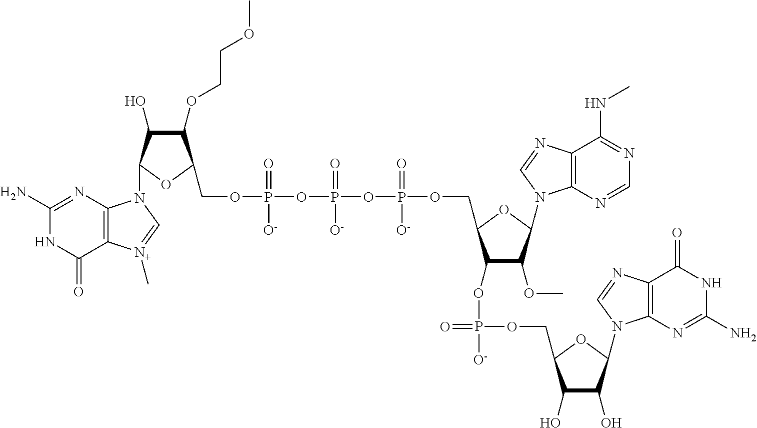

Compound 50 was synthesized as described according to General Procedure 1 from N-7-Me-3′-(2-Methoxyethoxy)-GDP (0.030 g, 0.37 mmol), EDC (0.12 g, 0.6 mmol) and imidazole (0.10 g, 1.5 mmol) in 3.5 mL 10% water in DMSO, magnesium chloride (0.15 mL of 3.15 MgCl₂ solution, 0.24 mmol) and p A^{M6}_{2′OMe}pG (0.11 g, 0.12 mmol). Solid product (0.11 g) was obtained (yield 65%). ¹H NMR δ 8.21 (s, 1), 8.06 (br s, 1), 7.89 (s, 1), 5.96 (d, 1, J=5.5 Hz), 5.82 (s, 1), 5.81 (d, 1, J=5.5 Hz), 4.88 (m, 1), 4.73 (t, 1, J=5.5 Hz), 4.60 (t, 1, J=4.7), 4.47 (m, 1), 4.46 (m, 1), 4.36 (m, 2), 4.35 (M, 2), 4.32 (M, 1), 4.22 (m, 1), 4.17 (m, 4), 3.98 (s, 3), 3.76 (m, 2), 3.62 (m, 2), 3.43 (s, 3), 3.37 (s, 3), 3.07 (br s, 3); 31P δ −0.40 (1P), −11.12 (2P), −22.45 (1P); MS m/z=1215.8 (M−H).

^{m7}G_{3′SMe}ppp^{m6}A_{2′OMe}pG (Compound 52):

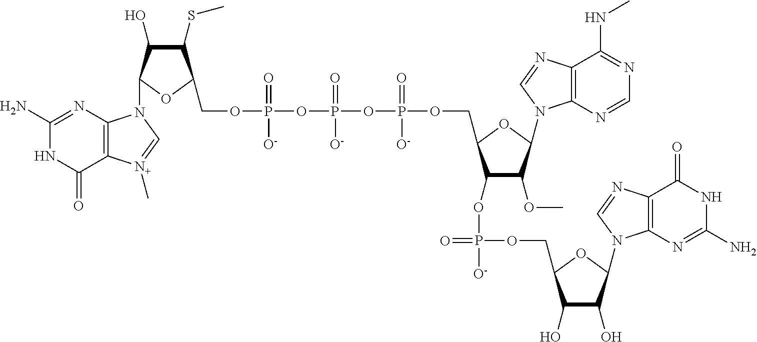

Compound 52 was synthesized as described according to General Procedure 1 from N7-Me-3′-SMe-GDP (0.18 g, 0.29 mmol), EDC (0.09 g, 0.47 mmol) and imidazole (0.06 g, 0.90 mmol) in 1.8 mL 10% water in DMSO, magnesium chloride (0.123 mL of 3.15M MgCl₂ solution, 0.39 mmol) and p^{m6}AₘpG (0.18 g, 0.19 mmol). Solid product (0.109 g, 0.087 mmol) was obtained. Final yield—86.5 μmol, 194 μmol starting scale, 44.6%. NMR (500 MHz, D₂O) δ 8.30 (s, 1H), 8.07 (s, 1H), 7.89 (s, 1H), 5.97 (d, J=5.4, 1H), 5.79 (d, J=5.8), 5.75 (s, 1H), 4.91 (m, 2H), 4.54 (d, J=4.7, 1H), 4.45-4.50 (m, 3H), 4.40 (t, J=5.0, 1H), 4.24-4.33 (m, 2H), 4.15-4.23 (m, 5H), 3.97 (s, 3H), 3.45 (s, 3H), 3.06 (bs, 3H), 2.1 (s, 3H). 31P NMR (200 MHz, D₂O) −0.42 (1P), −11.02 (1P), −11.37 (1P), −22.31 (1P). MS m/z=1187.8 (M−H)

^{m7}(L-sugar isomer)GpppA_{2′OMe}pG (Compound 75):

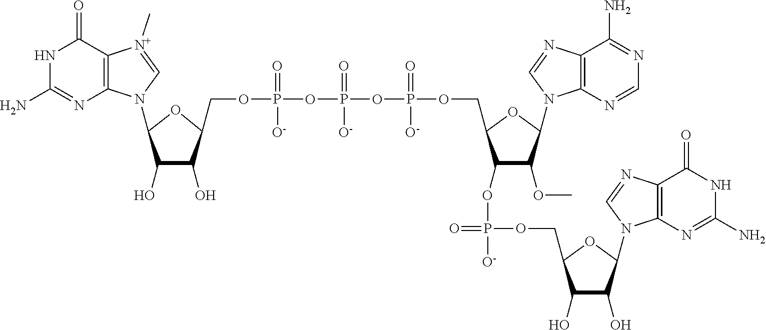

Compound 75 was synthesized according to general procedure 1 from N7-Me-L-GDP (0.31 g, 0.5 mmol), EDC (0.153 g, 0.8 mmol) and imidazole (0.106 g, 1.550 mmol) in 3.1 mL 10% water in DMSO, magnesium chloride (0.212 g, 0.667 mmol) and pAmpG (0.30 g, 0.333 mmol). 218.07 umol product was obtained (yield: 65.5%). ¹H NMR (500 MHz, D₂O): δ 8.30 (s, 1H), 8.06 (s, 1H), 7.87 (s, 1H), 6.01 (d, J=4.4, 1H), 5.77 (m, 2H), 4.86 (m, 1H), 4.73 (m, 1H), 4.48-4.46 (m, 2H), 4.46-4.45 (m, 3H), 4.43-4.42 (m, 1H) 4.38-4.19 (m, 4H) 4.00 (s, 3H), 3.47 (s, 3H), 1.90 (s, 1H), 1.17 (t, J=7.1 Hz, 1H). 31P NMR (200 MHz, D₂O): δ 0.47 (s, 1P), −10.87 (m, 1P), −22.06 (m, 1P). Calculated for C₃₂H₄₂N₁₅O₂₄P₄ 1144.66, found 1144.0 [M−H] negative mode.

^{m7}G_{2′OH,2′Me}ppp^{m6}A_{2′OMe}pG (Compound 76):

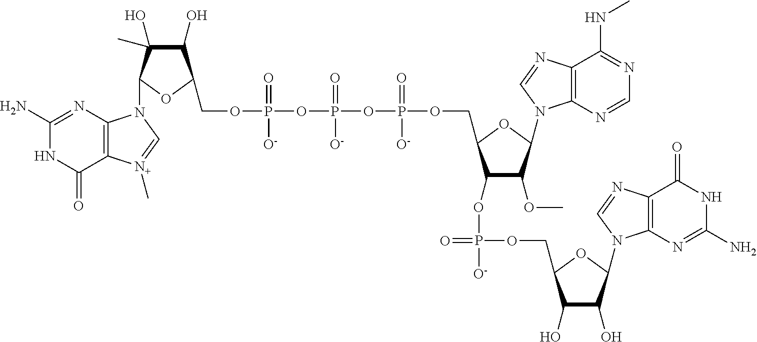

Compound 76 was synthesized according to General Procedure 1 from N7-Me-2′Me 2′OH-GDP (0.502 g, 0.85 mmol), EDC (0.230 g, 1.2 mmol) and imidazole (0.153 g, 2.25 mmol) in 5 mL 10% water in DMSO, magnesium chloride (0.317 mL of 3.15 M of MgCl₂ Solution, 1 mmol) and pAmpG (0.406 g, 0.5 mmol). Solid product (0.522 g) was obtained (89% yield). 1H NMR (500 MHz, D₂O): δ 8.28 (S, 1H), 8.05 (s, 1H), 7.89 (s, 1H), 5.95 (d, J=5.4 Hz, 1H), 5.80 (d, J=5.75 Hz, 1H), 5.77 (S, 1H), 4.91-4.88 (m, 1H), 4.81-4.72 (m, 3H), 4.50-4.46 (m, 2H), 4.37 (t, J=4.95 Hz, 1H), 4.33-4.26 (m, 3H), 4.25-4.18 (m, 3H), 4.12-4.10 (m, 1H), 4.00 (s, 3H), 3.43 (s, 3H), 3.06 (br s, 3H), 1.00 (s, 3H). 31P NMR (200 MHz, D₂O): δ −0.40 (1P), −10.99 (2P), −22.31 (1P). MS m/z=1195 (M+Na).

^{m7}(L-sugar isomer)Gppp^{m6}A_{2′OMe}pG (Compound 77):

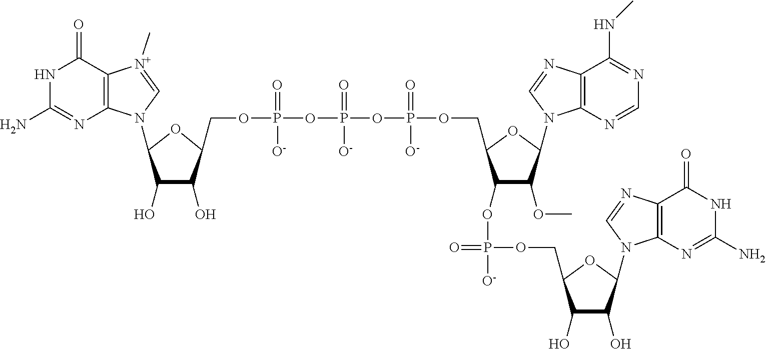

Compound 77 was synthesized according to general procedure 1 from N₇-Me-L-GDP (0.31 g, 0.5 mmol), EDC (0.153 g, 0.8 mmol) and imidazole (0.106 g, 1.550 mmol) in 3.1 mL 10% water in DMSO, magnesium chloride (0.212 g, 0.667 mmole) and p^{m6}AmpG (0.31 g, 0.336 mmol). 257.86 umole product was obtained (yield: 76.7%). ¹H NMR (500 MHz, D₂O): δ 8.26 (s, 1H), 8.05 (s, 1H), 7.83 (s, 1H), 6.03 (d, J=3.6 Hz, 1H), 5.76 (d, J=6.05 Hz, 1H) 5.67 (d, 2.8 Hz, 1H), 4.86 (m, 1H), 4.69-4.67 (t, J=5.5 Hz, 1H), 4.46-4.41 (m, 5H), 4.39-4.37 (m, 3H), 4.34-4.31 (m, 5H), 3.98 (s, 3H), 4.52 (s, 3H), 3.00 (bs, 3H), 1.90 (s, 1H), 1.18 (t, J=7.05 Hz, 1H).

31P NMR (200 MHz, D₂O): δ 0.47 (s, 1P), −10.92 (m, 1P), −22.11 (m, 1P) Calculated for C₃₃H₄₄N₁₅O₂₄P₄ 1158.69, found 1158.0 and 1159.9 [M−H] negative mode.

^{m7}(acyclo)Gppp^{m6}A_{2′OMe}pG: Compound 78

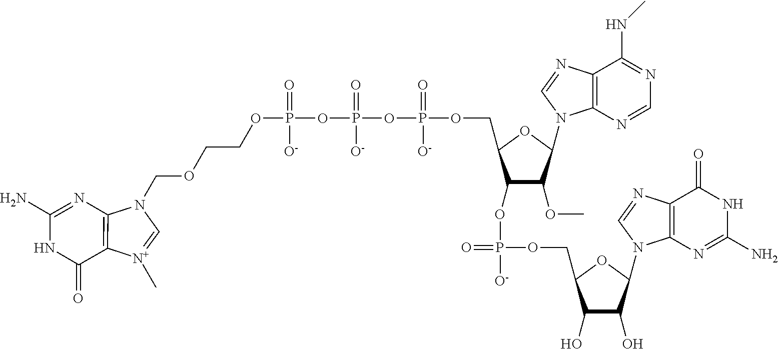

Compound 78 was synthesized according to general procedure 1 from N7-methyl acyclic GDP (0.100 g, 0.251 mmol), EDC (0.061 g, 320 mmol) and imidazole (0.054 g, 0.80 mmol) in 1.6 mL 10% water in DMSO, magnesium chloride (0.110 mL of 3.15 M of MgCl₂ Solution, 1 mmol) and p^{m6}AmpG (0.125 g, 0.173 mmol). Solid product (0.105 g) was obtained (55.0% yield). ¹H NMR (500 MHz, D₂O): δ 8.30 (s, 1H), 8.08 (s, 1H), 7.92 (s, 1H), 6.00 (d, J=5.8 Hz, 1H), 5.82 (d, J=5.7 Hz, 1H), 5.52-5.45 (q, J=10.95 Hz, 2H), 4.95-4.91 (m, 1H), 4.78-4.72 (m, 2H), 4.53 (br s, 1H), 4.48-4.44 (m, 2H), 4.34-4.29 (m, 2H), 4.25-4.21 (m, 3H), 4.20-4.09 (m, 2H), 3.99 (s, 3H), 3.79 (m, 2H), 3.45 (s, 3H), 3.03 (br s, 3H). ³¹P NMR (200 MHz, D₂O): δ −0.35 (1P), −10.68 (2P), −22.23 (1P). MS m/z=1168.6 (M+3Na).

### Synthesis of Compounds According to Formula III:

Compounds according to Formula III, terminating in an adenosine nucleotide, were prepared according to the general method shown in Scheme 2.

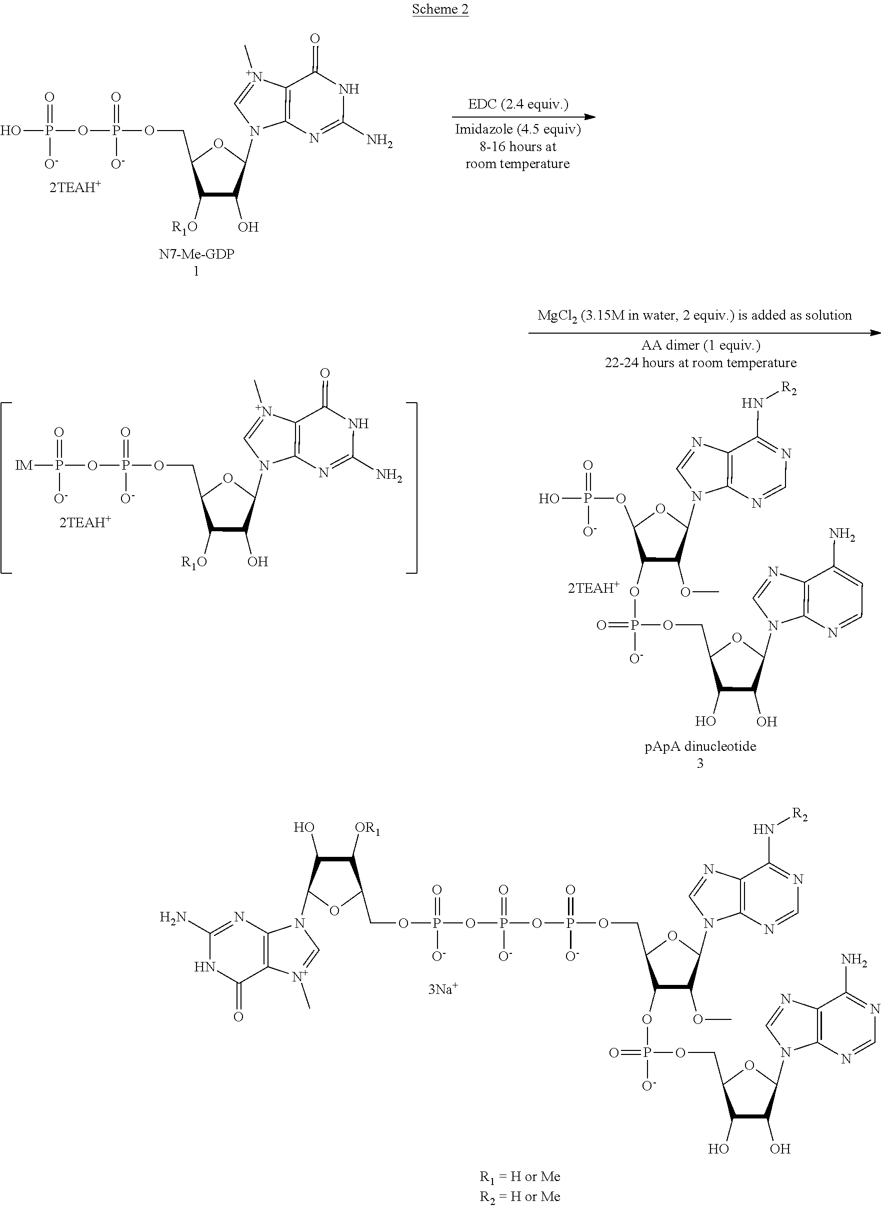

General Procedure 2 (Using a p^{m6}A_{2′OMe}pA Dinucleotide as pApA Dinucleotide 3):

To a stirred solution of modified N7-Me-GDP 1 (TEA salt, 1.5 mol equivalent based on p^{m6}A_{2′OMe}pA dinucleotide) in 10% water/DMSO (volume is 10× of weight of N7-Me-GDP 1) solution at room temperature was added EDC·HCl salt (2.4 mole equivalent based on p^{m6}A_{2′OMe}pA dinucleotide 3), followed by imidazole (4.5 mole equivalent based on p^{m6}A_{2′OMe}pA dinucleotide 3). The resulting mixture was allowed to stir at room temperature overnight (approximately 16-24 hours). The second day, magnesium chloride (3.15 M in water, 2.0 mole equivalent based on p^{m6}A_{2′OMe}pA dinucleotide 3) was added to the above solution, followed by modified p^{m6}A_{2′OMe}pA dinucleotide 3 (TEA salt, 1 mole equivalent). The resulting solution was allowed to stir at room temperature overnight (approximately 16 to 24 hours). The crude reaction mixture was then diluted with 10× water and purified by anion exchange chromatography (QFF Resin, 20% acetonitrile in water as buffer A, 1.5 M TEAA in water as buffer B, using a linear gradient from 25% to 45% buffer B for 4 CV and holding at 45% for 1.5 CV). The desired product was pooled and concentrated under vacuum, and the final product was precipitated as a sodium salt with sodium acetate and 95% absolute ethanol in water.

The syntheses of the following compounds followed the procedure described above and depicted in Scheme 2.

^{m7}Gppp^{m6}A_{2′OMe}pA (Compound 10):

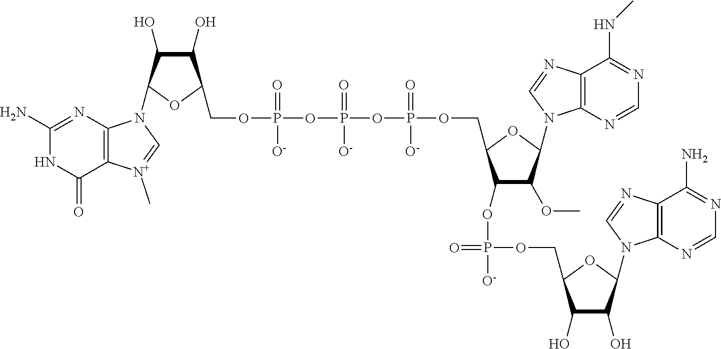

Compound 10 was synthesized as described according to General Procedure 2 from N7-Me-GDP (0.88 g, 1.5 mmol), EDC (0.46 g, 2.4 mmol) and imidazole (0.31 g, 4.5 mmol) in 13 mL DMSO, magnesium chloride (0.190 g in 0.64 mL water, 2 mmol) and p^{m6}A_{2′OMe}pA (0.906 g, 1 mmol). Solid product (0.355 g) was obtained (yield: 29%) ¹H NMR (500 MHz, D₂O): δ 8.26 (s, 1H), 8.23 (s, 1H), 8.15 (s, 1H), 7.91 (s, 1H), 6.008 (d, J=4.5 Hz, 1H), 5.881 (d, J=4.5 Hz, 1H), 5.761 (d, J=3.5 Hz, 1H), 4.84-4.88 (m, 1H), 4.25-4.49 (m, 14H), 3.96 (s, 3H), 3.53 (s, 3H). ³¹P NMR (200 MHz, D₂O): δ −0.59 (1P), −10.99, −11.04, −11.09, −11.13 (overlapping doublet, 2P), −22.50 (t, J=16.2 Hz, 1P). MS (ESI) calculated for C₃₃H₄₅N₁₅O₂₃P₄ 1143.70, found 1144.7 [M+H]⁻.

^{m7}G_{3′OMe}ppp^{m6}A_{2′OMe}pA (Compound 11):

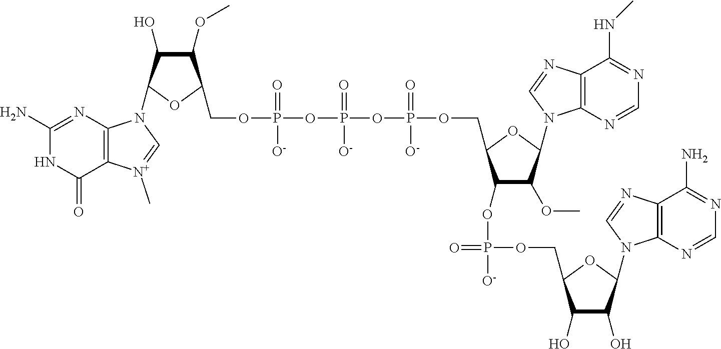

Compound 11 was synthesized as described according to General Procedure 2 from N7-Me-3′-OMe-GDP (0.63 g, 0.96 mmol), EDC (0.294 g, 1.536 mmol) and imidazole (0.196, 2.88 mmol) in 6.3 ml 10% water in DMSO, magnesium chloride (122 mg in 0.41 mL water, 1.28 mmol) and p^{m6}A_{2′OMe}pA (0.58 g, 0.64 mmol). Solid product (0.46 g) was obtained (yield: 53%)¹H NMR (500 MHz, D₂O): δ 8.28 (s, 1H), 8.24 (s, 1H), 8.14 (s, 1H), 7.93 (s, 1H), 6.013 (d, J=4.5 Hz, 1H), 5.911 (d, J=5 Hz, 1H), 5.741 (d, J=4.5 Hz, 1H), 4.87-4.90 (m, 1H), 4.59 (t, J=4.5 Hz, 1H), 4.45-4.52 (m, 3H), 4.32-4.40 (m, 5H), 4.16-4.25 (m, 4H), 4.04 (t, J=5 Hz, 1H), 3.96 (s, 3H), 3.52 (s, 3H), 3.44 (s, 3H). ³¹P NMR (200 MHz, D₂O): δ −0.49 (1P), −10.95, −11.02, −11.04, −11.11 (overlapping doublet, 2P), −22.37 (t, J=16.2 Hz, 1P). calculated for C₃₄H₄₇N₁₅O₂₃P₄ 1157.73, found 1156.7 [M−H].

### Synthesis of Compounds According to Formula V, Formula VII, and Formula VIII:

Compounds according to Formula V, terminating in a uridine nucleotide, were prepared according to the general method shown in Scheme 3. Compounds according to Formula VII and VIII are prepared similarly.

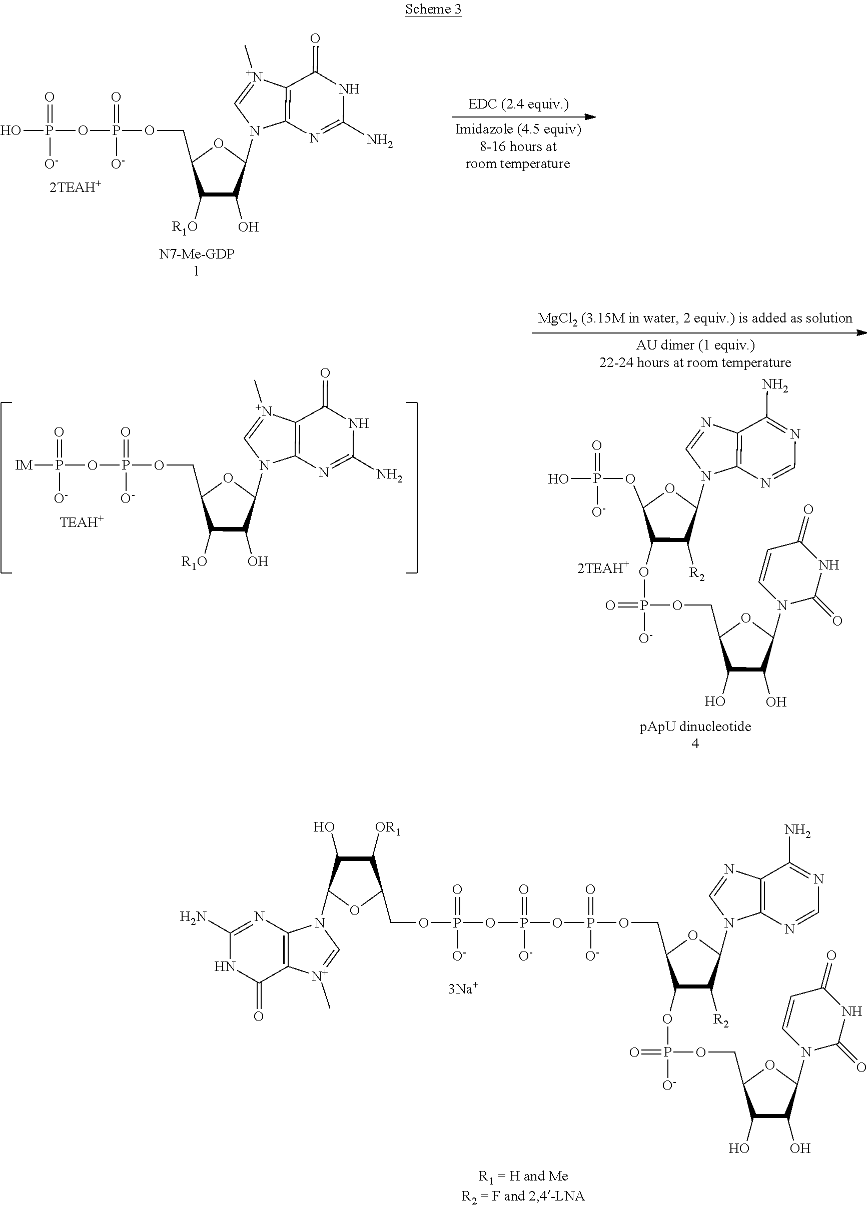

### General Procedure 3:

To a stirred solution of N7-Me-GDP 1 (TEA salt, 1.5 mol equivalent based on pApU dinucleotide) in 10% water/DMSO (volume is 10× of weight of N7-Me-GDP 1) solution at room temperature was added EDC·HCl salt (2.4 mol equivalent based on pApU dinucleotide 4), followed by imidazole (4.5 mol equivalent based on pApU dinucleotide 4). The resulting mixture was allowed to stir at room temperature overnight (approximately 16-24 hours). The second day, magnesium chloride (3.15 M in water, 2.0 mol equivalent based on pApU dinucleotide 4) was added to the above solution, followed by modified pApU dinucleotide 4 (TEA salt, 1 mol equivalent). The resulting solution was allowed to stir at room temperature overnight (approximately 16 to 24 hours). The crude reaction mixture was then diluted with 10× water and purified by anion exchange chromatography (QFF Resin, 20% acetonitrile in water as buffer A, 1.5M TEAA in water as buffer B, using a linear gradient from 25% to 45% buffer B for 4 CV and holding at 45% for 1.5 CV). The desired product was pooled and concentrated under vacuum, and the final product was precipitated as a sodium salt with sodium acetate and 95% absolute ethanol in water.

^{m7}GpppA_{2′4′LNA}pU (Compound 15):

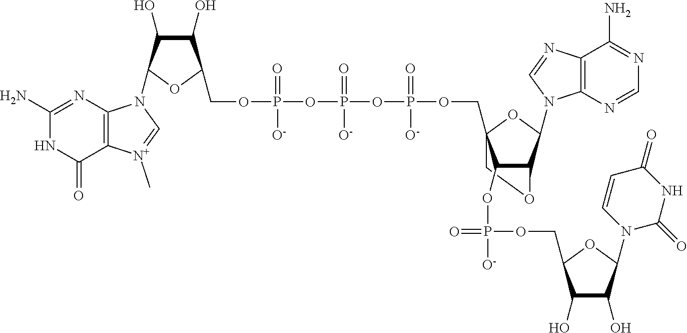

Compound 15 was synthesized as described according to General Procedure 3 from N7-Me-GDP (0.341 g, 0.52 mmol), EDC (0.161 g, 0.84 mmol) and imidazole (0.109 g, 1.6 mmol) in 3.4 ml 10% water in DMSO, magnesium chloride (0.254 mL of 3.15 M MgCl₂ solution, 0.8 mmol) and p(LNA)ApU (0.355 g, 0.4 mmol). Solid product (0.2976 g) was obtained (yield: 63%). ¹H NMR (500 MHz, D₂O): δ 8.13 (s, 2H), 7.63 (d, J=8.15 Hz, 1H), 5.90 (d, J=4.25 Hz, 1H), 5.78 (s, 1H), 5.65 (d, J=2.5 Hz, 1H), 5.30 (d, J=8.1 Hz, 1H), 4.64 (m, 1H), 4.56-4.25 (m, 12H), 4.12 (s, 3H), 4.07 (m, 1H). ³¹P NMR (200 MHz, D₂O): δ −1.85 (1P), −11.02 (2P), −22.37 (1P). MS m/z=1102.7 (M−H).

^{m7}G_{3′OMe}pppA_{2′4′LNA}PU (Compound 16):

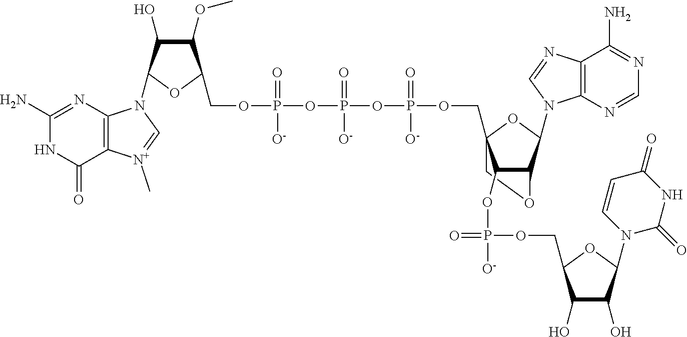

Compound 16 was synthesized as described according to General Procedure 3 from N7-Me-3′-OMe-GDP (0.42 g, 0.60 mmol), EDC (0.184 g, 0.96 mmol) and imidazole (0.122 g, 1.8 mmol) in 4.2 ml 10% water in DMSO, magnesium chloride (0.254 mL of 3.15 M MgCl₂ solution, 0.8 mmol) and p(LNA)ApU (0.355 g, 0.4 mmol). Solid product (0.3281 g) was obtained (yield: 69%). ¹H NMR (500 MHz, D₂O): δ 8.14 (s, 2H), 7.64 (d, J=8.15 Hz, 1H), 5.88 (d, J=4.7 Hz, 1H), 5.80 (s, 1H), 5.66 (d, J=2.55 Hz, 1H), 5.31 (d, J=8.15 Hz, 1H), 4.82 (m, 2H), 4.71-4.67 (m, 2H), 4.54-4.4.51 (m, 7H), 4.48-4.27 (m, 3H), 4.07 (s, 3H), 4.05 (m, 2H), 3.50 (s, 3H). ³¹P NMR (200 MHz, D₂O): δ −1.83 (1P), −10.98 (2P), −22.32 (1P). MS m/z=1117.9 (M−H).

^{m7}G_{3′OMe}pppA_{2′F}pU (Compound 17):

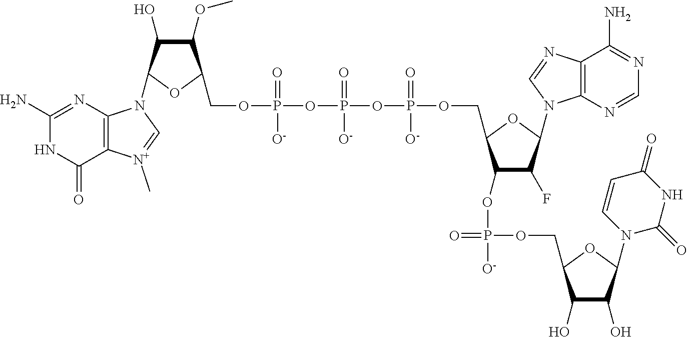

Compound 17 was synthesized as described according to General Procedure 3 from N7-Me-3′-OMe-GDP (0.42 g, 0.60 mmol), EDC (0.184 g, 0.96 mmol) and imidazole (0.122 g, 1.8 mmol) in 4.2 ml 10% water in DMSO, magnesium chloride (0.254 mL of 3.15 M MgCl₂ solution, 0.8 mmol) and pA_{2′F}pU (0.348 g, 0.4 mmol). Solid product (0.2788 g) was obtained (yield: 59%). ¹H NMR (500 MHz, D₂O): δ 8.33 (s, 1H), 8.16 (s, 1H), 7.70 (d, J=8.1 Hz, 1H), 6.23 (dd, J=2.3 Hz, 14.8 Hz, 1H), 5.84 (m, 2H), 5.60 (d, J=8.15 Hz, 1H), 5.48-5.36 (m, 1H), 4.88 (m, 1H), 4.78-4.11 (m, 13H), 4.07 (s, 3H), 3.48 (s, 3H). ³¹P NMR (200 MHz, D₂O): δ −0.85 (1P), −10.97 (2P), −22.31 (1P). MS m/z=1106.7 (M−H).

^{m7}G_{3′OMe}pppA_{2′OMe}pU (Compound 22):

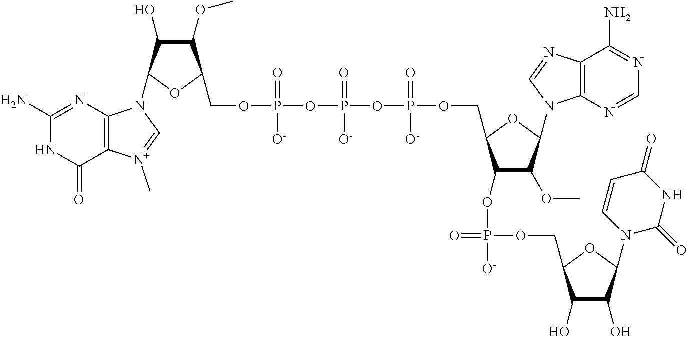

Compound 22 was synthesized as described according to General Procedure 3 from N₇-Me-3′-OMe-GDP (2.81 g, 4.5 mmol), EDC (1.38 g, 7.2 mmol) and imidazole (0.95 g, 13.95 mmol) in 30 mL 10% water in DMSO, magnesium chloride (1.9 mL of 3.15M MgCl₂ solution, 6.0 mmol) and pA_{2′OMe}pU (2.61 g, 3.0 mmol). Solid product (1.95 g, 1.64 mmol) was obtained (yield: 55%). NMR (500 MHz, D₂O) δ 8.40 (s, 1H), 8.13 (s, 1H), 7.85 (d, J=8.2, 1H), 6.04 (d, J=5.8, 1H), 5.92 (d, J=4.7), 5.78 (m, 2H), 4.61 (t, J=4.6, 1H), 4.55 (m, 1H), 4.45 (t, J=5.2, 1H), 4.17-4.38 (m, 10H), 4.07 (t, J=4.9, 1H), 4.03 (s, 3H), 3.49 (s, 3H), 3.46 (s, 3H). 31P NMR (200 MHz, D₂O) −0.56 (1P), −11.05 (2P), −22.38 (1P). MS m/z=1118.9 (M−H).

^{m7}Gppp^{m6}A_{2′OMe}pU (Compound 23):

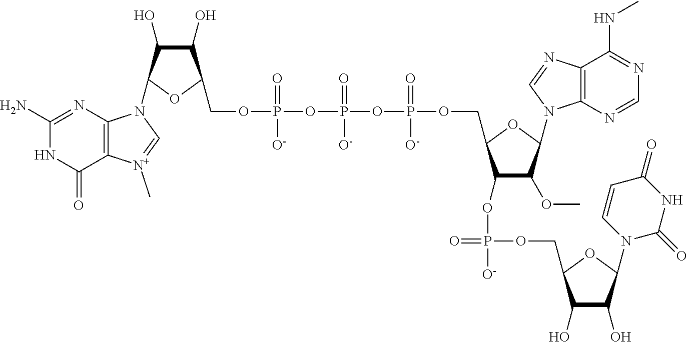

Compound 23 was synthesized as described according to General Procedure 3 from N7-Me-GDP (2.93 g, 4.5 mmol), EDC (1.38 g, 7.2 mmol) and imidazole (0.95 g, 13.95 mmol) in 30 mL 10% water in DMSO, magnesium chloride (1.9 mL of 3.15 M MgCl₂ solution, 6.0 mmol) and p^{m6}A_{2′OMe}pU (2.65 g, 3.0 mmol). Solid product (1.11 g, 0.94 mmol) was obtained (yield: 31%). NMR (500 MHz, D₂O) δ 8.34 (s, 1H), 8.13 (s, 1H), 7.81 (d, J=8.2, 1H), 6.04 (d, J=5.7, 1H), 5.89 (d, J=4.6, 1H), 5.81 (d, J=3.7, 1H), 5.72 (d, J=8.1, 1H), 4.55 (m, 1H), 4.40-4.46 (m, 2H), 4.32-4.39 (m, 4H), 4.23-4.29 (m, 5H), 4.17 (m, 1H), 3.99 (s, 3H), 3.50 (s, 3H), 3.08 (bs, 3H). 31P NMR (200 MHz, D₂O) −0.57 (1P), −11.01 (2P), −22.41 (1P). MS m/z=1118.7 (M−H).

^{m7}G_{3′OMe}ppp^{m6}A_{2′OMe}pU (Compound 24):

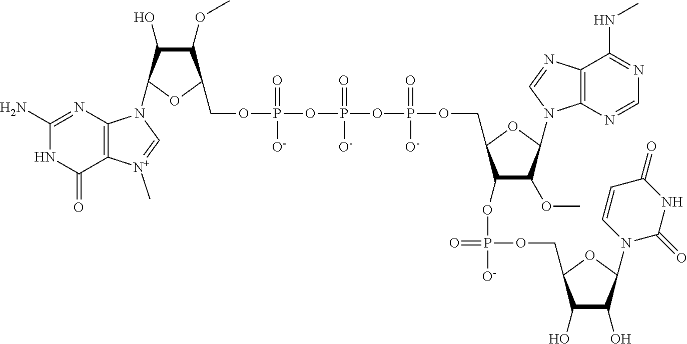

Compound 24 was synthesized as described according to General Procedure 3 from N7-Me-3′-OMe-GDP (5.8 g, 9.4 mmol), EDC (2.9 g, 15.0 mmol) and imidazole (2.0 g, 28.9 mmol) in 58 mL 10% water in DMSO, magnesium chloride (4.0 mL of 3.15 M MgCl₂ solution, 12.4 mmol) and p^{m6}A_{2′OMe}pU (5.5 g, 6.3 mmol). Solid product (3.71 g, 3.08 mmol) was obtained (yield: 49%). Final yield—3084 μmol, 49.5%. NMR (500 MHz, D₂O) δ 8.34 (s, 1H), 8.14 (bs, 1H), 7.82 (d, J=8.2, 1H), 6.04 (d, J=5.8, 1H), 5.90 (d, J=4.6, 1H), 5.79 (d, J=4.2, 1H), 5.73 (d, J=8.2, 1H), 4.95 (m, 1H), 4.60 (t, J=4.5, 1H), 4.55 (m, 1H), 4.36-4.44 (m, 4H), 4.33 (m, 2H), 4.15-4.29 (m, 7H), 4.05 (t, J=4.9, 1H), 3.99 (s, 3H), 3.49 (s, 3H), 3.45 (s, 3H), 3.07 (bs, 3H). 31P NMR (200 MHz, D₂O) −0.56 (1P), −11.08 (2P), −22.40 (1P). MS m/z=1132.8 (M−H).

^{m7}GpppA_{2′F}pU (Compound 33):

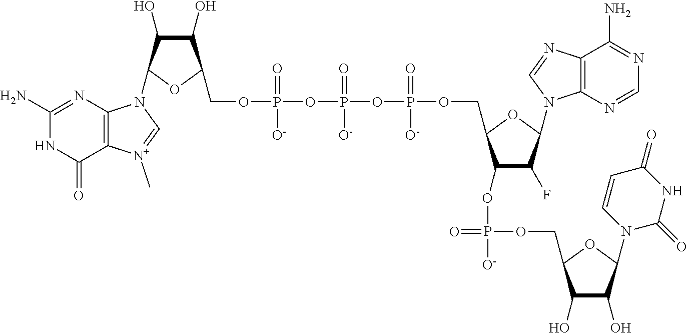

Compound 33 was synthesized as described according to General Procedure 3 by coupling N7-Me-GDP imidazolide to the pA_{2′F}pU dinucleotide. ¹H NMR (500 MHz, D₂O): δ 8.30 (s, 1H), 8.14 (s, 1H), 7.68 (d, J=8.1 Hz, 1H), 6.18 (dd, J=2.1 Hz, 14.7 Hz, 1H), 5.82 (dd, J=3.8 Hz, 11.2 Hz, 2H), 5.58 (d, J=8.1 Hz, 1H), 5.45 (m, 1H), 5.35 (m, 1H), 4.56-4.11 (m, 11H), 4.06 (s, 3H). ³¹P NMR (200 MHz, D₂O): δ −0.89 (1P), −11.01 (2P), −22.36 (1P).

### Synthesis of Additional Compounds According to Formula IV:

Certain compounds according to Formula IV were prepared according to the following general method (General Procedure 4), including the synthesis of an activated imidazolide of pp^{m6}ApG dinucleotide and the synthesis of an N7-Me 3′OMe guanosine 5′-thiophosphate.

### General Procedure 4:

Synthesis of Activated Imidazolide of pp^{m6}ApG Dinucleotide:

A large-scale activation of p^{m6}ApG dinucleotide (5.0 g, 6.94 mmoles) using imidazole (2.20 g, 32.27 mmoles) and EDC (2.59 g, 16.66 mmoles) in 20 mL of 90% DMSO 10% H₂O was performed by combining the reagents and stirring at room temperature for 22 hours. After activation, addition of β-phosphate was accomplished by the addition of 1M TBAP in DMF (85 mL, 85 mmoles) with mixing at room temperature for 18 hours. The reaction solution was then double purified by reverse phase and anion exchange chromatography. The pp^{m6}ApG dinucleotide (0.5 g, 0.625 mmole) was activated with EDC (0.146 g, 0.94 mmole) and imidazole(0.128 g, 1.88 mmole) and used for subsequent coupling reactions.

### Synthesis of N7-Me 3′OMe Guanosine 5′-Thiophosphate:

5′OH 3′OMe Guanosine was converted into 5′iodo 3′OMe guanosine using an Appel reaction. Specifically, the starting material 5′OH 3′OMe guanosine (1.00 g, 3.36 mmol) was dissolved in anhydrous NMP (10 mL), followed by the addition of imidazole (1.38 g, 20.2 mmol), TPP (2.65 g, 10.1 mmol), and iodine (2.55 g, 10.1 mmol). The reaction was stirred at ° C. for 3 hours, then transferred to a 120 mL dichloromethane/water mix (3:1). The mixture was kept at 4° C. overnight, at which point a white precipitate formed. The precipitate was filtered off under reduced pressure and dried overnight in a vacuum desiccator to yield 5′iodo 3′OMe guanosine. The material was used without further purification.

The 5′thiophosphorylation of 5′iodo 3′OMe guanosine was accomplished using sodium thiophosphate. 5′Iodo 3′OMe guanosine (1.00 g, 2.45 mmol) was dissolved in 15 mL of 100 mM NaOH, followed by the addition of sodium thiophosphate (2.21 g, 12.30 mmol). The reaction mixture was heated at 50° C. for 3 hours. The product was purified by reverse-phase HPLC.

The N-7 methylation of 5′thiophosphate 3′OMe guanosine was accomplished by using dimethyl sulfate. 5′Thiophosphate 3′OMe guanosine (1.00 g, 2.54 mmol) was dissolved in 10 mL 0.5 M NaOAc pH 4.0 Buffer, followed by the portion wise addition of dimethyl sulfate (0.64 g, 5.09 mmol) over an hour. The pH was maintained around 4.0 using 1M NaOH as needed. The reaction volume was diluted 10× using deionized water, and then washed twice with equal volumes (100 mL) of ethyl acetate. The reaction was purified by anion exchange chromatography (QFF Resin) and the product was obtained as a triethylamine (TEA) salt.

Synthesis of ^{m7}G_{3′OMe5′-thio}ppp^{m6}A_{2′OMe}pG (Compound 12):

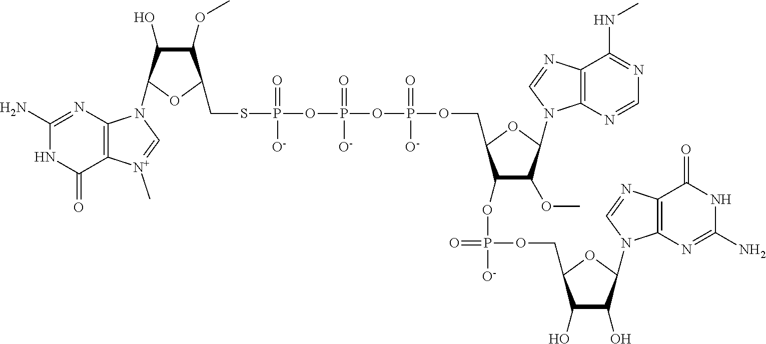

Compound 12 was synthesized as described in General Procedure 4 by coupling N7-Me 3′OMe guanosine 5′-thiophosphate with activated imidazolide of pp^{m6}A_{2′OMe}pG dinucleotide in the presence of MgCl₂. The activated imidazolide of pp^{m6}A_{2′OMe}pG dinucleotide was utilized as a solution in 90% DMSO 10% H₂O 3.44 mL at [0.625 mmol/5 mL], along with the addition of N₇-Me 3′OMe guanosine 5′thiophosphate (150.0 mg, 367 umoles) and magnesium chloride 3.15M (232 uL, 734 umol) as described above. The components were mixed and stirred at room temperature overnight. The product was purified by anion exchange chromatography using QFF resin and then by QHP anion exchange chromatography. Final yield after precipitation was 45.6%, 196.53 mg of a white solid product was obtained. ¹H NMR (500 MHz, D₂O): δ 8.29 (s, 1H), 8.04 (s, 1H), 7.93 (s, 1H), 5.996 (d, J=6.2 Hz, 1H), 5.821 (d, J=5.9 Hz, 1H), 5.704 (d, J=5.5 Hz, 1H), 4.89-4.92, (m, 1H), 4.74 (t, J=3.6 Hz), 4.48-4.52, (m, 1H), 4.43-4.47 (m, 1H), 4.38-4.40 (m, 2H), 4.30-4.34 (m, 2H), 4.21-4.24 (m, 1H), 4.18-4.20 (m, 2H), 4.00 (t, J=3.75 Hz, 1H), 3.97 (s, 3H), 3.47 (s, 3H), 3.39 (s, 3H), 3.26, (q, J=6.4 Hz, 2H), 3.05, (s, 3H). ³¹P NMR (200 MHz, D₂O): δ 7.85 (d, J=16.36 Hz, 1P), 0.34 (s, 1P), −11.17, (d, J=18.18 Hz, 1P), −23.22 (d, J=18.12 Hz, 1P). Calculated for C₃₄H₄₈N₁₅O₂₃P₄S 1190.17, found 1189.17 [M−H].

Synthesis of ^{m7}G_{3′OMe}pp(s)p^{m6}A_{2′OMe}pG* (Compound 13):

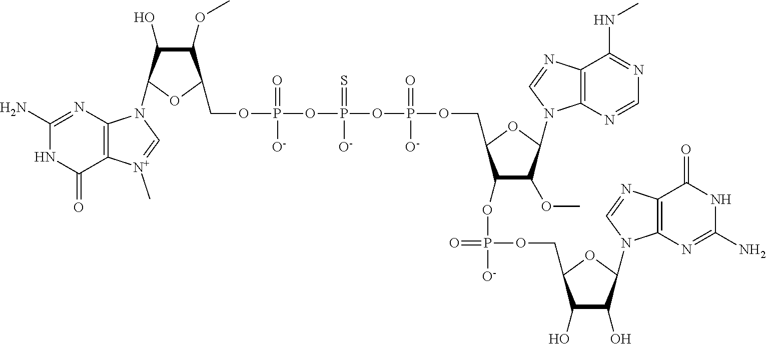

^{m7}G_{3′OMe}pp(S)p^{m6}A_{2′OMe}pG contains a chiral phosphorothioate moiety and is separated into two diastereomers: ^{m7}G_{3′OMe}pp(s_{Rp})p^{m6}A_{2′OMe}pG and ^{m7}G_{3′OMe}pp(s_{Sp})p^{m6}A_{2′OMe}pG

To a stirred suspension of β-S-3′-OMe-GDP (1.11 g, 1.61 mmol) in DMSO (8 mL) at room temperature was added p^{m6}AmpG-imidazolide (0.62 g, 0.81 mmol) followed by magnesium chloride (0.51 mL of 3.15 M MgCl₂ solution, 1.61 mmol). The reaction progressed and was purified by anion exchange chromatography as described in General Procedure 1. A pure mixture of the diastereomers was obtained and concentrated under vacuum to remove acetonitrile. The resulting solution was diluted with 5× water and purified using reverse-phase chromatography (Waters Nova-Pak C18 Prep Column, 60A, 6 μm, 19 mm×300 mm, WAT025822, 50 mM ammonium acetate, pH 6.0 as buffer A, acetonitrile as buffer B, using a linear gradient from 0% to 5% buffer B for 10 column volumes). The desired product was pooled and concentrated under vacuum, and the final product was precipitated as a sodium salt with sodium acetate and 95% absolute ethanol in water. Solid product was obtained as pure diastereomers, D1: 0.04 mmol, D2: 0.05 mmol, 11.5% total yield for D1+D2.

^{m7}G_{3′OMe}pp(s)p^{m6}A_{2′OMe}pG D1

¹H NMR (500 MHz, D₂O) δ 8.37 (s, 1H), 8.11 (s, 1H), 7.94 (s, 1H), 6.02 (d, J=5.7, 1H), 5.86 (d, J=4.1, 1H), 5.83 (d, J=5.6, 1H), 4.96 (m, 1H), 4.71 (m, 1H), 4.47-4.53 (m, 3H), 4.42-4.44 (m, 2H), 4.36 (m, 1H), 4.21-4.30 (m, 5H), 4.13 (t, J=4.6, 1H), 4.02 (s, 3H), 3.47 (s, 3H), 3.46 (s, 3H), 3.08 (bs, 3H); ³¹P NMR (200 MHz, D₂O) 30.49 (1P), −0.37 (1P), −11.92 (2P); MS m/z=1187.5 (M−H).

^{m7}G_{3′OMe}pp(s)p^{m6}A_{2′OMe}pG D2

¹H NMR (500 MHz, D₂O) δ 8.34 (s, 1H), 8.12 (s, 1H), 7.95 (s, 1H), 6.01 (d, J=5.8, 1H), 5.83 (m, 2H), 4.96 (m, 1H), 4.42-4.55 (m, 3H), 4.32-4.39 (m, 4H), 4.19-4.28 (m, 4H), 4.14 (t, J=4.6, 1H), 4.03 (s, 3H), 3.48 (s, 3H), 3.47 (s, 3H), 3.08 (bs, 3H). ³¹P NMR (200 MHz, D₂O) 30.38 (1P), −0.41 (1P), −11.95 (2P); MS m/z=1187.6 (M−H).

### Synthesis of Additional Compounds According to Formula VI:

Certain compounds according to Formula VI were prepared according to the following general method (General Procedure 5), including the synthesis of an activated imidazolide of ppA_{2′OMe}pG dinucleotide and the synthesis of an N7-Me 3′OMe guanosine 5′-thiophosphate.

### General Procedure 5:

Synthesis of Activated Imidazolide of ppA_{2′OMe}pG Dinucleotide:

Activation of pA_{2′OMe}pG dinucleotide (2.0 g, 2.20 mmoles) using imidazole (696.5 mg, 10.23 mmoles) and EDC (1.01 g, 5.28 mmoles) in 10 mL of 90% DMSO 10% H₂O was performed by combining the reagents and stirring at room temperature for 2 hours. After activation, addition of 0-phosphate was accomplished by the addition of 1M TBAP in DMF (38.20 mL, 38.20 mmoles) with mixing at room temperature for 18 hours to yield ppA_{2′OMe}pG. The reaction solution is then double purified by reverse phase and anion exchange chromatography. The ppA_{2′OMe}pG (1 g, 0.917 mmole) was activated with EDC(0.214 g, 1.375 mmole) and imidazole(0.187 g, 2.75 mmole) and used for the subsequent coupling reactions.

### Synthesis of N7-Me 3′OMe Guanosine 5′-Thiophosphate:

5′OH 3′OMe Guanosine was converted into 5′iodo 3′OMe guanosine using an Appel reaction. The starting material 5′OH 3′OMe Guanosine (1.00 g, 3.36 mmol), was dissolved in anhydrous NMP (10 mL), followed by the addition of imidazole (1.38 g, 20.2 mmol), TPP (2.65 g, 10.1 mmol), and iodine (2.55 g, 10.1 mmol). The reaction was stirred at 25° C. for 3 hours, then transferred to a 120 mL dichloromethane/water mix (3:1). The mixture was kept at 4° C. overnight, at which point a white precipitate formed. The precipitate was filtered off under reduced pressure and dried overnight in a vacuum desiccator to yield 5′iodo 3′OMe guanosine. The material was used without further purification.

The 5′thiophosphorylation of 5′iodo 3′OMe guanosine was accomplished using sodium thiophosphate. 5′Iodo 3′OMe guanosine (1.00 g, 2.45 mmol) was dissolved in 15 mL of 100 mM NaOH, followed by the addition of sodium thiophosphate (2.21 g, 12.30 mmol). The reaction mixture was heated at 50° C. for 3 hours. The product was purified by reverse-phase HPLC.

The N-7 methylation of 5′thiophospate 3′OMe guanosine was accomplished by using dimethyl sulfate. 5′Thiophosphate 3′OMe guanosine (1.00 g, 2.54 mmol) was dissolved in 10 mL 0.5 M NaOAc pH 4.0 Buffer, followed by the portion wise addition of dimethyl sulfate (0.64 g, 5.09 mmol) over an hour. The pH was maintained around 4.0 using 1M NaOH as needed. The reaction volume was diluted 10× using deionized water, and then washed twice with equal volumes (100 mL) of ethyl acetate. The reaction was purified by anion exchange chromatography (QFF Resin) and the product was obtained as a triethylamine (TEA) salt.

Synthesis of ^{m7}G_{3′OMe}p(s)ppA_{2′OMe}pG (Compound 18):

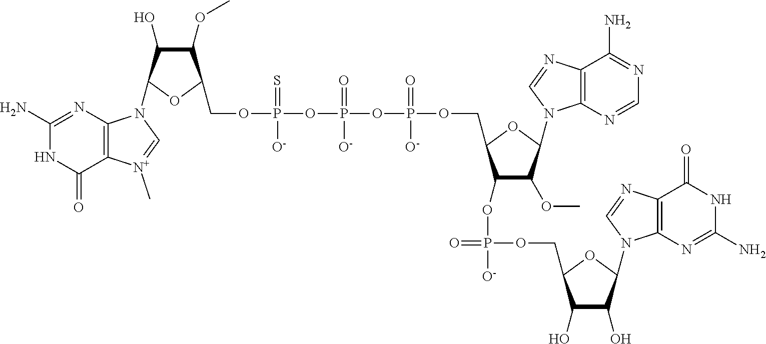

Compound 18 was synthesized as described in General Procedure 5 by coupling N7-Me 3′OMe guanosine 5′-thiophosphate with activated imidazolide of ppA_{2′OMe}pG dinucleotide in the presence of MgCl₂. The activated imidazolide of ppA_{2′OMe}pG dinucleotide was utilized as a solution in 90% DMSO 10% H₂O 1.1 mL at [0.917 mmol/10 mL], along with the addition of N7-Me 3′OMe guanosine 5′thiophosphate (43 mg, 67 umoles) and magnesium chloride 3.15 M (43 uL, 134 umol) as described above. The components were mixed and stirred at room temperature overnight. The product was purified by anion exchange chromatography using QFF resin and then by QHP anion exchange chromatography. Final yield after precipitation was 43.3%, 29 umol of product was obtained. ¹H NMR (500 MHz, D₂O): δ 8.40 (s, 1H), 8.17 (d, J=3.1 Hz, 1H), 7.98 (d, J=5.8 Hz, 1H), 6.00 (d, J=5.85 Hz, 1H), 5.86-5.81 (m, 2H), 4.97-4.88 (m, 1H), 4.69-4.68 (m, 2H), 4.66-4.65 (m, 2H), 4.45-4.37 (m, 4H), 4.33-4.30 (m, 2H), 4.28-4.22 (m, 2H), 4.15-4.10 (m, 1H), 4.05-4.03 (d, J=8.15, 3H), 3.48-3.42 (m, 6H), 1.93 (s, 1H). ³¹P NMR (200 MHz, D₂O): δ 43.90-43.28 (dd, 1P) 0.39 (s, 1P), −11.23 (t, 1P), −23.66 (m, 1P). Calculated for C₃₃H₄₄N₁₅O₂₃P₄S 1174.75, found 1174 & 1175.7 [M−H] negative mode.

Synthesis of ^{m7}G_{3′OMe}pp(s)pA_{2′OMe}pG* (Compound 19):

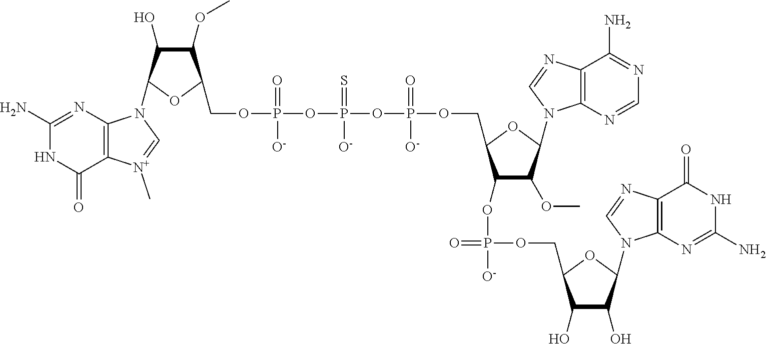

^{m7}G_{3′OMe}pp(s)pA_{2′OMe}pG contains a chiral phosphorothioate moiety and is separated into two diastereomers: ^{m7}G_{3′OMe}pp(s_{Rp})pA_{2′OMe}pG and ^{m7}G_{3′OMe}pp(s_{Sp})pA_{2′OMe}pG

To a stirred suspension of β-S-3′-OMe-GDP (0.98 g, 1.42 mmol) in DMSO (7 mL) at room temperature was added pA_{2′OMe}pG-imidazolide (0.54 g, 0.71 mmol) followed by magnesium chloride (0.45 mL of 3.15 M MgCl₂ solution, 1.42 mmol). The reaction progressed and was purified by anion exchange chromatography as described in General Procedure 1. A pure mixture of the diastereomers was obtained and concentrated under vacuum to remove acetonitrile. The resulting solution was diluted with 5× water and purified using reverse-phase chromatography (Waters Nova-Pak C18 Prep Column, 60A, 6 μm, 19 mm×300 mm, WAT025822, 50 mM ammonium acetate, pH 6.0 as buffer A, acetonitrile as buffer B, using a linear gradient from 0% to 5% buffer B for 10 column volumes. The desired product was pooled and concentrated under vacuum, and the final product was precipitated as a sodium salt with sodium acetate and 95% absolute ethanol in water. Solid product was obtained as pure diastereomers, D1: 0.06 mmol, D2: 0.07 mmol, Final yield—19% (D1+D2).

^{m7}G_{3′OMe}pp(s)pA_{2′OMe}pG D1

¹H NMR (500 MHz, D₂O) δ 8.45 (s, 1H), 8.14 (s, 1H), 7.96 (s, 1H), 6.04 (d, J=6.0, 1H), 5.88 (d, J=4.2, 1H), 5.85 (d, J=5.8, 1H), 4.98 (m, 1H), 4.49-4.53 (m, 3H), 4.41-4.45 (m, 2H), 4.36 (m, 1H), 4.19-4.28 (m, 5H), 4.15 (t, J=4.8, 1H), 4.04 (s, 3H), 3.48 (s, 3H), 3.44 (s, 3H); 31P NMR (200 MHz, D₂O) 30.46 (1P), −0.35 (1P), −11.90 (2P); MS m/z=1173.8 (M−H).

^{m7}G_{3′OMe}pp(s)pA_{2′OMe}pG D2:

¹H NMR (500 MHz, D₂O) δ 8.42 (s, 1H), 8.14 (s, 1H), 7.97 (s, 1H), 6.03 (d, J=6.0, 1H), 5.85 (m, 2H), 4.98 (m, 1H), 4.51-4.55 (m, 3H), 4.36-4.48 (m, 3H), 4.19-4.30 (m, 5H), 4.16 (t, J=4.7, 1H), 4.06 (s, 3H), 3.49 (s, 3H), 3.45 (s, 3H); ³¹P NMR (200 MHz, D₂O) 30.36 (1P), −0.41 (1P), −11.91 (2P); MS m/z=1173.8 (M−H).

Synthesis of ^{m7}G_{3′OMe}ppp(s)A_{2′OMe}pG (Compound 20):

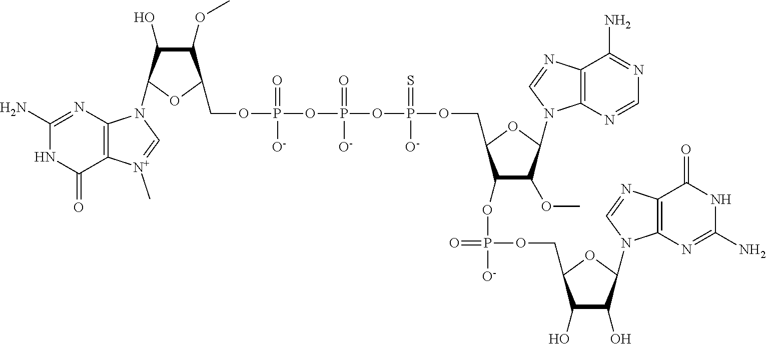

In a clean glass vessel, N7-Methyl-3′-O-methyl-GDP (480 mol) was suspended in 10% water in DMSO using vigorous stirring at 25° C. EDC-HCl was then added, promptly followed by adding imidazole. The reaction was then stirred at 25° C., monitoring by analytical anion exchange ultra-high-performance liquid chromatography (AX-UHPLC), and was complete between 8 hours to 18 hours. To the activated N₇-Methyl-3′-O-methyl-GDP was added 5′-O-thiophosphoryl-2′-O-methyladenylyl-(3′-O-phosphoryl-O-5′)-guanosine (240 mol) and aqueous magnesium chloride. The coupling reaction was stirred at 25° C., monitoring by analytical AX-UHPLC. Reaction completion was determined by no more than 5% of dinucleotide starting material remaining. The resulting crude reaction was diluted with water to 20-fold crude volume and purified by anion exchange chromatography using QFF Sepharose resin and 30-60% 1.5 M TEAA in 20% acetonitrile with water. Fractions with >80% purity by analytical AX-UHPLC were pooled and partially evaporated to remove acetonitrile. The combined fractions were then diluted with 6-8 volumes of water and further purified by reverse phase chromatography using C18 resin and 0-10% acetonitrile in 100 mM TEAA, pH 6-7. Fractions with >97% purity by analytical AX-UHPLC were pooled and evaporated, reducing the product to a viscous oil. The trinucleotide was converted to the sodium salt form by first adding 3 M sodium acetate to the post-RP oil and gradually adding 95% ethanol in water to precipitate the trinucleotide as a sodium salt. The precipitate was washed twice with 95% ethanol, decanting the supernatants, and the pellet was resuspended in nuclease-free water. Residual ethanol was removed by rotary evaporation and the solution was adjusted to 100 mM final concentration in water (130 mol, 54% yield). ¹H NMR δ (D₂O, 500 MHz) 8.44 (s, 1H), 8.08 (s, 1H), 7.93 (s, 1H), 6.00 (m, 1H), 5.83 (m, 2H), 4.95 (m, 1H), 4.68 (m, 1H), 4.50 (m, 2H), 4.42 (m, 1H), 4.37 (m, 1H), 4.33-4.25 (m, 3H), 4.20 (m, 3H), 4.12 (m, 1H), 4.01 (s, 3H), 3.45 (s, 3H), 3.41 (s, 3H). ³¹P NMR δ (D₂O, 200 MHz) 43.70 (d, J=24.2 Hz, 1P), −0.41 (s, 1P), −11.12 (d, J=24.2 Hz, 1P), −23.56 (dd, J=24 Hz, 24 Hz, 1P).

Synthesis of ^{m7}G_{3′OMe}pppA_{2′OMe5′thio}pG (Compound 73):

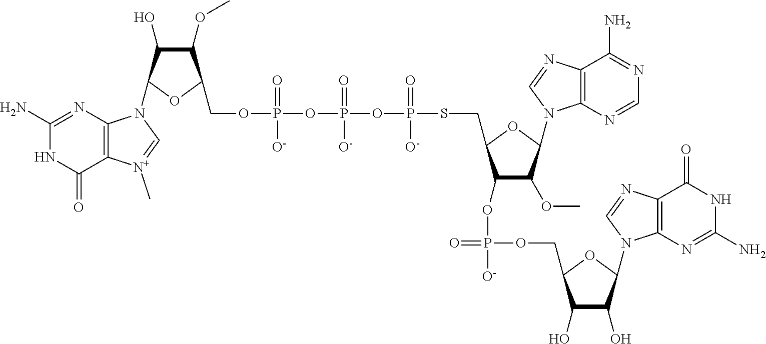

The synthesis and processing for the α-phosphorothiolate trinucleotide cap Compound 73 was performed similarly to the procedure described for the α-phosphorothioate (α-PS) analogue-Compound 20. ¹H NMR δ (D₂O, 500 MHz) 8.21 (s, 1H), 8.02 (s, 1H), 7.93 (s, 1H), 5.93 (d, J=5 Hz, 1H), 5.79 (m, 2H), 4.81 (m, 1H), 4.70 (m, 1H), 4.64 (m, 1H), 4.50 (m, 2H), 4.42 (m, 3H), 4.33 (m, 1H), 4.21 (m, 3H), 4.08 (t, J=5 Hz, 1H), 4.00 (s, 3H), 3.46 (s, 3H), 3.42 (s, 3H). ³¹P NMR δ (D₂O, 200 MHz) 7.66 (d, J=26.2 Hz, 1P), −0.56 (s, 1P), −11.08 (d, J=18.2 Hz, 1P), −23.15 (dd, J=20 Hz, 26.2 Hz, 1P).

Synthesis of ^{m7}G_{3′OMe}ppp^{m6}A_{2′OMe}p(s)G* (Compound 74)

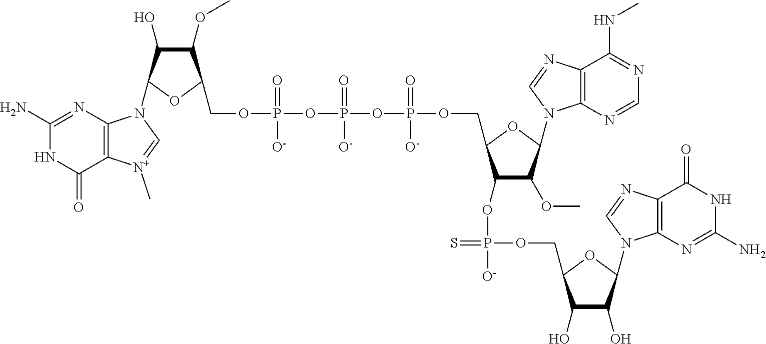

^{m7}G_{3′OMe}ppp^{m6}A_{2′OMe}p(s)G contains a chiral phosphorothioate moiety and is separated into two diastereomers: ^{m7}G_{3′OMe}ppp^{m6}A_{2′OMe}p(s_{Rp})G and ^{m7}G_{3′OMe}ppp^{m6}A_{2′OMe}p(s)G

p^{m6}A_{2′OMe}p_{S}G dinucleotide was purified by reverse-phase chromatography to isolate the diastereomers (Daisogel SP-100-15-ODS-P, 10 0 mM TEAB as buffer A, acetonitrile as buffer B, using a linear gradient from 0% to 11% buffer B for 11 column volumes).

Compound 74 (D1) was synthesized as described according to General Procedure 1 from N₇-Me-3′-OMe-GDP (0.19 g, 0.30 mmol), EDC (0.07 g, 0.39 mmol) and imidazole (0.03 g, 0.40 mmol) in 1.9 mL 10% water in DMSO, magnesium chloride (0.13 mL of 3.15M MgCl₂ solution, 0.40 mmol) and p^{m6}A_{2′OMe}p_{S}G (0.2 g, 0.20 mmol). Solid product (0.12 g, 0.09 mmol) was obtained. Final yield—94.8 μmol, 47.7%. ¹H NMR (500 MHz, D₂O) δ 8.29 (s, 1H), 8.07 (s, 1H), 8.02 (s, 1H), 5.98 (d, J=5.8, 1H), 5.83 (d, J=5.8, 1H), 5.79 (d, J=4.2, 1H), 5.08 (m, 1H), 4.70 (t, J=5.5, 1H), 4.65 (t, J=4.6, 1H), 4.55 (m, 1H), 4.51 (t, J=4.1, 1H), 4.46 (t, J=5.3, 1H), 4.37 (m, 3H), 4.34 (m, 1H), 4.25 (m, 3H), 4.18 (m, 1H), 4.06 (t, J=4.7, 1H), 3.98 (s, 3H), 3.48 (s, 3H), 3.45 (s, 3H), 3.05 (bs, 3H). ³¹P NMR (200 MHz, D₂O) 55.91 (1P), −11.09 (2P), −22.42 (1P), MS m/z=1188.8 (M−H)

Compound 74 (D2) was synthesized as described according to General Procedure 1 from N7-Me-3′-OMe-GDP (0.19 g, 0.30 mmol), EDC (0.07 g, 0.39 mmol) and imidazole (0.03 g, 0.40 mmol) in 1.9 mL 10% water in DMSO, magnesium chloride (0.13 mL of 3.15 M MgCl₂ solution, 0.40 mmol) and p^{m6}A_{2′OMe}p_{S}G (0.2 g, 0.20 mmol). Solid product (0.14 g, 0.11 mmol) was obtained. Final yield—112.3 μmol, 55.9%. NMR (500 MHz, D₂O) δ 8.29 (s, 1H), 8.06 (s, 1H), 7.96 (s, 1H), 6.00 (d, J=5.5, 1H), 5.79 (m, 2H), 5.06 (m, 1H), 4.69 (t, J=5.2, 1H), 4.64 (t, J=4.6, 1H), 4.56 (m, 1H), 4.47 (t, J=4.5, 1H), 4.29 (m, 1H), 4.39 (m, 1H), 4.35 (m, 3H), 4.18-4.27 (m, 4H), 4.07 (t, J=4.7, 1H), 4.00 (s, 3H), 3.46 (s, 3H), 3.44 (s, 3H), 3.054 (bs, 3H).

³¹P NMR (200 MHz, D₂O) 56.79 (1P), −11.05 (2P), −22.40 (1P). MS m/z=1187.8 (M−H).

Synthesis of ^{m7}G_{3′OMe}ppppA_{2′OMe}pG (Compound 79):

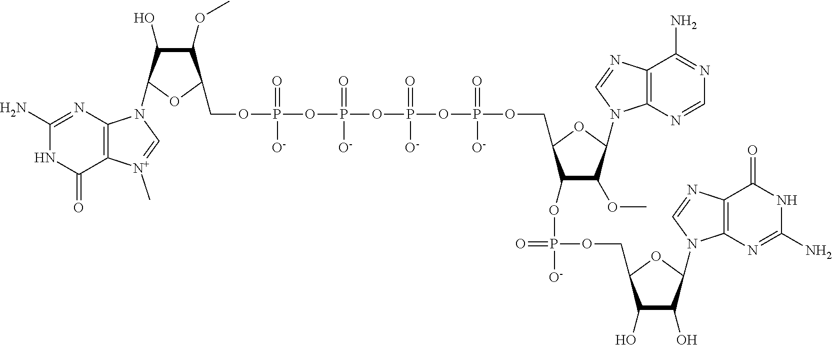

Compound 79 was synthesized according to this procedure: A large-scale activation of p^{m6}A_{2′OMe}pG dinucleotide (2.0 g, 2.20 mmoles) using imidazole (696.50 mg, 10.23 mmoles) and EDC (1.01 g, 5.28 mmoles) in 8 mL of 90% DMSO 10% H₂O was performed by combining the reagents and stirring at room temperature for 22 hours. After activation, addition of β-phosphate was accomplished by the addition of 1M TBAP in DMF (38.20 mL, 38.20 mmoles) with mixing at room temperature for 18 hours. The reaction solution was then double purified by reverse phase and anion exchange chromatography. The diphosphate ^{m6}A_{2′OMe}pG dinucleotide was used as needed for subsequent coupling reactions.

N7-Me 3′-OMe GDP (369.60 mg, 0.764 mmoles) was activated with imidazole (161.35 mg, 2.37 mmoles) and EDC (234 mg, 1.22 mmoles) in 90% DMSO 10% H₂O (3.70 mL). The activation reaction was allowed for proceed for approximately 18 hours to yield 97% conversion of imidazole activated N7-Me 3′-OMe GDP. pp^{m6}A_{2′OMe}pG dinucleotide (400 mg, 0.500 mmoles) and magnesium chloride (324 uL, 3.15 M) were added to the same solution. The coupling reaction was stirred for ˜48 hours. The reaction mixture was purified using anion exchange chromatography. ¹H NMR (500 MHz, D₂O): δ 8.40 (s, 1H), 8.10 (s, 1H), 7.88 (s, 1H), 5.97 (d, J=6 Hz, 1H), 5.85 (d, J=4.25 Hz, 1H), 5.80 (d, J=5.8 Hz, 1H) 4.90 (m, 1H) 4.86 (t, J=5.5 Hz, 1H) 4.69 (t, J=4.65 Hz, 1H) 4.48 (m, 2H), 4.49-4.40 (m, 3H) 4.38 (bs, 1H) 4.32-4.26 (m, 1H) 4.23-4.16 (m, 4H) 4.15-4.11 (m, 1H) 4.04 (s, 3H) 3.46 (s, 3H) 3.36 (s, 3H) 3.34 (s, 3H) 1.9 (s, 1H)

³¹P NMR (200 MHz, D₂O): δ 0.40 (s, 1P), 10.84-1.98 (t, 1P), 22.15-22.47 (m, 1P)

Calculated for C₃₃H₄₅N₁₅O₂₇P₅ 1238.67, found 1237.3 & 1238.6 [M−H] negative mode.

Synthesis of ^{m7}G_{3′OMe}pppp^{m6}A_{2′OMe}pG (Compound 80):

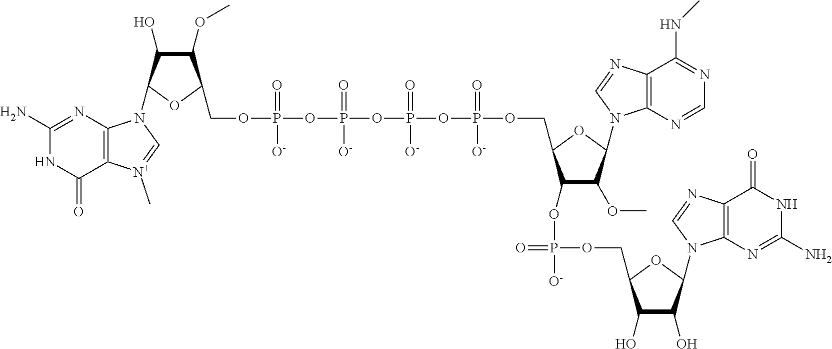

N7-Me-3′Ome Guanosine pppp^{m6}A_{2′OMe}pG was synthesized starting with the large-scale activation of p^{m6}A_{2′OMe}pG dinucleotide (5.0 g, 6.94 mmoles) using imidazole (2.20 g, 32.27 mmoles) and EDC (2.59 g, 16.66 mmoles) in 20 mL of 90% DMSO 10% H₂O stirring at room temperature for 22 hours. After activation, the addition of β-phosphate was accomplished by the addition of 1M TBAP in DMF (85 mL, 85 mmoles) mixing at room temperature for 18 hours. The reaction solution was then double purified by reverse phase and anion exchange chromatography. The diphosphate ^{m6}A_{2′OMe}pG dinucleotide is used as needed for the coupling reaction to yield the final compound.

N7-Me 3′-OMe GDP (354.2 mg, 0.750 mmoles) was activated with imidazole (153.6 mg, 2.325 mmoles) and EDC (230 mg, 1.2 mmoles) in 90% DMSO 10% H₂O (3.54 mL). The activation reaction was left for approximately 18 hours to yield 97% conversion of imidazole activated N7-Me 3′-OMe GDP. pp^{m6}A_{2′OMe}pG dinucleotide (400 mg, 0.500 mmoles) and magnesium chloride (317.46 uL, 3.15 M) was added to the same solution coupling reaction was left for ˜48 hours. The reaction mixture was purified using anion exchange chromatography. ¹H NMR (500 MHz, D₂O): δ 8.32 (s, 1H), 8.08 (s, 1H), 7.86 (s, 1H), 6.44 (s, 1H), 5.97 (d, J=5.5 Hz, 1H) 5.83 (d, J=4.15 Hz, 1H), 5.78 (d, J=5.75 Hz, 1H) 4.48-4.45 (m, 4H), 4.42-4.32 (m, 2H) 4.10-4.09 (m, 1H) 4.01 (s, 3H), 3.45 (s, 3H) 3.40 (s, 2H), 3.06 (bs, 1H), 1.9 (s, 1H). ³¹P NMR (200 MHz, D₂O): δ 0.43 (s, 1P), −10.85 (m, 1P), −22.21 (m, 1P). Calculated for C₃₄H₄₇N₁₅O₂₇P₅ 1252.70, found 1251.6 [M−H] negative mode.

Synthesis of ^{m7}G_{3′OMe}p(s)pp^{m6}A_{2′OMe}pG (Compound 81):

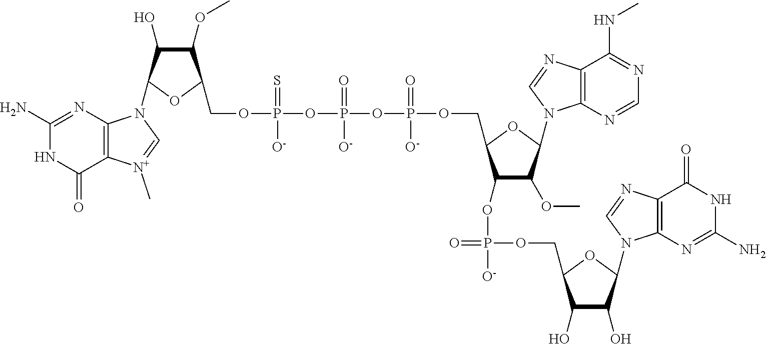

### Synthesis of Activated Imidazolide of Pp^{m6}ApG Dinucleotide:

A large-scale activation of p^{m6}A_{2′OMe}pG dinucleotide (5.0 g, 6.94 mmoles) using imidazole (2.20 g, 32.27 mmoles) and EDC (2.59 g, 16.66 mmoles) in 20 mL of 90% DMSO 10% H₂O was performed by combining the reagents and stirring at room temperature for 22 hours. After activation, addition of β-phosphate was accomplished by the addition of 1M TBAP in DMF (85 mL, 85 mmoles) with mixing at room temperature for 18 hours. The reaction solution was then double purified by reverse phase and anion exchange chromatography. The material was then precipitated using NaClO₄ (100 mg) 2% by volume of starting material and 500 mL of acetone. The precipitate was a white solid (6 g). The diphosphate ^{m6}A_{2′OMe}pG dinucleotide was used as needed for subsequent coupling reactions.

### Synthesis of N7-Me 3′OMe Guanosine 5′-Thiophosphate:

N7-Methylation of 5′ thiophosphoryl guanosine was performed using 5′OH 3′Ome guanosine (1 g, 3.36 mmol). The starting material was methylated using CH₃I (1.25 mL, 20.07 mmol) in 10 mL of DMF. The reaction was allowed to stir at room temperature for over four days. The reaction mixture changed from a cloudy suspension to a clear yellow liquid over the four day period. The reaction mixture was then concentrated to 2.5 mL and triturated with 50 mL of dichloromethane to precipitate out a white/yellow solid. The solid was vacuum filtered until no visible liquid was seen.

The 5′ thiophosphorylation of N7-Me 3′Ome guanosine was accomplished using N7-Me 3′OMe guanosine (1 g, 3.2 mmole). The reaction was conducted under argon using dry materials. The addition of the PSCl₃ (487 uL, 4.8 mmoles) and 2,6 lutidine (1118 uL, 4.6 mmoles) in TMP was done at 0° C. The reaction was complete after 8-9 hours. The reaction was then quenched with 1.5M TEAA to afford pH 5-6. The product was purified by anion exchange chromatography using QFF resin and the product was obtained as a TEA salt.

Compound 81 was synthesized by coupling N7-Me 3′OMe guanosine 5′-thiophosphate with activated imidazolide of pp^{m6}A_{2′OMe}pG dinucleotide in the presence of MgCl₂. The activated imidazolide of pp^{m6}A_{2′OMe}pG dinucleotide was utilized as a solution in 90% DMSO 10% H₂O 3.44 mL at [0.625 mmol/5 mL], along with the addition of N7-Me 3′OMe guanosine 5′thiophosphate (184 mg, 287 umoles) and magnesium chloride 3.15 M (182 uL, 574 umol). The components were mixed and stirred at room temperature for 4.5 days. The product was purified by anion exchange chromatography using QFF resin and then by QHP anion exchange chromatography. Final yield after precipitation was 1.805 mL (100 mM), 177 umol (5494.42 ODs). ¹H NMR (500 MHz, D₂O): δ 8.30 (s, 1H), 8.05 (s, 1H), 7.92 (d, J=4.4 Hz, 1H), 5.96 (d, J=5.75 Hz, 1H), 5.81 (t, J=4.91 Hz, 1H) 5.77-5.76 (dd, J=3.9 Hz, 3.15 Hz, 1H) 4.94-4.89 (m, 1H), 4.63-4.58 (m, 1H), 4.58-4.52 (m, 2H) 4.46-4.34 (m, 4H), 4.3-4.2 (m, 4H), 4.09-4.05 (m, 1H), 3.97-3.95 (d, J=9.95 Hz, 3H), 3.44-42 (m, J=2.35 Hz, 6H), 3.05 (bs, 1H), 1.9 (s, 1H). ³¹P NMR (200 MHz, D₂O): δ 43.86-43.27 (dd, 1P) 0.40 (s, 1P), −11.16 (t, 1P), 23.51 (m, 1P). Calculated for C₃₄H₄₇N₁₅O₂₃P₄S 1189.79, found 1189 & 1187.8 [M−H] negative mode.

### Example 2: mRNA Synthesis

mRNAs including each of the Cap analogs, including compounds as described herein along with comparative examples, were synthesized for further testing. See Table 1 for synthesized analogs.

TABLE 1
Synthesized mRNAs

| Cap Analog | Synthesized mRNA Structure |
|---|---|
| ^{m7}GpppA_{2′OMe}pG (Control) | 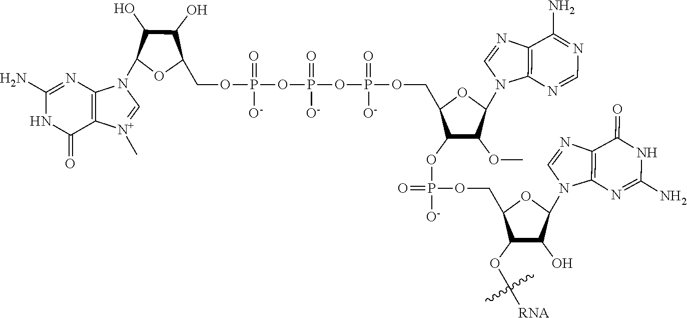 |
| ^{m7}GpppA_{2′,4′-LNA}PG (Compound 1) | 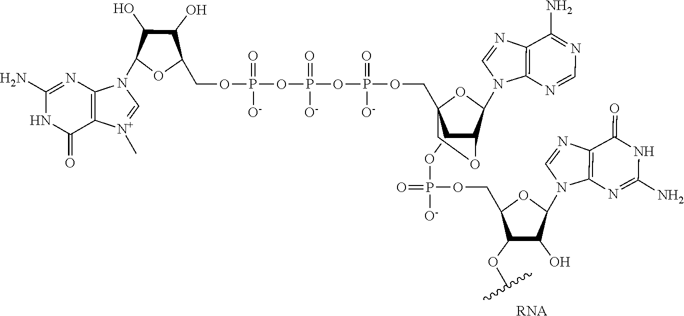 |
| ^{m7}G_{3′OMe}pppA_{2′,4′-LNA}PG (Compound 2) | 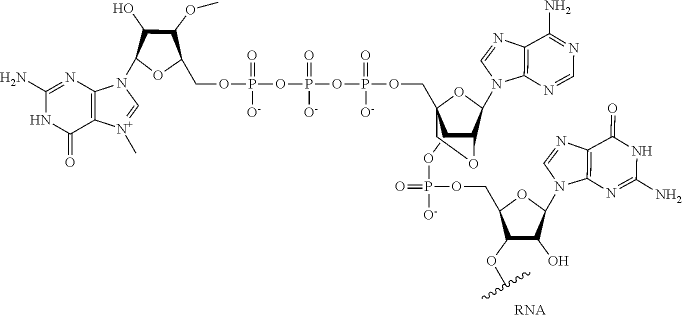 |
| ^{m7}GpppA_{2′,4′-LNA}p(s)G (Compound 3) | 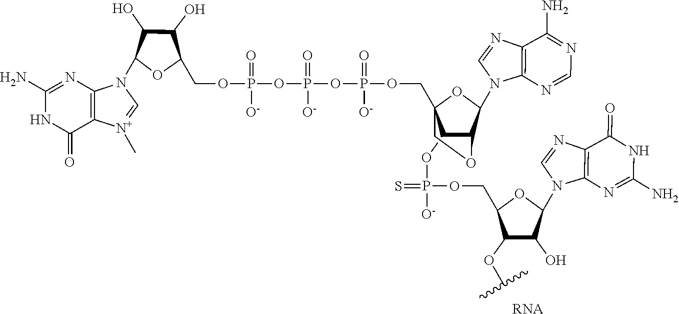 |
| ^{m7}GpppA_{2′OMe-5′vinyl}pG (Compound 4) | 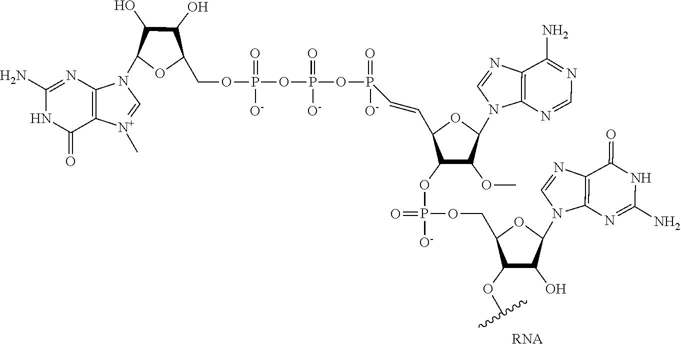 |
| ^{m7}G_{3′-OMe}pppA_{2′OMe- 5′vinyl}pG (Compound 5) | 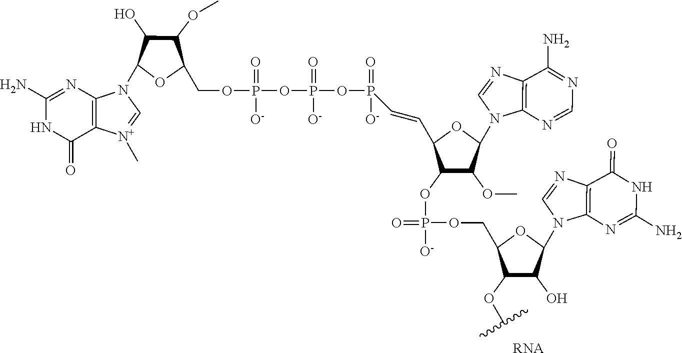 |
| ^{m7}GpppA_{2′F}p(s)G (Compound 6) | 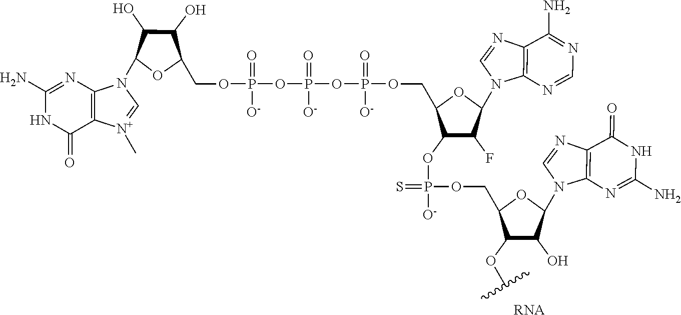 |
| ^{m7}Gppp(diamino- purine)_{2′OMe}pG (Compound 7) | 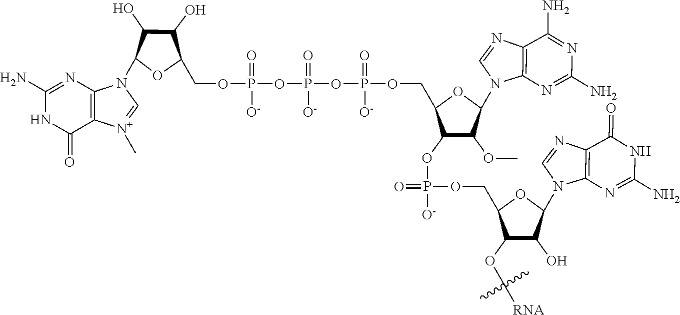 |
| ^{m7}G_{3′OMe}ppp(diamino- purine)_{2′OMe}pG (Compound 8) | 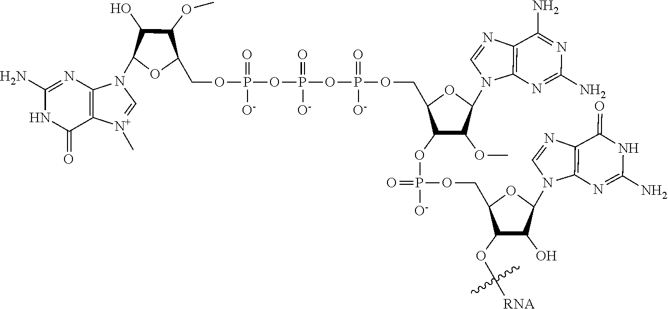 |
| ^{m7}G_{3′OMe}ppp(diamino- purine)_{2′OMe}p(s)G (Compound 9) | 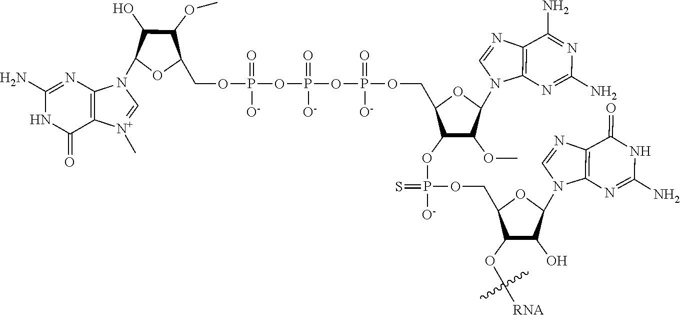 |
| ^{m7}Gpp^{m6}A_{2′OMe}pA (Compound 10) | 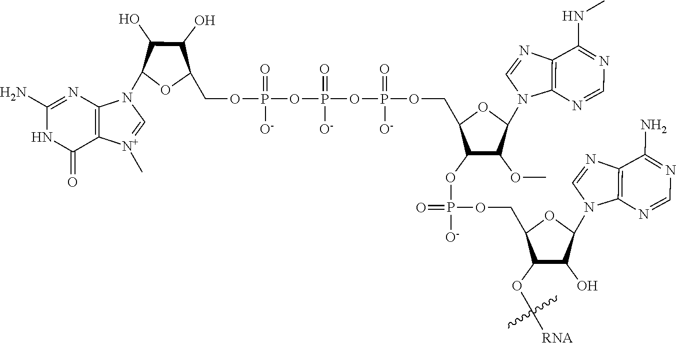 |
| ^{m7}G_{3′OMe}ppp^{m6}A_{2′OMe}pA (Compound 11) | 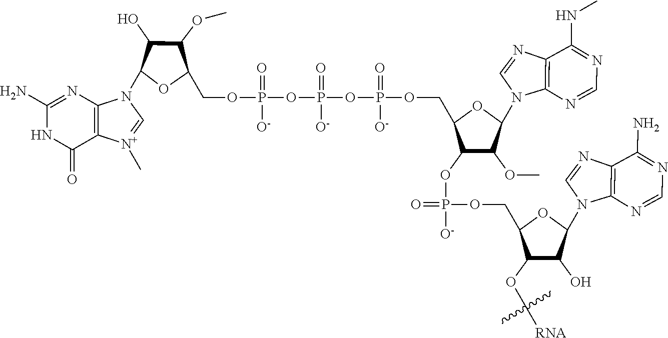 |
| ^{m7}G_{3′OMe5′-thio}ppp^{m6}A_{2′OMe}pG (Compound 12) | 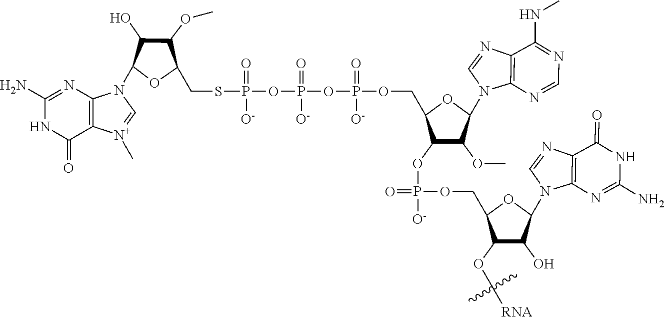 |
| ^{m7}G_{3′OMe}pp(s)p^{m6}A_{2′OMe}pG* (Compound 13) *Two diastereomers separated: ^{m7}G_{3′OMe}pp(SRp)p^{m6}A_{2′OMe}pG and ^{m7}G_{3′OMe}pp(Ssp)p^{m6}A_{2′OMe}pG | 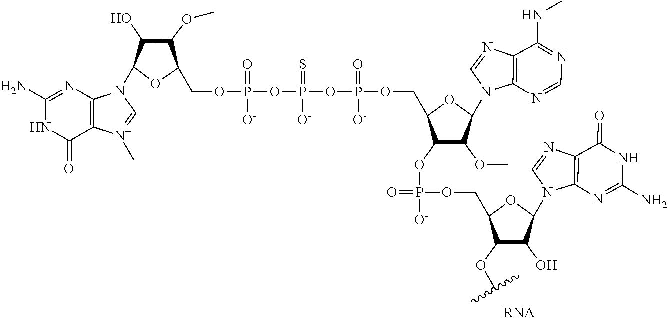 |
| ^{m7}GpppA_{2′4′LNA}PU (Compound 15) | 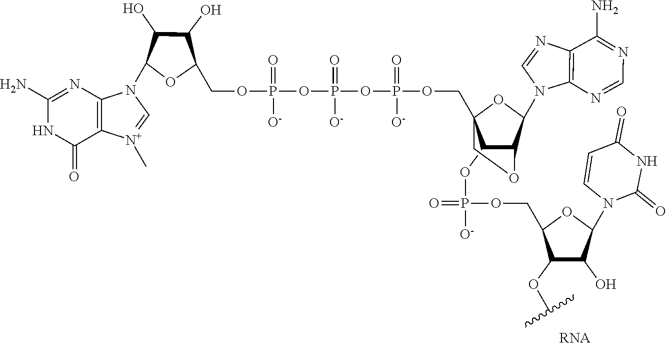 |
| ^{m7}G_{3′OMe}pppA_{2′4′LNA}pU (Compound 16) | 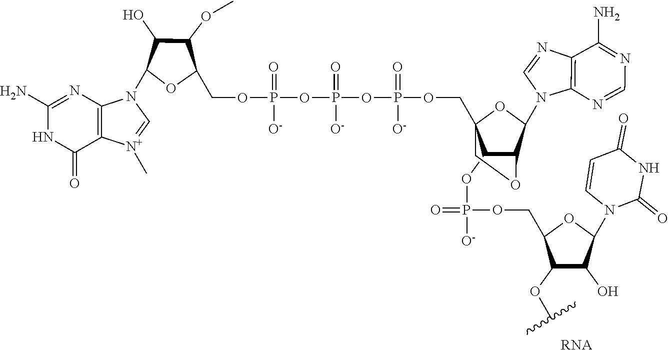 |
| ^{m7}G_{3′OMe}pppA_{2′F}pU (Compound 17) | 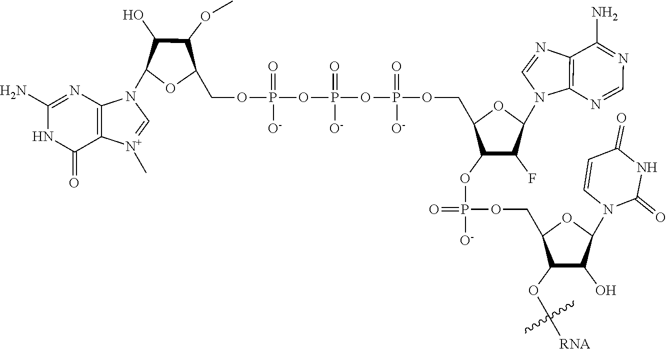 |
| ^{m7}G_{3′OMe}p(s)ppA_{2′OMe}pG (Compound 18) | 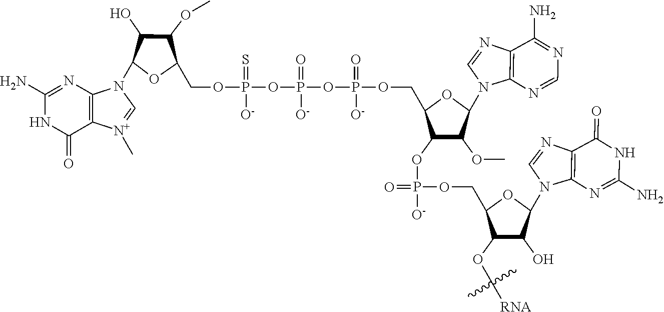 |
| ^{m7}G_{3′OMe}pp(s)pA_{2′OMe}pG* (Compound 19) *Two diastereomers separated: ^{m7}G_{3′OMe}pp(S_{Rp})pA_{2′OMe}pG and ^{m7}G_{3′OMe}pp(S_{Sp})pA_{2′OMe}pG | 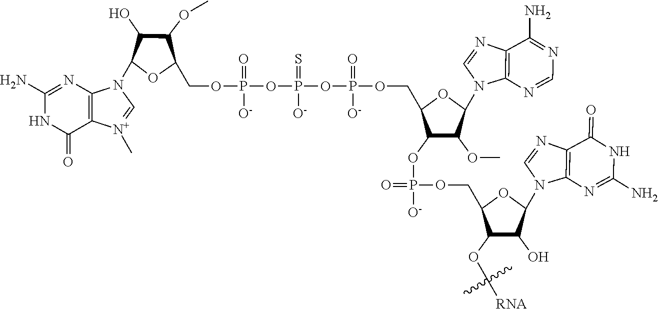 |
| ^{m7}G_{3′OMe}ppp(s)A_{2′OMe}pG (Compound 20) | 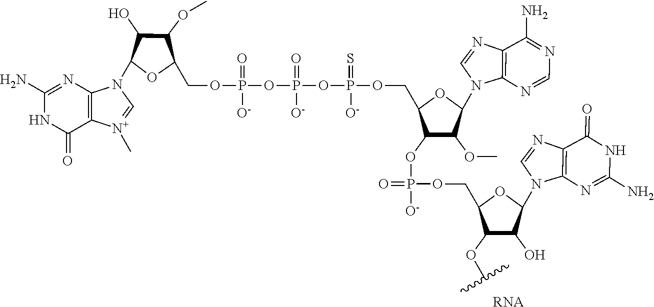 |
| ^{m7}G_{3′OMe}ppA_{2′OMe}p(s)G* (Compound 21) *Two diastereomers separated: ^{m7}G_{3′OMe}pppA_{2′OMe}p(S_{Rp})G and ^{m7}G_{3′OMe}pppA_{2′OMe}p(S_{Sp})G | 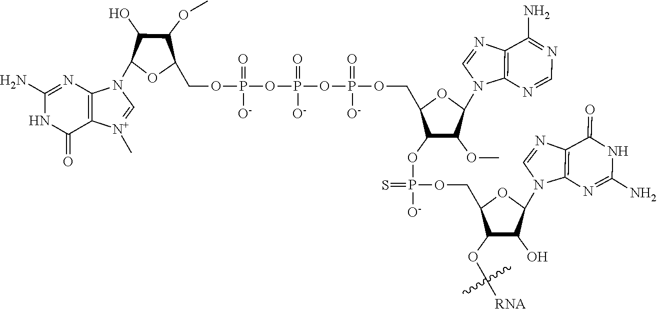 |
| ^{m7}G_{3′OMe}pppA_{2′OMe}pU (Compound 22) | 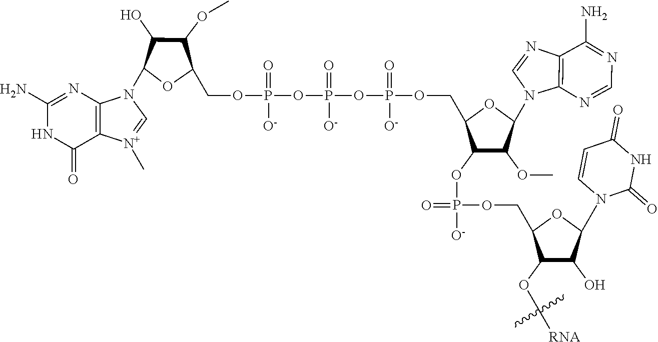 |
| ^{m7}Gpppm^{m6}A_{2′OMe}pU (Compound 23) | 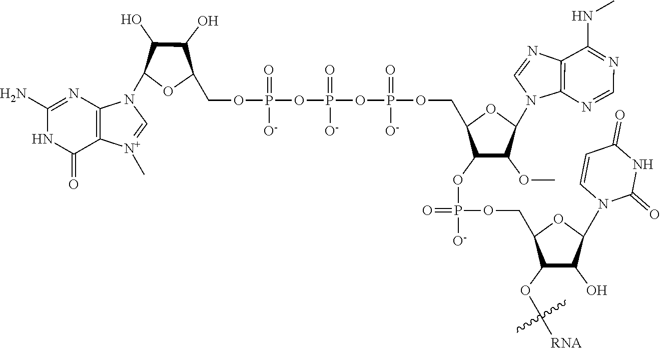 |
| ^{m7}G_{3′OMe}ppp^{m6}A_{2′OMe}pU (Compound 24) | 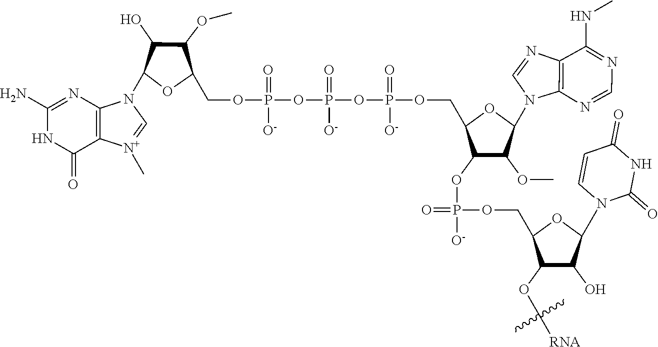 |
| ^{m7}G_{2′OMe3′OMe}pppA_{2′OMe}pG (Compound 25) | 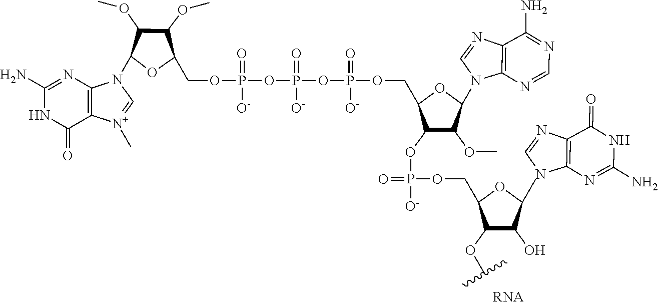 |
| ^{m7}G_{2′OMe}pppA_{2′OMe}pG (Compound 26) | 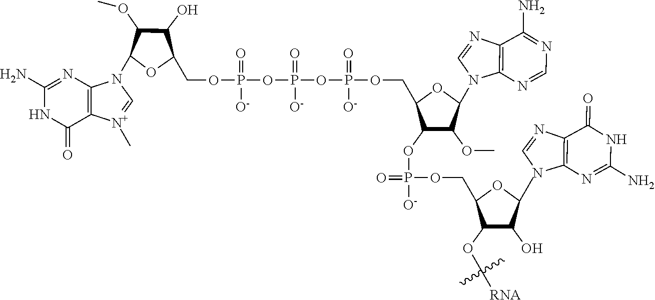 |
| ^{m7}G_{2′OMe3′OMe}ppp^{m6}A_{2′OMe}pG (Compound 27) | 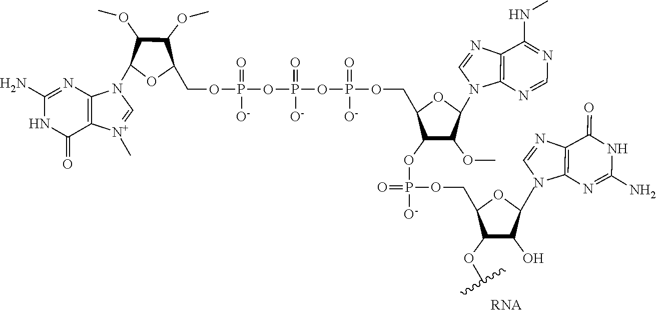 |
| ^{m7}G_{2′OMe}ppp^{m6}A_{2′OMe}pG (Compound 28) | 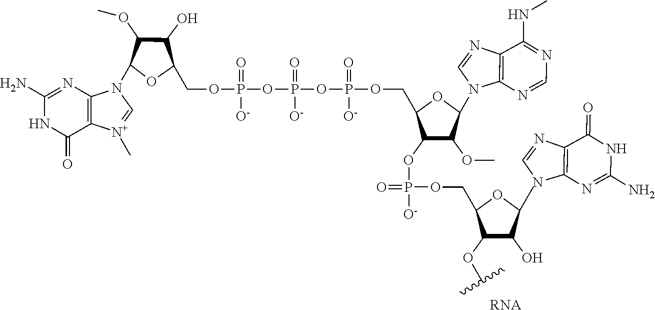 |
| ^{m7}G_{3′OMe}ppA_{2′OMe}pG (Compound 29) | 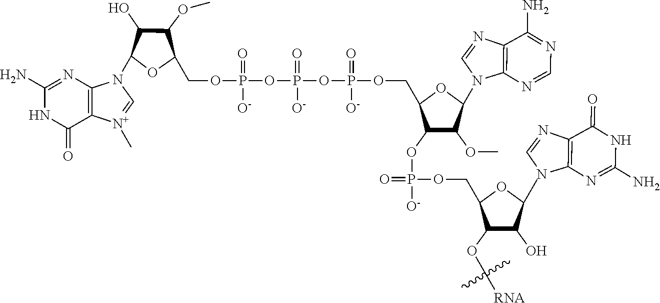 |
| ^{m7}G_{3′OMe}ppp^{m6}A_{2′OMe}pG (Compound 30) | 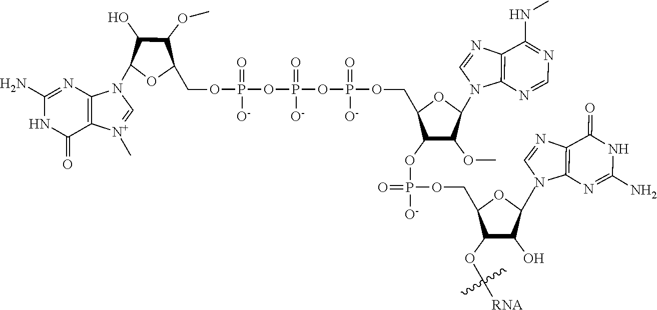 |
| ^{m7}Gppp^{m6}A_{2′OMe}pG (Compound 31) | 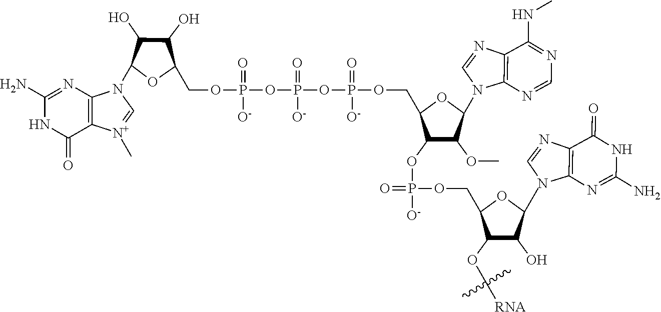 |
| ^{m7}G_{2′4′-LNA}pppA_{2′OMe}pG (Compound 32) | 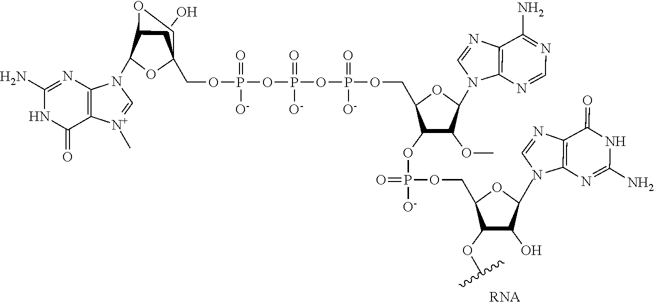 |
| ^{m7}GpppA_{2′F}PU (Compound 33) | 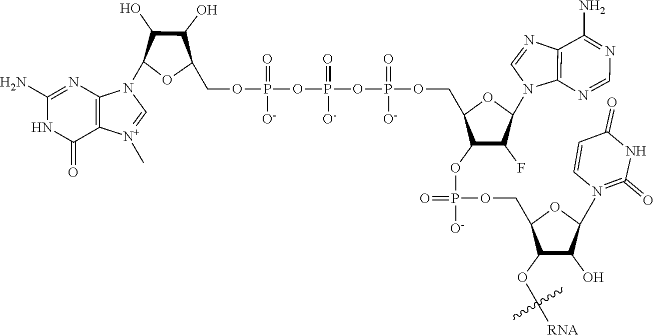 |
| ^{m7}G_{3′SMe}pppA_{2′OMe}pG (Compound 42) | 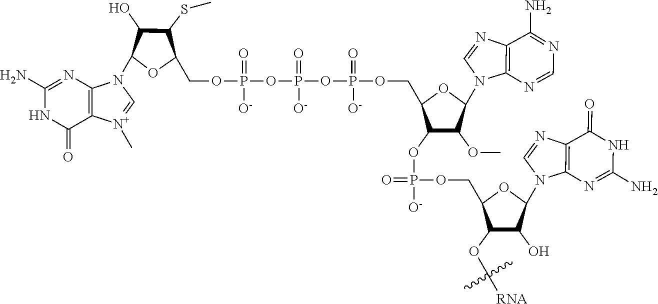 |
| ^{m7}G_{2′OH, 2′Me}pppA_{2′OMe}pG (Compound 43) | 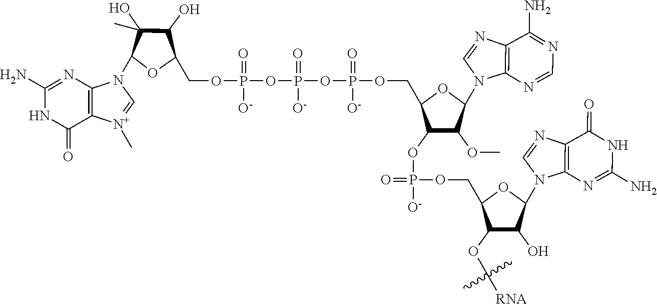 |
| ^{m7}G_{3′AZM}ppp^{m6}A_{2′OMe}pG (Compound 45) | 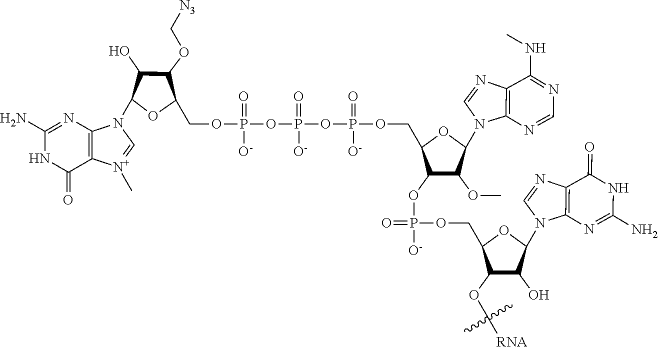 |
| ^{m7}G_{3′F}ppp^{m6}A_{2′OMe}pG (Compound 49) | 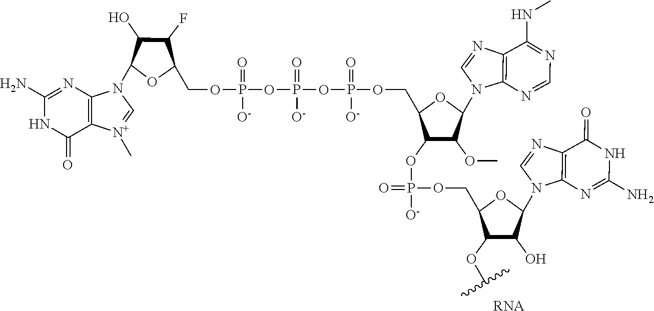 |
| ^{m7}G_{3′MOE}ppp^{m6}A_{2′OMe}pG (Compound 50) | 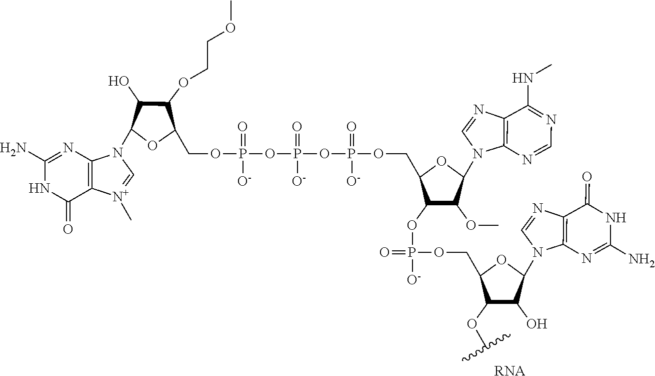 |
| ^{m7}G_{3′SMe}pppm6 A_{2′OMe}pG (Compound 52) | 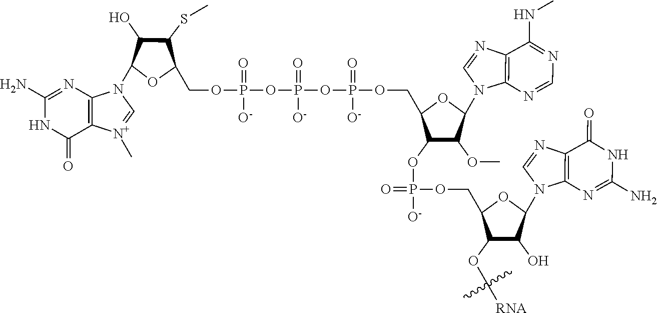 |
| ^{m7}G_{3′OMe}pppA_{2′OMe5′thio}pG (Compound 73) | 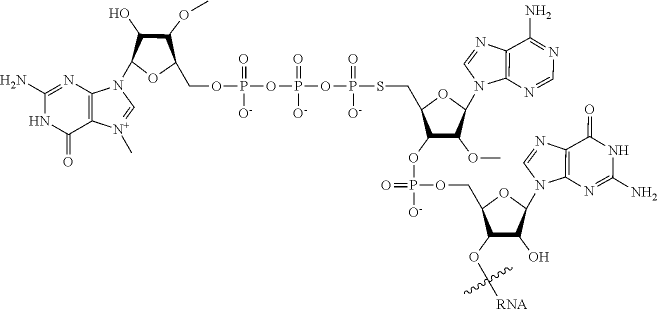 |
| ^{m7}G_{3′OMe}ppp^{m6}A_{2′OMe}p(s)G* (Compound 74) *Two diastereomers separated: ^{m7}G_{3′OMe}ppp^{m6}A_{2′OMe}p(S_{Rp})G and ^{m7}G_{3′OMe}ppp^{m6}A_{2′OMe}p(S_{Sp})G | 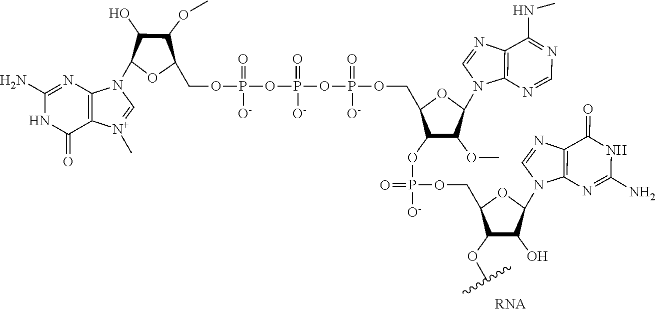 |
| ^{m7}(L-sugar isomer)GpppA_{2′OMe}pG (Compound 75) | 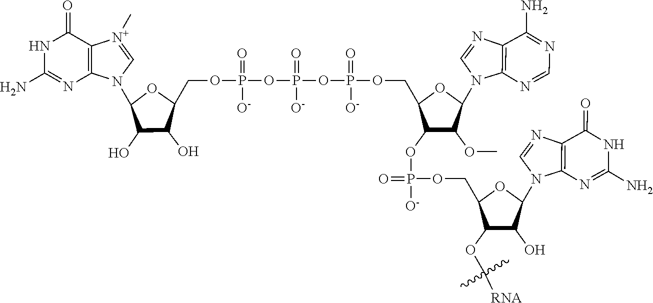 |
| ^{m7}G_{2′OH, 2′Me}ppp^{m6}A_{2′OMe}pG (Compound 76) | 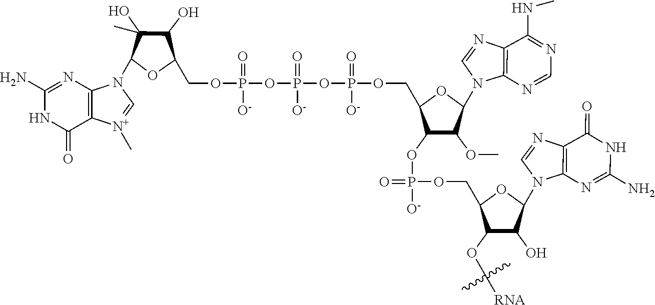 |
| ^{m7}(L-sugar isomer)Gppp^{m6}A_{2′OMe}pG (Compound 77) | 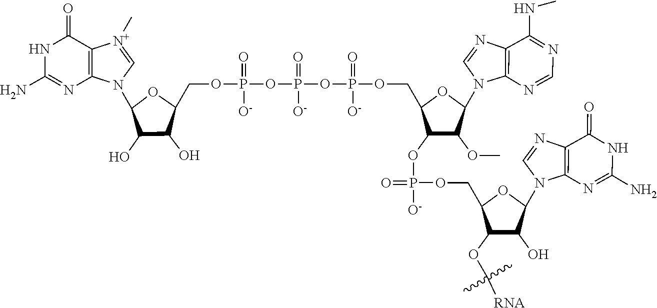 |
| ^{m7}(acyclo)Gppp^{m6}A_{2′OMe}pG (Compound 78) | 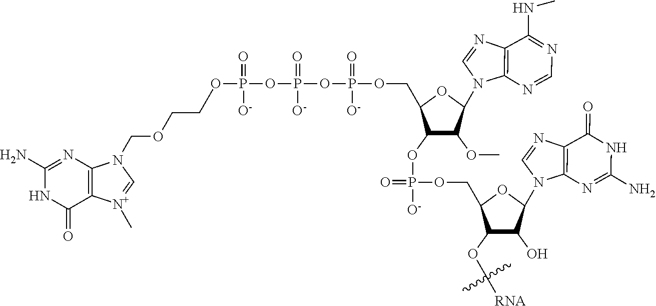 |
| ^{m7}G_{3′OMe}ppppA_{2′OMe}pG (Compound 79) | 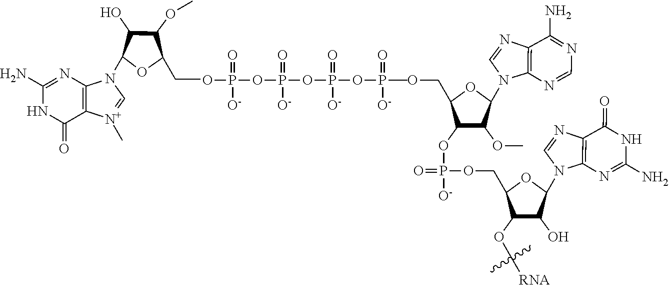 |
| ^{m7}G_{3'OMe}pppp^{m6}A_{2′OMe}pG (Compound 80) | 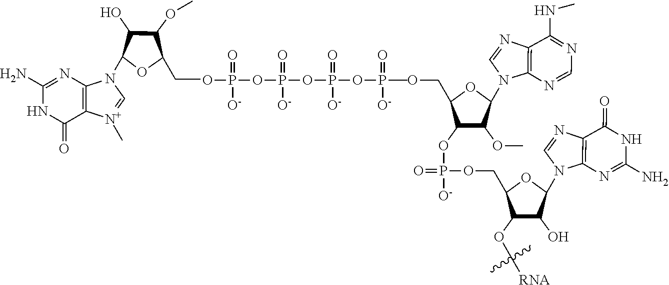 |
| ^{m7}G_{3'OMe}p(s)pp^{m6}A_{2′OMe}pG (Compound 81) | 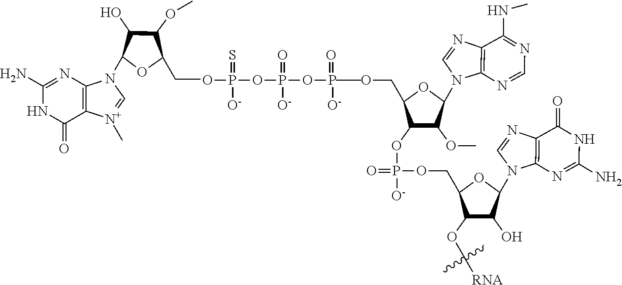 |

### Example 3: In Vitro Performance of Cap Analogs

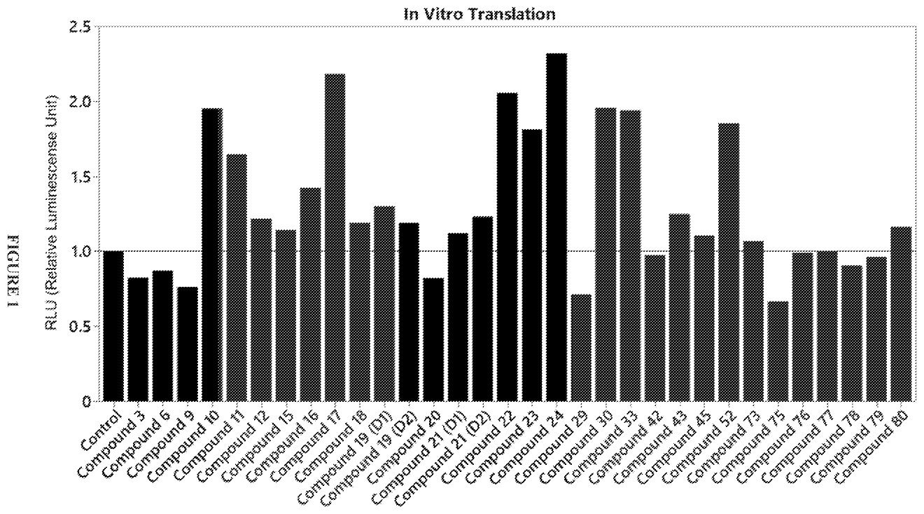

In vitro testing was performed on the cap analogs described herein using a commercially available wheat germ extract cell-free translation system (PROMEGA L4380; Promega Corporation, Madison, WI). For the assays, 20 ng/μL of luciferase mRNA capped using a cap analog recited in Table 1 was mixed with translation components of the wheat germ extract system. The components reacted for two hours at room temperature to allow for protein production. The crude protein mix was subjected to a luciferin substrate and incubated in the dark for 10 minutes. The total luciferase signal was collected in a 96-well plate format as relative luminescence units (RLU). Blank wells with luciferase alone and wheat germ extract controls without luciferase mRNA were used as system controls. The measured relative luminescence results are shown in FIG. 1, showing the RLU values.

### Example 4: In Vivo Performance of Cap Analogs

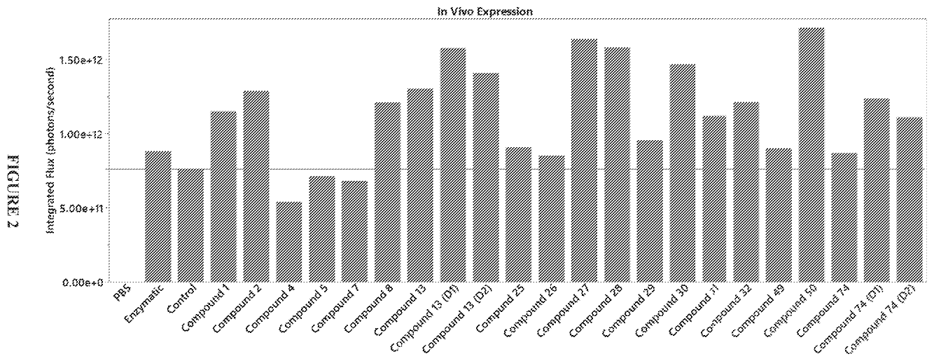

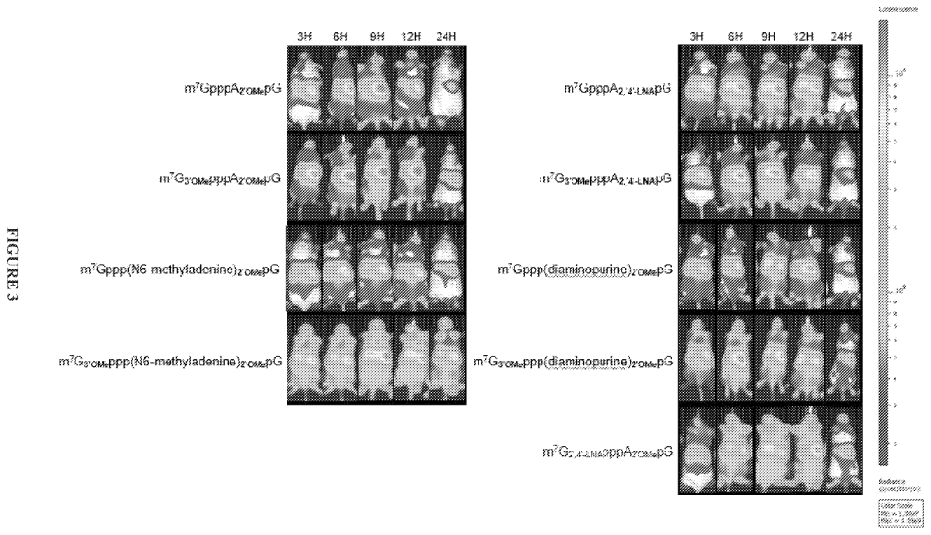

Female CD-1 mice of 8-9 weeks old were randomized into groups of five animals based on body weight at the beginning of the study (Charles River Laboratories, Discovery Research Services North Carolina). Lipid nanoparticle formulations including mRNA (capped using a cap analog recited in Table 1, N1-methylpseudouridine-5′-Triphosphate (N1-me-PsU) modified firefly luciferase (Fluc) mRNA) (LNP:mRNA) test articles were diluted in PBS to achieve 1 mg/kg delivery in a single bolus by tail-vein injection. The body weight of each mouse was measured once a day for the duration of the study and two blood draws were taken for serum analysis by Mouse Cytokine/Chemokine 26-Plex ProcartaPlex Panel 1 by Luminex platform (Thermo Fisher Scientific; Waltham, MA). Luciferase activity was measured by whole-body bioluminescence imaging on IVIS Spectrum CT system by Perkin Elmer (Greenville, SC) at six time points post mRNA injection (3 hours, 6 hours, 9 hours, 12 hours, 24 hours, and 48 hours hours post-injection). D-luciferin was delivered by intraperitoneal injection 10 minutes prior to each imaging session (150 mg/kg total). The measured luciferase expression results are shown in FIG. 2, showing the integrated flux from 3-48 hours. FIG. 3 shows, for representative compounds, a representative animal from each cohort of five animals shown across five imaging time points post LNP:mRNA and luciferin injection (3 hours, 6 hours, 9 hours, 12 hours, and 24 hours post-injection). Luminescence intensity scale is on the left.

When using lipid nanoparticle formulations including the mRNA produced according to Example 2 and luciferin were injected into mice, in vivo luciferase expression was either equivalent to or increased in the mice injected with the LNPs comprising mRNA including the novel cap analogs described herein as compared to mRNA including cap analog ^{m7}GpppA_{2′OMe}pG (Control). See FIG. 2.

^{m7}G_{3′OMe}ppp^{m6}A_{2′OMe}pG (Compound 30), ^{m7}G_{3′OMe}pppA_{2′OMe}pG (Compound 29), ^{m7}G_{3′OMe}pppA_{2′,4′-LNA}pG (Compound 2), ^{m7}G_{2′4′LNA}pppA_{2′OMe}pG (Compound 32), ^{m7}G_{3′OMe}ppp(diaminopurine)_{2′OMe}pG (Compound 8), ^{m7}GpppA_{2′,4′-LNA}pG (Compound 1), and ^{m7}Gppp^{m6}A_{2′OMe}pG (Compound 31) exhibited increased in vivo luciferase expression as compared to ^{m7}GpppA_{2′OMe}pG (Control). ^{m7}Gppp^{m6}A_{2′OMe}pG (Compound 31) exhibited equivalent in vivo luciferase expression. The data demonstrate that the analogs described herein exhibit increased or equivalent in vivo translation as compared to ^{m7}GpppA_{2′OMe}pG (Control).

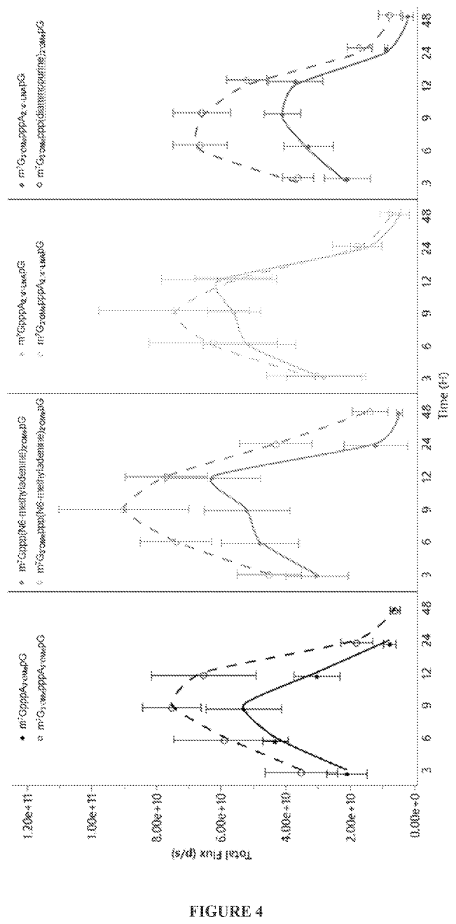

Select data obtained from the luciferase expression experiment described above were categorized to show direct comparisons between the compounds having a 3′OH group on the ^{m7}G moiety, and the same compounds having a 3′OMe modification on the ^{m7}G moiety. See FIG. 4. Surprisingly, for each of the tested and analyzed comparisons, the 3′OMe modification on the ^{m7}G results in increased in vivo translation. As shown in FIG. 4, ^{m7}GpppA_{2′OMe}pG (Control) was measured relative to ^{m7}G_{3′OMe}pppA_{2′OMe}pG (Compound 29), which is ^{m7}GpppA_{2′OMe}pG (Control) with a 3′OMe group on the ^{m7}G (first column); ^{m7}Gppp^{m6}A_{2′OMe}pG (Compound 31) was measured relative to ^{m7}G_{3′OMe}ppp^{m6}A_{2′OMe}pG (Compound 30), which is ^{m7}Gppp^{m6}A_{2′OMe}pG (Compound 31) with a 3′OMe group on the ^{m7}G (second column); ^{m7}GpppA_{2′,4′-LNA}pG (Compound 1) was measured relative to ^{m7}G_{3′OMe}pppA_{2′,4′-LNA}pG (Compound 2), which is ^{m7}GpppA_{2′,4′-LNA}pG (Compound 1) with a 3′OMe group on the ^{m7}G (third column); and ^{m7}Gppp(diaminopurine)_{2′OMe}pG (Compound 7) was measured relative to ^{m7}G_{3′OMe}ppp(diaminopurine)_{2′OMe}pG (Compound 8), which is ^{m7}Gppp(diaminopurine)_{2′OMe}pG (Compound 7) with a 3′OMe group on the ^{m7}G (fourth column). In each of these examples, the 3′OMe modification on the ^{m7}G results in increased luciferase expression in vivo, and therefore, increased translation.

A similar increased luciferase expression in vivo was obtained for ^{m7}G_{3′-OMe}pppA_{2′OMe-5′vinyl}pG (Compound 5) as compared to ^{m7}GpppA_{2′OMe-5′vinyl}pG (Compound 4; lacking a 3′OMe group on the ^{m7}G); ^{m7}G_{2′OMe3′OMe}pppA_{2′OMe}pG (Compound 25) as compared to ^{m7}G_{2′OMe}pppA_{2′OMe}pG (Compound 26; lacking a 3′OMe group on the ^{m7}G); and ^{m7}G_{2′OMe3′OMe}ppp^{m6}A_{2′OMe}pG (Compound 27) and compared to ^{m7}G_{2′OMe}ppp^{m6}A_{2′OMe}pG (Compound 28; lacking a 3′OMe group on the ^{m7}G). See FIG. 2.

The compounds and methods of the appended claims are not limited in scope by the specific compounds and methods described herein, which are intended as illustrations of a few aspects of the claims and any compounds and methods that are functionally equivalent are within the scope of this disclosure. Various modifications of the compounds and methods in addition to those shown and described herein are intended to fall within the scope of the appended claims. Further, while only certain representative compounds, methods, and aspects of these compounds and methods are specifically described, other compounds and methods are intended to fall within the scope of the appended claims. Thus, a combination of steps, elements, components, or constituents can be explicitly mentioned herein; however, all other combinations of steps, elements, components, and constituents are included, even though not explicitly stated.

1. A compound selected from the group consisting of: 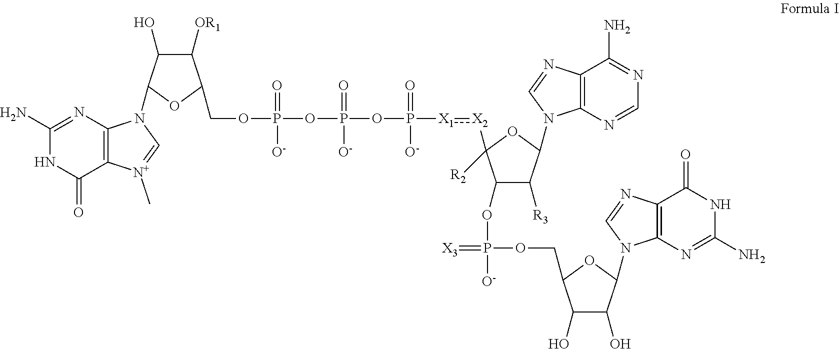
or a stereoisomer thereof, wherein: [symbol]
is a single bond or a double bond;
R₁ is H or CH₃;
R₂ is H and R₃ is OCH₃ or F, or R₂ and R₃ are covalently bonded together and, together with intermediate atoms, form a 2′-O, 4′-C methylene bridge;
X₁ is O or CH;
X₂ is CH₂ or CH;
X₃ is O or S;
each independent Y is H⁺ or a cation; and
n is 0, 1, 2, 3, or 4,
wherein when X₁ is CH, X₂ is CH and [symbol] is a double bond; 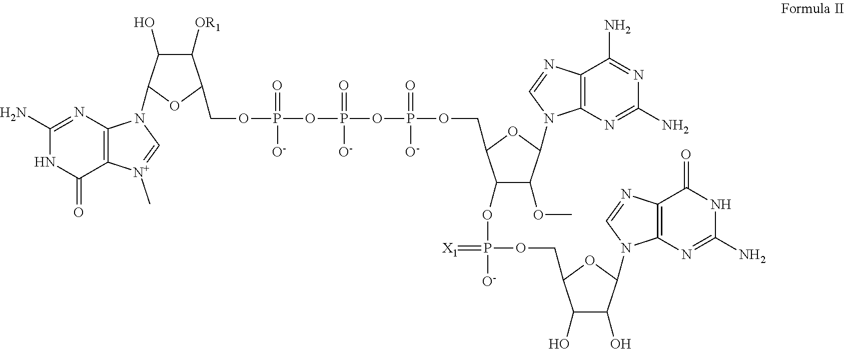
or a stereoisomer thereof, wherein:
R₁ is H or CH₃;
X₁ is O or S;
each independent Y is H⁺ or a cation; and
n is 0, 1, 2, 3, or 4,
wherein when X₁ is S, R₁ is CH₃; 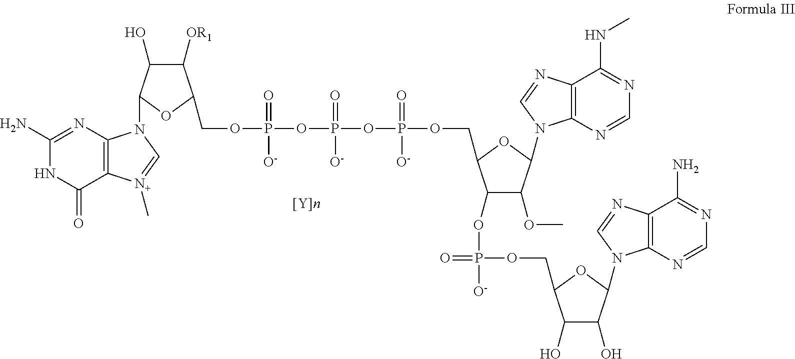
or a stereoisomer thereof, wherein:
R₁ is H or CH₃;
each independent Y is H⁺ or a cation; and
n is 0, 1, 2, 3, or 4; 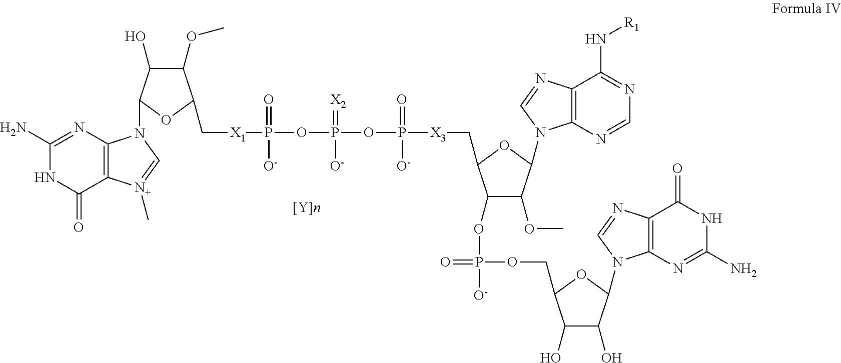
or a stereoisomer thereof, wherein:
R₁ is H or CH₃;
X₁, X₂, and X₃ are each independently selected from O and S, wherein when one of X₁, X₂, or X₃ is S, the remaining of X₁, X₂, and X₃ are O;
each independent Y is H⁺ or a cation; and
n is 0, 1, 2, 3, or 4; 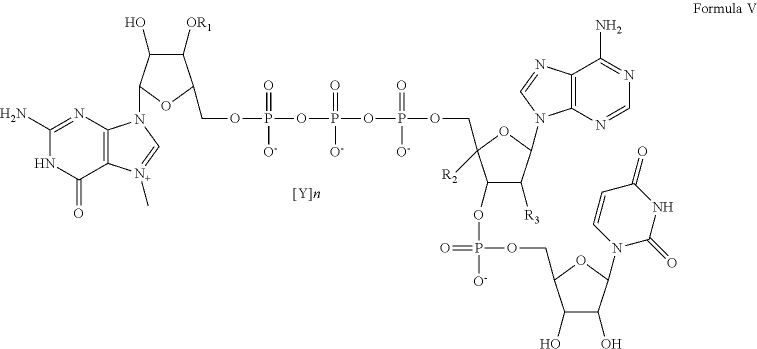
or a stereoisomer thereof, wherein:
R₁ is H or CH₃;
R₂ is H and R₃ is F, or R₂ and R₃ are covalently bonded together and, together with intermediate atoms, form a 2′-O, 4′-C methylene bridge;
each independent Y is H⁺ or a cation; and
n is 0, 1, 2, 3, or 4,
wherein when R₂ is H and R₃ is F, R₁ is not H; 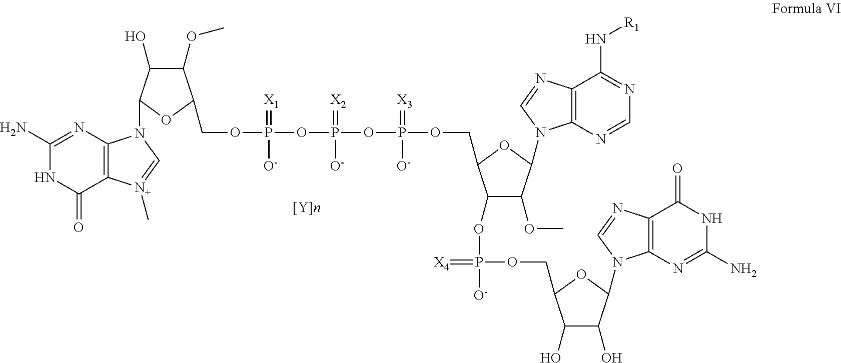
or a stereoisomer thereof, wherein:
R₁ is H or CH₃;
X₁, X₂, X₃, and X₄ are each independently selected from O and S;
each independent Y is H⁺ or a cation; and
n is 0, 1, 2, 3, or 4,
wherein one of X₁, X₂, X₃, and X₄ is S; 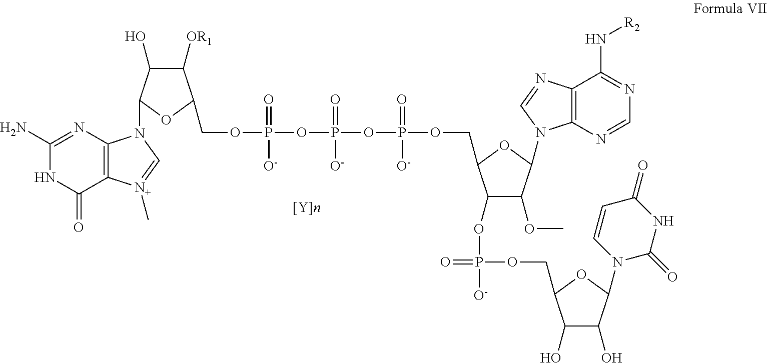
or a stereoisomer thereof, wherein:
R₁ and R₂ are each independently selected from H and CH₃;
each independent Y is H⁺ or a cation; and
n is 0, 1, 2, 3, or 4,
wherein at least one of R₁ or R₂ is CH₃; 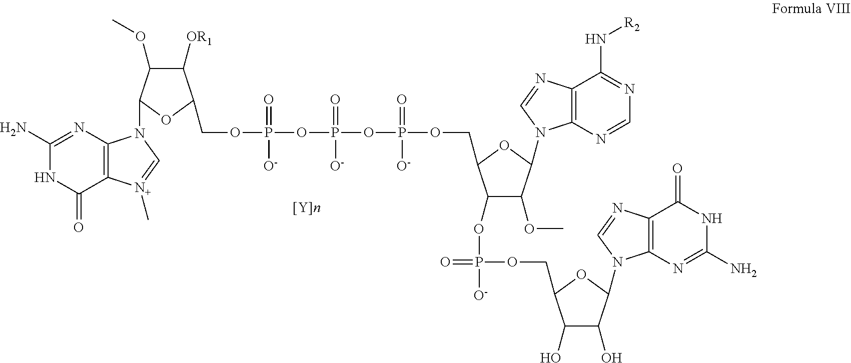
or a stereoisomer thereof, wherein:
R₁ and R₂ are each independently selected from H and CH₃;
each independent Y is H⁺ or a cation; and
n is 0, 1, 2, 3, or 4; 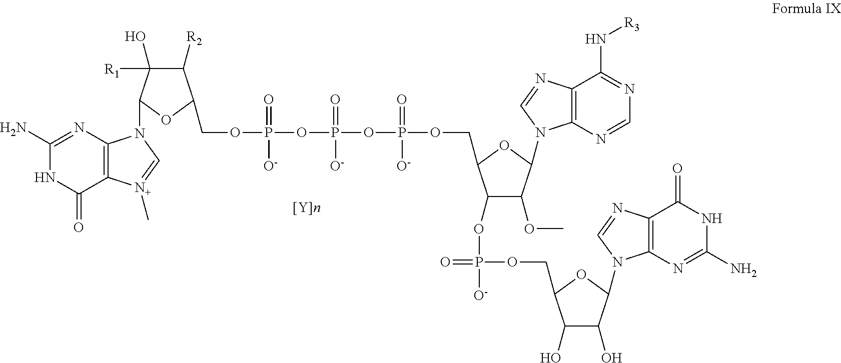
or a stereoisomer thereof, wherein:
R₁ is H or CH₃;
R₂ is OH, F, substituted or unsubstituted alkoxy, or thio;
R₃ is H or CH₃;
each independent Y is H⁺ or a cation;
and n is 0, 1, 2, 3, or 4,
wherein when R₂ is OH, R₁ is not H; 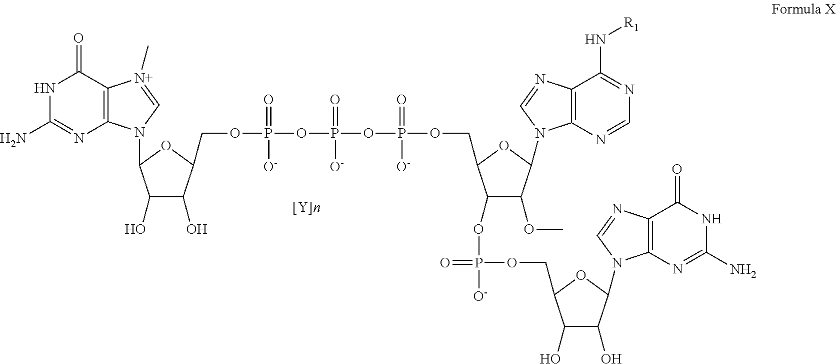
or a stereoisomer thereof, wherein:
R₁ is H and CH₃;
each independent Y is H⁺ or a cation; and
n is 0, 1, 2, 3, or 4; 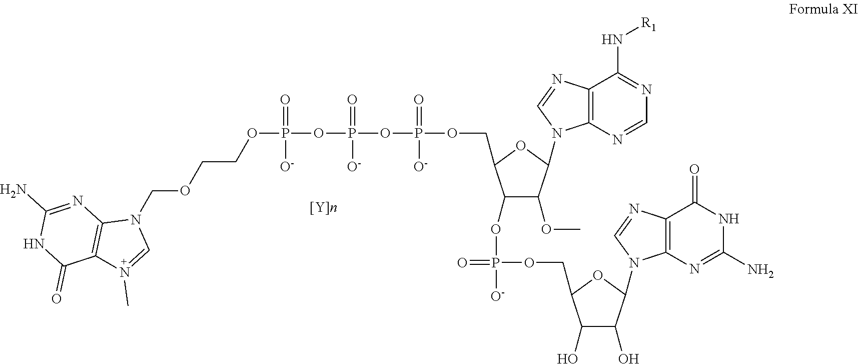
or a stereoisomer thereof, wherein:
R₁ is H and CH₃;
each independent Y is H⁺ or a cation; and
n is 0, 1, 2, 3, or 4; 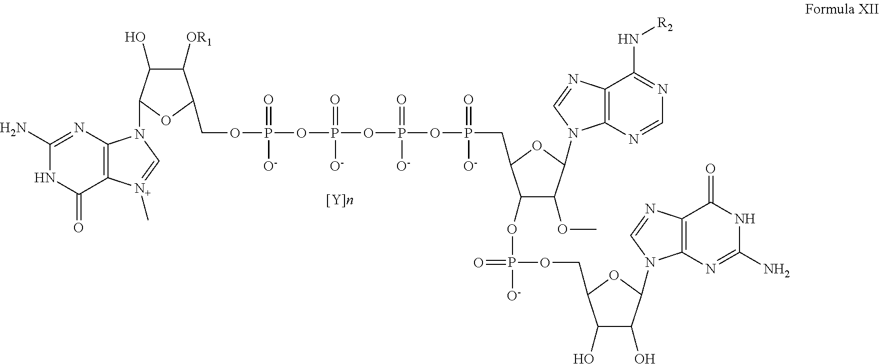
or a stereoisomer thereof, wherein:
R₁ and R₂ are each independently selected from H and CH₃;
each independent Y is H⁺ or a cation; and
n is 0, 1, 2, 3, 4, or 5; 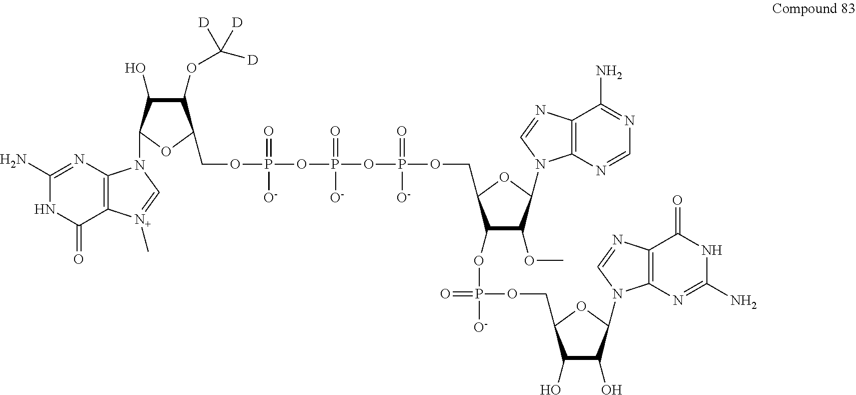
or a stereoisomer thereof;
and 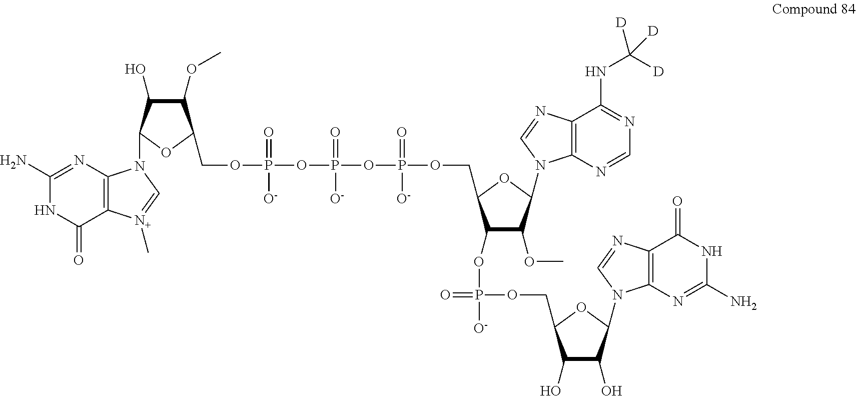
or a stereoisomer thereof.

2. The compound of claim 1, wherein the compound is selected from the group consisting of: 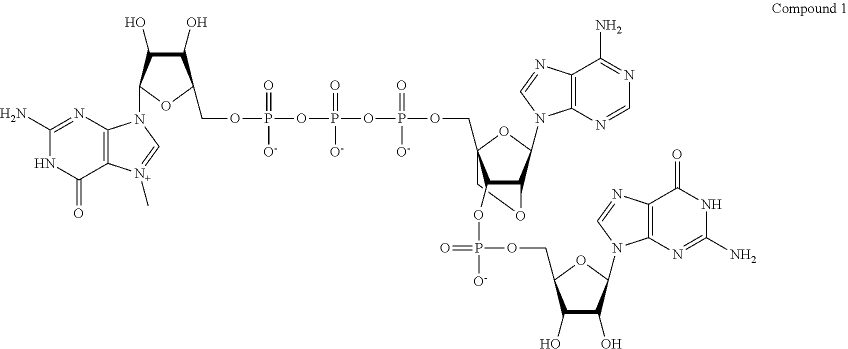
or a stereoisomer thereof, 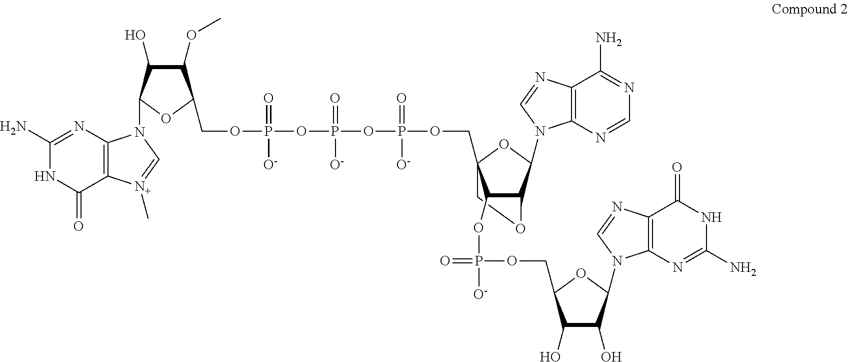
or a stereoisomer thereof, 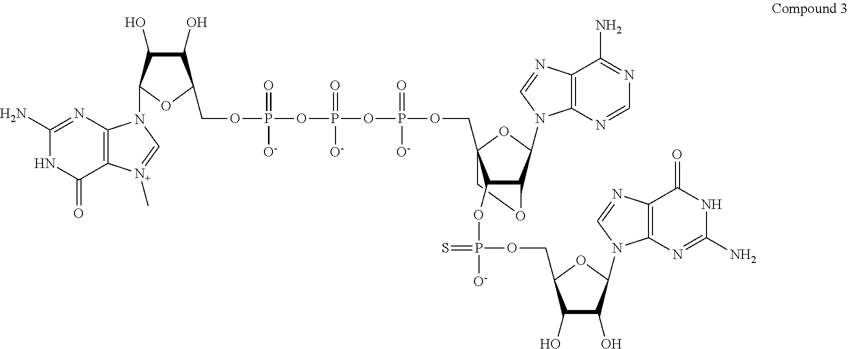
or a stereoisomer thereof, 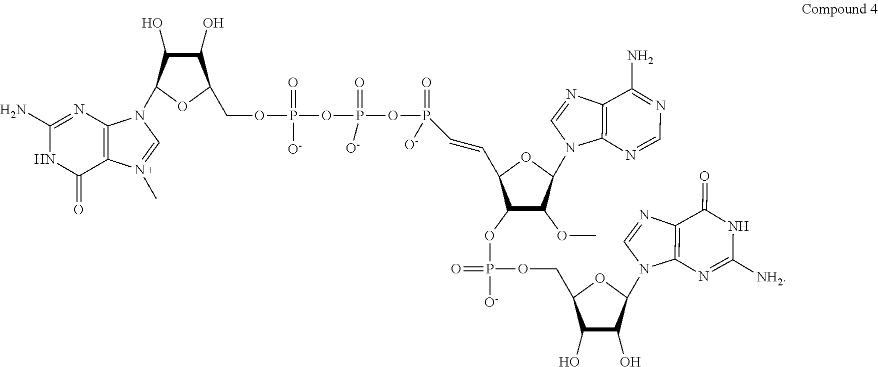
or a stereoisomer thereof, 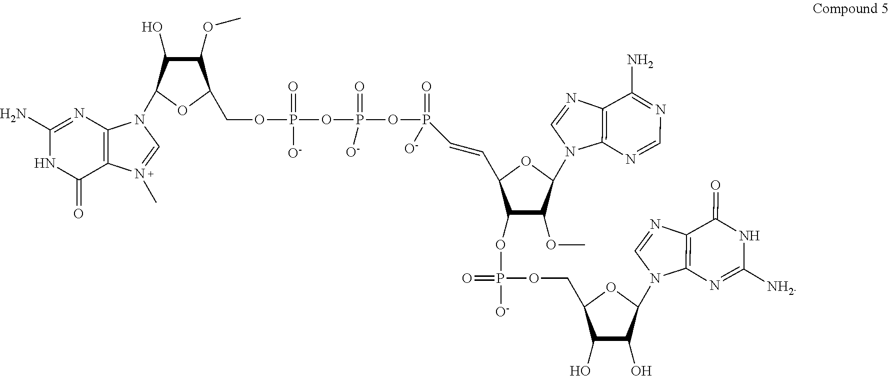
or a stereoisomer thereof, and 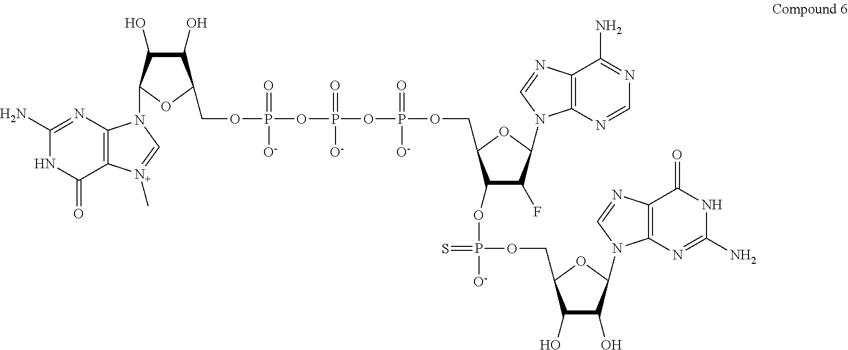
or a stereoisomer thereof.

3. (canceled)

4. (canceled)

5. (canceled)

6. (canceled)

7. (canceled)

8. (canceled)

9. The compound of claim 1, wherein the compound is selected from the group consisting of: 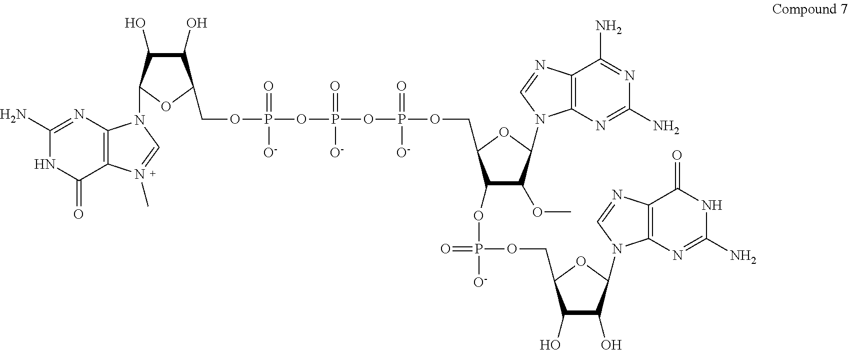
or a stereoisomer thereof, 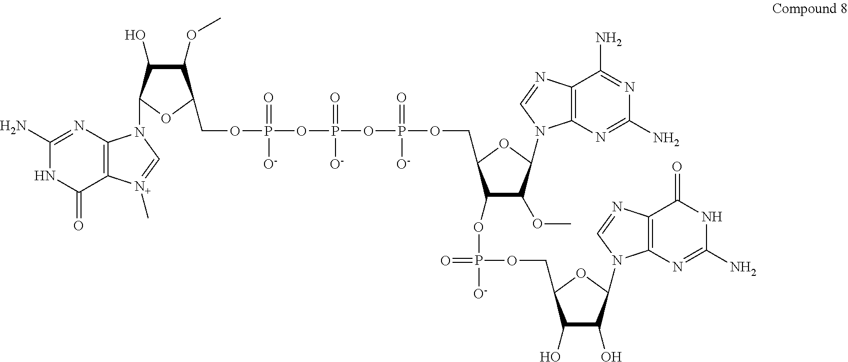
or a stereoisomer thereof, and 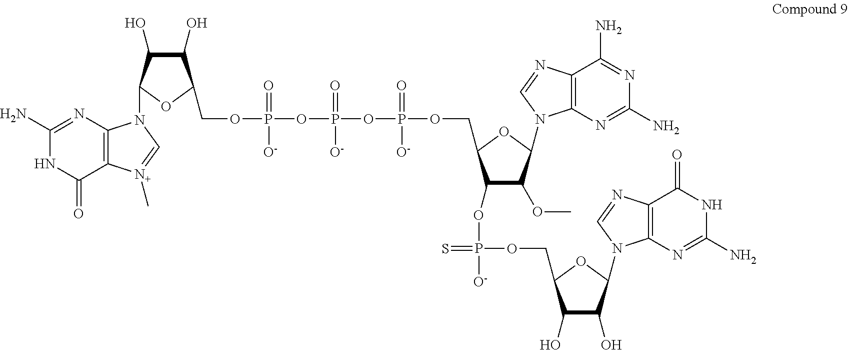
or a stereoisomer thereof.

10. (canceled)

11. (canceled)

12. (canceled)

13. The compound of claim 1, wherein the compound has the following structure: 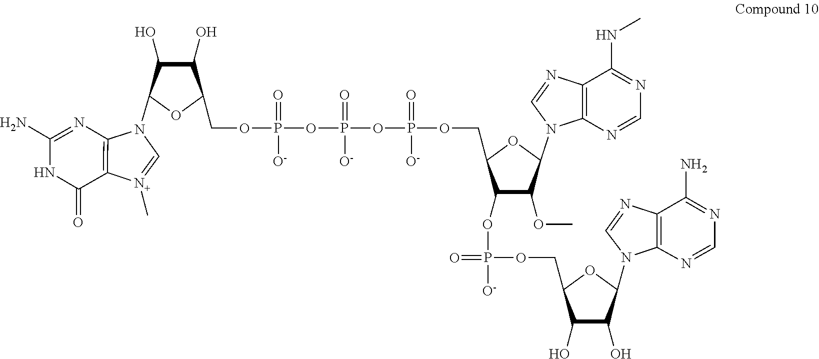
or a stereoisomer thereof, or 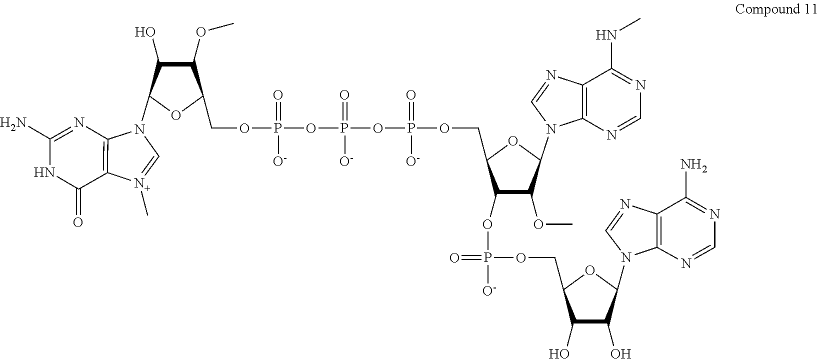
or a stereoisomer thereof.

14. (canceled)

15. (canceled)

16. The compound of claim 1, wherein the compound is selected from the group consisting of: 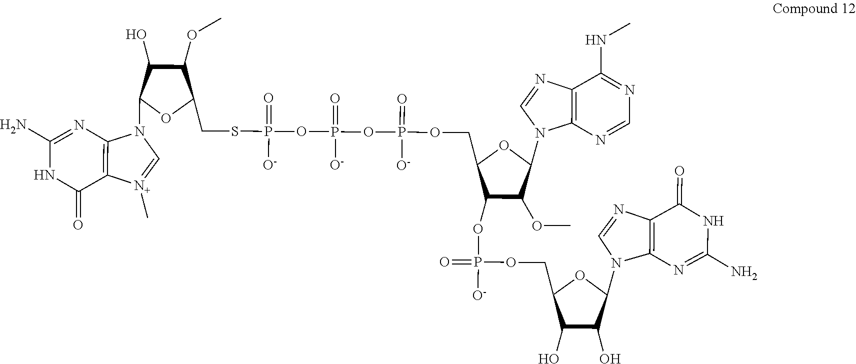
or a stereoisomer thereof, 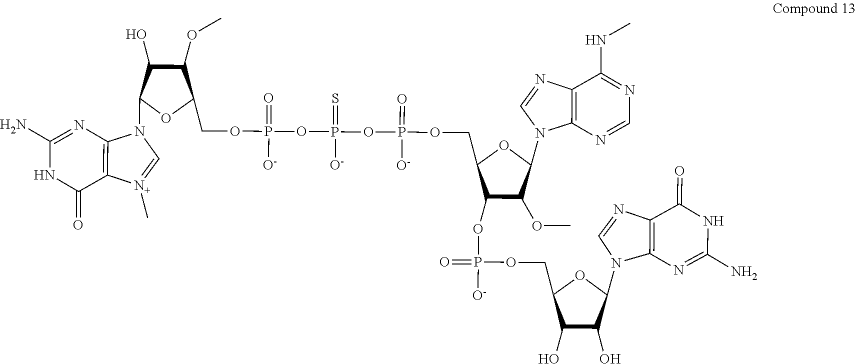
or a stereoisomer thereof, 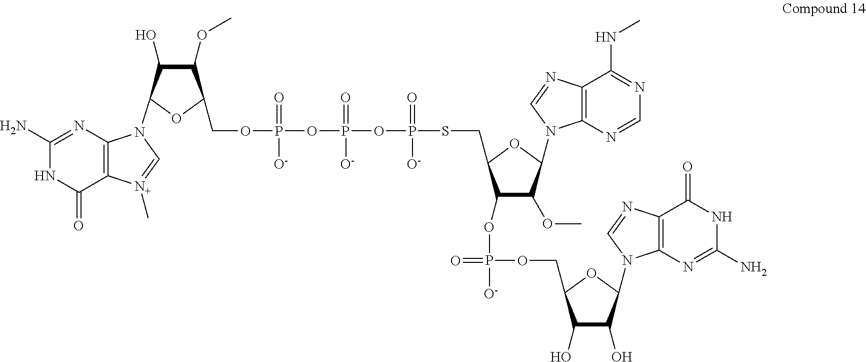
or a stereoisomer thereof, and 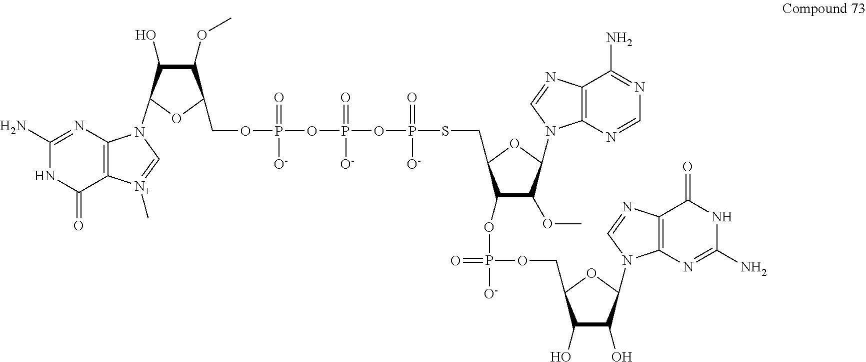
or a stereoisomer thereof.

17. (canceled)

18. (canceled)

19. (canceled)

20. (canceled)

21. The compound of claim 1, wherein the compound is selected from the group consisting of: 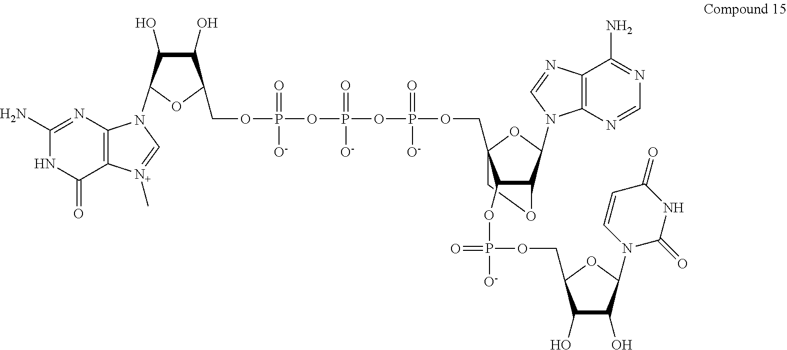
or a stereoisomer thereof, 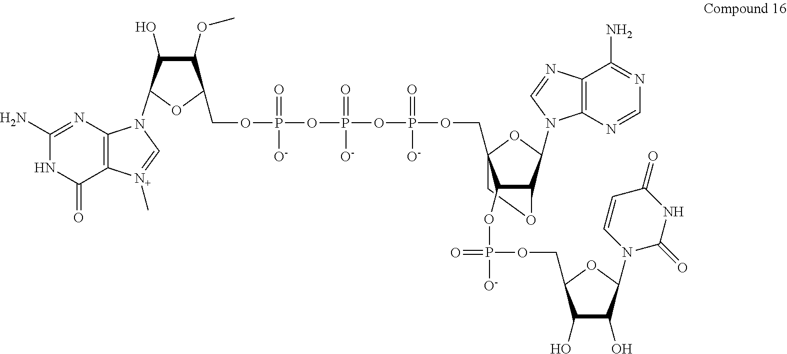
or a stereoisomer thereof, and 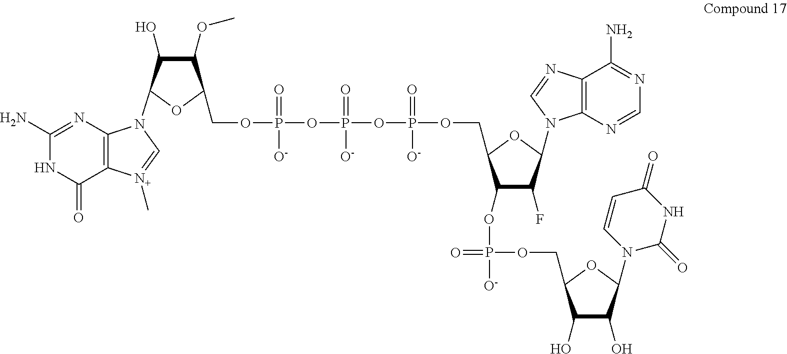
or a stereoisomer thereof.

22. (canceled)

23. (canceled)

24. (canceled)

25. The compound of claim 1, wherein the compound is selected from the group consisting of: 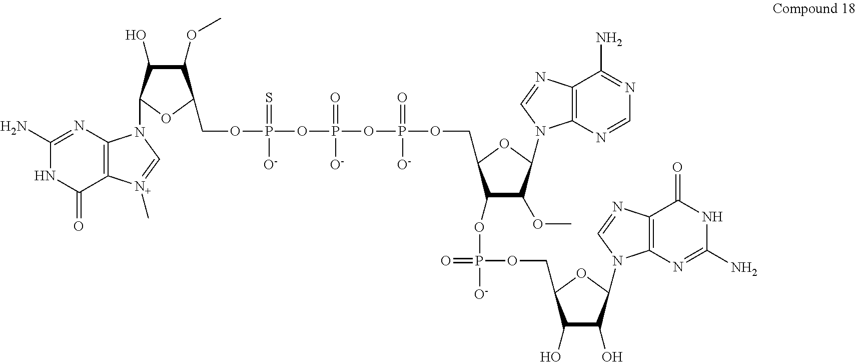
or a stereoisomer thereof, 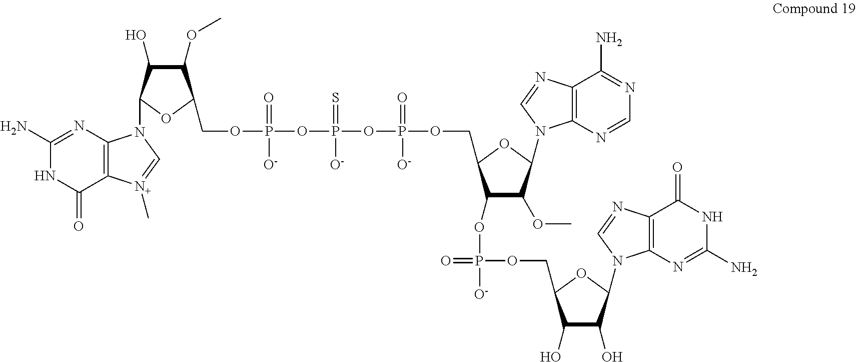
or a stereoisomer thereof, 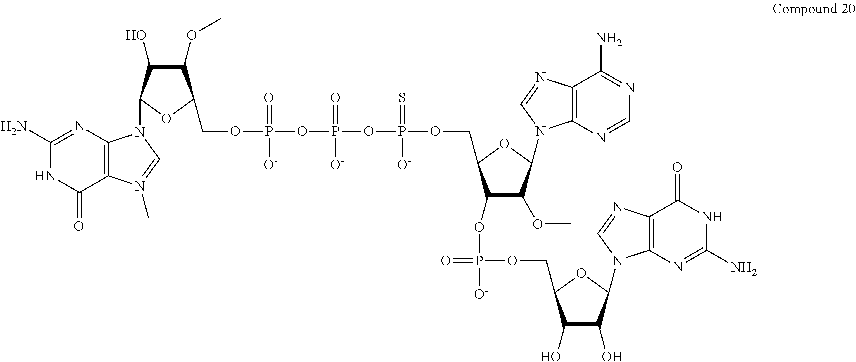
or a stereoisomer thereof, 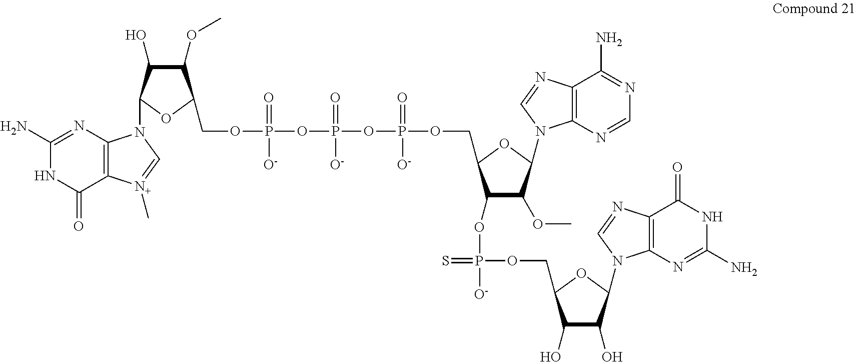
or a stereoisomer thereof, 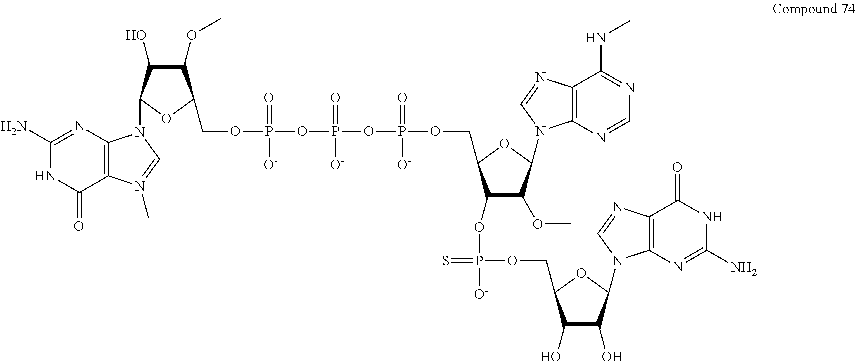
or a stereoisomer thereof, 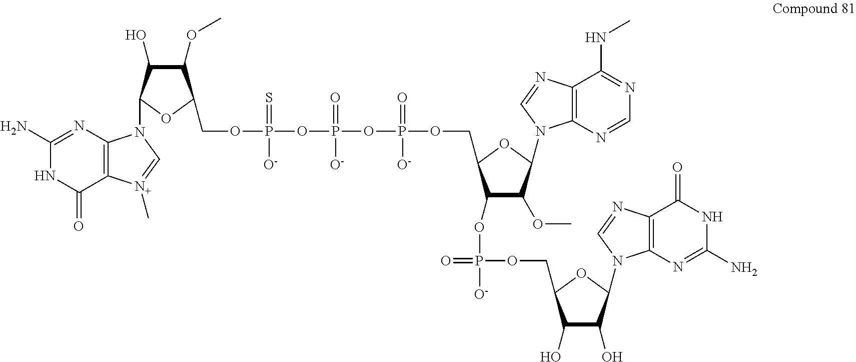
or a stereoisomer thereof.

26. (canceled)

27. (canceled)

28. (canceled)

29. (canceled)

30. (canceled)

31. (canceled)

32. The compound of claim 1, wherein the compound is selected from the group consisting of: 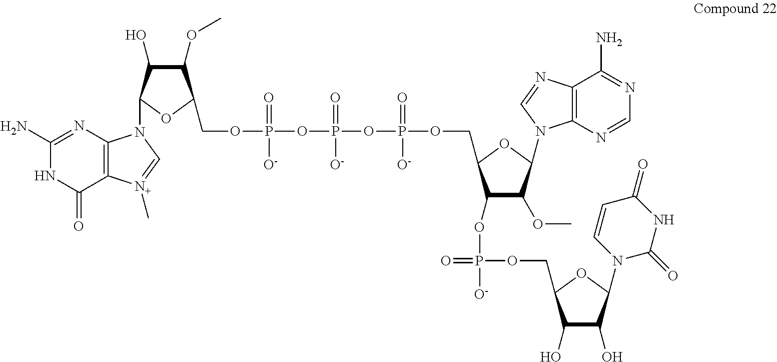
or a stereoisomer thereof, 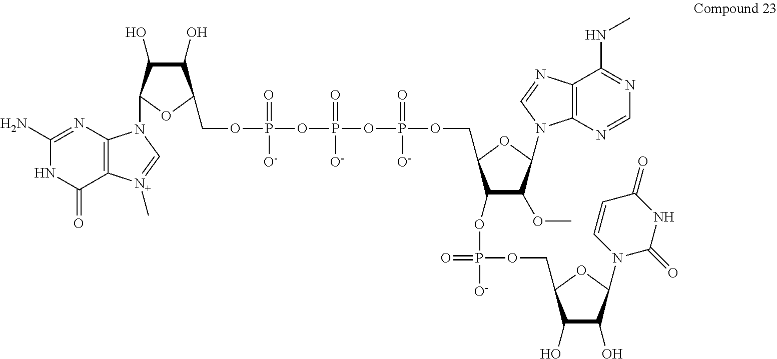
or a stereoisomer thereof, and 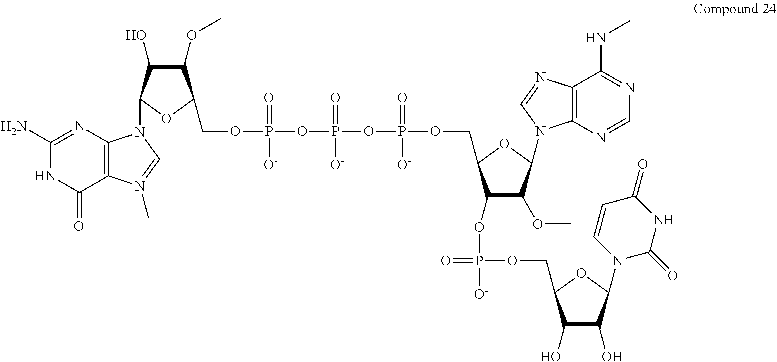
or a stereoisomer thereof.

33. (canceled)

34. (canceled)

35. (canceled)

36. The compound of claim 1, wherein the compound is selected from the group consisting of: 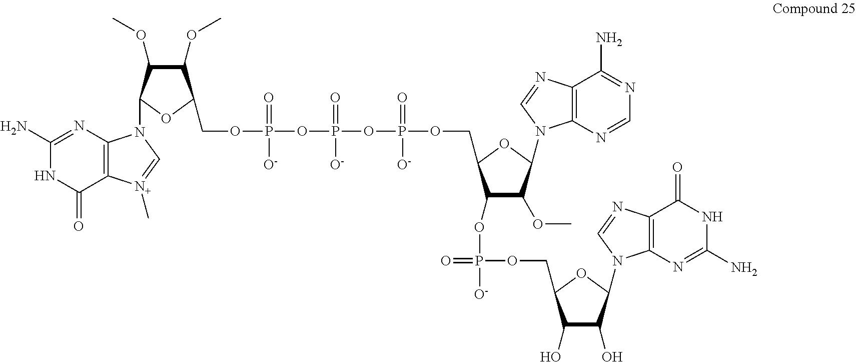
or a stereoisomer thereof, 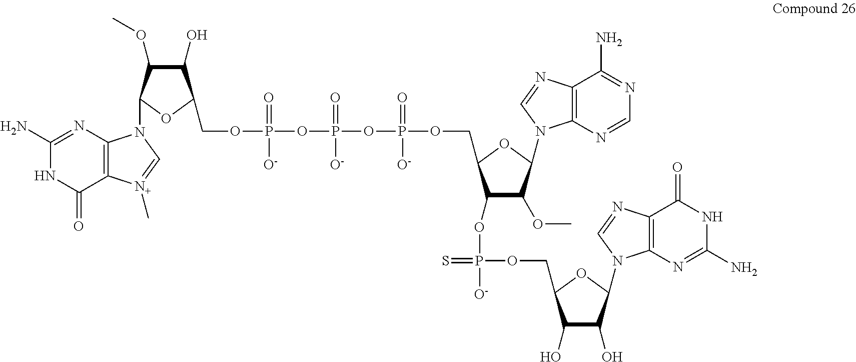
or a stereoisomer thereof, 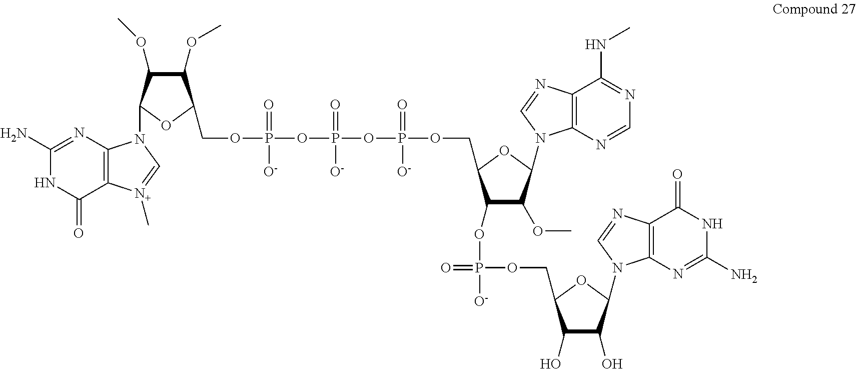
or a stereoisomer thereof, and 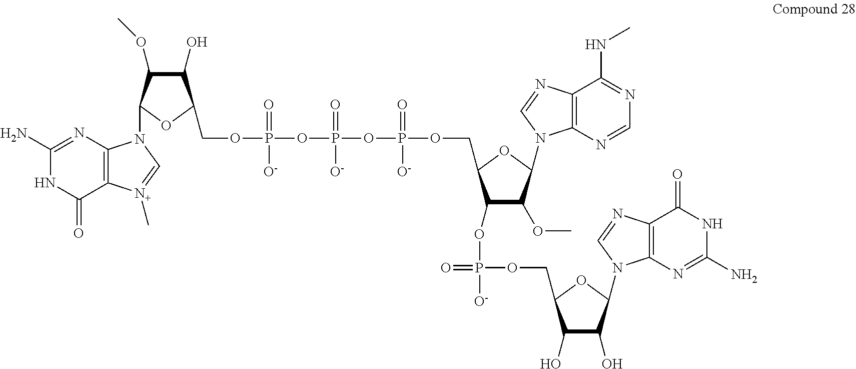
or a stereoisomer thereof.

37. (canceled)

38. (canceled)

39. (canceled)

40. (canceled)

41. The compound of claim 1, wherein the compound is selected from the group consisting of: 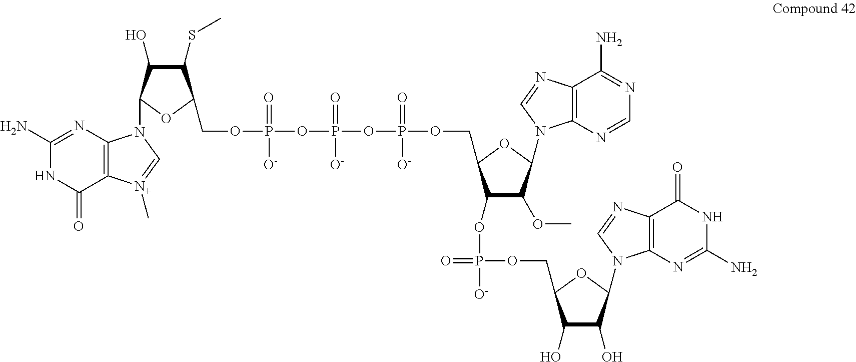
or a stereoisomer thereof, 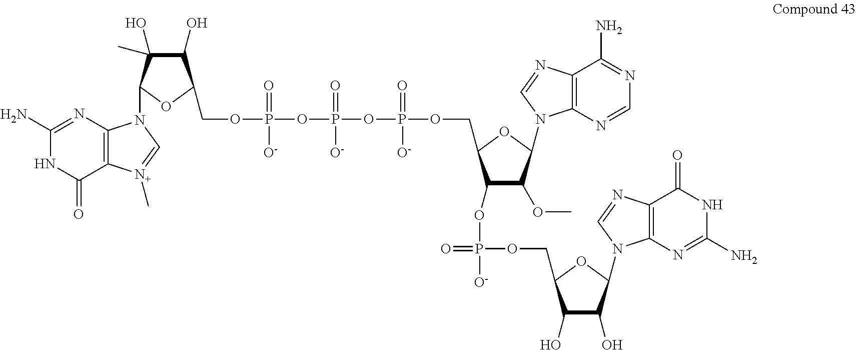
or a stereoisomer thereof. 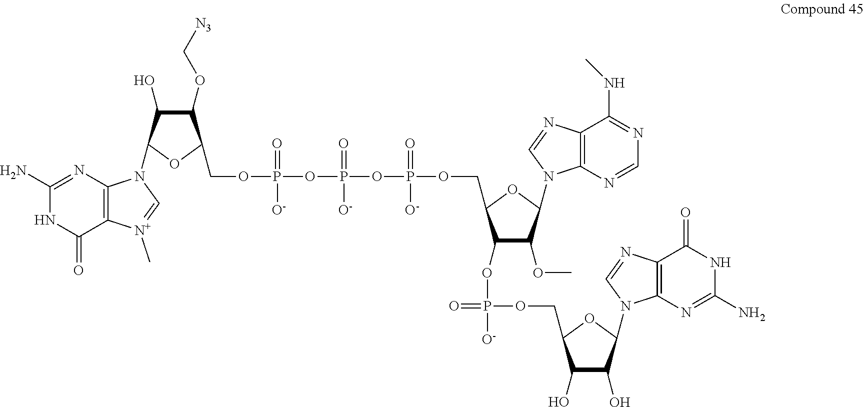
or a stereoisomer thereof, 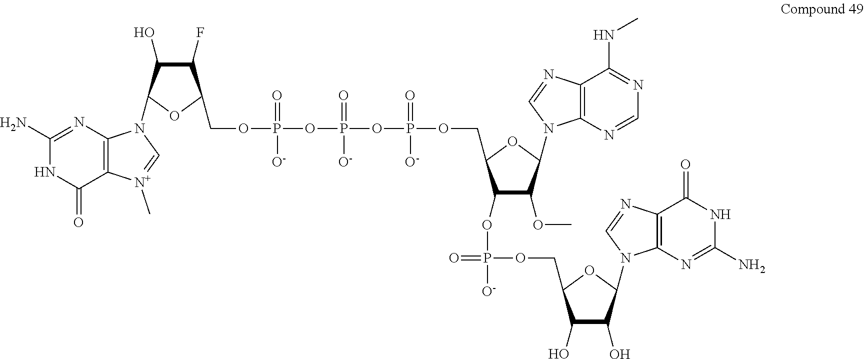
or a stereoisomer thereof, 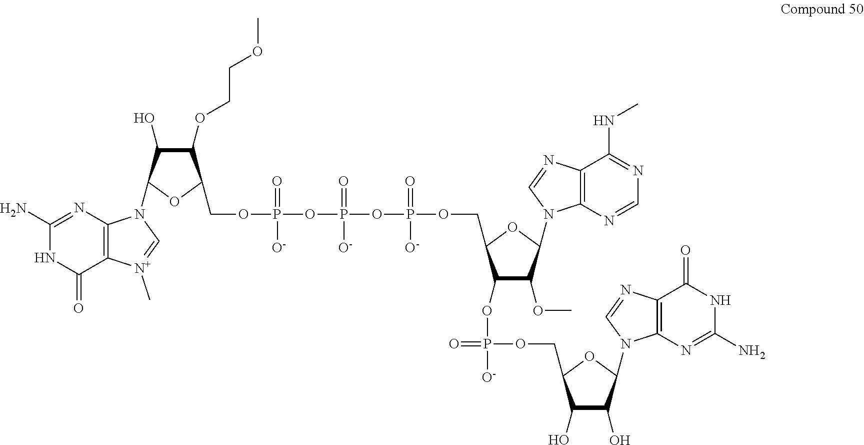
or a stereoisomer thereof, 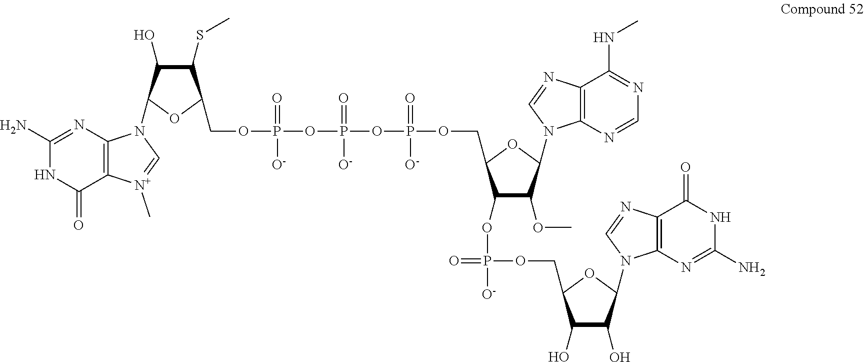
or a stereoisomer thereof, and 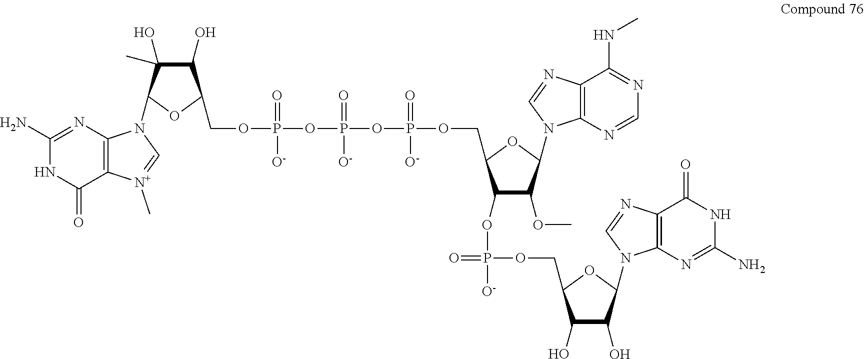
or a stereoisomer thereof.

42. (canceled)

43. (canceled)

44. (canceled)

45. (canceled)

46. (canceled)

47. (canceled)

48. (canceled)

49. The compound of claim 1, wherein the compound has the following structure: 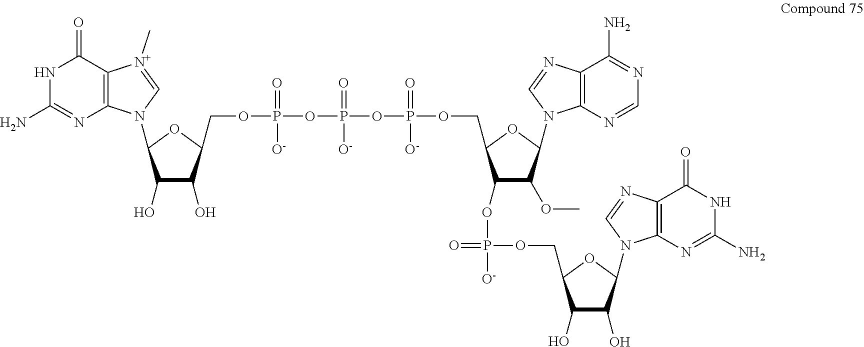
or a stereoisomer thereof, or 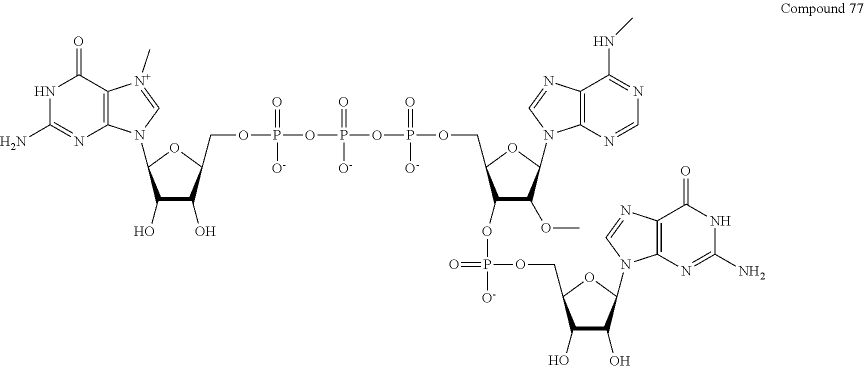
or a stereoisomer thereof.

50. (canceled)

51. (canceled)

52. The compound of claim 1, wherein the compound has the following structure: 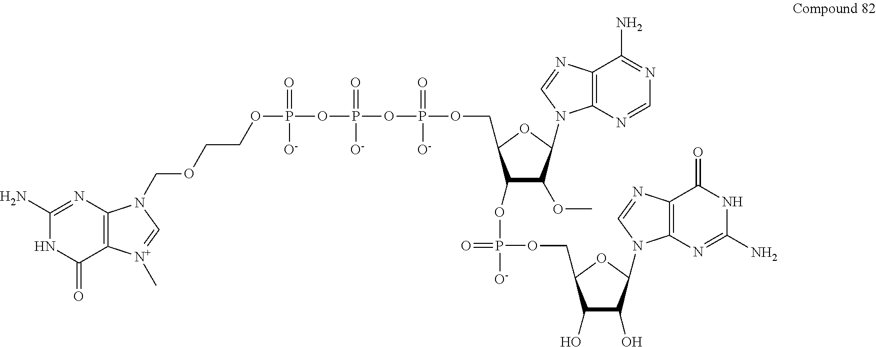
or a stereoisomer thereof or 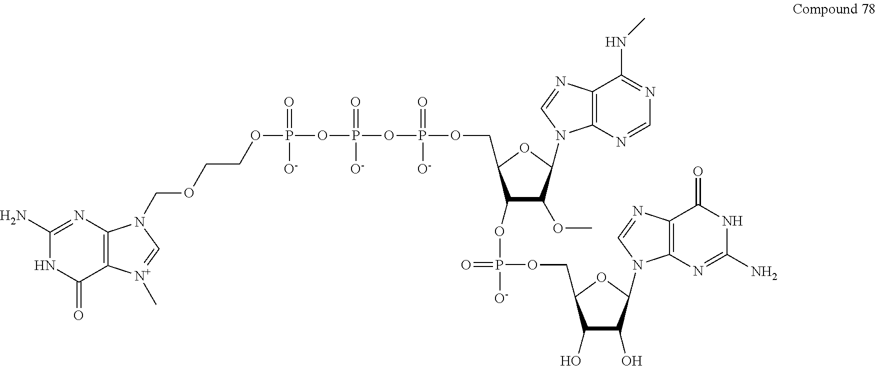
or a stereoisomer thereof.

53. (canceled)

54. (canceled)

55. The compound of claim 1, wherein the compound has the following structure: 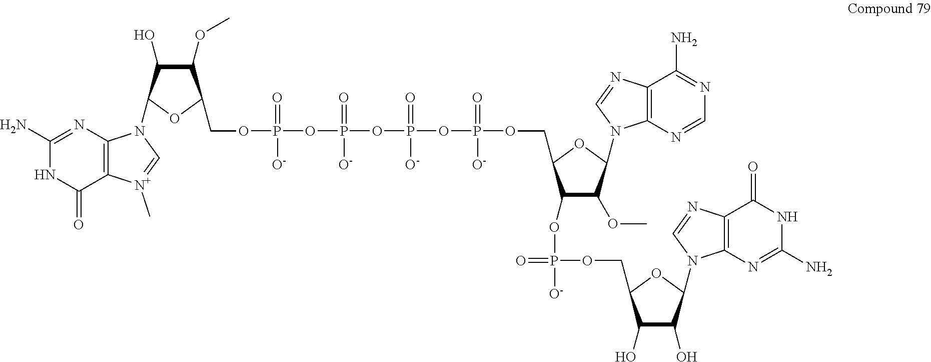
or a stereoisomer thereof, or 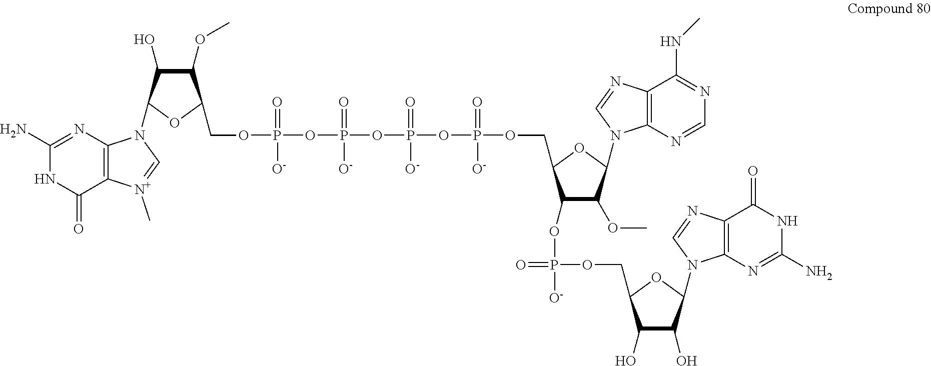
or a stereoisomer thereof.

56. (canceled)

57. The compound of claim 1, wherein the compound is a deuterated form of the compound.

58. (canceled)

59. A pharmaceutical composition, comprising a compound of claim 1 and a pharmaceutically acceptable carrier.

60. An RNA molecule comprising a 5′-cap, wherein the 5′-cap comprises a compound of claim 1.

61. The RNA molecule of claim 60, wherein the RNA molecule is a messenger RNA (mRNA) molecule or a self-amplifying RNA (saRNA) molecule.

62. A method of inducing a therapeutic effect in a subject, comprising administering to the subject an RNA molecule according to claim 60.

63. A method of administering to an animal a therapeutic dose unit of an mRNA molecule comprising a 5′-cap, wherein the 5′-cap comprises a compound of claim 1.

64-69. (canceled)

70. A method of administering to an animal a therapeutic dose unit of an mRNA molecule comprising a 5′-cap, wherein the 5′-cap comprises a compound selected from the group consisting of: 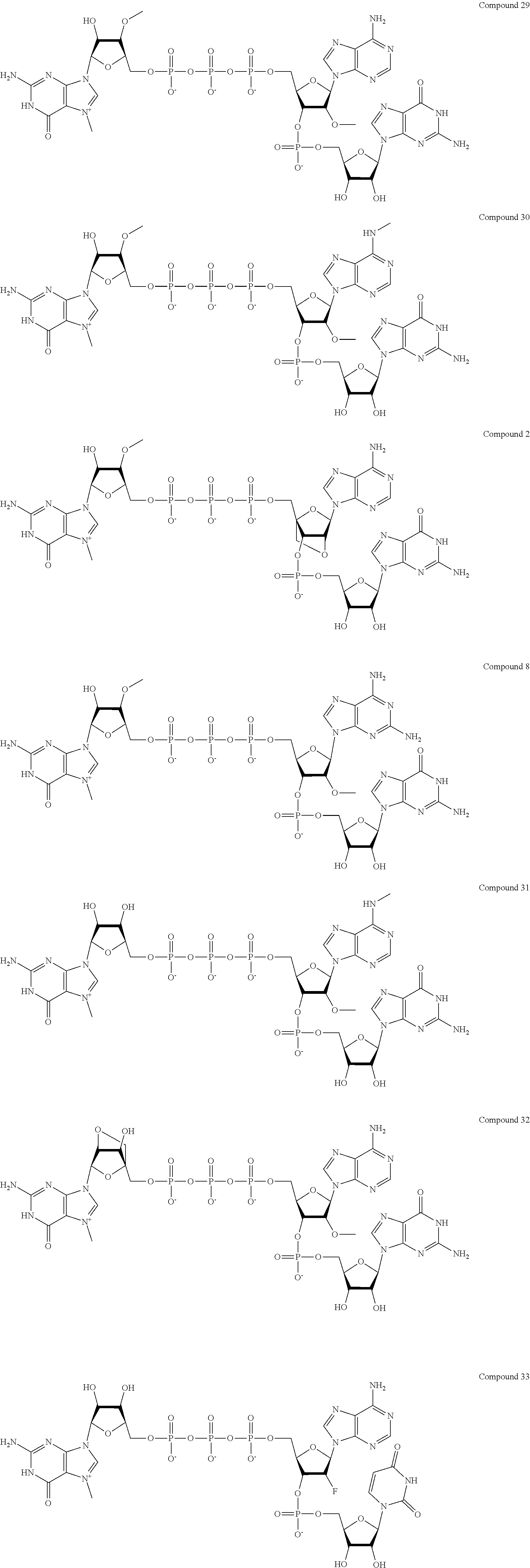
or a stereoisomer thereof,
wherein the therapeutic dose unit of the mRNA molecule comprises less than 7 ng/μg of double stranded RNA (dsRNA),
wherein the subject exhibits increased tolerability to the administered therapeutic dose unit of the mRNA molecule as compared to an equivalent therapeutic dose unit of the mRNA molecule comprising 7 ng/μg or greater dsRNA.

71-74. (canceled)

75. A method for increasing in vivo translation of a polypeptide in a subject, comprising administering to the subject a therapeutic dose unit comprising an effective amount of an mRNA encoding a polypeptide, wherein the mRNA comprises a 5′-cap having the following formula: 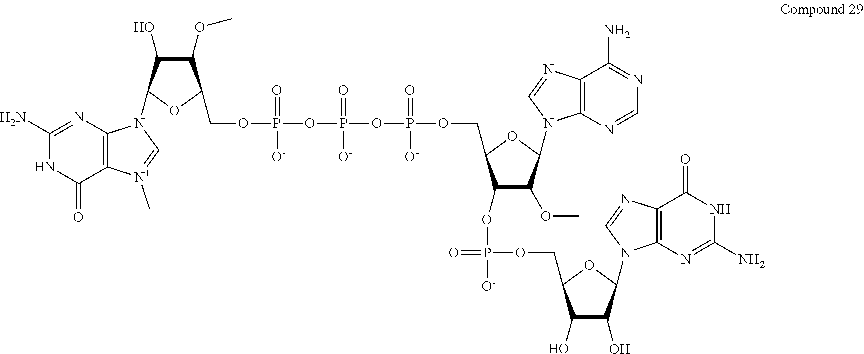
or a stereoisomer thereof,
wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula: 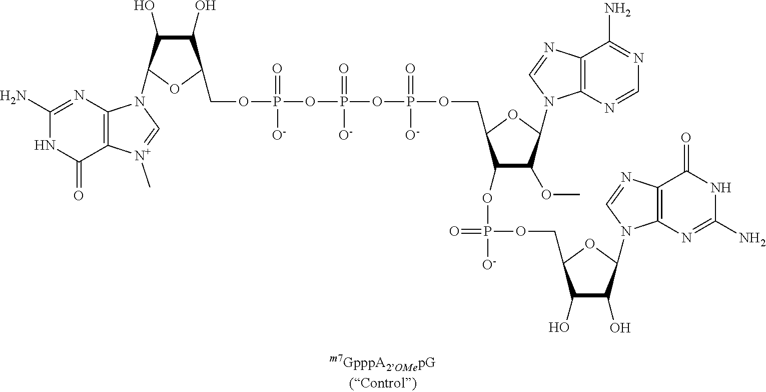
or a stereoisomer thereof; or
a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula: 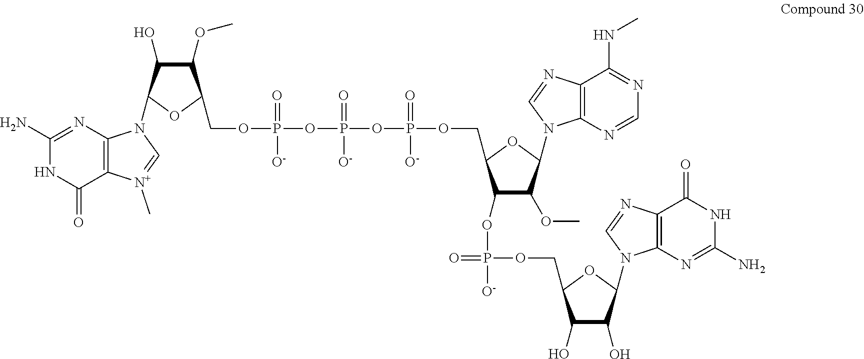
or a stereoisomer thereof,
wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula: 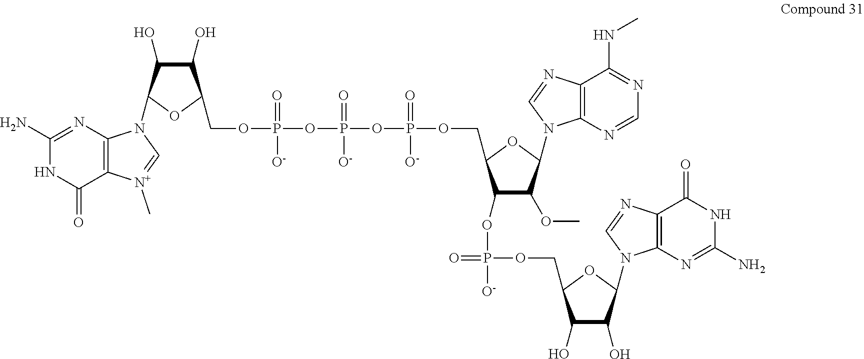
or a stereoisomer thereof; or
a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula: 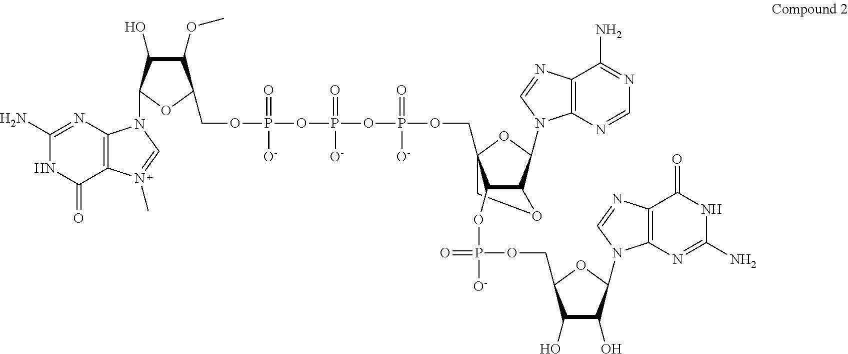
or a stereoisomer thereof,
wherein the administered therapeutic dose is at least 20% lower than the therapeutic dose unit required to elicit the same response in the subject when administered a comparative mRNA encoding the polypeptide, wherein the comparative mRNA comprises a 5′-cap having the following formula: 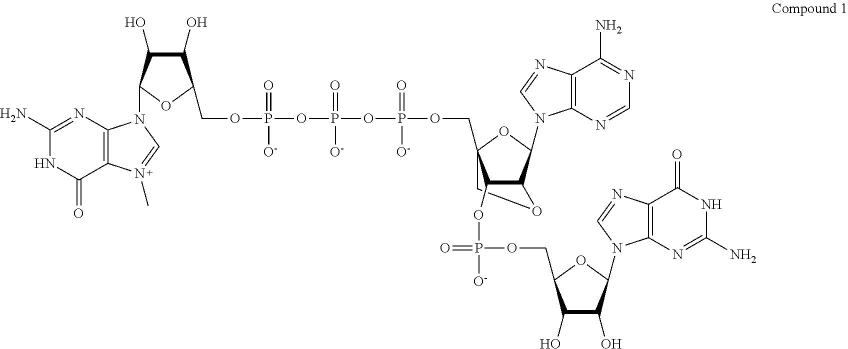
or a stereoisomer thereof; or
a method for increasing in vivo translation of a polypeptide in a subject, comprising administering to the subject a therapeutic dose unit comprising an effective amount of an mRNA encoding a polypeptide, wherein the mRNA comprises a 5′-cap having the following formula: 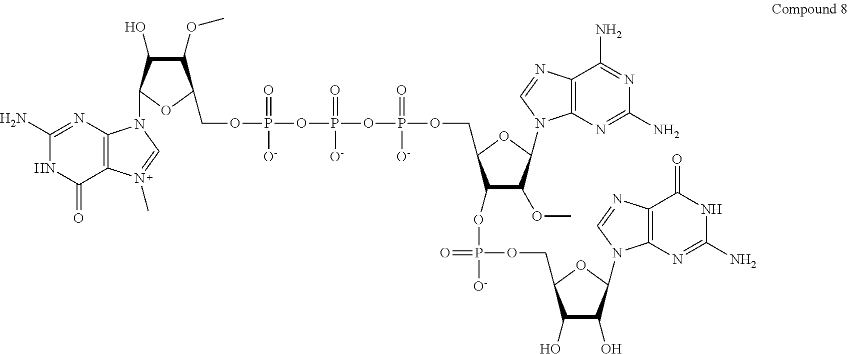
or a stereoisomer thereof,
wherein the administered therapeutic dose unit is at least 20% lower than the therapeutic dose unit required to elicit the same response in the subject when administered a comparative mRNA encoding the polypeptide, wherein the comparative mRNA comprises a 5′-cap having the following formula: 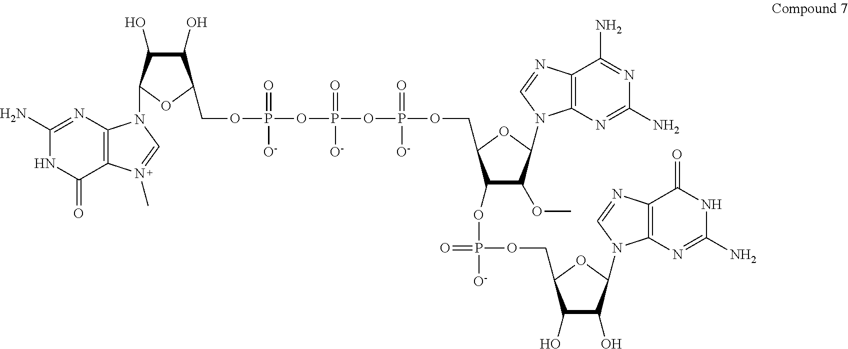
or a stereoisomer thereof.

76-81. (canceled)

82. A method of synthesizing a trinucleotide compound, comprising:
(1) mixing an N₇-methyl-guanosine-5′-diphosphate of the following structure: 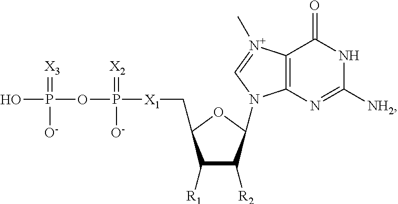
wherein
X₁ and X₂ are each independently selected from the group consisting of O and S;
X₃ is O;
R₁ and R₂ are each independently selected from H, OH, N₃, F, substituted or unsubstituted alkoxy, and thio, wherein R₁ and R₂ optionally combine to form a heterocycle, with an activating reagent and an imidazole to form an activated intermediate; and
(2) adding a salt reagent and a dinucleotide of the following structure: 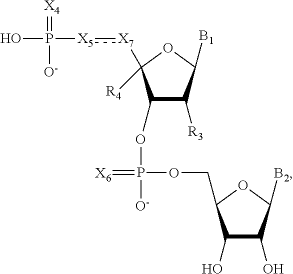
wherein [symbol]
is a single bond or a double bond;
X₄ and X₆ are each independently selected from the group consisting of O and S;
X₅ is O, S, or CH;
X₇ is CH or CH₂;
R₃ is OH, OMe, F and R₄ is H, or wherein R₃ and R₄ are covalently bonded together and, together with intermediate atoms, form a 2′-O, 4′-C methylene bridge; and
B₁ and B₂ are each independently selected from the group consisting of a purine ring and a pyrimidine ring
to the activated intermediate to form the trinucleotide compound of the following structure: 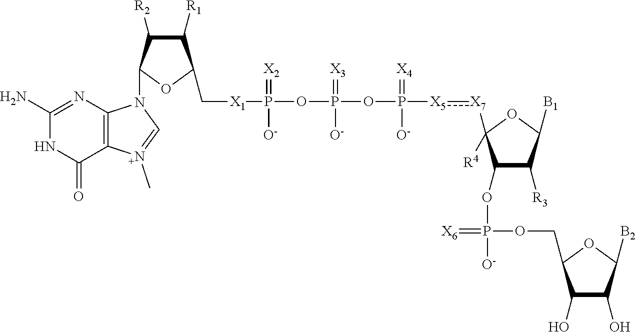
wherein the method is a one-pot synthesis; or
a method of synthesizing a trinucleotide compound, comprising:
(1) mixing a dinucleotide of the following structure: 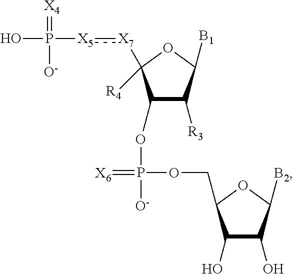
wherein [symbol]
is a single bond or a double bond;
X₄ is O;
X₆ is O or S;
X₅ is O, S, or CH;
X₇ is CH or CH₂;
R₃ is OH, OMe, F and R₄ is H, or wherein R₃ and R₄ are covalently bonded together and, together with intermediate atoms, form a 2′-O, 4′-C methylene bridge; and
B₁ and B₂ are each independently selected from the group consisting of a purine ring and a pyrimidine ring,
with an activating reagent and an imidazole to form an activated phosphate imidazolide of the following structure: 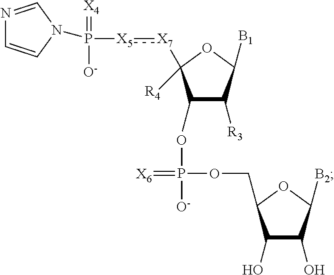
and
(2) adding a salt reagent and a compound of the following structure: 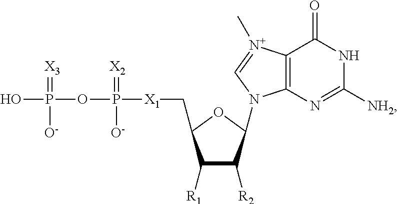
wherein
X₁ and X₂ are each independently selected from the group consisting of O and S;
X₃ is S;
R₁ and R₂ are each independently selected from H, OH, N₃, F, substituted or unsubstituted alkoxy, and thio, wherein R₁ and R₂ optionally combine to form a heterocycle, to the activated phosphate imidazolide to form the trinucleotide compound of the following structure: 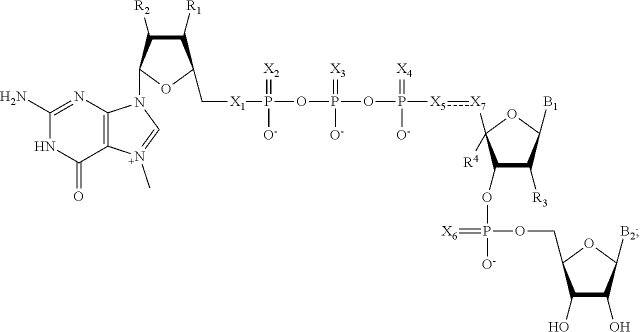
or
a method of synthesizing a trinucleotide compound, comprising:
(1) mixing a diphosphate dinucleotide of the following structure: 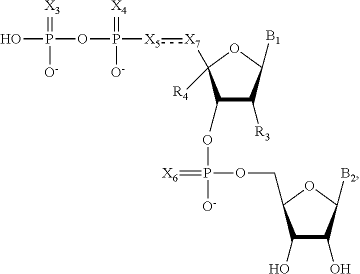
wherein [symbol]
is a single bond or a double bond;
X₃ is O;
X₄ and X₆ are each independently selected from O and S;
X₅ is O, S, or CH: X₇ is CH or CH₂;
R₃ is OH, OMe, F and R₄ is H, or wherein R₃ and R₄ are covalently bonded together and, together with intermediate atoms, form a 2′-O, 4′-C methylene bridge; and
B₁ and B₂ are each independently selected from the group consisting of a purine ring and a pyrimidine ring,
with an activating reagent and an imidazole to form an activated phosphate imidazolide of the following structure: 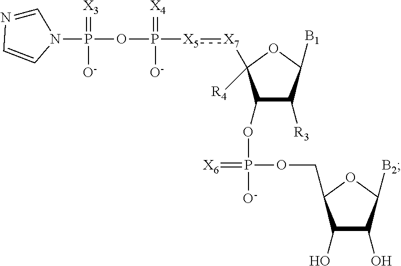
and
(2) adding a salt reagent and a compound of the following structure: 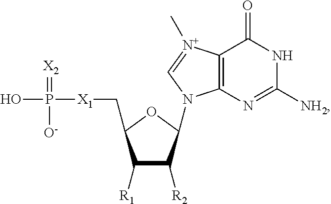
wherein
X₁ and X₂ are each independently selected from O and S; and
R₁ and R₂ are each independently selected from H, OH, N₃, F, substituted or unsubstituted alkoxy, and thio, wherein R₁ and R₂ optionally combine to form a heterocycle, to the activated phosphate imidazolide to form the trinucleotide compound of the following structure: 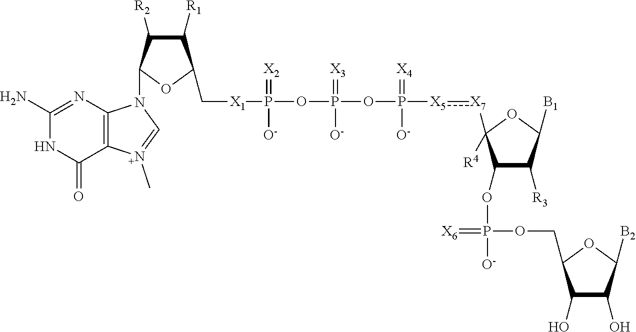
wherein the method is a one-pot synthesis.

83-84. (canceled)
